(19) United States
(12) Patent Application Publication
(10) Pub. No.: US 2004/0186151 A1
(43) Pub. Date: Sep. 23, 2004

(54) Substituted azole derivatives as therapeutic agents

(76) Inventors: Adnan M.M. Mjalli, Jamestown, NC (US); Robert C. Andrews, Jamestown, NC (US); Ravindra R. Yarragunta, High Point, NC (US); Rongyuan Xie, Greensboro, NC (US); Tan Ren, High Point, NC (US); Govindan Subramanian, High Point, NC (US); James C. Quada JR., High Point, NC (US)

Correspondence Address: Samuel B. Rollins, Kilpatrick Stockton LLP, 1001 West Fourth Street, Winston-Salem, NC, 27101, US

(21) Appl. No.: 10/777,471

(22) Filed: Feb. 12, 2004

Related U.S. Application Data
(60) Provisional application No. 60/446,924, filed on Feb. 12, 2003.

(51) Int. Cl. C07D 277/28; C07D 263/02

(52) U.S. Cl. 514/365; 514/374; 514/396; 548/205; 548/215; 548/335.5

(57) ABSTRACT

This invention provides azoles which may be useful as inhibitors of protein tyrosine phosphatases (PTPases). The present invention provides compounds of Formula (I), methods of their preparation, pharmaceutical compositions comprising the compounds and their use in treating human or animal disorders. The compounds of the invention may be useful as inhibitors of protein tyrosine phosphatases and thus can be useful for the management, treatment, control and adjunct treatment of diseases mediated by PTPase activity. Such diseases include Type I diabetes, Type II diabetes.

### STATEMENT OF RELATED APPLICATION

The present application claims priority under 35 USC 119 from U.S. Provisional Application Serial No. 60/446,924, filed Feb. 12, 2003, the disclosure of which is incorporated by reference.

### FIELD OF THE INVENTION

This invention relates to compounds which may be inhibitors of protein tyrosine phosphatases (PTPases), which can be useful for the management, treatment, control, or adjunct treatment of diseases caused by over-activity of PTPases.

### BACKGROUND OF THE INVENTION

The process of protein phosphorylation is now recognized as central to the fundamental processes of cellular signal transduction. Alterations in protein phosphorylation, may therefore constitute either a physiological or pathological change in an in vivo system. Protein de-phosphorylation, mediated by phosphatases, is also central to certain signal transduction processes.

The two major classes of phosphatases are (a) protein serine/threonine phosphatases (PSTPases), which catalyze the dephosphorylation of serine and/or threonine residues on proteins or peptides; and (b) the protein tyrosine phosphatases (PTPases), which catalyze the dephosphorylation of tyrosine residues on proteins and/or peptides. A third class of phosphatases is the dual specificity phosphatases, or DSP's, which possess the ability to act both as PTPases and as PSTPases.

Among the PTPases there exist two important families, the intracellular PTPases, and the transmembrane PTPases. The intracellular PTPases include PTP1B, STEP, PTPD1, PTPD2, PTPMEG1, T-cell PTPase, PTPH1, FAP-1/BAS, PTP1D, and PTP1C. The transmembrane PTPases include LAR, CD45, PTPα, PTPP, PTPβ, PTPε, PTPξ, PTPκ, PTPμ, PTPσ, HePTP, SAP-1, and PTP-U2. The dual—specificity phosphatases include KAP, cdc25, MAPK phosphatase, PAC-1, and rVH6.

The PTPases, especially PTP1B, are implicated in insulin insensitivity characteristic of type II diabetes (Kennedy, B. P.; Ramachandran, C. Biochem. Pharm. 2000, 60, 877-883). The PTPases, notably CD45 and HePTP, are also implicated in immune system function, and in particular T-cell function. Certain PTPases, notably TC-PTP, DEP-1, SAP-1, and CDC25, are also implicated in certain cancers. Certain PTPases, notably the bone PTPase OST-PTP, are implicated in osteoporosis. PTPases are implicated in mediating the actions of somatostatin on target cells, in particular the secretion of hormone and/or growth factor secretion.

Thus, there is a need for agents which inhibit the action of protein tyrosine phosphatases. Such agents would be useful for the treatment of Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### SUMMARY OF THE INVENTION

This invention provides azoles which may be useful as inhibitors of PTPases. In an embodiment, the present invention provides compounds of Formula (I) as depicted below, methods of their preparation, pharmaceutical compositions comprising the compounds, and their use in treating human or animal disorders. The compounds of the invention may be useful as inhibitors of protein tyrosine phosphatases and thus can be useful for the management, treatment, control and adjunct treatment of diseases mediated by PTPase activity. Such diseases include Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### DETAILED DESCRIPTION OF THE INVENTION

In a first aspect, the present invention provides azole inhibitors of protein tyrosine phosphatases (PTPases) which can be useful for the management and treatment of disease caused by PTPases.

In a another aspect, the present invention provides compounds of Formula (I):

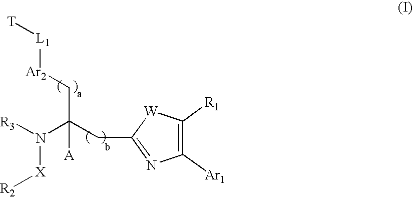

wherein a and b are equal to 0 and 1; wherein the values of 0 and 1 represent a direct bond and —CH₂—, respectively, and wherein the —CH₂— group is optionally substituted 1 to 2 times with a substituent group, wherein said substituent group(s) comprise: -alkyl, -aryl, -alkylene-aryl, -arylene-alkyl, -alkylene-arylene-alkyl, —O-alkyl, —O-aryl, or -hydroxyl. In another embodiment, a is equal to 0 and b is equal to 1. In another embodiment, a is equal to 1 and b is equal to 0.

W comprises —O—, —S—, or —N(R₄)—;

wherein

R₄ comprises

a) -hydrogen;

b) -alkyl;

c) -L₂-D-G;

d) -L₂-D-alkyl;

e) -L₂-D-aryl;

f) -L₂-D-heteroaryl;

g) -L₂-D-cycloalkyl;

h) -L₂-D-heterocyclyl;

i) -L₂-D-arylene-alkyl;

j) -L₂-D-alkylene-cycloalkyl;

k) -L₂-D-alkylene-heterocyclyl;

l) -L₂-D-alkylene-aryl;

m) -L₂-D-alkylene-heteroaryl;

n) -L₂-D-alkylene-arylene-alkyl;

o) -L₂-D-alkylene-heteroarylene-alkyl;

p) -L₂-D-alkyl-G;

q) -L₂-D-aryl-G;

r) -L₂-D-heteroaryl-G;

s) -L₂-D-cycloalkyl-G;

t) -L₂-D-heterocyclyl-G;

u) -L₂-D-arylene-alkyl-G;

v) -L₂-D-alkylene-cycloalkyl-G;

w) -L₂-D-alkylene-heterocyclyl-G;

x) -L₂-D-alkylene-aryl-G;

y) -L₂-D-alkylene-heteroaryl-G;

z) -L₂-D-alkylene-arylene-alkyl-G; or

aa) -L₂-D-alkylene-heteroarylene-alkyl-G;

wherein

L₂ comprises a direct bond, -alkylene, -alkenylene, or -alkynylene;

D comprises a direct bond, —CH₂—, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, —N═N—, or —N(R₅)—N(R₆)—,

wherein R₅ and R₆ independently comprise: -hydrogen, -alkyl, -aryl, -arylene-alkyl, -alkylene-aryl, or -alkylene-arylene-alkyl;

G comprises —H, -alkyl, —CN, —SO₃H, —P(O)(OH)₂, —P(O)(O-alkyl)(OH), —CO₂H, —CO₂-alkyl, an acid isostere, —NR₇R₈,

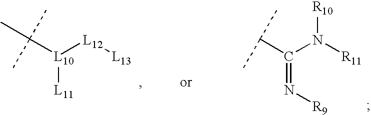

wherein

L₁₀ comprises alkyline, cycloalkyline, heteroaryline, aryline, or heterocyclyline;

L₁₂ comprises —O—, —C(O)—N(R₄₀)—, —C(O)—O—, —C(O)—, or —N(R₄₀)—CO—N(R₄₁)—;

L₁₃ comprises hydrogen, alkyl, alkenyl, alkynyl, heterocyclyl, heteroaryl, or -alkylene-aryl;

L₁₁ comprises hydrogen, alkyl, alkenyl, alkynyl, -alkylene-aryl, -alkylene-heteroaryl, alkylene-O-alkylene-aryl, -alkylene-S-alkylene-aryl, -alkylene-O-alkyl, -alkylene-S-alkyl, -alkylene-NH₂, -alkylene-OH, -alkylene-SH, -alkylene-C(O)—OR₄₂, -alkylene-C(O)—NR₄₂R₄₃, -alkylene-NR₄₂R₄₃, -alkylene-N(R₄₂)—C(O)—R₄₃, -alkylene-N(R₄₂)—S(O₂)—R₄₃, or the side chain of a natural or non-natural amino acid;

R₄₂ and R₄₃ independently comprise hydrogen, aryl, alkyl, or alkylene-aryl;

wherein

R₄₂ and R₄₃ may be taken together to form a ring having the formula —(CH₂)_{q}—Y—(CH₂)ᵣ— bonded to the nitrogen atom to which R₁₁ and R₁₂ are attached, wherein q and r are, independently, 1, 2, 3, or 4; Y is —CH₂—, —C(O)—, —O—, —N(H)—, —S—, —S(O)—, —SO₂—, —CON(H)—, —NHC(O)—, —NHCON(H)—, —NHSO₂—, —SO₂N(H)—, —(O)CO—, —NHSO₂NH—, —OC(O)—, —N(R₄₄)—, —N(C(O)R₄₄)—, —N(C(O)NHR₄₄)—, —N(SO₂NHR₄₄)—, —N(SO₂R₄₄)—, or —N(C(O)OR₄₄)—; or

R₄₂ and R₄₃ may be taken together, with the nitrogen atom to which they are attached, to form a heterocyclyl or heteroaryl ring.

R₄₀, R₄₁, and R₄₄ independently comprise: hydrogen, aryl, alkyl, or alkylene-aryl.

and wherein

R₇ and R₈ independently comprise hydrogen, -alkyl, -L₃-E-alkyl, -L₃-E-aryl, —C(O)-alkyl, —C(O)-aryl, —SO₂-alkyl, —SO₂-aryl, or

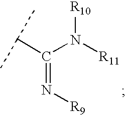

wherein

R₉, R₁₀, and R₁₁ independently comprise: -hydrogen, -alkyl, -aryl, -arylene-alkyl, -alkylene-aryl, or -alkylene-arylene-alkyl;

L₃ comprises a direct bond, -alkylene, -alkenylene, or -alkynylene;

E comprises a direct bond, —CH₂—, —O—, —N(R₁₂)—, —C(O)—, —CON(R,₂)—, —N(R₁₂)C(O)—, —N(R₁₂)CON(R₁₃)—, —N(R₁₂)C(O)O—, —OC(O)N(R₁₂)—, —N(R₁₂)SO₂—, —SO₂N(R₁₂)—, —C(O)—O—, —O—C(O)—, —S—, —S(O)—, —S(O₂)—, —N(R₁₂)SO₂N(R₁₃)—, —N═N—, or —N(R₁₂)—N(R₁₃)—,

wherein

R₁₂ and R₁₃ independently comprise: -hydrogen, -alkyl, -aryl, -arylene-alkyl, -alkylene-aryl, or -alkylene-arylene-alkyl.

In another embodiment, W comprises —N(R₄)—, wherein R₄ comprises -alkyl, -L₂-D-alkyl, or -L₂-D-aryl, wherein L₂ comprises alkylene, and D comprises a direct bond, —C(O)— or —O—. In another embodiment, W comprises —N(R₄)—, wherein R₄ is hydrogen. In another embodiment, W comprises —N(R₄)—, wherein R₄ comprises -L₂-D-G, wherein L₂ comprises alkenyl or alkynyl, wherein D comprises a direct bond and G comprises hydrogen or alkyl.

A comprises hydrogen, -alkyl, -alkenyl, or -alkynyl. In an embodiment, A comprises hydrogen.

X comprises

a) —C(O)—;

b) —CH₂—;

wherein the —CH₂— group is optionally substituted 1 to 2 times with a substituent group, wherein said substituent group(s) are selected from the group consisting of: -alkyl, -aryl, -alkylene-aryl, -arylene-alkyl, -alkylene-arylene-alkyl, —O-alkyl, —O-aryl, and -hydroxyl.

c) a direct bond; or

d) —SO₂—.

In an embodiment, X comprises —C(O)— or CH₂.

R₁ comprises

a) -hydrogen;

b) -fluoro

c) -chloro

d) -bromo

e) -iodo

f) -cyano

g) -alkyl;

h) -aryl;

i) -alkylene-aryl;

j) -heteroaryl;

k) -alkylkene-heteroaryl;

l) -cycloalkyl;

m) -alkylene-cycloalkyl

n) -heterocyclyl; or

o) -alkylene-heterocyclyl;

In an embodiment, R₁ comprises hydrogen or aryl. In another embodiment, R₁ comprises hydrogen.

R₂ comprises

a) -perfluoroalkyl;

b) -J-R₁₄;

c) -alkyl;

d) -aryl;

e) -heteroaryl;

f -heterocyclyl;

g) -cycloalkyl;

h) -L₄-aryl;

i) -L₄-arylene-aryl;

j) -L₄-arylene-alkyl;

k) -arylene-alkyl;

l) -arylene-arylene-alkyl;

m) -J-alkyl;

n) -J-aryl;

o) -J-alkylene-aryl;

p) -J-arylene-alkyl;

q) -J-alkylene-arylene-aryl;

r) -J-arylene-arylene-aryl;

s) -J-alkylene-arylene-alkyl;

t) -L₄-J-alkylene-aryl;

u) -arylene-J-alkyl;

v) -L₄-J-aryl;

w) -L₄-J-heteroaryl;

x) -L₄-J-cycloalkyl;

y) -L₄-J-cycloalkylene-alkyl;

z) -L₄-J-heterocyclyl;

aa) -L₄-J-arylene-alkyl;

bb) -L₄-J-alkylene-arylene-alkyl;

cc) -L₄-J-alkyl;

dd) -L₄-J-R₁₄;

ee) -L₄-J-alkylene-R₁₄;

ff) -J-L₄-R₁₄;

gg) -arylene-J-R₁₄;

hh) -L₄-arylene-J-alkyl;

ii) -L₄-alkylene-J-alkyl;

jj) -L₄-arylene-J-aryl; or

kk) -hydrogen;

wherein

L₄ comprises a direct bond, -alkylene, -alkenylene, -alkynylene. heterocyclylene, cycloalkylene, arylene, or heteroarylene;

J comprises a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—,

wherein

R₁₅ and R₁₆ independently comprise: -hydrogen, -alkyl, -aryl, -arylene-alkyl, -alkylene-aryl, or -alkylene-arylene-alkyl.

R₁₄ comprises: -hydrogen, -alkyl, -aryl, -arylene-alkyl, -alkylene-aryl, -alkylene-arylene-alkyl, or

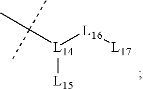

wherein

L₁₄ comprises alkyline, cycloalkyline, heteroaryline, aryline, or heterocyclyline;

L₁₆ comprises —O—, —C(O)—N(R₄₅)—, —C(O)—O—, —C(O)—, or —N(R₄₅)—CO—N(R₄₆)—;

L₁₇ comprises hydrogen, alkyl, alkenyl, alkynyl, heterocyclyl, heteroaryl, or -alkylene-aryl;

L₁₅ comprises hydrogen, alkyl, alkenyl, alkynyl, -alkylene-aryl, -alkylene-heteroaryl, alkylene-O-alkylene-aryl, -alkylene-S-alkylene-aryl, -alkylene-O-alkyl, -alkylene-S-alkyl, -alkylene-NH₂, -alkylene-OH, -alkylene-SH, -alkylene-C(O)—OR₄₇, -alkylene-C(O)—NR₄₇R₄₈, -alkylene-NR₄₇R₄₈, -alkylene-N(R₄₇)—C(O)—R₄₈,

-alkylene-N(R₄₇)—S(O₂)—R₄₈, or the side chain of a natural or non-natural amino acid;

R₄₇ and R₄₈ independently comprise hydrogen, aryl, alkyl, or alkylene-aryl;

R₄₇ and R₄₈ may be taken together, with the nitrogen atom to which they are attached, to form a heterocyclyl or heteroaryl ring.

R₄₅ and R₄₆ independently comprise hydrogen, aryl, alkyl, or alkylene-aryl.

In another embodiment, R₂ comprises: -alkyl, -aryl, -L₄-J-cycloalkyl, arylene-alkyl, -L₄-arylene-J-alkyl, or -J-alkyl, wherein L₄ comprises alkylene or alkenylene, and J comprises a direct bond or —O—.

In another embodiment,

R₃ comprises —H;

X comprises —C(O)—;

R₂ comprises -L₄-arylene-J-alkyl;

-L₄-J-cycloalkylene-alkyl; or

-L₄-J-alkylene-aryl;

wherein

L₄ comprises alkylene, alkenylene, or a direct bond; and

J comprises a direct bond, —O—, or —NH—.

R₃ comprises

a) -hydrogen

b) -alkyl

c) -aryl;

d) -alkylene-cycloalkyl;

e) -arylene-alkyl;

f) -alkylene-aryl; or

g) -alkylene-heteroaryl;

In another embodiment, R₃ is hydrogen.

Ar₁ comprises an aryl, heteroaryl, fused cycloalkylaryl, fused cycloalkylheteroaryl, fused heterocyclylaryl, or fused heterocyclylheteroaryl group optionally substituted 1 to 7 times. In an embodiment, Ar₁ comprises a mono- or bicyclic aryl group optionally substituted 1 to 7 times. In another embodiment, Ar₁ comprises a phenyl or naphthyl group optionally having 1 to 5 substituents, wherein the substituents independently comprise:

a) -fluoro;

b) -chloro;

c) -bromo;

d) -iodo;

e) -cyano;

f) -nitro;

g) -perfluoroalkyl;

h) —K—R₁₇;

i) -alkyl;

j) -aryl;

k) -heteroaryl;

l) -heterocyclyl;

m) -cycloalkyl;

n) -L₅-aryl;

o) -L₅-arylene-aryl;

p) -L₅-arylene-alkyl;

q) -arylene-alkyl;

r) -arylene-arylene-alkyl;

s) —K-alkyl;

t) —K-aryl;

u) —K-alkylene-aryl;

v) —K-arylene-alkyl;

w) —K-alkylene-arylene-aryl;

x) —K-arylene-arylene-aryl;

y) —K-alkylene-arylene-alkyl;

z) -L₅-K-alkylene-aryl;

aa) -arylene-K-alkyl;

bb) -L₅-K-aryl;

cc) -L₅-K-heteroaryl;

dd) -L₅-K-cycloalkyl;

ee) -L₅-K-heterocyclyl;

ff) -L₅-K-arylene-alkyl;

gg) -L₅-K-alkylene-arylene-alkyl;

hh) -L₅-K-alkyl;

ii) -L₅-K-R₁₇;

jj) -arylene-K-R₁₇; or

kk) -hydrogen;

wherein

L₅ comprises a direct bond, -alkylene, -alkenylene, or -alkynylene;

K comprises a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₈)SO₂N(R₁₉)—, —N═N—, or —N(R₁₈)—N(R₁₉)—,

wherein

R₁₇, R₁₈, and R₁₉ independently comprise: -hydrogen, -alkyl, -aryl, -arylene-alkyl, -alkylene-aryl, or -alkylene-arylene-alkyl.

In other another embodiment, Ar₁ comprises a phenyl group substituted 1 to 5 times with substituents independently comprising:

a) -fluoro;

b) -chloro;

c) -bromo;

d) -iodo; or

e) -nitro.

Ar₂ comprises an arylene, heteroarylene, fused arylcycloalkylene, fused cycloalkylarylene, fused cycloalkylheteroarylene, fused heterocyclylarylene, or fused heterocyclylheteroarylene group optionally substituted 1 to 7 times. In an embodiment,

Ar₂ comprises an arylene group optionally substituted 1 to 7 times. In another embodiment, Ar₂ comprises a phenylene or naphthylene group optionally having 1 to 5 substituents, wherein the substituents independently comprise:

a) -fluoro;

b) -chloro;

c) -bromo;

d) -iodo;

e) -cyano;

f) -nitro;

g) -perfluoroalkyl;

h) -Q-R₂₀;

i) -alkyl;

j) -aryl;

k) -heteroaryl;

l) -heterocyclyl;

m) -cycloalkyl;

n) -L₆-aryl;

o) -L₆-arylene-aryl;

p) -L₆-arylene-alkyl;

q) -arylene-alkyl;

r) -arylene-arylene-alkyl;

s) -Q-alkyl;

t) -Q-aryl;

u) -Q-alkylene-aryl;

v) -Q-arylene-alkyl;

w) -Q-alkylene-arylene-aryl;

x) -Q-arylene-arylene-aryl;

y) -Q-alkylene-arylene-alkyl;

z) -L₆-Q-alkylene-aryl;

aa) -arylene-Q-alkyl;

bb) -L₆-Q-aryl;

cc) -L₆-Q-heteroaryl;

dd) -L₆-Q-cycloalkyl;

ee) -L₆-Q-heterocyclyl;

ff) -L₆-Q-arylene-alkyl;

gg) -L₆-Q-alkylene-arylene-alkyl;

hh) -L₆-Q-alkyl;

ii) -L₆-Q-alkylene-aryl-R₂₀;

jj) -L₆-Q-alkylene-heteroaryl-R₂₀;

kk) -arylene-Q-alkylene-R₂₀;

ll) -heteroarylene-Q-alkylene-R₂₀;

mm) -L₆-Q-aryl-R₂₀;

nn) -L₆-Q-heteroarylene-R₂₀;

oo) -L₆-Q-heteroaryl-R₂₀;

pp) -L₆-Q-cycloalkyl-R₂₀;

qq) -L₆-Q-heterocyclyl-R₂₀;

rr) -L₆-Q-arylene-alkyl-R₂₀;

ss) -L₆-Q-heteroarylene-alkyl-R₂₀;

tt) -L₆-Q-alkylene-arylene-alkyl-R₂₀;

uu) -L₆-Q-alkylene-heteroarylene-alkyl-R₂₀;

vv) -L₆-Q-alkylene-cycloalkylene-alkyl-R₂₀;

ww) -L₆-Q-alkylene-heterocyclylene-alkyl-R₂₀;

xx) -L₆-Q-alkyl-R₂₀;

yy) -L₆-Q-R₂₀;

zz) -arylene-Q-R₂₀;

aaa) -heteroarylene-Q-R₂₀;

bbb) -heterocyclylene-Q-R₁₈;

ccc) -Q-alkylene-R₂₀;

ddd) -Q-arylene-R₂₀;

eee) -Q-heteroarylene-R₂₀;

fff) -Q-alkylene-arylene-R₂₀;

ggg) -Q-alkylene-heteroarylene-R₂₀;

hhh) -Q-heteroarylene-alkylene-R₂₀;

iii) -Q-arylene-alkylene-R₂₀;

jjj) -Q-cycloalkylene-alkylene-R₂₀;

kkk) -Q-heterocyclylene-alkylene-R₂₀;

lll) -Q-alkylene-arylene-alkyl-R₂₀;

mmm) -Q-alkylene-heteroarylene-alkyl-R₂₀;

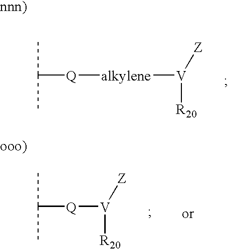

ppp) -hydrogen,

wherein

L₆ comprises a direct bond, -alkylene, -alkenylene, or -alkynylene;

Q comprises a direct bond, —CH₂—, —O—, —N(R₂₁)—, —C(O)—, —CON(R₂₁)—, —N(R₂₁)C(O)—, —N(R₂₁)CON(R₂₂)—, —N(R₂₁)C(O)O—, —OC(O)N(R₂₁)—, —N(R₂₁)SO₂—, —SO₂N(R₂₁)—, —C(O)—O—, —O—C(O)—, —S—, —S(O)—, —S(O₂)—, —N(R₂₁)SO₂N(R₂₂)—, N═N—, or —N(R₂₁)—N(R₂₂)—;

wherein

R₂, and R₂₂ independently comprising: -hydrogen, -alkyl, -aryl, -arylene-alkyl, -alkylene-aryl, or -alkylene-arylene-alkyl;

V comprises

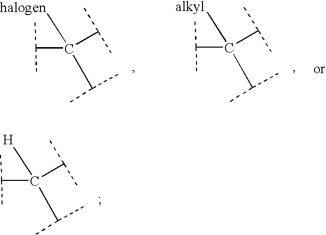

Z comprises hydrogen, —CO₂H, -alkylene-aryl, -alkyl, -aryl, -heteroaryl, -heterocyclyl, -cycloalkyl, -alkylene-heteroaryl, or -alkylene-cycloalkyl;

R₂₀ comprises —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, or -alkylene-arylene-alkyl.

In another embodiment, Ar₂ comprises a phenyl or naphthyl group optionally substituted 1 to 5 times, wherein the substituents independently comprise:

a) -fluoro;

b) -chloro;

c) -bromo;

d) -iodo;

e) -Q-R₂₀;

f) -alkyl;

g) -aryl;

h) -arylene-alkyl;

i) -Q-alkyl; or

j) -arylene-Q-alkyl;

wherein

Q comprises: —CH₂—, —O—, —C(O)—, or —C(O)—O—; and

R₂₀ comprises: -hydrogen, -alkyl, -aryl, cycloalkyl, -alkenyl, —CO₂H, or an acid isostere.

In another embodiment, Ar₂ comprises a phenyl group substituted 1 to 5 times, wherein the substituents independently comprise:

a) -fluoro;

b) -chloro;

c) -bromo;

d) -iodo;

e) -Q-R₂₀;

f -alkyl;

g) -phenyl;

h) -phenylene-alkyl;

i) -Q-alkyl; or

j) -phenylene-Q-alkyl;

wherein

Q comprises: —CH₂—, —O—, —C(O)—, or —C(O)—O—; and

R₂₀ comprises: -hydrogen, -alkyl, -phenyl, -cycloalkyl, alkenyl, or —CO₂H.

In another embodiment, Ar₂ comprises a phenyl group substituted 1 to 5 times, wherein the substituents independently comprise:

a) -Q-alkyl;

b) -Q-arylene-R₂₀;

c) -Q-alkylene-arylene-R₂₀; or

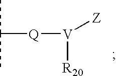

wherein

Q comprises: —CH₂—, —O—, —C(O)—, or —C(O)—O—;

Z comprises —CO₂H and an acid isostere;

V comprises

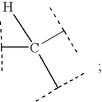

and

R₂₀ comprises: —CO₂H or an acid isostere.

L₁ comprises a direct bond, —CH₂—, —O—, alkylene, alkenylene, —O-alkylene-, -alkylene-O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;

wherein

R₂₃ and R₂₄ independently comprise: -hydrogen, -alkyl, -aryl, -arylene-alkyl, alkylene-aryl, -alkylene-arylene-alkyl, or a direct bond.

In an embodiment, L₁ comprises —O—, —O-alkylene-, -alkylene-O—, or a direct bond. In another embodiment, L₁ comprises —O-alkylene- or a direct bond.

T comprises hydrogen, alkyl, cycloalkyl, heterocyclyl, aryl, heteroaryl, fused cycloalkylaryl, fused cycloalkylheteroaryl, fused heterocyclylaryl, or fused heterocyclylheteroaryl group optionally substituted 1 to 7 times, wherein the substituents independently comprise:

a) -fluoro;

b) -chloro;

c) -bromo;

d) -iodo;

e) -cyano;

f) -nitro;

g) -perfluoroalkyl;

h) —U—R₂₅;

i) -alkyl;

j) -aryl;

k) -heteroaryl;

I) -heterocyclyl;

m) -cycloalkyl;

n) -L₇-aryl;

o) -L₇-arylene-aryl;

p) -L₇-arylene-alkyl;

q) -arylene-alkyl;

r) -arylene-arylene-alkyl;

s) —U-alkyl;

t) —U-aryl;

u) —U-alkylene-aryl;

v) —U-arylene-alkyl;

w) —U-alkylene-arylene-aryl;

x) —U-arylene-arylene-aryl;

y) —U-alkylene-arylene-alkyl;

z) -L₇-U-alkylene-aryl;

aa) -arylene-U-alkyl;

bb) -L₇-U-aryl;

cc) -L₇-U-heteroaryl;

dd) -L₇-U-cycloalkyl;

ee) -L₇-U-heterocyclyl;

ff) -L₇-U-arylene-alkyl;

gg) -L₇-U-alkylene-arylene-alkyl;

hh) -L₇-U-alkyl;

ii) -L₇-U-alkylene-aryl-R₂₅;

jj) -L₇-U-alkylene-heteroaryl-R₂₅;

kk) -arylene-U-alkylene-R₂₅;

ll) -heteroarylene-U-alkylene-R₂₅;

mm) -L₇-U-aryl-R₂₅;

nn) -L₇-U-heteroarylene-R₂₅;

oo) -L₇-U-heteroaryl-R₂₅;

pp) -L₇-U-cycloalkyl-R₂₅;

qq) -L₇-U-heterocyclyl-R₂₅;

rr) -L₇-U-arylene-alkyl-R₂₅;

ss) -L₇-U-heteroarylene-alkyl-R₂₅;

tt) -L₇-U-alkylene-arylene-alkyl-R₂₅;

uu) -L₇-U-alkylene-heteroarylene-alkyl-R₂₅;

vv) -L₇-U-alkylene-cycloalkylene-alkyl-R₂₅;

ww) -L₇-U-alkylene-heterocyclylene-alkyl-R₂₅;

xx) -L₇-U-alkyl-R₂₅;

yy) -L₇-U—R₂₅;

zz) -arylene-U—R₂₅;

aaa) -heteroarylene-U—R₂₅;

bbb) -heterocyclylene-U—R₂₅;

ccc) —U-alkylene-R₂₅;

ddd) —U-arylene-R₂₅;

eee) —U-heteroarylene-R₂₅;

fff) —U-alkylene-arylene-R₂₅;

ggg) —U-alkylene-heteroarylene-R₂₅;

hhh) —U-heteroarylene-alkylene-R₂₅;

iii) —U-arylene-alkylene-R₂₅;

jjj) —U-cycloalkylene-alkylene-R₂₅;

kkk) —U-heterocyclylene-alkylene-R₂₅;

lll) —U-alkylene-arylene-alkyl-R₂₅;

mmm) —U-alkylene-heteroarylene-alkyl-R₂₅;

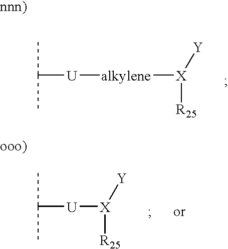

ppp) -hydrogen;

wherein

L₇ comprises a direct bond, -alkylene, -alkenylene, or -alkynylene;

U comprises a direct bond, —CH₂—, —O—, —N(R₂₆)—, —C(O)—, —CON(R₂₆)—, —N(R₂₆)C(O)—, —N(R₂₆)CON(R₂₇)—, —N(R₂₆)C(O)O—, —OC(O)N(R₂₆)—, —N(R₂₆)SO₂—, —SO₂N(R₂₆)—, —C(O)—O—, —O—C(O)—, —S—, —S(O)—, —S(O₂)—, —N(R₂₆)SO₂N(R₂₇)—, N═N—, or —N(R₂₆)—N(R₂₇)—;

wherein

R₂₆ and R₂₇ independently comprise: -hydrogen, -alkyl, -aryl, -arylene-alkyl, -alkylene-aryl, or -alkylene-arylene-alkyl;

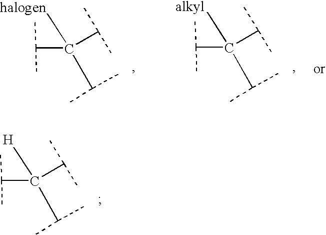

Y comprises hydrogen, —CO₂H, -alkylene-aryl, -alkyl, -aryl, -heteroaryl, -heterocyclyl, -cycloalkyl, -alkylene-heteroaryl, or -alkylene-cycloalkyl;

R₂₅ comprises —SO₃H, —P(O)(OH)₂, —P(b)(O-alkyl)(OH), —CO₂H, —CO₂-alkyl, an acid isostere, -hydrogen, -alkyl, -aryl, -arylene-alkyl, -alkylene-aryl, or -alkylene-arylene-alkyl.

In an embodiment, T comprises an alkyl, -alkylene-aryl, or aryl group optionally substituted 1 to 7 times.

In another embodiment, T comprises an aryl group substituted by —U-alkylene-R₂₅, wherein U comprises —O— or a direct bond and R₂₅ comprises —CO₂H or an acid isostere.

In another embodiment, X and R₂ together form a group comprising: tert-butoxycarbonyl, tert-butyl-methyl-carbonyl, 4-cyclohexyl-butyryl, 3-cyclohexyl-propionyl, 2-cyclohexyl-acetyl,4-tert-butyl-phenyl)-carbonyl, 4-(4′-methoxyphenyl)-butyryl, 4-(4′-methoxyphenyl)-butyryl, 3-(4′-methoxyphenyl)-propionyl, 3-(3′-methoxyphenyl)-propionyl, 3-(4′-methoxy-phenyl)-acryl, 3-(4′-chloro-phenyl)-acryl, 2-(4′-methoxy-phenyl)-acetyl, 2-(4′-chloro-phenyl)-acetyl, 2-(4′-methylsulfonyl-phenyl)-acetyl, 2-(4′-methylsulfonyl-phenyl)-acetyl, 4-(4′-chloro-2′-methyl-phenoxy)-butyryl, 4-(4′-methoxyphenyl)-butyryl, or 4-(4′-cyclohexyl)-propyl.

In another embodiment, a equals 0, and the groups T, L₁, and Ar₂ form a group comprising: 4′-n-butoxy-3′-n-butoxy carbonyl phenyl, or 4′-n-butoxy-3′-carboxyl phenyl.

In another embodiment, Ar₁ comprises phenyl, naphthyl, 4-nitrophenyl, 4-chlorophenyl, 3-chlorophenyl, 3,4-dichlorophenyl, 2,4-dichlorophenyl, 2,5-dichlorophenyl, 2,6-dichlorophenyl, 4-cyanophenyl, 4-bromophenyl, or pentafluorophenyl.

In a another aspect, the present invention provides a pharmaceutically acceptable salt, solvate, or prodrug of compounds of Formula (I).

In the compounds of Formula (I), the various functional groups represented should be understood to have a point of attachment at the functional group having the hyphen. In other words, in the case of -alkylene-aryl, it should be understood that the point of attachment is the alkylene group; an example would be benzyl. In the case of a group such as —C(O)—NH— alkylene-aryl, the point of attachment is the carbonyl carbon.

Also included within the scope of the invention are the individual enantiomers of the compounds represented by Formula (I) above as well as any wholly or partially racemic mixtures thereof. The present invention also covers the individual enantiomers of the compounds represented by formula above as mixtures with diastereoisomers thereof in which one or more stereocenters are inverted.

Compounds of the present invention which are currently preferred for their biological activity are listed by name below in Table 1.

The ability of compounds Formula (I) to potentially treat or inhibit disorders related to insulin resistance or hyperglycemia was established with representative compounds of Formula (I) listed in Table 1 using a standard assay test procedure that measures the inhibition of PTP-1B activity.

The compounds of this invention are potentially useful in treating metabolic disorders related to insulin resistance or hyperglycemia, typically associated with obesity or glucose intolerance. The compounds of this invention may therefore be particularly useful in the treatment or inhibition of type II diabetes. The compounds of this invention are also potentially useful in modulating glucose levels in disorders such as type I diabetes.

TABLE 1

| Ex. | Structure | Name |
|---|---|---|
| 1 | 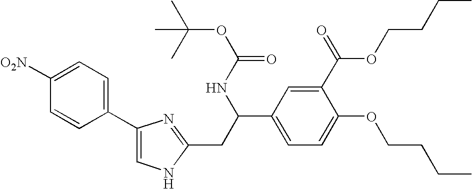 | 2-[(Tert-butoxycarbonyl) amino]-2-(4′-n-Butoxy-3′-n- butoxy carbonyl phenyl)-2- ethyl[4-(4′-nitro phenyl)]imidazole |
| 2 | 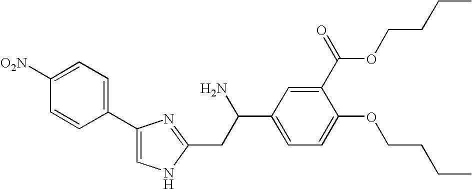 | 5-{1-Amino-2-[4-(4-nitro- phenyl)-1H-imidazol-2-yl]- ethyl}-2-butoxy-benzoic acid butyl ester hydrochloride |
| 3 | 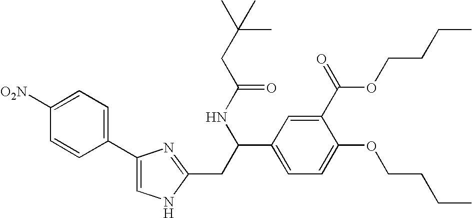 | 2-[(Tert-butyl-methyl- carbonyl)amino]-2-(4′-n- butoxy-3′-n-butoxycarbonyl phenyl)-2-ethyl[4-(4′- nitrophenyl)]imidazole |
| 4 | 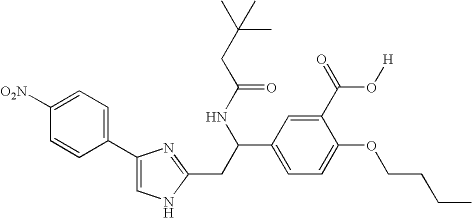 | 2-[(Tert-butyl-methyl- carbonyl)amino]-2-(4′-n- butoxy-3′-carboxyl phenyl)- 2-ethyl[4-(4′-nitrophenyl)]imidazole |
| 5 | 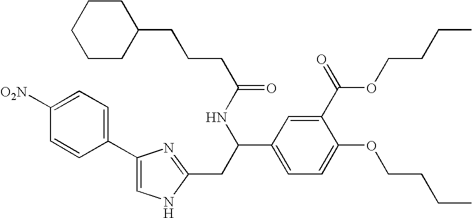 | 2-[(4-Cyclohexyl- butyryl)amino]-2-(4′-n- butoxy-3′-n-butoxycarbonyl phenyl)-2-ethyl[4-(4′- nitrophenyl)]imidazole |
| 6 | 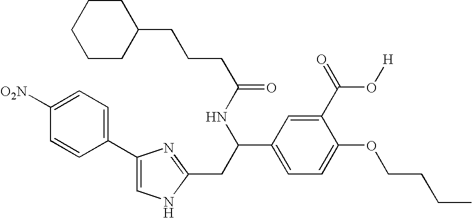 | 2-[(4-Cyclohexyl-butyryl)- amino]-2-(4′-n-butoxy-3′- carboxyphenyl)-2-ethyl[4-(4′- nitrophenyl)]imidazole |
| 7 | 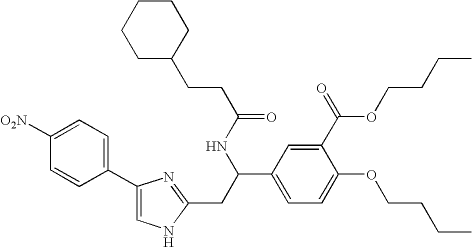 | 2-[(3-Cyclohexyl-propionyl) amino]-2-(4′-n-butoxy-3′-n- butoxycarbonyl phenyl)-2- ethyl[4-(4′-nitrophenyl)]imidazole |
| 8 | 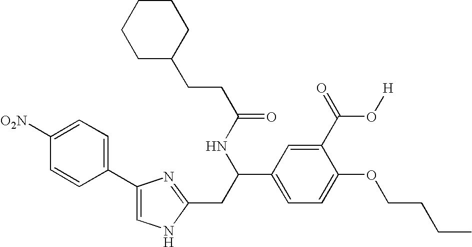 | 2-[(3-Cyclohexyl-propionyl) amino]-2-(4′-n-butoxy-3′- carboxy phenyl)-2-ethyl[4- (4′-nitrophenyl)]imidazole |
| 9 | 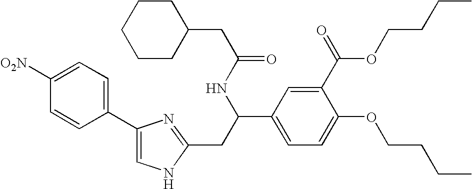 | 2-[(2-Cyclohexyl-acetyl) amino]-2-(4′-n-butoxy-3′-n- butoxycarbonyl phenyl)-2- ethyl[4-(4′-nitrophenyl)]imidazole |
| 10 | 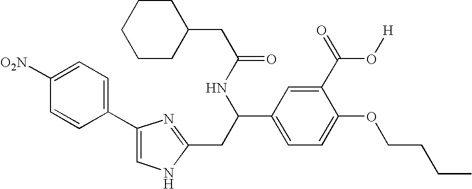 | 2-[(2-Cyclohexyl-acetyl)- amino]-2-(4′-n-butoxy-3′- carboxy phenyl)-2-ethyl[4- (4′-nitrophenyl)]imidazole |
| 11 | 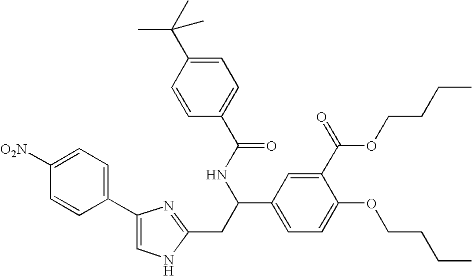 | 2-[(4-tert-butyl-phenyl)- carbonylamino]-2-(4′-n- butoxy-3′-n- butoxycarbonylphenyl)-2- ethyl[4-(4′-nitrophenyl)]imidazole |
| 12 | 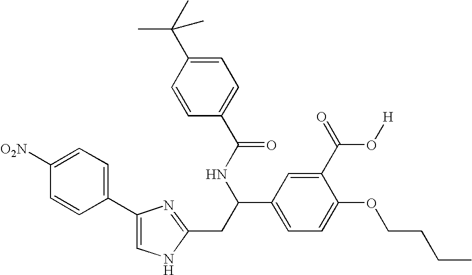 | 2-Butoxy-5-{1-(4-tert-butyl- benzoylamino)-2-[4-(4-nitro- phenyl)-1H-imidazol-2-yl]- ethyl}-benzoic acid |
| 13 | 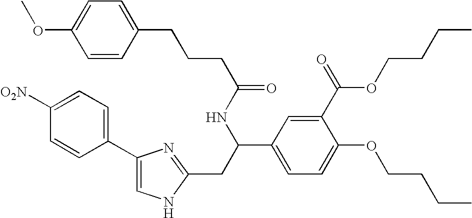 | 2-[4-(4′-Methoxyphenyl)- butyrylamino]-2-(4′-n- butoxy-3′-n- butoxycarbonylphenyl)-2- ethyl[4-(4′-nitrophenyl)]imidazole |
| 14 | 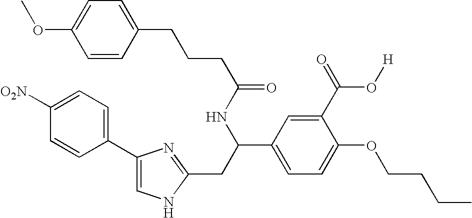 | 2-[4-(4′-Methoxyphenyl)- butyryl amino]-2-(4′-n- butoxy-3′-carboxyphenyl)-2- ethyl[4-(4′-nitrophenyl)]imidazole |
| 15 | 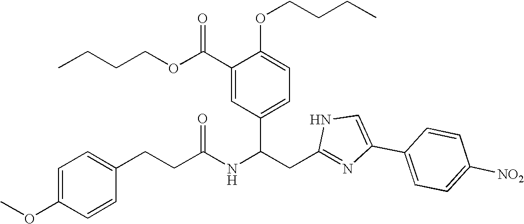 | 2-[3-(4′-Methoxyphenyl)- propionylamino]-2-(4′-n- butoxy-3′-n- butoxycarbonylphenyl)-2- ethyl[4-(4′-nitrophenyl)]imidazole |
| 16 | 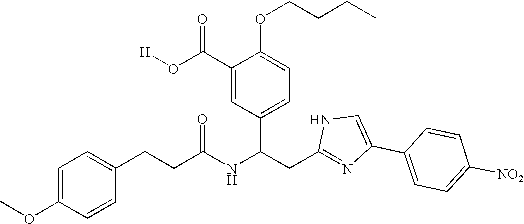 | 2-[3-(4′-Methoxyphenyl)- propionylamino]-2-(4′-n- butoxy-3′-carboxyphenyl)-2- ethyl[4-(4′- nitrophenyl)]imidazole |
| 17 | 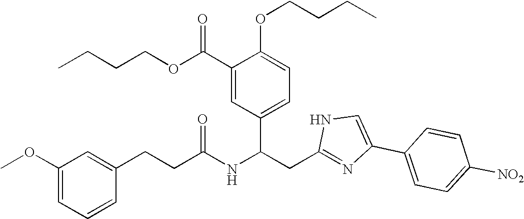 | 2-[3-(3′-Methoxyphenyl)- propionylamino]-2-(4′-n- butoxy-3′-n- butoxycarbonylphenyl)-2- ethyl[4-(4′-nitrophenyl)]imidazole |
| 18 | 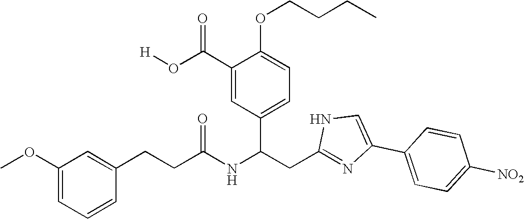 | 2-[3-(3′-Methoxyphenyl)- propionylamino]-2-(4′-n- butoxy-3′-carboxyphenyl)-2- ethyl[4-(4′-nitrophenyl)]imidazole |
| 19 | 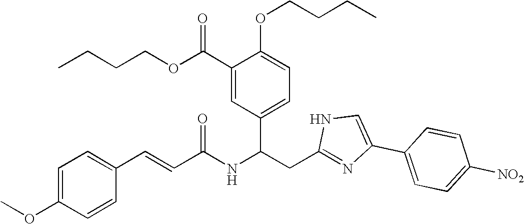 | 2-[3-(4′-Methoxy-phenyl)- acryl amino]-2-(4′-n-butoxy- 3′-n-butoxycarbonyl phenyl)-2-ethyl[4-(4′- nitrophenyl)]imidazole |
| 20 | 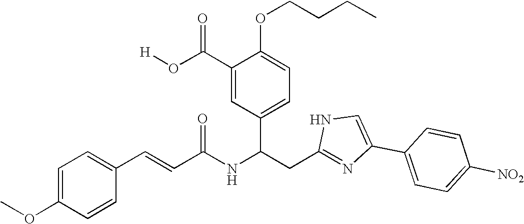 | 2-[3-(4′-Methoxy-phenyl)- acrylamino]-2-(4′-n-butoxy- 3′-carboxyphenyl)-2-ethyl[4- (4′-nitrophenyl)]imidazole |
| 21 | 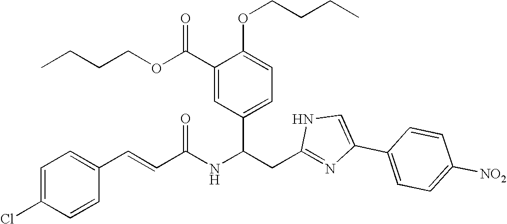 | 2-[3-(4′-Chloro-phenyl)- acrylamino]-2-(4′-n-butoxy- 3′-n-butoxycarbonylphenyl)- 2-ethyl[4-(4′-nitrophenyl)]imidazole |
| 22 | 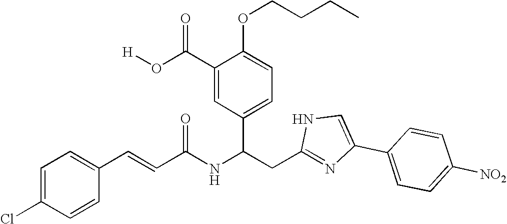 | 2-[3-(4′-Chloro-phenyl)- acrylamino]-2-(4′-n-butoxy- 3′-carboxyphenyl)-2-ethyl[4- (4′-nitrophenyl)]imidazole |
| 23 | 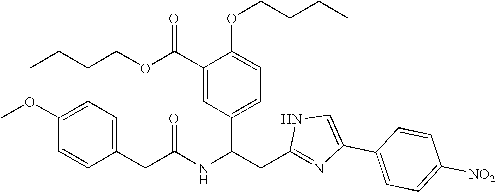 | 2-[2-(4′-Methoxy-phenyl)- acetylamino]-2-(4′-n-butoxy- 3′-n-butoxycarbonylphenyl)- 2-ethyl[4-(4′- nitrophenyl)]imidazole |
| 24 | 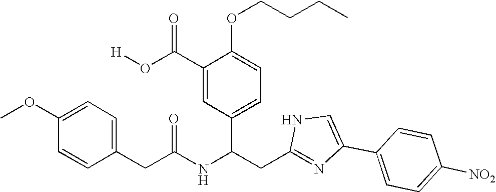 | 2-[2-(4′-Methoxy-phenyl)- acetylamino]-2-(4′-n-butoxy- 3′-carboxyphenyl)-2-ethyl[4- (4′-nitrophenyl)]imidazole |
| 25 | 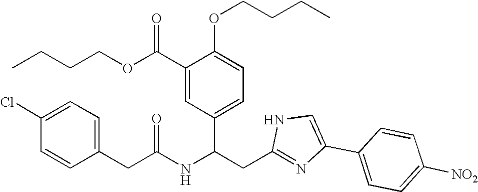 | 2-[2-(4′-Chloro-phenyl)- acetyl amino]-2-(4′-n- butoxy-3′-n-butoxycarbonyl phenyl)-2-ethyl[4-(4′- nitrophenyl)]imidazole |
| 26 | 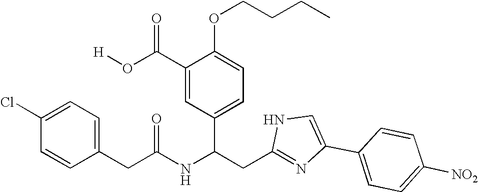 | 2-[2-(4′-Chloro-phenyl)- acetyl amino]-2-(4′-n- butoxy-3′-carboxy phenyl)- 2-ethyl[(4′- nitrophenyl)]imidazole |
| 27 | 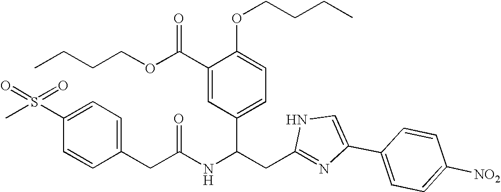 | 2-[2-(4′-Methylsulphonyl- phenyl)-acetyl amino]-2-(4′- n-butoxy-3′-n- butoxycarbonyl phenyl)-2- ethyl[4-(4′- nitrophenyl)]imidazole |
| 28 | 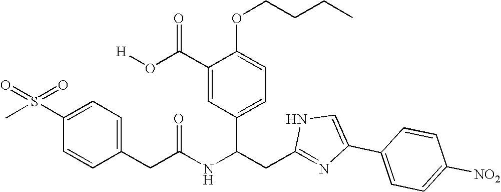 | 2-[2-(4′-Methylsulphonyl- phenyl)-acetyl amino]-2-(4′- n-butoxy-3′-carboxy phenyl)-2-ethyl[4-(4′- nitrophenyl)]imidazole |
| 29 | 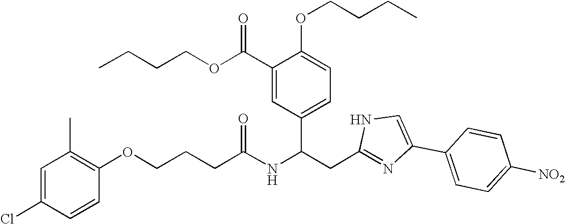 | 2-[4-(4′-chloro-2′-methyl- phenoxy)-butyryl amino]-2- (4′-n-butoxy-3′-n- butoxycarbonyl phenyl)-2- ethyl[4-(4′- nitrophenyl)]imidazole |
| 30 | 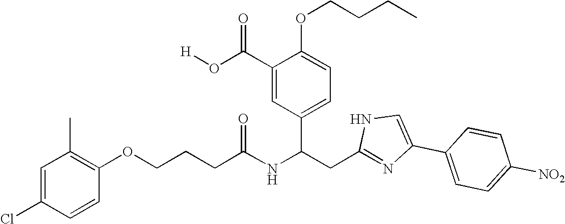 | 2-[4-(4′-chloro-2′-methyl- phenoxy)-butyryl amino]-2- (4′-n-butoxy-3′-carboxy phenyl)-2-ethyl[4-(4′- nitrophenyl)]imidazole |
| 31 | 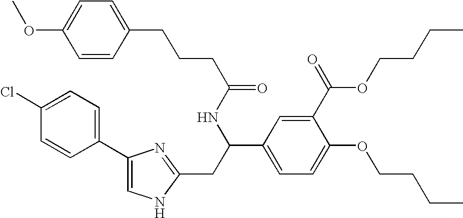 | 2-[4-(4′-Methoxyphenyl)- butyrylamino]-2-(4′-n- butoxy-3′-n-butoxycarbonyl phenyl)-2-ethyl[4-(4′- chlorophenyl)]imidazole |
| 32 | 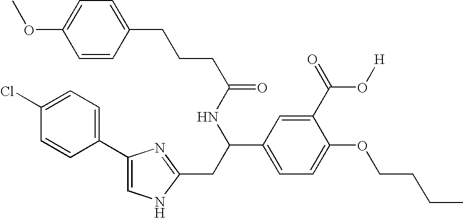 | 2-[4-(4′-Methoxyphenyl)- butyryl amino]-2-(4′-n- butoxy-3′-carboxy phenyl)- 2-ethyl[4-(4′-chlorophenyl)]imidazole |
| 33 | 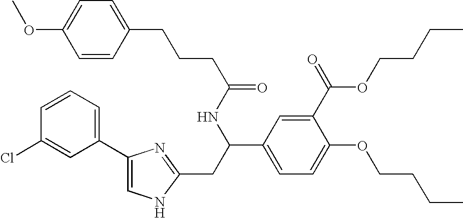 | 2-[4-(4′-Methoxyphenyl)- butyryl amino]-2-(4′-n- butoxy-3′-n-butoxycarbonyl phenyl)-2-ethyl[4-(3′- chlorophenyl)]imidazole |
| 34 | 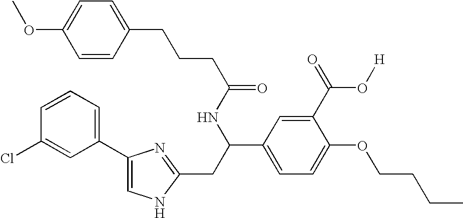 | 2-[4-(4′-Methoxyphenyl)- butyrylamino]-2-(4′-n- butoxy-3′-carboxyphenyl)-2- ethyl[4-(3′- chlorophenyl)]imidazole |
| 35 | 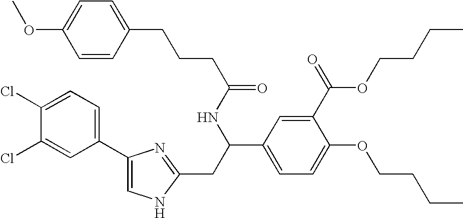 | 2-[4-(4′-Methoxyphenyl)- butyryl amino]-2-(4′-n- butoxy-3′-n-butoxycarbonyl phenyl)-2-ethyl[4-(3′,4′- dichlorophenyl)]imidazole |
| 36 | 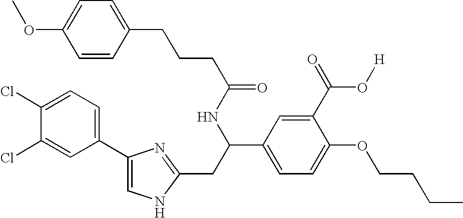 | 2-[4-(4′-Methoxyphenyl)- butyryl amino]-2-(4′-n- butoxy-3′-carboxy phenyl)- 2-ethyl[4-(3′,4′- dichlorophenyl)]imidazole |
| 37 | 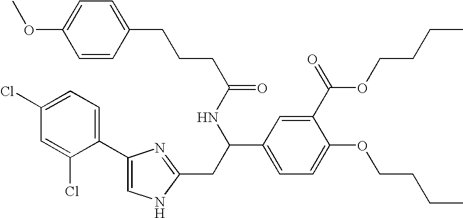 | 2-[4-(4′-Methoxyphenyl)- butyryl amino]-2-(4′-n- butoxy-3′-n-butoxycarbonyl phenyl)-2-ethyl[4-(2′,4′- dichlorophenyl)]imidazole |
| 38 | 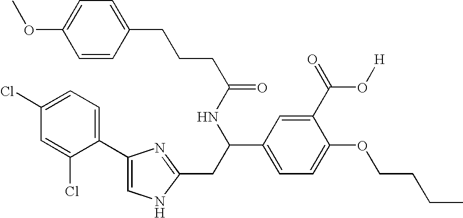 | 2-[4-(4′-Methoxyphenyl)- butyryl amino]-2-(4′-n- butoxy-3′-carboxy phenyl)- 2-ethyl[4-(2′,4′- dichlorophenyl)]imidazole |
| 39 | 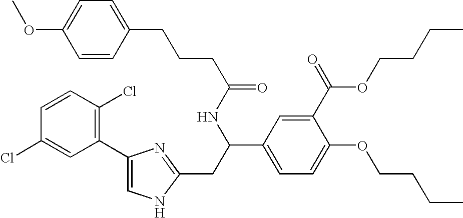 | 2-[4-(4′-Methoxyphenyl)- butyryl amino]-2-(4′-n- butoxy-3′-n-butoxycarbonyl phenyl)-2-ethyl[4-(2′,5′- dichlorophenyl)]imidazole |
| 40 | 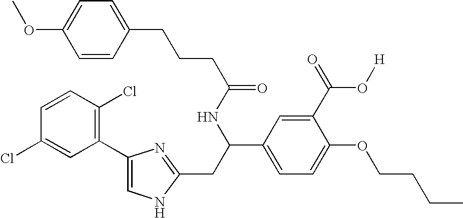 | 2-[4-(4′-Methoxyphenyl)- butyrylamino]-2-(4′-n- butoxy-3′-carboxyphenyl)-2- ethyl[4-(2′,5′- dichlorophenyl)]imidazole |
| 41 | 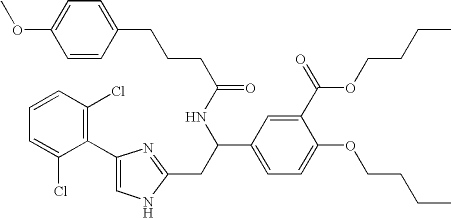 | 2-[4-(4′-Methoxyphenyl)- butyryl amino]-2-(4′-n- butoxy-3′-n-butoxycarbonyl phenyl)-2-ethyl[4-(2′,6′- dichlorophenyl)]imidazole |
| 42 | 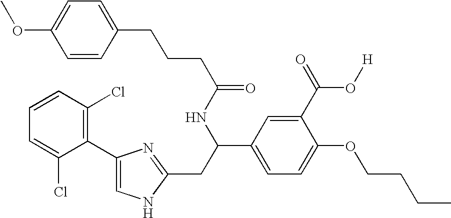 | 2-[4-(4′-Methoxyphenyl)- butyryl amino]-2-(4′-n- butoxy-3′-carboxy phenyl)- 2-ethyl[4-(2′,6′- dichlorophenyl)]imidazole |
| 43 | 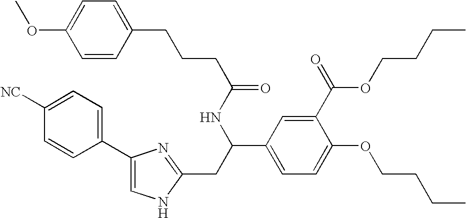 | 2-[4-(4′-Methoxyphenyl)- butyrylamino]-2-(4′-n- butoxy-3′-n- butoxycarbonylphenyl)-2- ethyl[4-(4′- cyanophenyl)]imidazole |
| 44 | 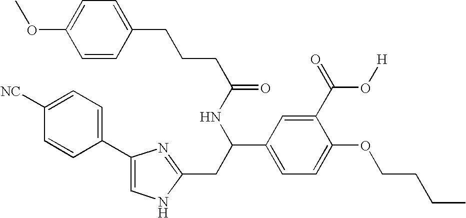 | 2-[4-(4′-Methoxyphenyl)- butyrylamino]-2-(4′-n- butoxy-3′-carboxyphenyl)-2- ethyl[4-(4′- cynophenyl)]imidazole |
| 45 | 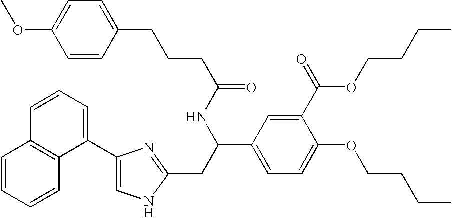 | 2-[4-(4′-Methoxyphenyl)- butyrylamino]-2-(4′-n- butoxy-3′-n- butoxycarbonylphenyl)-2- ethyl[4-(naphthyl)]imidazole |
| 46 | 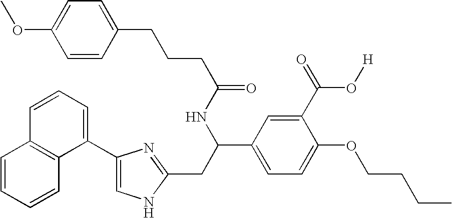 | 2-[4-(4′-Methoxyphenyl)- butyrylamino]-2-(4′-n- butoxy-3′-carboxyphenyl)-2- ethyl[4-(1-naphthyl)]imidazole |
| 47 | 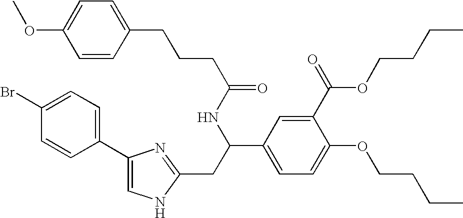 | 2-[4-(4′-Methoxyphenyl)- butyryl amino]-2-(4′-n- butoxy-3′-n- butoxycarbonylphenyl)-2- ethyl[4-(4′-bromo phenyl)]imidazole |
| 48 | 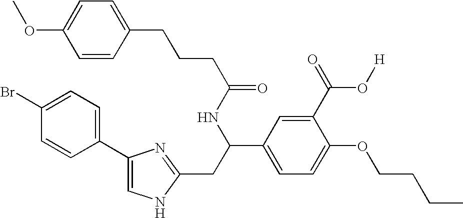 | 2-[4-(4′-Methoxyphenyl)- butyrylamino]-2-(4′-n- butoxy-3′-carboxyphenyl)-2- ethyl[4-(4′-bromophenyl)]imidazole |
| 49 | 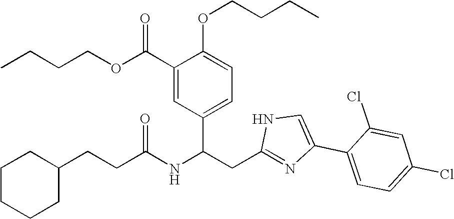 | 2-[4-(4′-Cyclohexyl)- propanoyl amino]-2-(4′-n- butoxy-3′-n- butoxycarbonylphenyl)-2- ethyl[4-(2′-4′-dichloro phenyl)]imidazole |
| 50 | 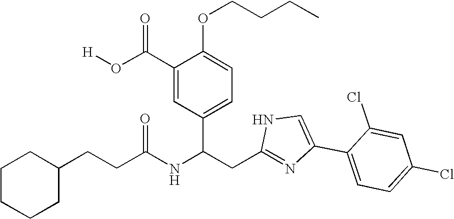 | 2-[4-(4′-Cyclohexyl)- propanoylamino]-2-(4′-n- butoxy-3′-carboxyphenyl)-2- ethyl[4-(2′,4′- dichlorophenyl)]imidazole |
| 51 | 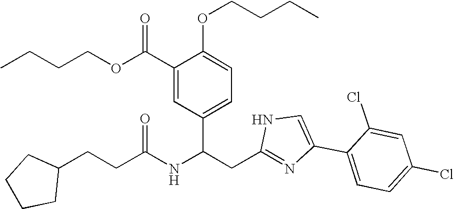 | 2-[4-(4′-Cyclopentyl)- propylamino]-2-(4′-n-butoxy- 3′-n-butoxycarbonylphenyl)- 2-ethyl[4-(2′,4′-dichloro phenyl)]imidazole |
| 52 | 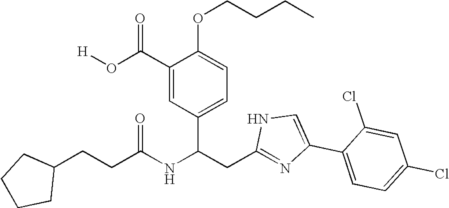 | 2-[4-(4′-Cyclopentyl)- propanoylamino]-2-(4′-n- butoxy-3′-carboxyphenyl)-2- ethyl[4-(2′,4′- dichlorophenyl)]imidazole |
| 53 | 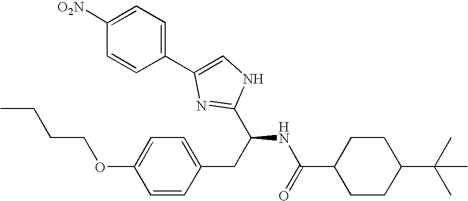 | N-{(1S)-2-(4-Butoxyphenyl)- 1-[4-(4-nitrophenyl)-1H- imidazol-2-yl]ethyl}-4-tert- butylcyclohexanecarboxamide |
| 54 | 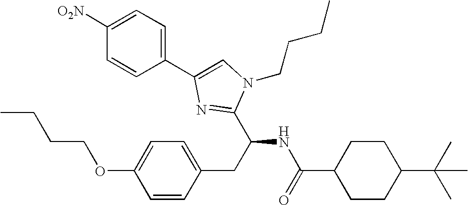 | N-{(1S)-2-(4-butoxyphenyl)- 1-[4-(4-nitrophenyl)-1H-1- butylimidazol-2-yl]ethyl}-4- tert-butylcyclohexanecarboxamide |
| 55 | 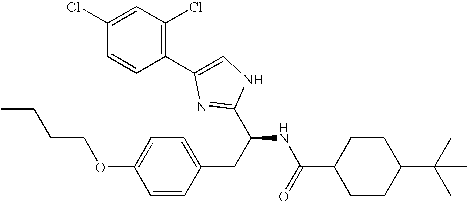 | N-{(1S)-2-(4-Butoxyphenyl)- 1-[4-(2,4-dichlorophenyl)- 1H-imidazol-2-yl]ethyl}-4- tert-butylcyclohexanecarboxamide |
| 56 | 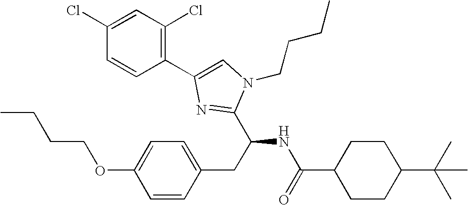 | N-{(1S)-2-(4-butoxyphenyl)- 1-[4-(2,4-dichlorophenyl)- 1H-1-butylimidazol-2- yl]ethyl}-4-tert- butylcyclohexanecarboxamide |
| 57 | 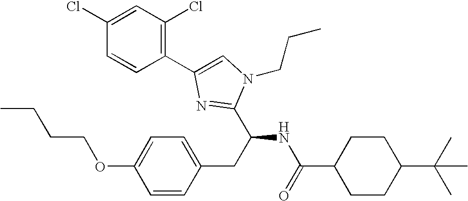 | N-{(1S)-2-(4-Butoxyphenyl)- 1-[4-(2,4-dichlorophenyl)- 1H-1-propoxyimidazol-2- yl]ethyl}-4-tert- butylcyclohexanecarboxamide |
| 58 | 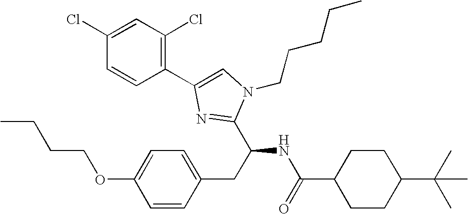 | N-{(1S)-2-(4-Butoxyphenyl)- 1-[4-(2,4-dichlorophenyl)- 1H-1-pentylimidazol-2- yl]ethyl}-4-tert- butylcyclohexanecarboxamide |
| 59 | 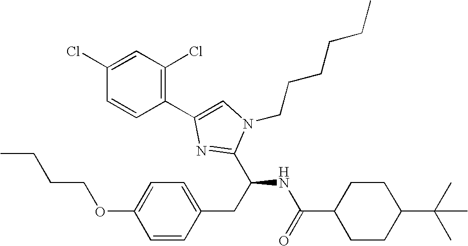 | N-{(1S)-2-(4-Butoxyphenyl)- 1-[4-(2,4-dichlorophenyl)- 1H-1-hexylimidazol-2- yl]ethyl}-4-tert- butylcyclohexanecarboxamide |
| 60 | 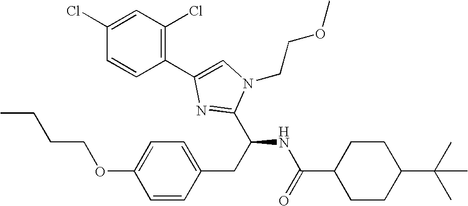 | N-{(1S)-2-(4-Butoxyphenyl)- 1-[4-(2,4-dichlorophenyl)- 1H-1-(2-methoxy-1- ethyl)imidazol-2-yl]ethyl}-4- tert-butylcyclohexanecarboxamide |
| 61 | 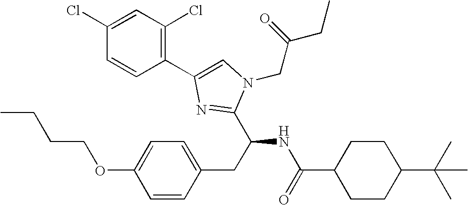 | N-{(1S)-2-(4-butoxyphenyl)- 1-[4-(2,4-dichlorophenyl)- 1H-1-(2-oxo-1- butyl)imidazol-2-yl]ethyl}-4- tert-butylcyclohexanecarboxamide |
| 62 | 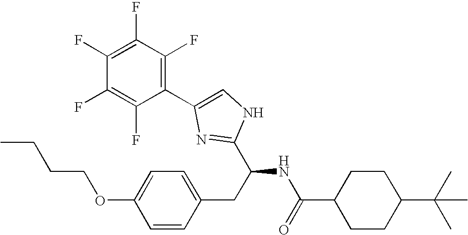 | N-{(1S)-2-(4-Butoxyphenyl)- 1-[4-(pentafluorophenyl)- 1H-imidazol-2-yl]ethyl}-4- tert-butylcyclohexanecarboxamide |
| 63 | 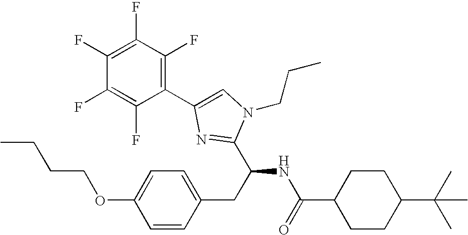 | N-{(1S)-2-(4-Butoxyphenyl)- 1-[4-(pentafluorophenyl)- 1H-1-propylimidazol-2- yl]ethyl}-4-tert- butylcyclohexanecarboxamide |
| 64 | 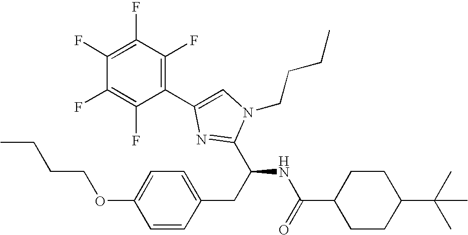 | N-{(1S)-2-(4-Butoxyphenyl)- 1-[4-(pentafluorophenyl)- 1H-1-butylimidazol-2- yl]ethyl}-4-tert- butylcyclohexanecarboxamide |
| 65 | 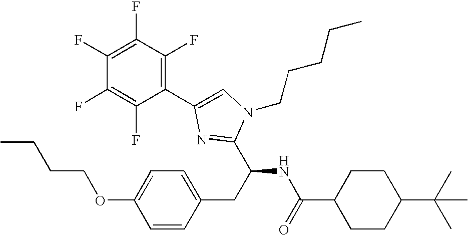 | N-{(1S)-2-(4-Butoxyphenyl)- 1-[4-(pentafluorophenyl)- 1H-1-pentylimidazol-2- yl]ethyl}-4-tert- butylcyclohexanecarboxamide |
| 66 | 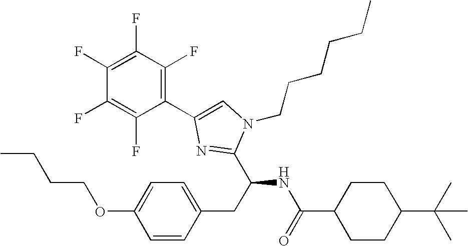 | N-{(1S)-2-(4-Butoxyphenyl)- 1-[4-(pentafluorophenyl)- 1H-1-hexylimidazol-2- yl]ethyl}-4-tert- butylcyclohexanecarboxamide |
| 67 | 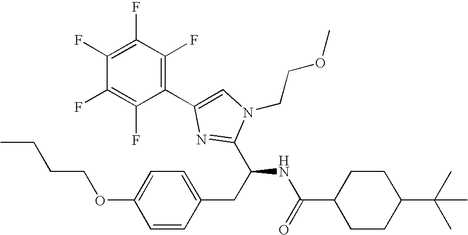 | N-{(1S)-2-(4-Butoxyphenyl)- 1-[4-(pentafluorophenyl)- 1H-1-(2-methoxy-1- ethyl)imidazol-2-yl]ethyl}-4- tert-butylcyclohexanecarboxamide |
| 68 | 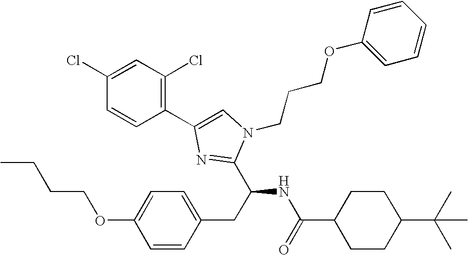 | N-{(1S)-2-(4-Butoxyphenyl)- 1-[4-(2,4-dichlorophenyl)- 1H-1-(3-phenoxy-1-propyl)- imidazol-2-yl]ethyl}-4-tert- butylcyclohexanecarboxamide |
| 69 | 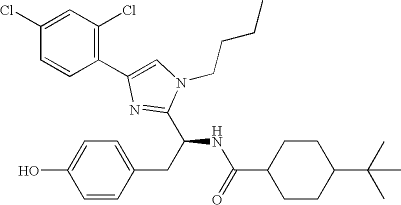 | 4-tert-Butyl- cyclohexanecarboxylic acid [(1S)-1-[1-butyl-4-(2,4- dichloro-phenyl)-1H- imidazol-2-yl]-2-(4-hydroxy- phenyl)-ethyl]-amide |
| 70 | 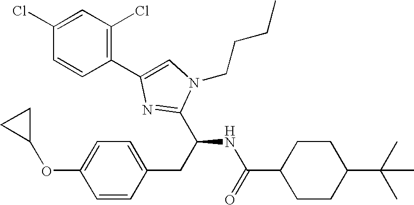 | N-{(1S)-2-(4- Cyclopropoxyphenyl)-1-[4- (2,4-dichlorophenyl)-1H-1- (1-butyl)imidazol-2-yl]ethyl}-4- tert-butylcyclohexanecarboxamide |
| 71 | 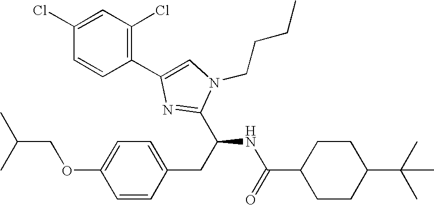 | N-{(1S)-2-(4- Isobutoxyphenyl)-1-[4-(2,4- dichlorophenyl)-1H-1-(1- butyl)imidazol-2-yl]ethyl}-4- tert-butylcyclohexanecarboxamide |
| 72 | 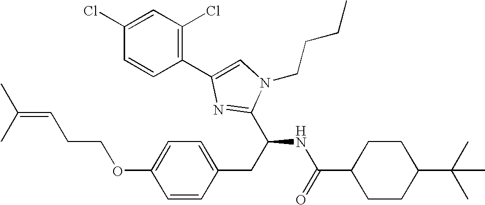 | N-{( 1S)-2-(4-(4-Methyl-3- pentene-1-yloxy)phenyl)-1- [4-(2,4-dichlorophenyl)-1H- 1-(1-butyl)imidazol-2- yl]ethyl}-4-tert- butylcyclohexanecarboxamide |
| 73 | 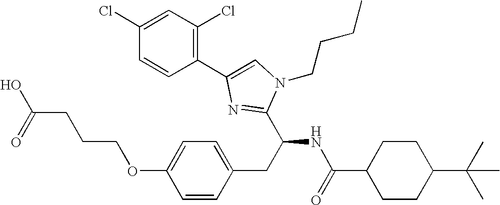 | N-{(1S)-2-(4-(3-Carboxy-1- propoxy)phenyl)-1-[4-(2,4- dichlorophenyl)-1H-1-(1- butyl)-imidazol-2-yl]ethyl}-4- tert-butylcyclohexanearboxamide |
| 74 | 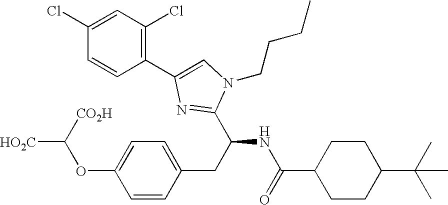 | N-{(1S)-2-(4-(1,1- Dicarboxymethoxy)phenyl)- 1-[4-(2,4-dichlorophenyl)- 1H-1-(1-butyl)imidazol-2- yl]ethyl}-4-tert- butylcyclohexanecarboxamide |
| 75 | 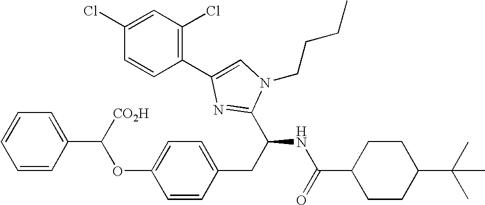 | N-{(1S)-2-(4-(1-carboxy-1- phenylmethoxy)phenyl)-1- [4-(2,4-dichlorophenyl)-1H- 1-(1-butyl)imidazol-2- yl]ethyl}-4-tert- butylcyclohexanecarboxamide |
| 76 | 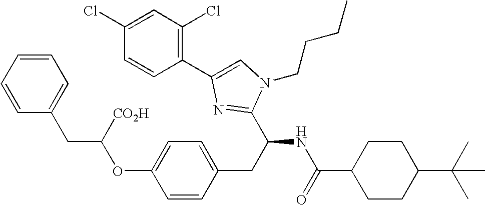 | N-{(1S)-2-(4-((2-Phenyl-1- carboxy)1-ethoxy)phenyl)-1- [4-(2,4-dichlorophenyl)-1H- 1-(1-butyl)imidazol-2- yl]ethyl}-4-tert- butylcyclohexanecarboxamide |
| 77 | 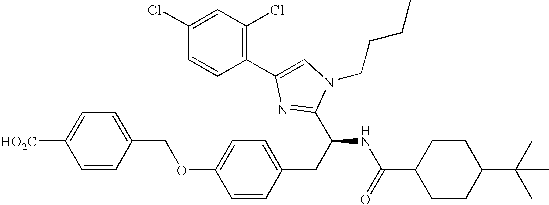 | 4-(4-{(2S)-2-[(4-tert-Butyl- cyclohexanecarbonyl)- amino]-2-[1-butyl-4-(2,4- dichloro-phenyl)-1H- imidazol-2-yl]-ethyl}- phenoxymethyl)-benzoic acid |
| 78 | 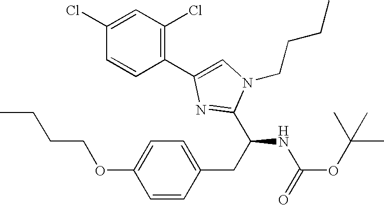 | {2-(4-Butoxy-phenyl)-(1S)-1- [1-butyl-4-(2,4-dichloro- phenyl)-1H-imidazol-2-yl]- ethyl}-carbamic acid tert butyl ester |
| 79 | 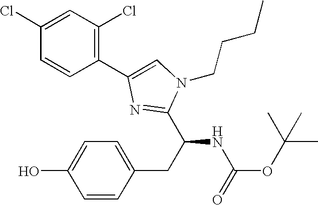 | [(1S)-1-[1-Butyl-4-(2,4- dichloro-phenyl)-1H- imidazol-2-yl]-2-(4-hydroxy- phenyl)-ethyl]-carbamic acid tert-butyl ester |
| 80 | 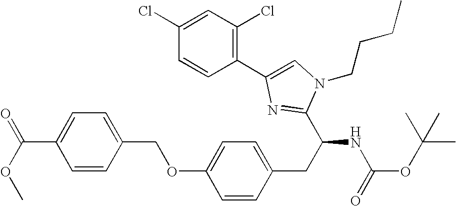 | 4-(4-{(2S)-2-tert- Butoxycarbonylamino-2-[1- butyl-4-(2,4-dichlorophenyl)- 1H-imidazol-2-yl]-ethyl}- phenoxymethyl)-benzoic acid methyl ester |
| 81 | 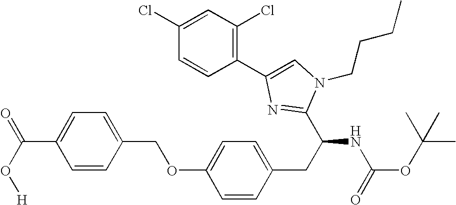 | 4-(4-{(2S)-2-tert- Butoxycarbonylamino-2-[1- butyl-4-(2,4-dichloro- phenyl)-1H-imidazol-2-yl]- ethyl}-phenoxymethyl)- benzoic acid |
| 82 | 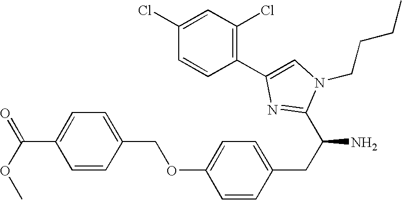 | 4-(4-{(2S)-2-Amino-2-[1- butyl-4-(2,4-dichloro- phenyl)-1H-imidazol-2-yl]- ethyl}-phenoxymethyl)- benzoic acid methyl ester hydrochloride |
| 83 | 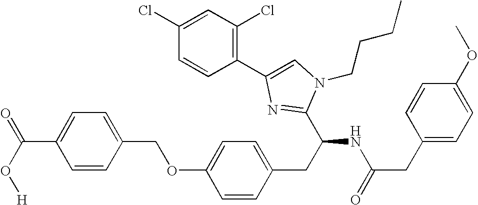 | 4-(4-{2-[1-Butyl-4-(2,4- dichloro-phenyl)-1H- imidazol-2-yl]-(2S)-2-[2-(4- methoxy-phenyl)- acetylamino]-ethyl}- phenoxymethyl)-benzoic acid |
| 84 | 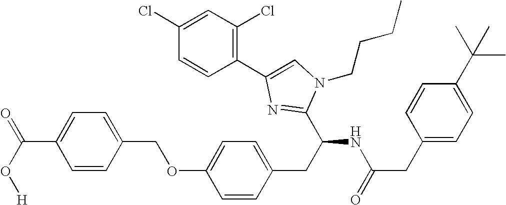 | 4-(4-{2-[1-Butyl-4-(2,4- dichloro-phenyl)-1H- imidazol-2-yl]-(2S)-2-[2-(4- tert-butyl-phenyl)- acetylamino]-ethyl}- phenoxymethyl)-benzoic acid |
| 85 | 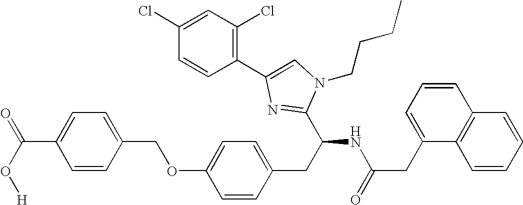 | 4-{4-[2-[1-Butyl-4-(2,4- dichloro-phenyl)-1H- imidazol-2-yl]-(2S)-2-(2- naphthalen-1-yl- acetylamino)-ethyl]- phenoxymethyl}-benzoic acid |
| 86 | 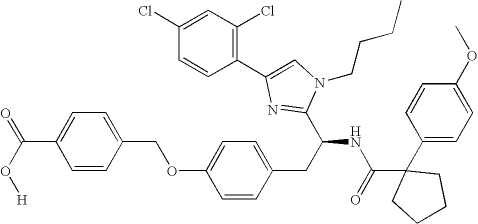 | 4-[4-(2-[1-Butyl-4-(2,4- dichloro-phenyl)-1H- imidazol-2-yl]-(2S)-2-{[1-(4- methoxy-phenyl)- cyclopentanecarbonyl]- amino}-ethyl)- phenoxymethyl]-benzoic acid |
| 87 | 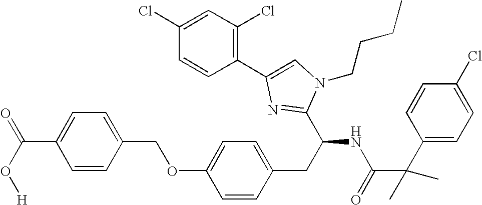 | 4-(4-{2-[1-Butyl-4-(2,4- dichloro-phenyl)-1H- imidazol-2-yl]-(2S)-2-[2-(4- chloro-phenyl)-2-methyl- propionylamino]-ethyl}- phenoxymethyl)-benzoic acid |
| 88 | 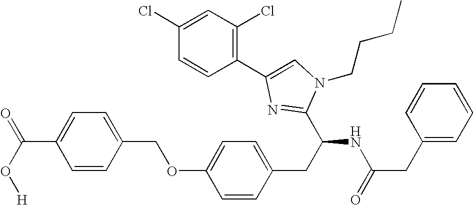 | 4-(4-{2-[1-Butyl-4-(2,4- dichloro-phenyl)-1H- imidazol-2-yl]-(2S)-2- phenylacetylamino-ethyl}- phenoxymethyl)-benzoic acid |
| 89 | 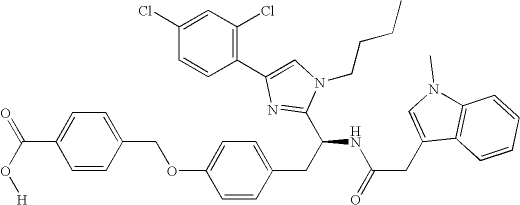 | 4-(4-{2-[1-Butyl-4-(2,4- dichloro-phenyl)-1H- imidazol-2-yl]-(2S)-2-[2-(1- methyl-1H-indol-2-yl)- acetylamino]-ethyl}- phenoxymethyl)-benzoic acid |
| 90 | 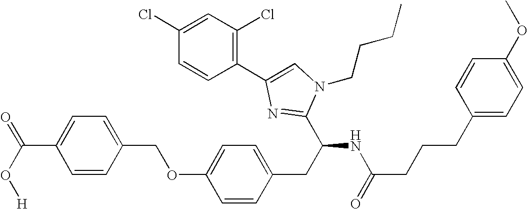 | 4-(4-{2-[1-Butyl-4-(2,4- dichloro-phenyl)-1H- imidazol-2-yl]-(2S)-2-[4-(4- methoxy-phenyl)- butyrylamino]-ethyl}- phenoxymethyl)-benzoic acid |
| 91 | 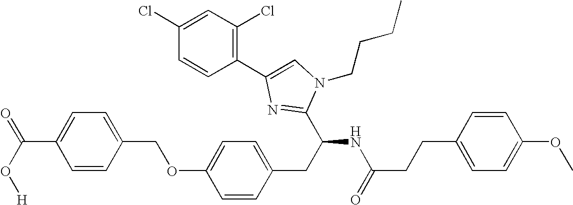 | 4-(4-{2-[1-Butyl-4-(2,4- dichloro-phenyl)-1H- imidazol-2-yl]-(2S)-2-[3-(4- methoxy-phenyl)-propionyl- amino]-ethyl}- phenoxymethyl)-benzoic acid |
| 92 | 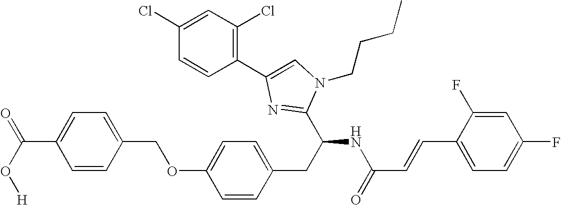 | 4-(4-{2-[1-Butyl-4-(2,4- dichloro-phenyl)-1H- imidazol-2-yl]-(2S)-2-[3- (2,4-difluoro-phenyl)- acryloylamino]-ethyl}- phenoxymethyl)-benzoic acid |
| 93 | 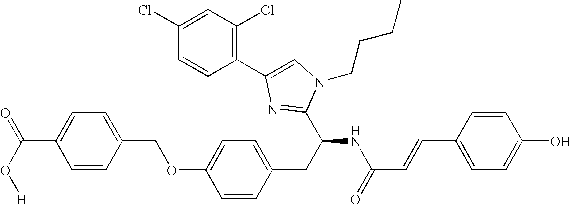 | 4-(4-{2-[1-Butyl-4-(2,4- dichloro-phenyl)-1H- imidazol-2-yl]-(2S)-2-[3-(4- hydroxy-phenyl)- acryloylamino]-ethyl}- phenoxymethyl)-benzoic acid |
| 94 | 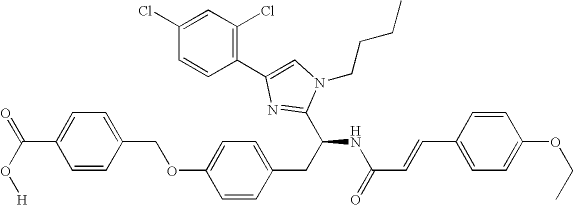 | 4-(4-{2-[1-Butyl-4-(2,4- dichloro-phenyl)-1H- imidazol-2-yl]-(2S)-2-[3-(4- ethoxy-phenyl)- acryloylamino]-ethyl}- phenoxymethyl)-benzoic acid |
| 95 | 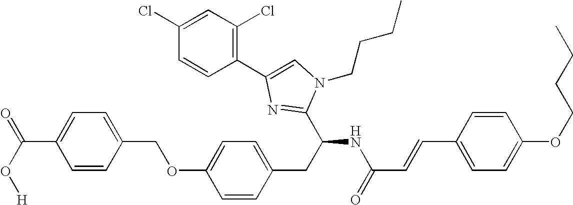 | 4-(4-{2-[3-(4-Butoxy- phenyl)-acryloylamino]- (2S)-2-[1-butyl-4-(2,4- (dichloro-phenyl)-1H- imidazol-2-yl]-ethyl}- phenoxymethyl)-benzoic acid |
| 96 | 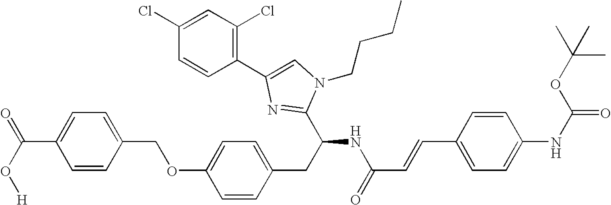 | 4-(4-{2-[3-(4-tert- Butoxycarbonylamino- phenyl)-acryloylamino]- (2S)-2-[1-butyl-4-(2,4- dichloro-phenyl)-1H- imidazol-2-yl]-ethyl}- phenoxymethyl)-benzoic acid |
| 97 | 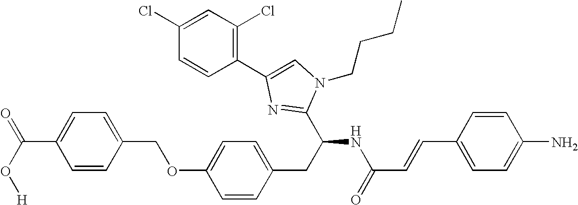 | 4-(4-{2-[3-(4-Amino-phenyl)- acryloylamino]-(2S)-2-[1- butyl-4-(2,4-dichloro- phenyl)-1H-imidazol-2-yl]- ethyl}-phenoxymethyl)- benzoic acid hydrochloride |
| 98 | 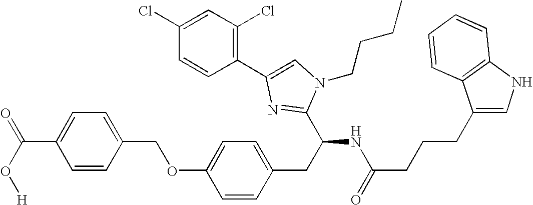 | 4-{4-[2-[1-Butyl-4-(2,4- dichloro-phenyl)-1H- imidazol-2-yl]-(2S)-2-(4- (1H-indol-2-yl)- butyrylamino)-ethyl]- phenoxymethyl}-benzoic acid |
| 99 | 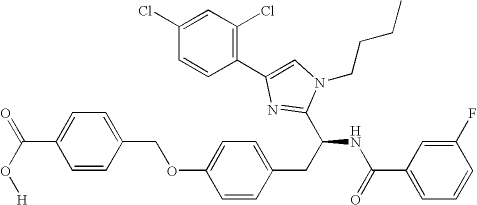 | 4-{4-[2-[1-Butyl-4-(2,4- dichloro-phenyl)-1H- imidazol-2-yl]-(2S)-2-(3- fluoro-benzoylamino)-ethyl]- phenoxymethyl}-benzoic acid |
| 100 | 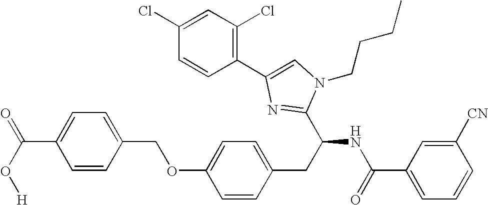 | 4-{4-[2-[1-Butyl-4-(2,4- dichloro-phenyl)-1H- imidazol-2-yl]-(2S)-2-(3- cyano-benzoylamino)-ethyl]- phenoxymethyl}-benzoic acid |
| 101 | 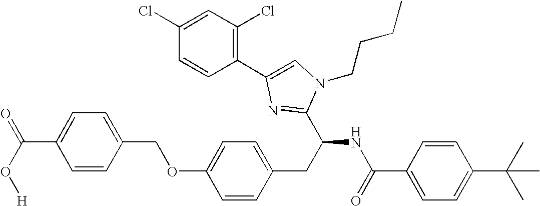 | 4-(4-{2-(4-tert-Butyl- benzoylamino)-(2S)-2-[1- butyl-4-(2,4-dichloro- phenyl)-1H-imidazol-2-yl]- ethyl}-phenoxymethyl)- benzoic acid |
| 102 | 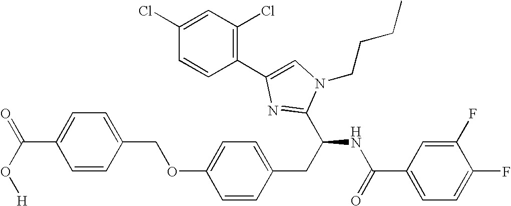 | 4-{4-[2-[1-Butyl-4-(2,4- dichloro-phenyl)-1H- imidazol-2-yl]-(2S)-2-(3,4- difluoro-benzoylamino)- ethyl]-phenoxymethyl}- benzoic acid |
| 103 | 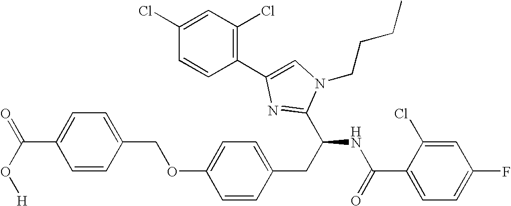 | 4-{4-[2-[1-Butyl-4-(2,4- dichloro-phenyl)-1H- imidazol-2-yl]-(2S)-2-(2- chloro-4-fluoro- benzoylamino)-ethyl]- phenoxymethyl}-benzoic acid |
| 104 | 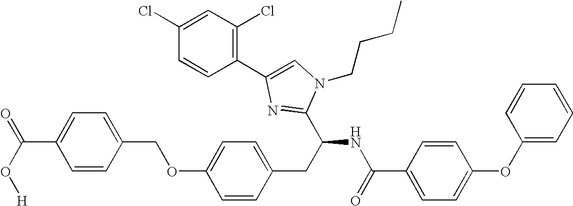 | 4-{4-[2-[1-Butyl-4-(2,4- dichloro-phenyl)-1H- imidazol-2-yl]-(2S)-2-(4- phenoxy-benzoylamino)- ethyl]-phenoxymethyl}- benzoic acid |
| 105 | 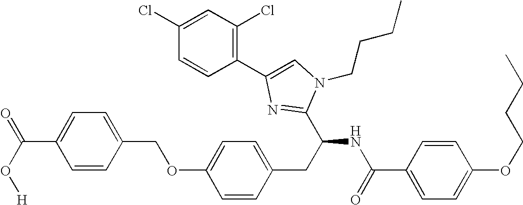 | 4-(4-{2-(4-Butoxy- benzoylamino)-(2S)-2-[1- butyl-4-(2,4-dichloro- phenyl)-1H-imidazol-2-yl]- ethyl}-phenoxymethyl)- benzoic acid |
| 106 | 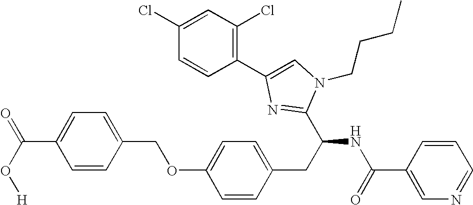 | 4-(4-{2-[1-Butyl-4-(2,4- dichloro-phenyl)-1H- imidazol-2-yl]-(2S)-2- [(pyridine-3-carbonyl)- amino]-ethyl}- phenoxymethyl)-benzoic acid |
| 107 | 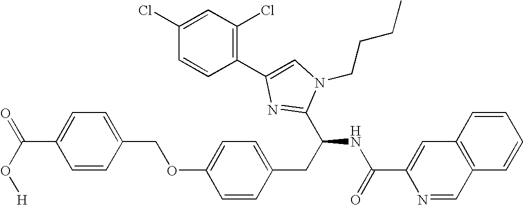 | 4-(4-{2-[1-Butyl-4-(2,4- dichloro-phenyl)-1H- imidazol-2-yl]-(2S)-2- [(isoquinoline-3-carbonyl)- amino]-ethyl}- phenoxymethyl)-benzoic acid |
| 108 | 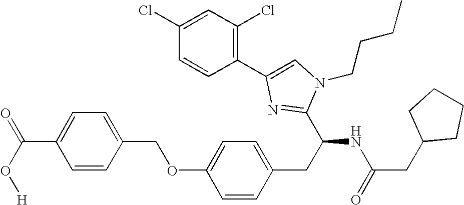 | 4-{4-[2-[1-Butyl-4-(2,4- dichloro-phenyl)-1H- imidazol-2-yl]-(2S)-2-(2- cyclopentyl-acetylamino)- ethyl]-phenoxymethyl}- benzoic acid |
| 109 | 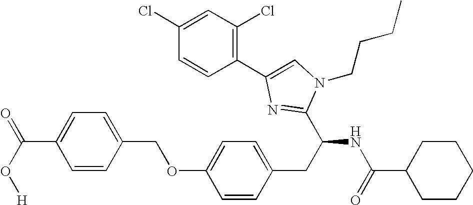 | 4-{4-[2-[1-Butyl-4-(2,4- dichloro-phenyl)-1H- imidazol-2-yl]-(2S)-2- (cyclohexanecarbonyl- amino)-ethyl]- phenoxymethyl}-benzoic acid |
| 110 | 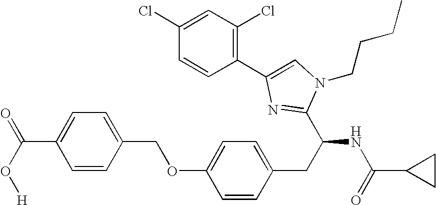 | 4-{4-[2-[1-Butyl-4-(2,4- dichloro-phenyl)-1H- imidazol-2-yl]-(2S)-2- (cyclopropanecarbonyl amino)-ethyl]- phenoxymethyl}-benzoic acid |
| 111 | 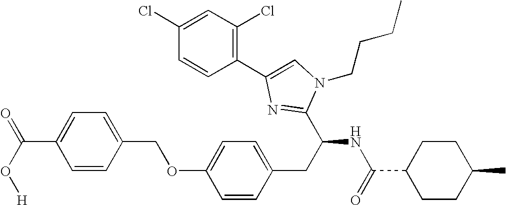 | 4-(4-{2-[1-Butyl-4-(2,4- dichloro-phenyl)-1H- imidazol-2-yl]-(2S)-2- [(trans-4-methyl- cyclohexanecarbonyl)- amino]-ethyl}- phenoxymethyl)-benzoic acid |
| 112 | 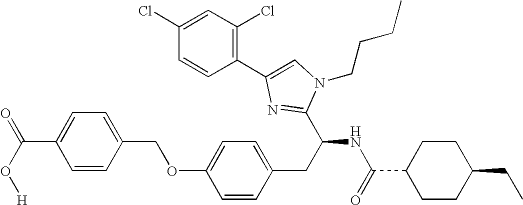 | 4-(4-{2-[1-Butyl-4-(2,4- dichloro-phenyl)-1H- imidazol-2-yl]-(2S)-2- [(trans-4-ethyl- cyclohexanecarbonyl)- amino]-ethyl}- phenoxymethyl)-benzoic acid |
| 113 | 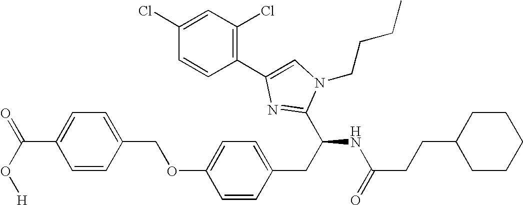 | 4-{4-[2-[1-Butyl-4-(2,4- dichloro-phenyl)-1H- imidazol-2-yl]-(2S)-2-(3- cyclohexyl-propionyl- amino)-ethyl]- phenoxymethyl}-benzoic acid |
| 114 | 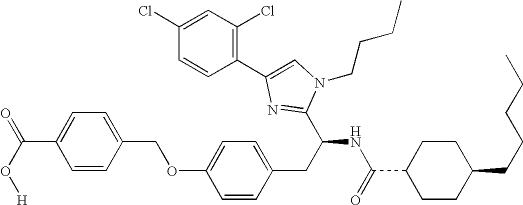 | 4-(4-{2-[1-Butyl-4-(2,4- dichloro-phenyl)-1H- imidazol-2-yl]-(2S)-2- [(trans-4-pentyl- cyclohexanecarbonyl)- amino]-ethyl}- phenoxymethyl)-benzoic acid |
| 115 | 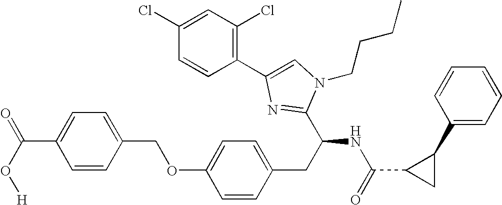 | 4-(4-{2-[1-Butyl-4-(2,4- dichloro-phenyl)-1H- imidazol-2-yl]-(2S)-2- [(trans-2-phenyl- cyclopropanecarbonyl)- amino]-ethyl}- phenoxymethyl)-benzoic acid |
| 116 | 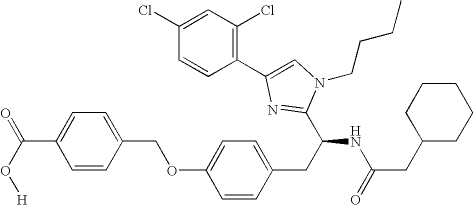 | 4-{4-[2-[1-Butyl-4-(2,4- dichloro-phenyl)-1H- imidazol-2-yl]-(2S)-2-(2- cyclohexyl-acetylamino)- ethyl]-phenoxymethyl}- benzoic acid |
| 117 | 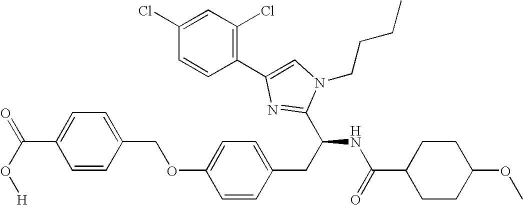 | 4-(4-{2-[1-Butyl-4-(2,4- dichloro-phenyl)-1H- imidazol-2-yl]-(2S)-2-[(4- methoxycyclohexanecarbonyl)- amino]-ethyl}- phenoxymethyl)-benzoic acid |
| 118 | 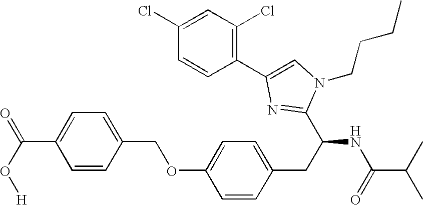 | 4-(4-{2-[1-Butyl-4-(2,4- dichloro-phenyl)-1H- imidazol-2-yl]-(2S)-2- isobutyrylamino-ethyl}- phenoxymethyl)-benzoic acid |
| 119 | 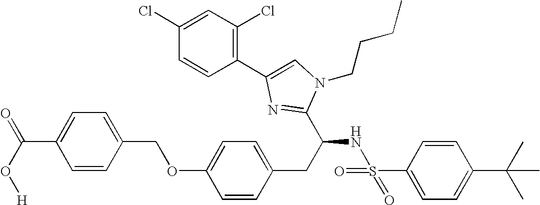 | 4-(4-{(2S)-2-(4-tert-Butyl- benzenesulfonylamino)-2- [1-butyl-4-(2,4-dichloro- phenyl)-1H-imidazol-2-yl]- ethyl}-phenoxymethyl)- benzoic acid |
| 120 | 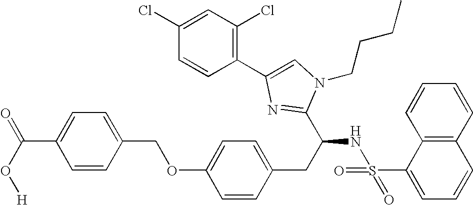 | 4-{4-[2-[1-Butyl-4-(2,4- dichloro-phenyl)-1H- imidazol-2-yl]-(2S)-2- (naphthalene-1- sulfonylamino)-ethyl]- phenoxymethyl}-benzoic acid |
| 121 | 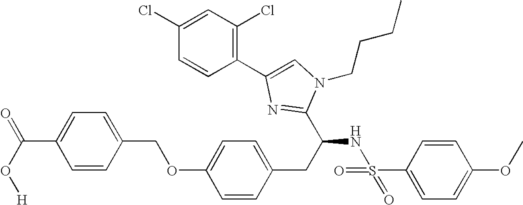 | 4-{4-[2-[1-Butyl-4-(2,4- dichloro-phenyl)-1H- imidazol-2-yl]-(2S)-2-(4- methoxy-benzene- sulfonylamino)-ethyl]- phenoxymethyl}-benzoic acid |
| 122 | 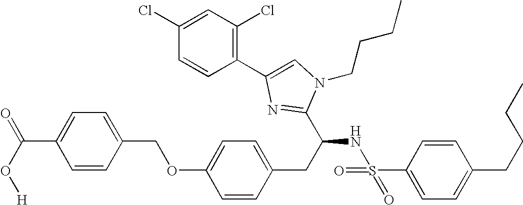 | 4-(4-{(2S)-2-(4-Butyl- benzenesulfonylamino)-2- [1-butyl-4-(2,4-dichloro- phenyl)-1H-imidazol-2-yl]- ethyl}-phenoxymethyl)- benzoic acid |
| 123 | 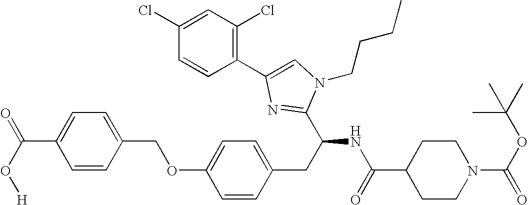 | 4-{(1S)-1-[1-Butyl-4-(2,4- dichloro-phenyl)-1H- imidazol-2-yl]-2-[4-(4- carboxy-benzyloxy)-phenyl]- ethylcarbamoyl}-piperidine- 1-carboxylic acid tert-butyl ester |
| 124 | 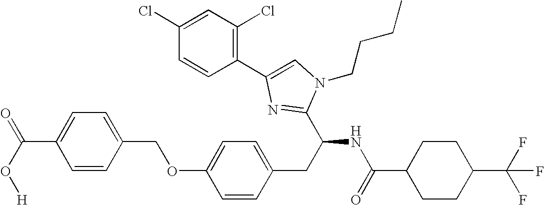 | 4-(4-{2-[1-Butyl-4-(2,4- dichloro-phenyl)-1H- imidazol-2-yl]-(2S)-2-[(4- trifluoromethyl- cyclohexanecarbonyl)- amino]-ethyl}- phenoxymethyl)-benzoic acid |
| 125 | 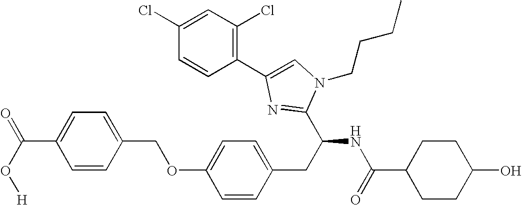 | 4-(4-{2-[1-Butyl-4-(2,4- dichloro-phenyl)-1H- imidazol-2-yl]-(2S)-2-[(4- hydroxy-cyclohexane- carbonyl-amino]-ethyl}- phenoxymethyl)-benzoic acid |
| 126 | 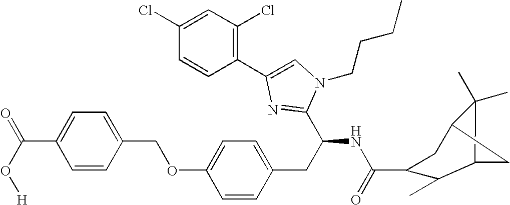 | 4-(4-{2-[1-Butyl-4-(2,4- dichloro-phenyl)-1H- imidazol-2-yl]-(2S)-2- [(2,6,6-trimethyl- bicyclo[3.1.1]heptane-3- carbonyl)-amino]-ethyl}- phenoxymethyl)-benzoic acid |
| 127 | 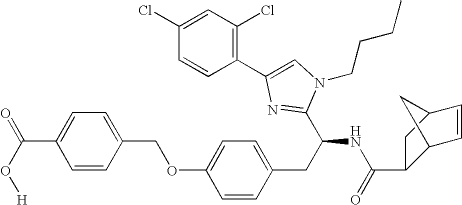 | 4-(4-{(2S)-2- [(Bicyclo[2.2.1]heptane-5- ene-2-carbonyl)-amino]-2- [1-butyl-4-(2,4-dichloro- phenyl)-1H-imidazol-2-yl]- ethyl}-phenoxymethyl)- benzoic acid |
| 128 | 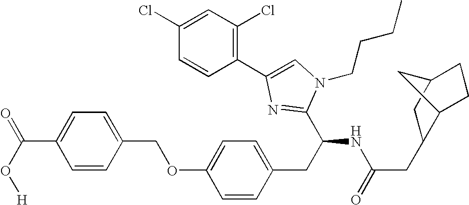 | 4-(4-{(2S)-2-(2- Bicyclo[2.2.1]hept-2-yl- acetylamino)-2-[1-butyl-4- (2,4-dichloro-phenyl)-1H- imidazol-2-yl]-ethyl}- phenoxymethyl)-benzoic acid |
| 129 | 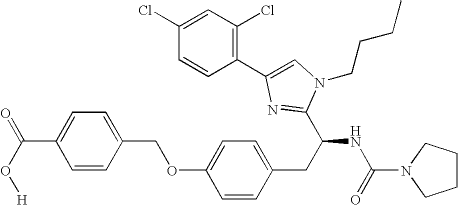 | 4-(4-{2-[1-Butyl-4-(2,4- dichloro-phenyl)-1H- imidazol-2-yl]-(2S)-2- [(pyrrolidine-1-carbonyl)- amino]-ethyl}- phenoxymethyl)-benzoic acid |
| 130 | 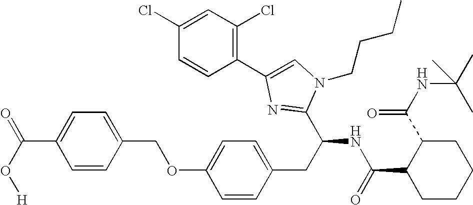 | 4-(4-{(2S)-2-[(Trans-2-tert- Butylcarbamoyl- cyclohexanecarbonyl)- amino]-2-[1-butyl-4-(2,4- dichloro-phenyl)-1H- imidazol-2-yl]-ethyl}- phenoxymethyl)-benzoic acid |
| 131 | 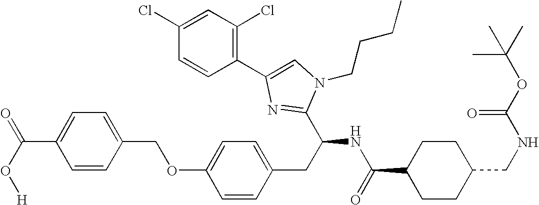 | 4-(4-{(2S)-2-{[trans-4-(tert- Butoxycarbonylamino- methyl)- cyclohexanecarbonyl]- amino}-2-[1-butyl-4-(2,4- dichloro-phenyl)-1H- imidazol-2-yl]-ethyl}- phenoxymethyl)-benzoic acid |
| 132 | 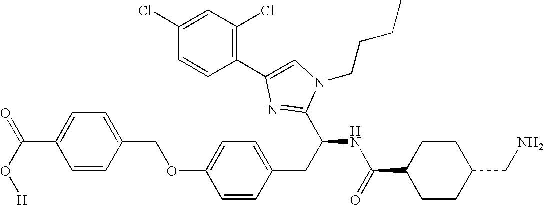 | 4-(4-{(2S)-2-[trans-(4- Aminomethyl- cyclohexanecarbonyl)- amino]-2-[1-butyl-4-(2,4- dichloro-phenyl)-1H- imidazol-2-yl]-ethyl}- phenoxymethyl)-benzoic acid hydrochloride |
| 133 | 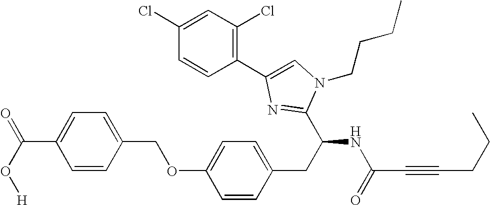 | 4-(4-{2-[1-Butyl-4-(2,4- dichloro-phenyl)-1H- imidazol-2-yl]-(2S)-2-hex-2- ynoylamino-ethyl}- phenoxymethyl)-benzoic acid |
| 134 | 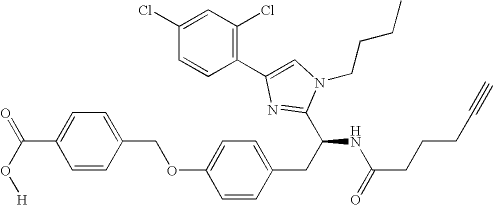 | 4-(4-{2-[1-Butyl-4-(2,4- dichloro-phenyl)-1H- imidazol-2-yl]-(2S)-2-hex-5- ynoylamino-ethyl}- phenoxymethyl)-benzoic acid |
| 135 | 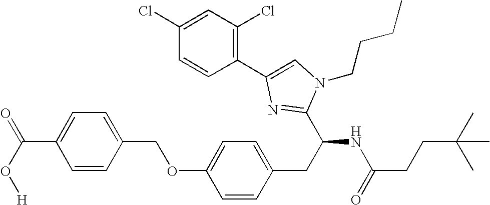 | 4-{4-[2-[1-Butyl-4-(2,4- dichloro-phenyl)-1H- imidazol-2-yl]-(2S)-2-(4,4- dimethyl-pentanoyl-amino)- ethyl]-phenoxymethyl}- benzoic acid |
| 136 | 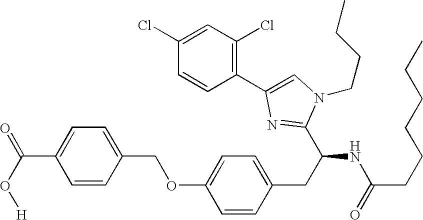 | 4-(4-{2-[1-Butyl-4-(2,4- dichloro-phenyl)-1H- imidazol-2-yl]-(2S)-2- heptanoylamino-ethyl}- phenoxymethyl)-benzoic acid |
| 137 | 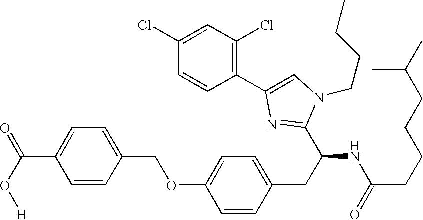 | 4-{4-[2-[1-Butyl-4-(2,4- dichloro-phenyl)-1H- imidazol-2-yl]-(2S)-2-(6- methyl-heptanoylamino)- ethyl]-phenoxymethyl}- benzoic acid |
| 138 | 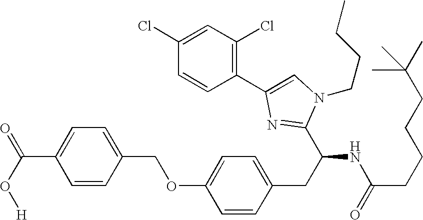 | 4-{4-[2-[1-Butyl-4-(2,4- dichloro-phenyl)-1H- imidazol-2-yl]-(2S)-2-(6,6- dimethylheptanoyl-amino)- ethyl]-phenoxymethyl}- benzoic acid |
| 139 | 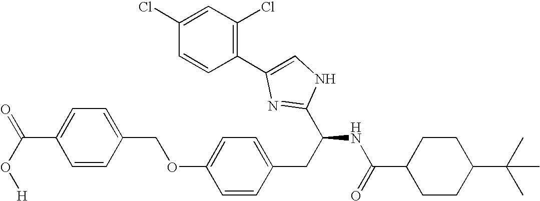 | 4-(4-{(2S)-2-[(4-tert-Butyl- cyclohexanecarbonyl)- amino]-2-[4-(2,4-dichloro- phenyl)-1H-imidazol-2-yl]- ethyl}-phenoxymethyl)- benzoic acid |
| 140 | 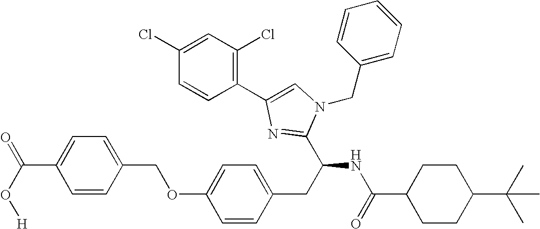 | 4-(4-{2-[1-Benzyl-4-(2,4- dichloro-phenyl)-1H- imidazol-2-yl]-(2S)-2-[(4- tert-butyl-cyclohexane- carbonyl)-amino]-ethyl}- phenoxymethyl)-benzoic acid |
| 141 | 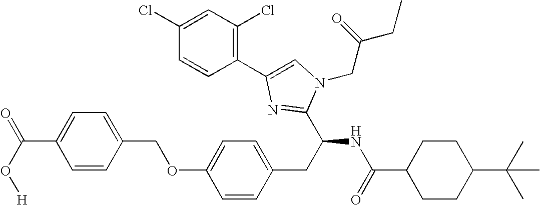 | 4-(4-{(2S)-2-[(4-tert-Butyl- cyclohexanecarbonyl)- amino]-2-[4-(2,4-dichloro- phenyl)-1-(2-oxo-butyl)-1H- imidazol-2-yl]-ethyl}- phenoxymethyl)-benzoic acid |
| 142 | 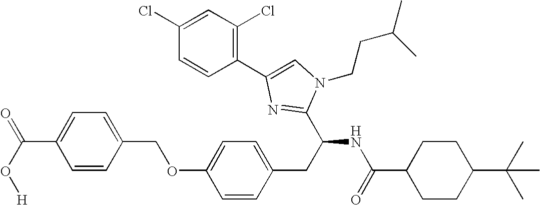 | 4-(4-{(2S)-2-[(4-tert-Butyl- cyclohexanecarbonyl)- amino]-2-[4-(2,4-dichloro- phenyl)-1-(3-methyl-butyl)- 1H-imidazol-2-yl]-ethyl}- phenoxymethyl)-benzoic acid |
| 143 | 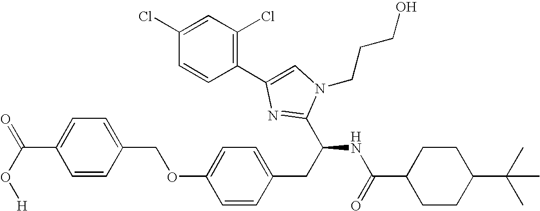 | 4-(4-{(2S)-2-[(4-tert-Butyl- cyclohexanecarbonyl)- amino]-2-[4-(2,4-dichloro- phenyl)-1-(3-hydroxy- propyl)-1H-imidazol-2-yl]- ethyl}-phenoxymethyl)- benzoic acid |
| 144 | 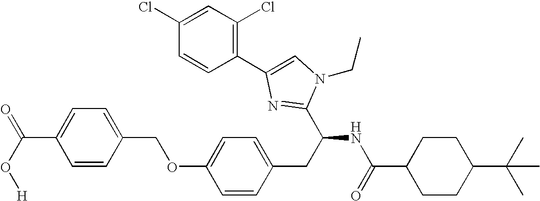 | 4-(4-{(2S)-2-[(4-tert-Butyl- cyclohexanecarbonyl)- amino]-2-[4-(2,4-dichloro- phenyl)-1-ethyl-1H-imidazol- 2-yl]-ethyl}-phenoxymethyl)- benzoic acid |
| 145 | 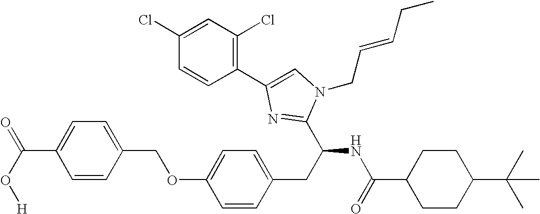 | 4-(4-{(2S)-2-[(4-tert-Butyl- cyclohexanecarbonyl)- amino]-2-[4-(2,4-dichloro- phenyl)-(E)-1-pent-2-enyl- 1H-imidazol-2-yl]-ethyl}- phenoxymethyl)-benzoic acid |
| 146 | 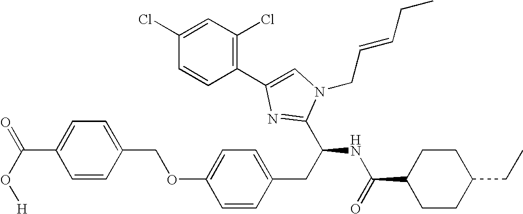 | 4-(4-{2-[4-(2,4-Dichloro- phenyl)-(E)-1-pent-2-enyl- 1H-imidazol-2-yl]-(2S)-2- [(trans-4-ethyl- cyclohexanecarbonyl)- amino]-ethyl}- phenoxymethyl)-benzoic acid |
| 147 | 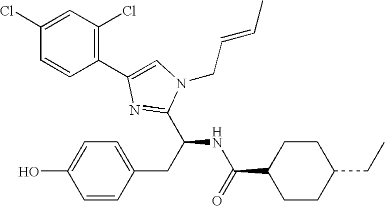 | Trans-4-Ethyl- cyclohexanecarboxylic acid [(1S)-1-[(E)-1-but-2-enyl-4- (2,4-dichloro-phenyl)-1H- imidazol-2-yl]-2-(4-hydroxy- phenyl)-ethyl]-amide |
| 148 | 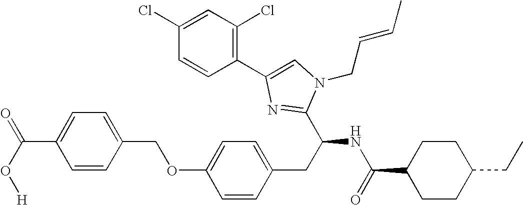 | 4-(4-{2-[(E)-1-But-2-enyl-4- (2,4-dichloro-phenyl)-1H- imidazol-2-yl]-(2S)-2- [(trans-4-ethyl- cyclohexanecarbonyl)- amino]-ethyl}- phenoxymethyl)-benzoic acid |
| 149 | 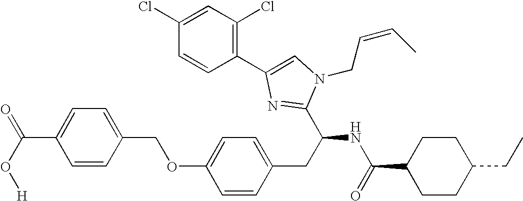 | 4-(4-{2-[(Z)-But-2-en-1-yl-4- (2,4-dichloro-phenyl)-1H- imidazol-2-yl]-(2S)-2- [(trans-4-ethyl- cyclohexanecarbonyl)- amino]-ethyl}- phenoxymethyl)-benzoic acid |
| 150 | 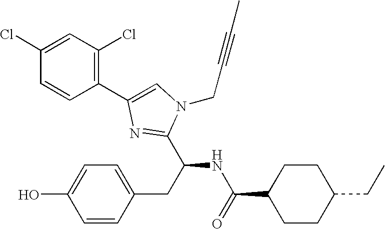 | Trans-4-Ethyl- cyclohexanecarboxylic acid [(1S)-1-[1-but-2-ynyl-4-(2,4- dichloro-phenyl)-1H- imidazol-2-yl]-2-(4-hydroxy- phenyl)-ethyl]-amide |
| 151 | 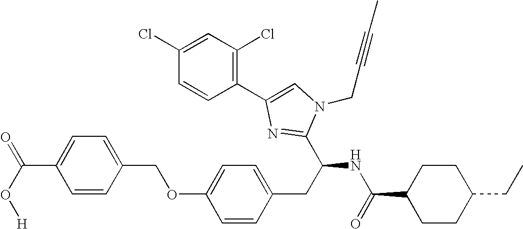 | 4-(4-{2-[1-But-2-ynyl-4-(2,4- dichloro-phenyl)-1H- imidazol-2-yl]-(2S)-2- [(trans-4-ethyl- cyclohexanecarbonyl)- amino]-ethyl}- phenoxymethyl)-benzoic acid |
| 152 | 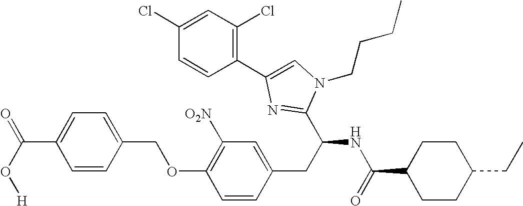 | 4-(4-{2-[1-Butyl-4-(2,4- dichloro-phenyl)-1H- imidazol-2-yl]-(2S)-2- [(trans-4-ethyl-cyclohexane- carbonyl)-amino]-ethyl}-2- nitro-phenoxymethyl)- benzoic acid |
| 153 | 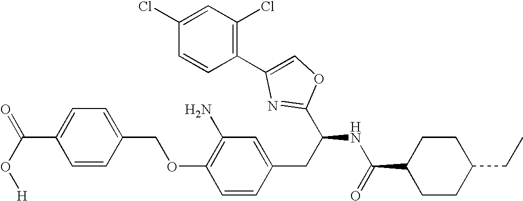 | 4-(2-Amino-4-{2-[4-(2,4- dichloro-phenyl)-oxazol-2- yl]-(2S)-2-[(trans-4-ethyl- cyclohexanecarbonyl)- amino]-ethyl}- phenoxymethyl)-benzoic acid |
| 154 | 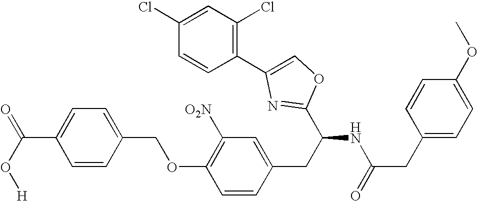 | 4-(4-{2-[4-(2,4-Dichloro- phenyl)-oxazol-2-yl]-(2S)-2- [2-(4-methoxy-phenyl)- acetylamino]-ethyl}-2-nitro- phenoxymethyl)-benzoic acid |
| 155 | 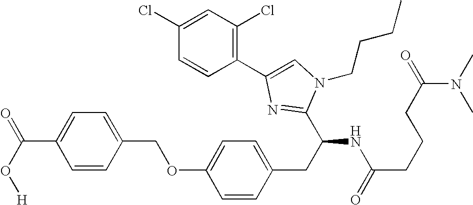 | 4-{4-[2-[1-Butyl-4-(2,4- dichloro-phenyl)-1H- imidazol-2-yl]-(2S)-2-(4- dimethyl-carbamoyl- butyrylamino)-ethyl]- phenoxymethyl}-benzoic acid |
| 156 | 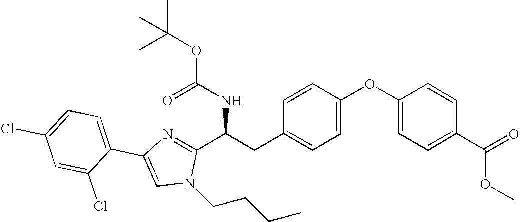 | 4-(4-{(2S)-2-tert- Butoxycarbonylamino-2-[1- butyl-4-(2,4-dichloro- phenyl)-1H-imidazol-2-yl]- ethyl}-phenoxy)-benzoic acid methyl ester |
| 157 | 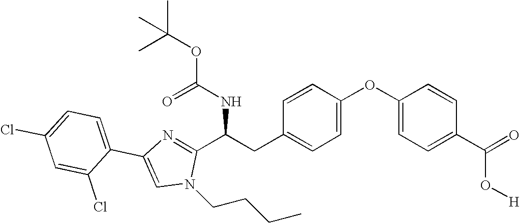 | 4-(4-{(2S)-2-tert- Butoxycarbonylamino-2-[1- butyl-4-(2,4-dichloro- phenyl)-1H-imidazol-2-yl]- ethyl}-phenoxy)-benzoic acid |
| 158 | 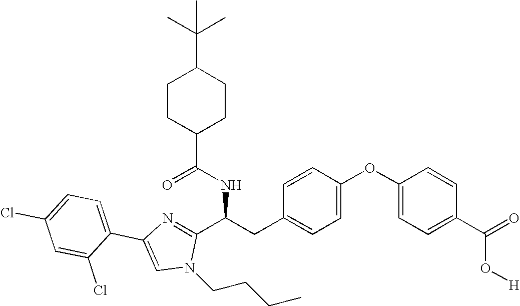 | 4-(4-{(2S)-2-[(4-tert-Butyl- cyclohexanecarbonyl)- amino]-2-[1-butyl-4-(2,4- dichloro-phenyl)-1H- imidazol-2-yl]-ethyl}- phenoxy)-benzoic acid |
| 159 | 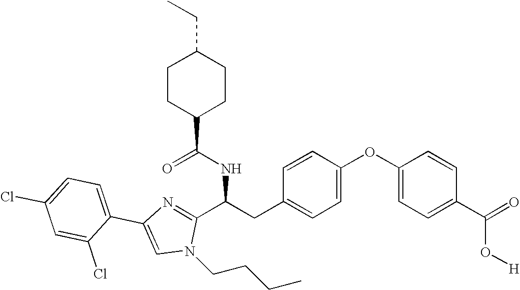 | 4-(4-{2-[1-Butyl-4-(2,4- dichloro-phenyl)-1H- imidazol-2-yl]-(2S)-2- [(trans-4-ethyl-cyclohexane- carbonyl)-amino]-ethyl}- phenoxy)-benzoic acid |
| 160 | 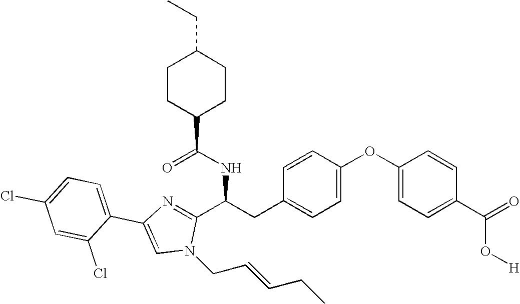 | 4-(4-{2-[4-(2,4-Dichloro- phenyl)-1-pent-2-enyl-1H- imidazol-2-yl]-(2S)-2- [(trans-4-ethyl-cyclohexane- carbonyl)-amino]-ethyl}- phenoxy)-benzoic acid |
| 161 | 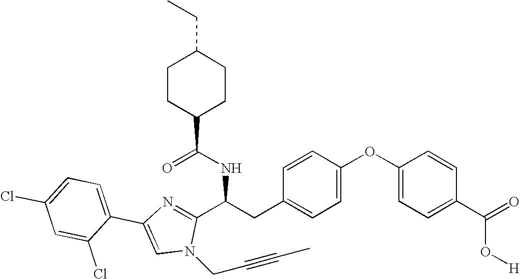 | 4-(4-{2-[1-But-2-ynyl-4-(2,4- dichloro-phenyl)-1H- imidazol-2-yl]-(2S)-2- [(trans-4-ethyl- cyclohexanecarbonyl)- amino]-ethyl}-phenoxy)- benzoic acid |
| 162 | 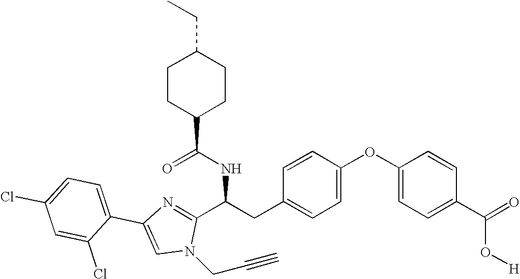 | 4-(4-{2-[4-(2,4-Dichloro- phenyl)-1-prop-2-ynyl-1H- imidazol-2-yl]-(2S)-2- [(trans-4-ethyl-cyclohexane- carbonyl)-amino]-ethyl}- phenoxy)-benzoic acid |
| 163 | 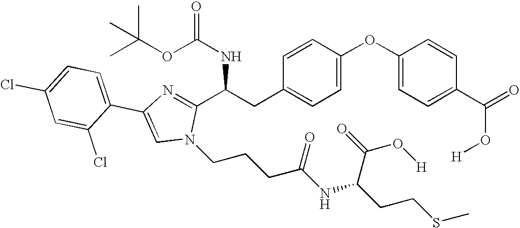 | 4-(4-{(2S)-2-tert- Butoxycarbonylamino-2-[1- [3-(1S)-1-carboxy-3- methylsulfanyl- propylcarbamoyl)-propyl]-4- (2,4-dichloro-phenyl)-1H- imidazol-2-yl]-ethyl}- phenoxy)-benzoic acid |
| 164 | 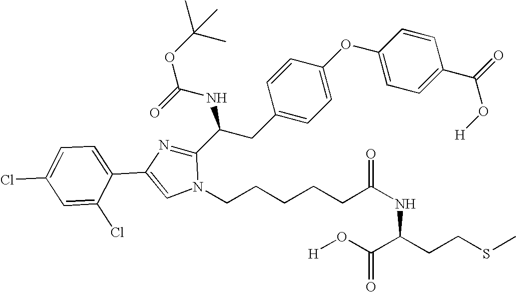 | 4-(4-{(2S)-2-tert- Butoxycarbonylamino-2-[1- [5-((1S)-1-carboxy-3- methylsulfanyl- propylcarbamoyl)-pentyl]-4- (2,4-dichloro-phenyl)-1H- imidazol-2-yl]-ethyl}- phenoxy)-benzoic acid |
| 165 | 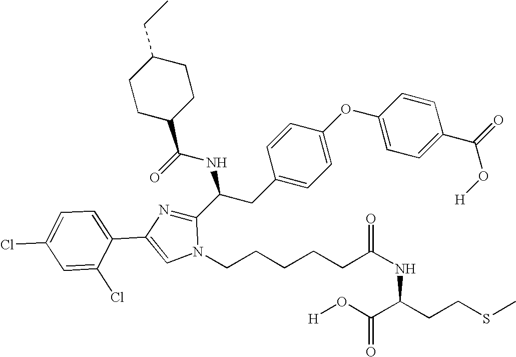 | 4-(4-{2-[1-(5-((1S)-1- Carboxy-3-methylsulfanyl- propylcarbamoyl)-pentyl]-4- (2,4-dichloro-phenyl)-1H- imidazol-2-yl]-(2S)-2- [(trans-4-ethyl- cyclohexanecarbonyl)- amino]-ethyl}-phenoxy)- benzoic acid |
| 166 | 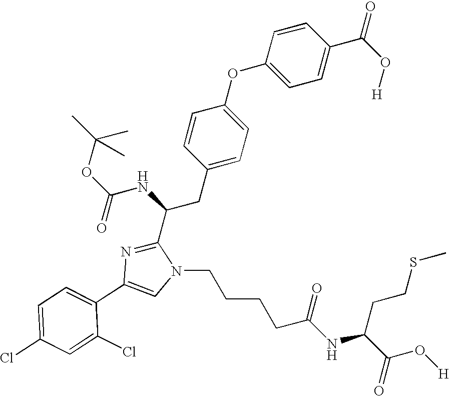 | 4-(4-{(2S)-2-tert- Butoxycarbonylamino-2-[1- [4-((1S)-1-carboxy-3- methylsulfanyl- propylcarbamoyl)-butyl]-4- (2,4-dichloro-phenyl)-1H- imidazol-2-yl]-ethyl}- phenoxy)-benzoic acid |
| 167 | 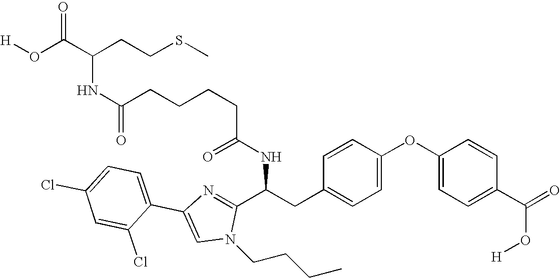 | 4-(4-{2-[1-Butyl-4-(2,4- dichloro-phenyl)-1H- imidazol-2-yl]-(2S)-2-[5-(1- carboxy-3-methylsulfanyl- propylcarbamoyl)- pentanoylamino]-ethyl}- phenoxy)-benzoic acid |
| 168 | 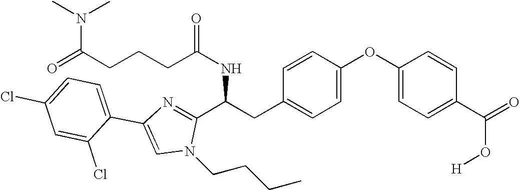 | 4-{4-[2-[1-Butyl-4-(2,4- dichloro-phenyl)-1H- imidazol-2-yl]-(2S)-2-(4- dimethylcarbamoyl- butyrylamino)-ethyl]- phenoxy}-benzoic acid |
| 169 | 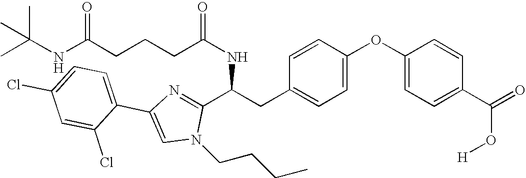 | 4-(4-{(2S)-2-(4-tert- Butylcarbamoyl- butyrylamino)-2-[1-butyl-4- (2,4-dichloro-phenyl)-1H- imidazol-2-yl]-ethyl}- phenoxy)-benzoic acid |
| 170 | 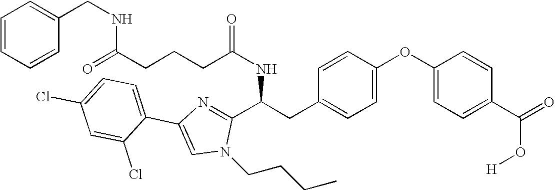 | 4-(4-{(2S)-2-(4- Benzylcarbamoyl- butyrylamino)-2-[1-butyl-4- (2,4-dichloro-phenyl)-1H- imidazol-2-yl]-ethyl}- phenoxy)-benzoic acid |
| 171 | 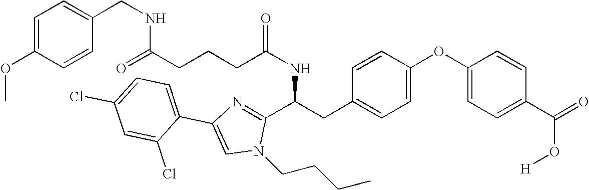 | 4-(4-{2-[1-Butyl-4-(2,4- dichloro-phenyl)-1H- imidazol-2-yl]-(2S)-2-[4-(4- methoxy-benzylcarbamoyl)- butyrylamino]-ethyl}- phenoxy)-benzoic acid |
| 172 | 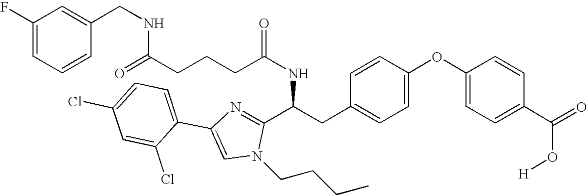 | 4-(4-{2-[1-Butyl-4-(2,4- dichloro-phenyl)-1H- imidazol-2-yl]-(2S)-2-[4-(3- fluorobenzylcarbamoyl)- butyrylamino]-ethyl}- phenoxy)-benzoic acid |
| 173 | 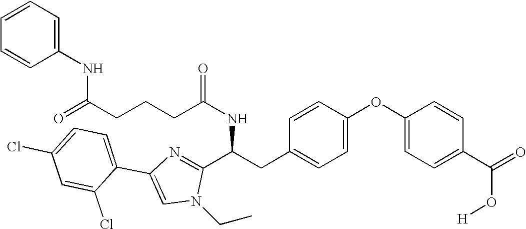 | 4-{4-[2-[4-(2,4-Dichloro- phenyl)-1-ethyl-1H-imidazol- 2-yl]-(2S)-2-(4- phenylcarbamoyl- butyrylamino)-ethyl]- phenoxy}-benzoic acid |
| 174 | 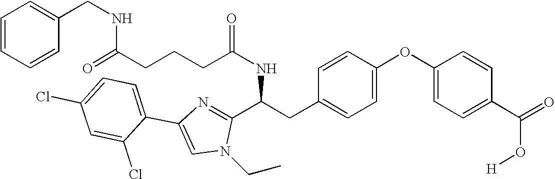 | 4-(4-{(2S)-2-(4- Benzylcarbamoyl- butyrylamino)-2-[4-(2,4- dichloro-phenyl)-1-ethyl-1H- imidazol-2-yl]-ethyl}- phenoxy)-benzoic acid |
| 175 | 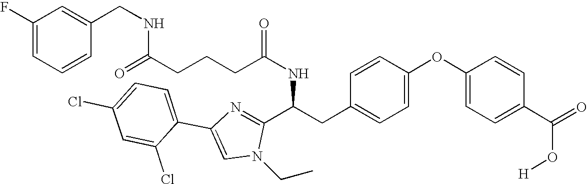 | 4-(4-{2-[4-(2,4-Dichloro- phenyl)-1-ethyl-1H-imidazol- 2-yl]-(2S)-2-[4-(3-fluoro- benzyl-carbamoyl)- butyrylamino]-ethyl}- phenoxy)-benzoic acid |
| 176 | 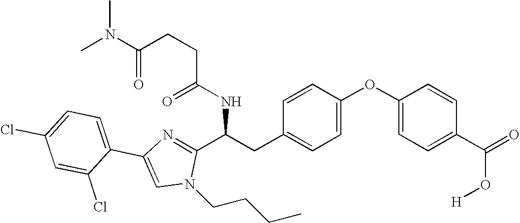 | 4-{4-[2-[1-Butyl-4-(2,4- dichloro-phenyl)-1H- imidazol-2-yl]-(2S)-2-(3- dimethylcarbamoyl- propionylamino)-ethyl]- phenoxy}-benzoic acid |
| 177 | 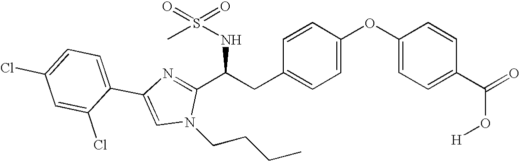 | 4-(4-{2-[1-Butyl-4-(2,4- dichloro-phenyl)-1H- imidazol-2-yl]-(2S)-2- methanesulfonylamino- ethyl}-phenoxy)-benzoic acid |
| 178 | 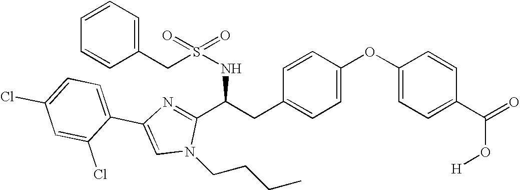 | 4-(4-{2-[1-Butyl-4-(2,4- dichloro-phenyl)-1H- imidazol-2-yl]-(2S)-2- phenylmethanesulfonyl- amino-ethyl}-phenoxy)- benzoic acid |
| 179 | 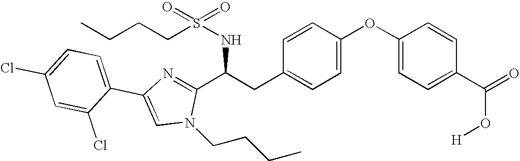 | 4-(4-{(2S)-2-(Butane-1- sulfonylamino)-2-[1-butyl-4- (2,4-dichloro-phenyl)-1H- imidazol-2-yl]-ethyl}- phenoxy)-benzoic acid |
| 180 | 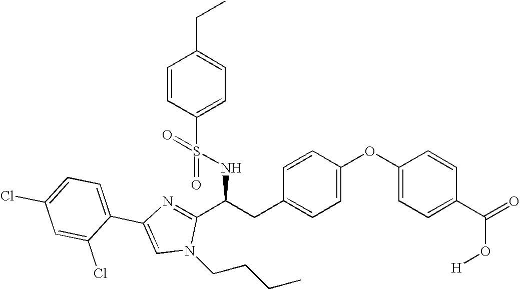 | 4-{4-[2-[1-Butyl-4-(2,4- dichloro-phenyl)-1H- imidazol-2-yl]-(2S)-2-(4- ethyl-benzenesulfonyl- amino)-ethyl]-phenoxy}- benzoic acid |
| 181 | 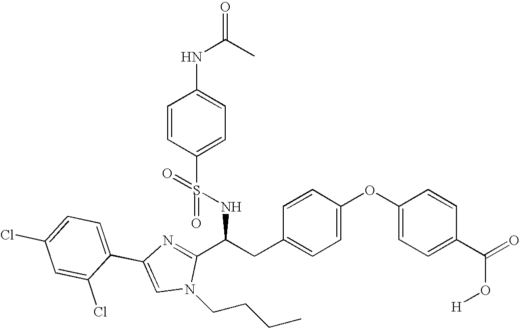 | 4-(4-{(2S)-2-(4-Acetylamino- benzenesulfonylamino)-2- [1-butyl-4-(2,4-dichloro- phenyl)-1H-imidazol-2-yl]- ethyl}-phenoxy)-benzoic acid |
| 182 | 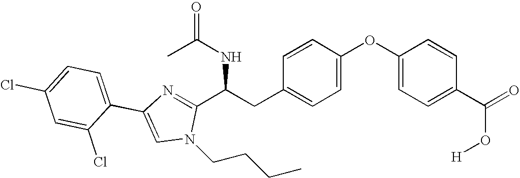 | 4-(4-{(2S)-2-Acetylamino-2- [1-butyl-4-(2,4-dichloro- phenyl)-1H-imidazol-2-yl]- ethyl}-phenoxy)-benzoic acid |
| 183 | 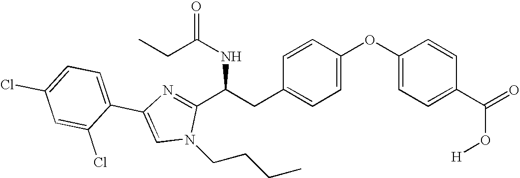 | 4-(4-{2-[1-Butyl-4-(2,4- dichloro-phenyl)-1H- imidazol-2-yl]-(2S)-2- propionylamino-ethyl}- phenoxy)-benzoic acid |
| 184 | 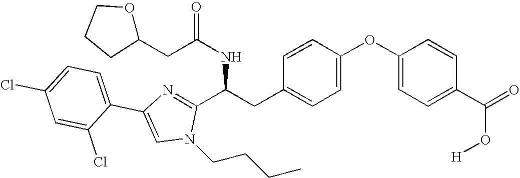 | 4-{4-[2-[1-Butyl-4-(2,4- dichloro-phenyl)-1H- imidazol-2-yl]-(2S)-2-(2- tetrahydro-furan-2-yl- acetylamino)-ethyl]- phenoxy}-benzoic acid |
| 185 | 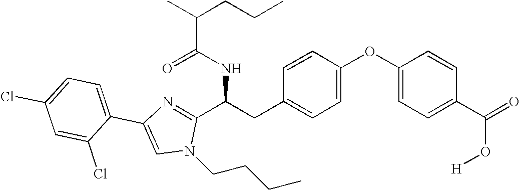 | 4-{4-[2-[1-Butyl-4-(2,4- dichloro-phenyl)-1H- imidazol-2-yl]-(2S)-2-(2- methyl-pentanoylamino)- ethyl]-phenoxy}-benzoic acid |
| 186 | 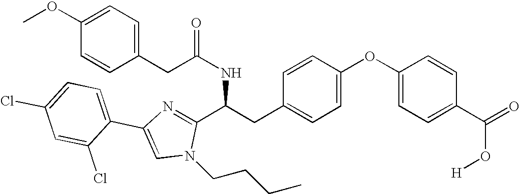 | 4-(4-{2-(1-Butyl-4-(2,4- dichloro-phenyl)-1H- imidazol-2-yl]-(2S)-2-[2-(4- methoxy-phenyl)- acetylamino]-ethyl}- phenoxy)-benzoic acid |
| 187 | 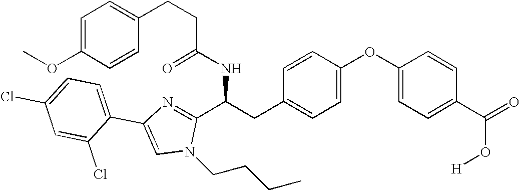 | 4-(4-{2-[1-Butyl-4-(2,4- dichloro-phenyl)-1H- imidazol-2-yl]-(2S)-2-[3-(4- methoxy-phenyl)- propionylamino]-ethyl}- phenoxy)-benzoic acid |
| 188 | 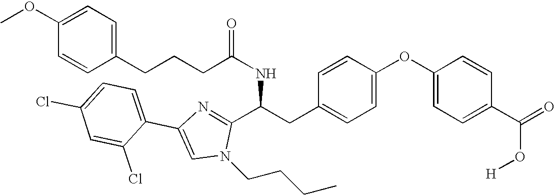 | 4-(4-{2-[1-Butyl-4-(2,4- dichloro-phenyl)-1H- imidazol-2-yl]-(2S)-2-[4-(4- methoxy-phenyl)- butyrylamino]-ethyl}- phenoxy)-benzoic acid |
| 189 | 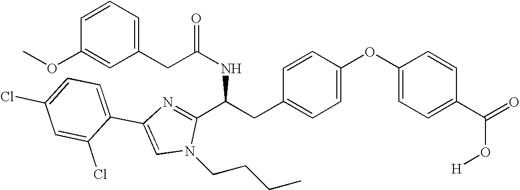 | 4-(4-{2-[1-Butyl-4-(2,4- dichloro-phenyl)-1H- imidazol-2-yl]-(2S)-2-[2-(3- methoxy-phenyl)- acetylamino]-ethyl}- phenoxy)-benzoic acid |
| 190 | 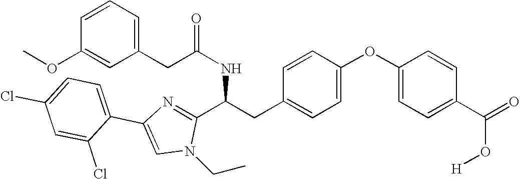 | 4-(4-{2-[4-(2,4-Dichloro- phenyl)-1-ethyl-1H-imidazol- 2-yl]-(2S)-2-[2-(3-methoxy- phenyl)-acetylamino]-ethyl}- phenoxy)-benzoic acid |
| 191 | 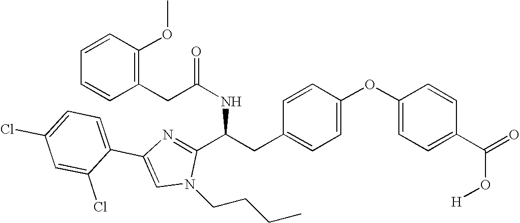 | 4-(4-{2-[1-Butyl-4-(2,4- dichloro-phenyl)-1H- imidazol-2-yl]-(2S)-2-[2-(2- methoxy-phenyl)- acetylamino]-ethyl}- phenoxy)-benzoic acid |
| 192 | 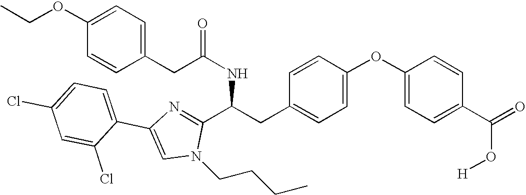 | 4-(4-{2-[1-Butyl-4-(2,4- dichloro-phenyl)-1H- imidazol-2-yl]-(2S)-2-[2-(4- ethoxy-phenyl)- acetylamino]-ethyl}- phenoxy)-benzoic acid |
| 193 | 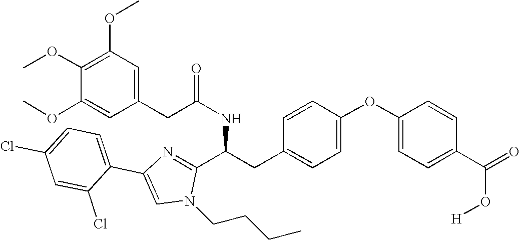 | 4-(4-{2-[1-Butyl-4-(2,4- dichloro-phenyl)-1H- imidazol-2-yl]-(2S)-2-[2- (3,4,5-trimethoxy-phenyl)- acetylamino]-ethyl}- phenoxy)-benzoic acid |
| 194 | 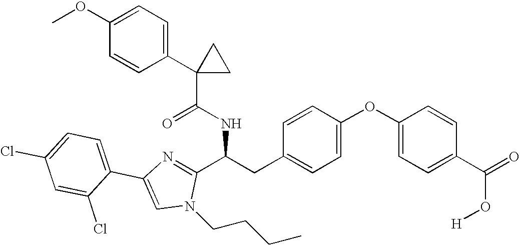 | 4-[4-(2-[1-Butyl-4-(2,4- dichloro-phenyl)-1H- imidazol-2-yl]-(2S)-2-{[1-(4- methoxy-phenyl)- cyclopropanecarbonyl]- amino}-ethyl)-phenoxy]- benzoic acid |
| 195 | 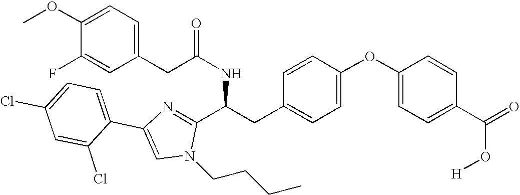 | 4-(4-{2-[1-Butyl-4-(2,4- dichloro-phenyl)-1H- imidazol-2-yl]-(2S)-2-[2-(3- fluoro-4-methoxy-phenyl)- acetylamino]-ethyl}- phenoxy)-benzoic acid |
| 196 | 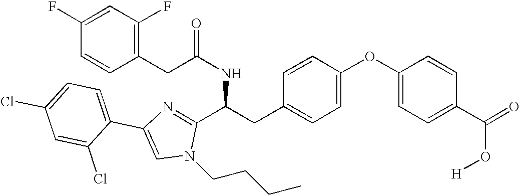 | 4-(4-{2-[1-Butyl-4-(2,4- dichloro-phenyl)-1H- imidazol-2-yl]-(2S)-2-[2- (2,4-difluorophenyl)- acetylamino]-ethyl}- phenoxy)-benzoic acid |
| 197 | 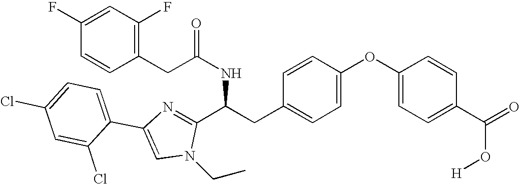 | 4-(4-{2-[4-(2,4-Dichloro- phenyl)-1-ethyl-1H-imidazol- 2-yl]-(2S)-2-[2-(2,4-difluoro- phenyl)-acetylamino]-ethyl}- phenoxy)-benzoic acid |
| 198 | 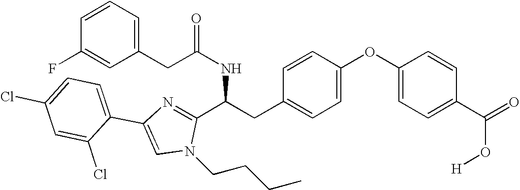 | 4-(4-{2-[1-Butyl-4-(2,4- dichloro-phenyl)-1H- imidazol-2-yl]-(2S)-2-[2-(3- fluoro-phenyl)-acetylamino]- ethyl}-phenoxy)-benzoic acid |
| 199 | 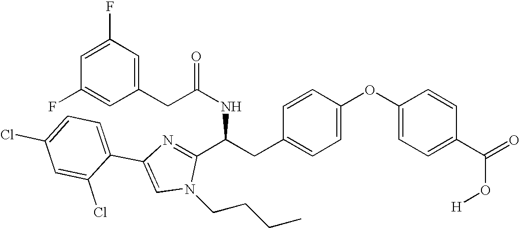 | 4-(4-{2-[1-Butyl-4-(2,4- dichloro-phenyl)-1H- imidazol-2-yl]-(2S)-2-[2- (3,5-difluoro-phenyl)- acetylamino]-ethyl}- phenoxy)-benzoic acid |
| 200 | 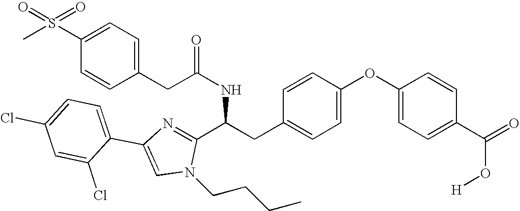 | 4-(4-{2-[1-Butyl-4-(2,4- dichloro-phenyl)-1H- imidazol-2-yl]-(2S)-2-[2-(4- methanesulfonyl-phenyl)- acetylamino]-ethyl}- phenoxy)-benzoic acid |
| 201 | 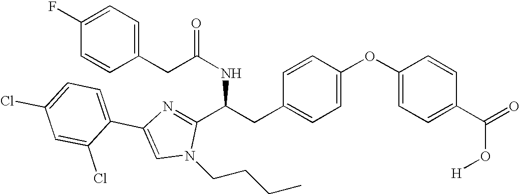 | 4-(4-{2-[1-Butyl-4-(2,4- dichloro-phenyl)-1H- imidazol-2-yl]-(2S)-2-[2-(4- fluoro-phenyl)-acetylamino]- ethyl}-phenoxy)-benzoic acid |
| 202 | 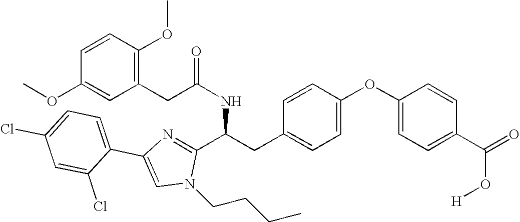 | 4-(4-{2-[1-Butyl-4-(2,4- dichloro-phenyl)-1H- imidazol-2-yl]-(2S)-2-[2- (2,5-dimethoxy-phenyl)- acetylamino]-ethyl}- phenoxy)-benzoic acid |
| 203 | 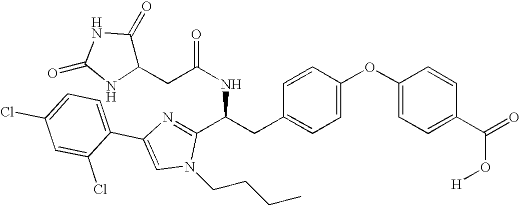 | 4-(4-{2-[1-Butyl-4-(2,4- dichloro-phenyl)-1H- imidazol-2-yl]-(2S)-2-[2- (2,5-dioxo-imidazolidin-4- yl)-acetylamino]-ethyl}- phenoxy)-benzoic acid |
| 204 | 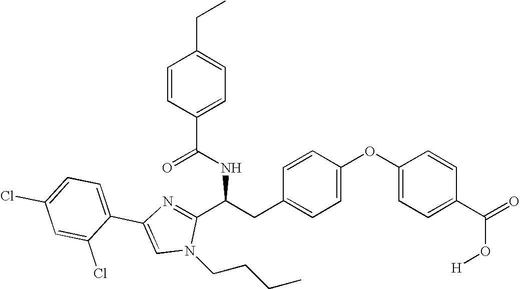 | 4-{4-[2-[1-Butyl-4-(2,4- dichloro-phenyl)-1H- imidazol-2-yl]-(2S)-2-(4- ethyl-benzoylamino)-ethyl]- phenoxy}-benzoic acid |
| 205 | 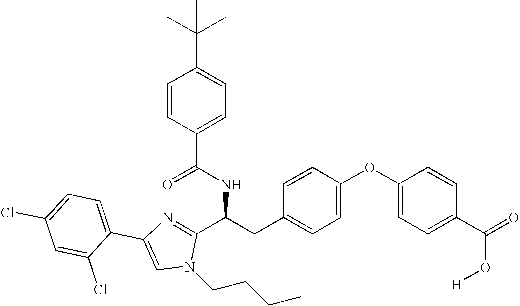 | 4-(4-{(2S)-2-(4-tert-Butyl- benzoylamino)-2-[1-butyl-4- (2,4-dichloro-phenyl)-1H- imidazol-2-yl]-ethyl}- phenoxy)-benzoic acid |
| 206 | 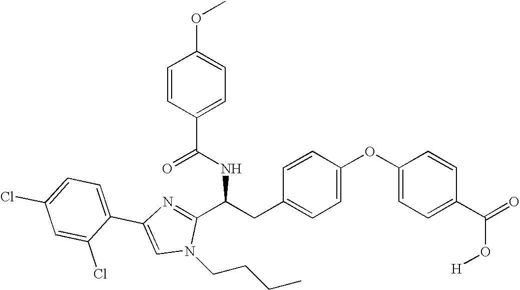 | 4-{4-[2-[1-Butyl-4-(2,4- dichloro-phenyl)-1H- imidazol-2-yl]-(2S)-2-(4- methoxy-benzoylamino)- ethyl]-phenoxy}-benzoic acid |
| 207 | 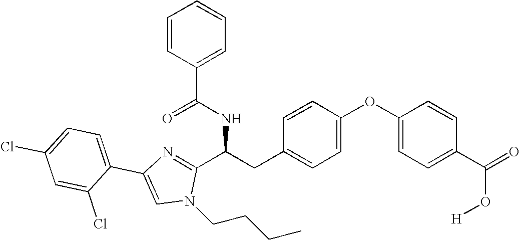 | 4-(4-{(2S)-2-Benzoylamino- 2-[1-butyl-4-(2,4-dichloro- phenyl)-1H-imidazol-2-yl]- ethyl}-phenoxy)-benzoic acid |
| 208 | 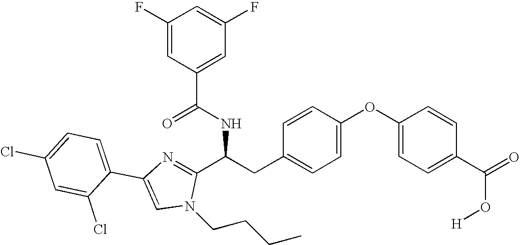 | 4-{4-[2-[1-Butyl-4-(2,4- dichloro-phenyl)-1H- imidazol-2-yl]-(2S)-2-(3,5- difluoro-benzoylamino)ethyl]- phenoxy}-benzoic acid |
| 209 | 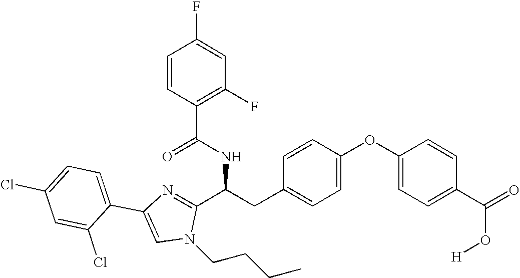 | 4-{4-[2-[1-Butyl-4-(2,4- dichloro-phenyl)-1H- imidazol-2-yl]-(2S)-2-(2,4- difluoro-benzoylamino)- ethyl]-phenoxy}-benzoic acid |
| 210 | 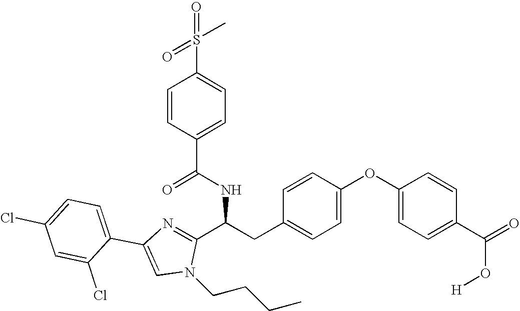 | 4-{4-[2-[1-Butyl-4-(2,4- dichloro-phenyl)-1H- imidazol-2-yl]-(2S)-2-(4- methanesulfonyl- benzoylamino)-ethyl]- phenoxy}-benzoic acid |
| 211 | 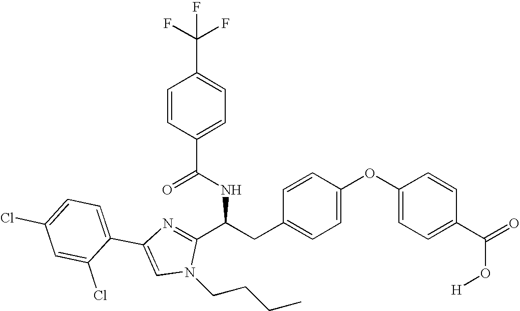 | 4-{4-[2-[1-Butyl-4-(2,4- dichloro-phenyl)-1H- imidazol-2-yl]-(2S)-2-(4- trifluoromethyl- benzoylamino)-ethyl]- phenoxy}-benzoic acid |
| 212 | 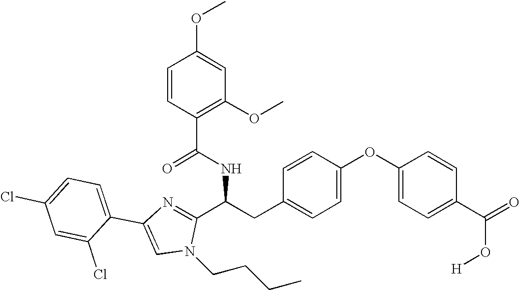 | 4-{4-[2-[1-Butyl-4-(2,4- dichloro-phenyl)-1H- imidazol-2-yl]-(2S)-2-(2,4- dimethoxy-benzoylamino)- ethyl]-phenoxy}-benzoic acid |
| 213 | 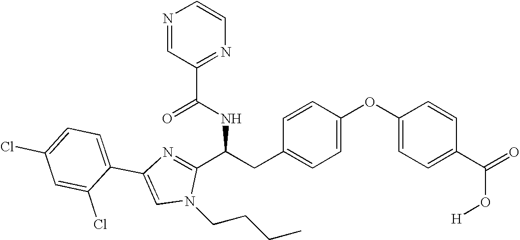 | 4-(4-{2-[1-Butyl-4-(2,4- dichloro-phenyl)-1H- imidazol-2-yl]-(2S)-2- [(pyrazine-2-carbonyl)- amino]-ethyl}-phenoxy)- benzoic acid |
| 214 | 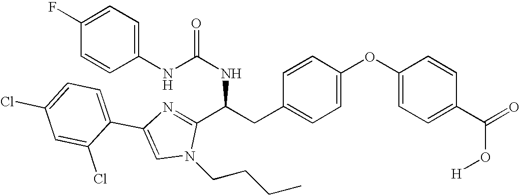 | 4-(4-{2-[1-Butyl-4-(2,4- dichloro-phenyl)-1H- imidazol-2-yl]-(2S)-2-[3-(4- fluoro-phenyl)-ureido]- ethyl}-phenoxy)-benzoic acid |
| 215 | 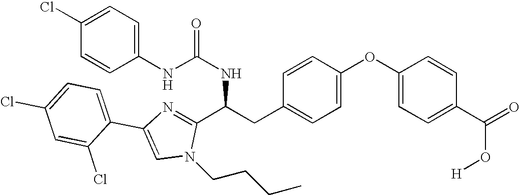 | 4-(4-{2-[1-Butyl-4-(2,4- dichloro-phenyl)-1H- imidazol-2-yl]-(2S)-2-[3-(4- chloro-phenyl)-ureido]- ethyl}-phenoxy)-benzoic acid |
| 216 | 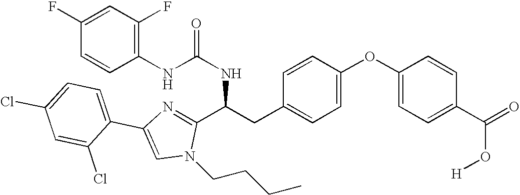 | 4-(4-{2-[1-Butyl-4-(2,4- dichloro-phenyl)-1H- imidazol-2-yl]-(2S)-2-[3- (2,4-difluoro-phenyl)- ureido]-ethyl}-phenoxy)- benzoic acid |
| 217 | 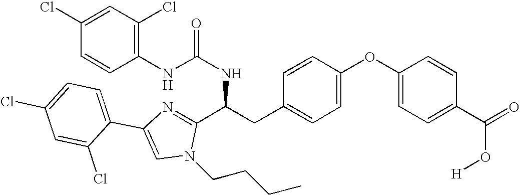 | 4-(4-{2-[1-Butyl-4-(2,4- dichloro-phenyl)-1H- imidazol-2-yl]-(2S)-2-[3- (2,4-dichloro-phenyl)- ureido]-ethyl}-phenoxy)- benzoic acid |
| 218 | 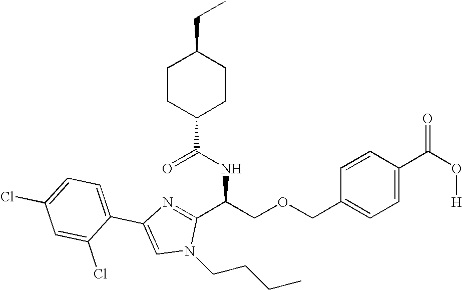 | 4-{2-[1-Butyl-4-(2,4- dichloro-phenyl)-1H- imidazol-2-yl]-(2S)-2- [(trans-4-ethyl- cyclohexanecarbonyl)- amino]-ethoxymethyl}- benzoic acid |
| 219 | 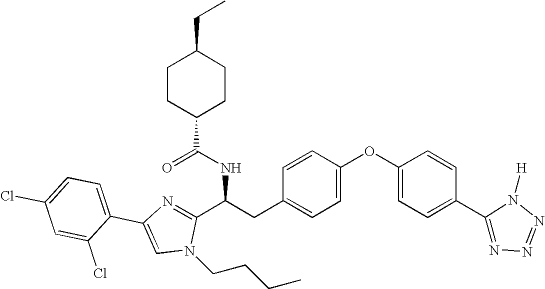 | Trans-4-Ethyl-cyclohexane- carboxylic acid((1S)-1-[1- butyl-4-(2,4-dichloro- phenyl)-1H-imidazol-2-yl]-2- {4-[4-(1H-tetrazol-5-yl)- phenoxy]-phenyl}-ethyl)-amide |
| 220 | 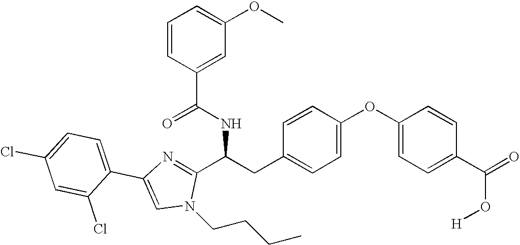 | 4-{4-[2-[1-Butyl-4-(2,4- dichloro-phenyl)-1H- imidazol-2-yl]-(2S)-2-(3- methoxy-benzoylamino)- ethyl]-phenoxy}-benzoic acid |
| 221 | 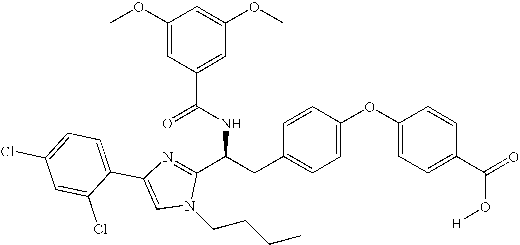 | 4-{4-[2-[1-Butyl-4-(2,4- dichloro-phenyl)-1H- imidazol-2-yl]-(2S)-2-(3,5- dimethoxy-benzoylamino)- ethyl]-phenoxy}-benzoic acid |
| 222 | 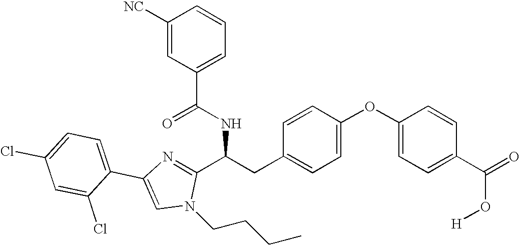 | 4-{4-[2-[1-Butyl-4-(2,4- dichloro-phenyl)-1H- imidazol-2-yl]-(2S)-2-(3- cyano-benzoylamino)-ethyl]- phenoxy}-benzoic acid |
| 223 | 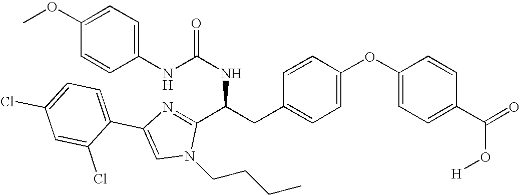 | 4-(4-{2-[1-Butyl-4-(2,4- dichloro-phenyl)-1H- imidazol-2-yl]-(2S)-2-[3-(4- methoxy-phenyl)-ureido]- ethyl}-phenoxy)-benzoic acid |
| 224 | 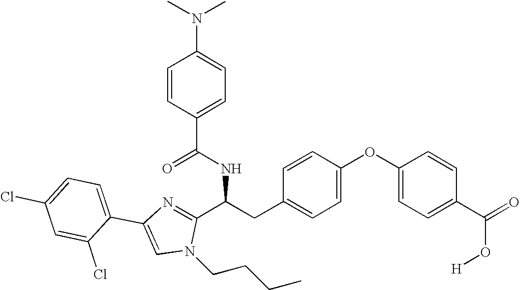 | 4-{4-[2-[1-Butyl-4-(2,4- dichloro-phenyl)-1H- imidazol-2-yl]-(2S)-2-(4- dimethylamino- benzoylamino)-ethyl]- phenoxy}-benzoic acid |
| 225 | 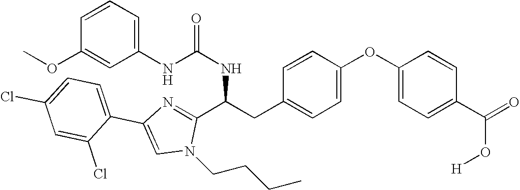 | 4-(4-{2-[1-Butyl-4-(2,4- dichloro-phenyl)-1H- imidazol-2-yl]-(2S)-2-[3-(3- methoxy-phenyl)-ureido]- ethyl}-phenoxy)-benzoic acid |
| 226 | 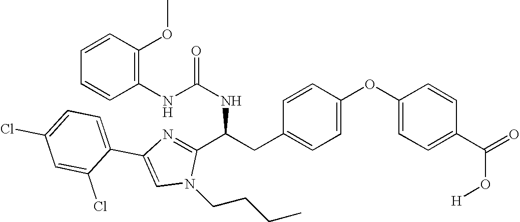 | 4-(4-{2-[1-Butyl-4-(2,4- dichloro-phenyl)-1H- imidazol-2-yl]-(2S)-2-[3-(2- methoxy-phenyl)-ureido]- ethyl}-phenoxy)-benzoic acid |
| 227 | 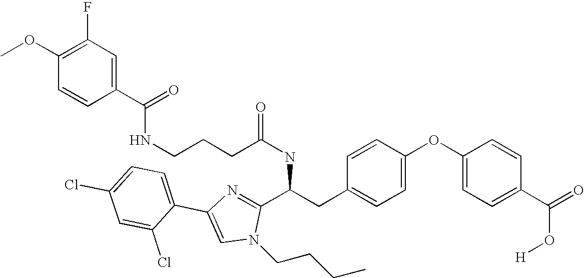 | 4-(4-{2-[1-Butyl-4-(2,4- dichloro-phenyl)-1H- imidazol-2-yl]-(2S)-2-[4-(3- fluoro-4-methoxybenzoyl- amino)-butyrylamino]-ethyl}- phenoxy)-benzoic acid |
| 228 | 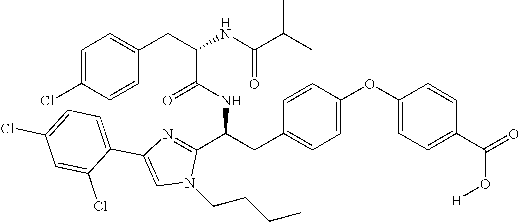 | 4-(4-{2-[1-Butyl-4-(2,4- dichloro-phenyl)-1H- imidazol-2-yl]-(2S)-2-[3-(4- chloro-phenyl)-(2S)-2- isobutyryl-amino-propionyl- amino]-ethyl}-phenoxy)- benzoic acid |
| 229 | 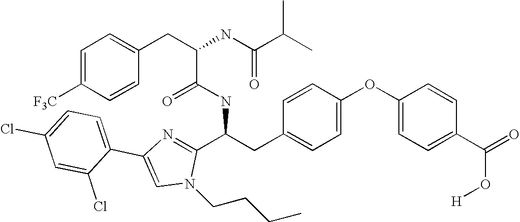 | 4-(4-{2-[1-Butyl-4-(2,4- dichloro-phenyl)-1H- imidazol-2-yl]-(2S)-2-[3-(4- trifluoromethyl-phenyl)-2- (2S)-isobutyrylamino- propionylamino]-ethyl}- phenoxy)-benzoic acid |
| 230 | 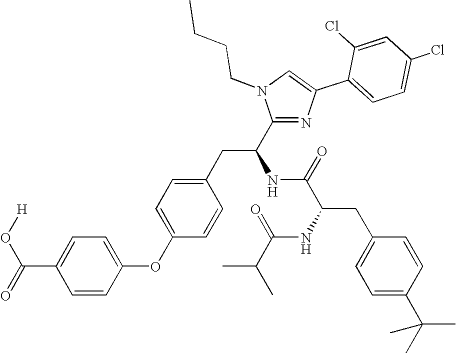 | 4-(4-{2-[1-Butyl-4-(2,4- dichloro-phenyl)-1H- imidazol-2-yl]-(2S)-2-[3-(4- tert-butyl-phenyl)-(2S)-2- isobutyrylamino- propionylamino]-ethyl}- phenoxy)-benzoic acid |
| 231 | 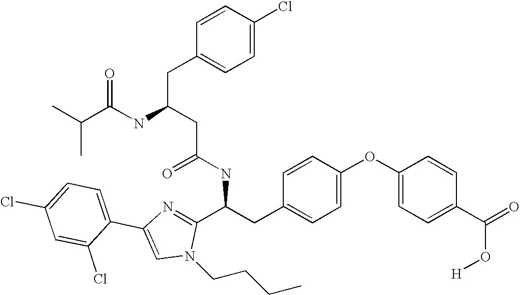 | 4-(4-{2-[1-Butyl-4-(2,4- dichloro-phenyl)-1H- imidazol-2-yl]-(2S)-2-[4-(4- chloro-phenyl)-(3S)-3- isobutyrylamino- butyrylamino]-ethyl}- phenoxy)-benzoic acid |
| 232 | 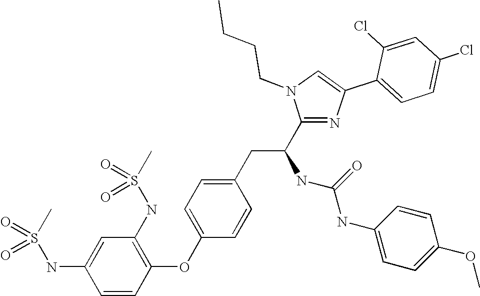 | N-[4-(4-{2-[1-Butyl-4-(2,4- dichloro-phenyl)-1H- imidazol-2-yl](2S)-2-[3-(4- methoxy-phenyl)-ureido]- ethyl}-phenoxy)-3- methanesulfonylamino- phenyl]methanesulfonamide |
| 233 | 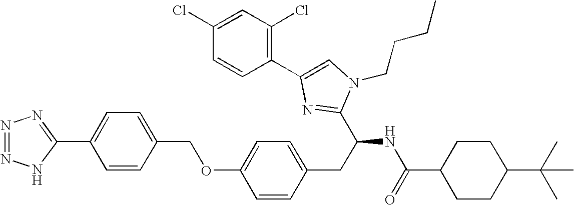 | 4-tert-Butyl- cyclohexanecarboxylic acid- ((1S)-1-[1-butyl-4-(2,4- dichloro-phenyl)-1H- imidazol-2-yl]-2-{4-[4-(1H- tetrazol-5-yl)-benzyloxy]- phenyl}-ethyl)-amide |

In the structures listed above, it is understood that where a heteroatom such as nitrogen or oxygen has an unfilled valence, a covalent bond exists between a hydrogen and the heteroatom.

In another aspect, the present invention comprises a pharmaceutical composition comprising the compound of Formula (I) and one or more pharmaceutically acceptable carriers, excipients, or diluents.

As used herein, the term “lower” refers to a group having between one and six carbons.

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “alkyl” as used herein include, but are not limited to, methyl, n-butyl, t-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ atoms. Examples of “alkylene” as used herein include, but are not limited to, methylene, ethylene, and the like.

As used herein, the term “alkyline” refers to a straight or branched chain trivalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “alkyline” as used herein include, but are not limited to, methine, ethyline, and the like.

As used herein, the term “alkenyl” refers to a hydrocarbon radical having from two to ten carbons and at least one carbon-carbon double bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ atoms.

As used herein, the term “alkenylene” refers to a straight or branched chain divalent hydrocarbon radical having from two to ten carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)2 atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ atoms.

As used herein, the term “alkynylene” refers to a straight or branched chain divalent hydrocarbon radical having from two to ten carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ atoms. Examples of “alkynylene” as used herein include, but are not limited to, ethyne-1,2-diyl, propyne-1,3-diyl, and the like.

As used herein, “cycloalkyl” refers to an alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “cycloalkyline” refers to an non-aromatic alicyclic trivalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkyline” as used herein include, but are not limited to, cyclopropyl-1,1,2-triyl, cyclohexyl-1,3,4-triyl, and the like.

As used herein, the term “heterocyclic” or the term “heterocyclyl” refers to a three to twelve-membered heterocyclic ring optionally possessing one or more degrees of unsaturation, containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, 2-tetrahydrofuryl, 1,4-dioxane-2-yl, 1,3-dioxane-2-yl, piperidin-1-yl, pyrrolidine-1-yl, morpholine-2-yl, piperazine-2-yl, and the like.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-dyil, and the like.

As used herein, the term “heterocyclyline” refers to a three to twelve-membered heterocyclic ring triradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclyline” include, but are not limited to, tetrahydrofuran-2,4,5-triyl, morpholine-2,3,4-triyl, pyran-2,4,5-triyl, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, alkoxycarbonylamino optionally substituted by alkyl, acylamino optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

As used herein, the term “aryline” refers to a benzene ring triradical or to a benzene ring system triradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “aryline” include, but are not limited to, benzene-1,2,4-triyl, naphthalene-1,4,8-triyl, and the like.

As used herein, the term “heteroaryl” refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1, 3-thiazole-2, 5-diyl, pyridine-2,4-diyl, pyridine-2, 3-diyl, pyridine-2,5-diyl, pyrimidine-2,4-diyl, quinoline-2,3-diyl, and the like.

As used herein, the term “heteroaryline” refers to a five- to seven-membered aromatic ring triradical, or to a polycyclic heterocyclic aromatic ring triradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroaryline” used herein are furan-2,4,5-triyl, thiophene-2,3,4-triyl, and the like.

As used herein, the term “fused cycloalkylaryl” refers to one or more cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl,

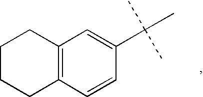

and the like.

As used herein, the term “fused cycloalkylarylene” refers to a fused cycloalkylaryl, wherein the aryl group is divalent. Examples include

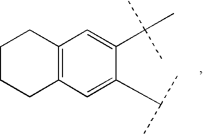

and the like.

As used herein, the term “fused arylcycloalkyl” refers to one or more aryl groups fused to a cycloalkyl group, the cycloalkyl and aryl groups having two atoms in common, and wherein the cycloalkyl group is the point of substitution. Examples of “fused arylcycloalkyl” used herein include 1-indanyl, 2-indanyl, 9-fluorenyl, 1-(1,2,3,4-tetrahydronaphthyl),

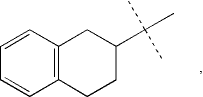

and the like.

As used herein, the term “fused arylcycloalkylene” refers to a fused arylcycloalkyl, wherein the cycloalkyl group is divalent. Examples include 9,1-fluorenylene,

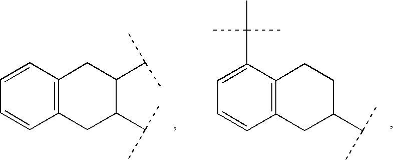

and the like.

As used herein, the term “fused heterocyclylaryl” refers to one or more heterocyclyl groups fused to an aryl group, the aryl and heterocyclyl groups having two atoms in common, and wherein the aryl group is the point of substitution. Examples of “fused heterocyclylaryl” used herein include 3,4-methylenedioxy-1-phenyl,

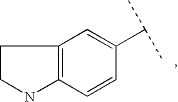

and the like

As used herein, the term “fused heterocyclylarylene” refers to a fused heterocyclylaryl, wherein the aryl group is divalent. Examples include

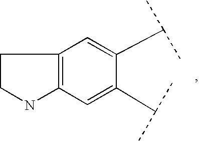

and the like.

As used herein, the term “fused arylheterocyclyl” refers to one or more aryl groups fused to a heterocyclyl group, the heterocyclyl and aryl groups having two atoms in common, and wherein the heterocyclyl group is the point of substitution. Examples of “fused arylheterocyclyl” used herein include 2-(1,3-benzodioxolyl),

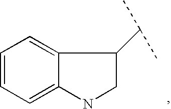

and the like.

As used herein, the term “fused arylheterocyclylene” refers to a fused arylheterocyclyl, wherein the heterocyclyl group is divalent. Examples include

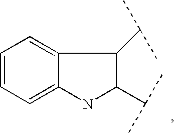

and the like.

As used herein, the term “fused cycloalkylheteroaryl” refers to one or more cycloalkyl groups fused to a heteroaryl group, the heteroaryl and cycloalkyl groups having two atoms in common, and wherein the heteroaryl group is the point of substitution. Examples of “fused cycloalkylheteroaryl” used herein include 5-aza-6-indanyl,

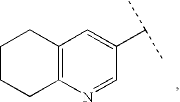

and the like.

As used herein, the term “fused cycloalkylheteroarylene” refers to a fused cycloalkylheteroaryl, wherein the heteroaryl group is divalent. Examples include

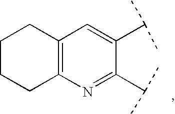

and the like.

As used herein, the term “fused heteroarylcycloalkyl” refers to one or more heteroaryl groups fused to a cycloalkyl group, the cycloalkyl and heteroaryl groups having two atoms in common, and wherein the cycloalkyl group is the point of substitution. Examples of “fused heteroarylcycloalkyl” used herein include 5-aza-1-indanyl,

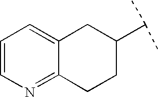

and the like.

As used herein, the term “fused heteroarylcycloalkylene” refers to a fused heteroarylcycloalkyl, wherein the cycloalkyl group is divalent. Examples include

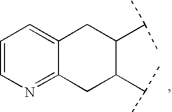

and the like.

As used herein, the term “fused heterocyclylheteroaryl” refers to one or more heterocyclyl groups fused to a heteroaryl group, the heteroaryl and heterocyclyl groups having two atoms in common, and wherein the heteroaryl group is the point of substitution. Examples of “fused heterocyclylheteroaryl” used herein include 1,2,3,4-tetrahydro-beta-carbolin-8-yl,

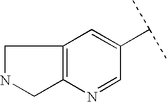

and the like.

As used herein, the term “fused heterocyclylheteroarylene” refers to a fused heterocyclylheteroaryl, wherein the heteroaryl group is divalent. Examples include

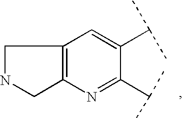

and the like.

As used herein, the term “fused heteroarylheterocyclyl” refers to one or more heteroaryl groups fused to a heterocyclyl group, the heterocyclyl and heteroaryl groups having two atoms in common, and wherein the heterocyclyl group is the point of substitution. Examples of “fused heteroarylheterocyclyl” used herein include -5-aza-2,3-dihydrobenzofuran-2-yl,

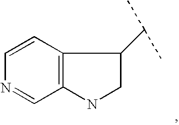

and the like.

As used herein, the term “fused heteroarylheterocyclylene” refers to a fused heteroarylheterocyclyl, wherein the heterocyclyl group is divalent. Examples include

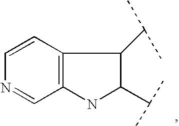

and the like.

As used herein, the term “acid isostere” refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, or 5-hydroxy-4H-pyran-4-on-2-yl.

As used herein, the term “side chain of a natural or non-natural amino acid” refers to the group “R” in a substance of formula HO₂C—CH(R)—NH₂. Examples of such substances bearing a group “R” include but are not limited to alanine, asparigine, arginine, aspartic acid, cystine, cysteine, glutamic acid, histidine, isoleucine, leucine, lysine, methionine, phenylalanine, serine, threonine, tryptophan, tyrosine, valine, alpha-aminoadipic acid, alpha-aminobutyric acid, norleucine, 3,4-dihydroxyphenylalanine, homoserine, and ornithine. Where such groups “R” bear carboxyl, hydroxyl, or amino functional groups, such functional groups may be protected. In addition, where groups “R” bear a sulfhydryl group, such a group may be protected in a form such as but not limited to a tert-butyl thioether, a benzyl thioether, or an alkanoyl thioester.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfenyl” refers to the group RₐS(O)—, where Rₐ is alkyl.

As used herein, the term “alkenylsulfenyl” refers to the group RₐS(O)—, where Rₐ is alkenyl.

As used herein, the term “alkynylsulfenyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “aroyloxy” refers to the group RₐC(O)O—, where Rₐ is aryl.

As used herein, the term “heteroaroyloxy” refers to the group RₐC(O)O—, where Rₐ is heteroaryl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” shall include iodine, bromine, chlorine and fluorine.

As used herein, the term “mercapto” shall refer to the substituent —SH.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)₂—.

The compounds can be prepared according to the following reaction Schemes (in which variables are as defined before or are defined) using readily available starting materials, reagents and conventional synthesis procedures. In these reactions, it is also possible to make use of variants which are themselves known to those of ordinary skill in this art, but are not mentioned in greater detail.

The present invention also provides a method for the synthesis of compounds useful as intermediates in the preparation of compounds of Formula (I) along with methods for the preparation of compounds of Formula (I). Variables refer to those defined for Formula (I) unless otherwise specified.

In Scheme 1 commercially available aromatic/substituted aromatic aldehydes (1) can be treated with malonic acid (2) in the presence of ammonium acetate at 80° C. to afford required β amino acids (3) (Johnson, T. B; Livak, J; J. Am Chem Soc, 1936 58, 299).

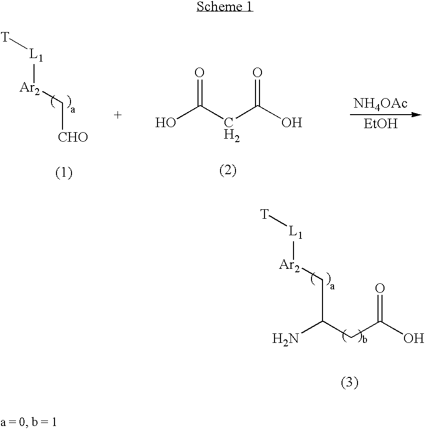

In Scheme 2, amino acids can be prepared by using Strecker synthesis, wherein commercially avialiable aromatic/substituted aromatic aldehydes (1) are treated with ammonium hydroxide, ammonium chloride and potassium cyanide to form cyano-hydrin (4), which are treated with 6N HCl to afford alpha-amino acids (5).

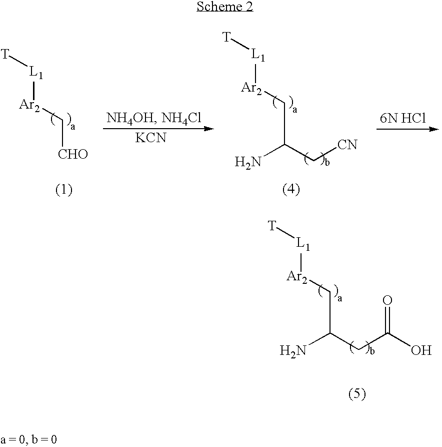

Scheme 3 refers to the attachment of an amino protecting group, PG₁, to an alpha-amino acid (5). For example, treatment of amino acids such as (3) and/or (5) with di-tert-butyl dicarbonate and a weak base such as sodium carbonate or sodium bicarbonate in an aqueous organic solvent such as dioxane/THF/EtOAc affords the N-tert-butyl carbamate (6).

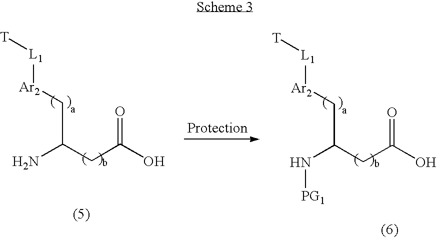

In Scheme 4, the protected amino acid (6) can be reacted with aryl acyl bromides (7) in the presence of base such as DIEA, triethyl amine, or DBU, in a polar solvents such as THF or DMF to afford intermediate keto-ester (8), which was treated with an amine source such as, but not limited to, ammonium acetate in acetic acid at temperatures ranging from 60-120° C., leading to corresponding mixture of oxazole (W═O) and imidazole (W═NH) (9) (Strzybny, P. P. E; van Es, T.; Backeberg, O. G. J. Org. Chem. 1963, 25, 1151). The ratio of oxazole and imidazole varies depending on the substitution and reaction conditions and the two compounds can be separated through silica gel chromatography. Alternatively other conditions may also be employed for cyclization of keto-esters (8), such as BF₃/Et₂O, methanolic ammonia, at temperatures ranging from room temperature to 120° C.

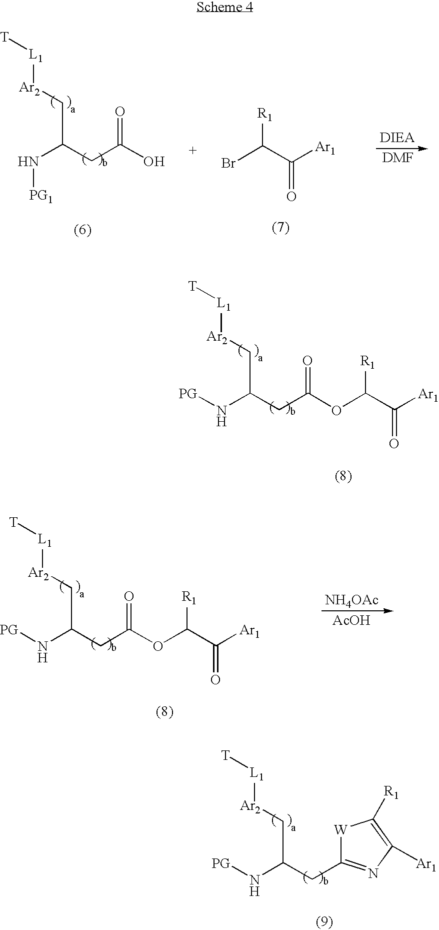

In Scheme 5, the protecting group (PG) may be removed by suitable methods known in the art. For example, where the PG comprises tert-butyl carbamate, treatment of (9) with 1,4-dioxane-HCl at room temperature affords (10).

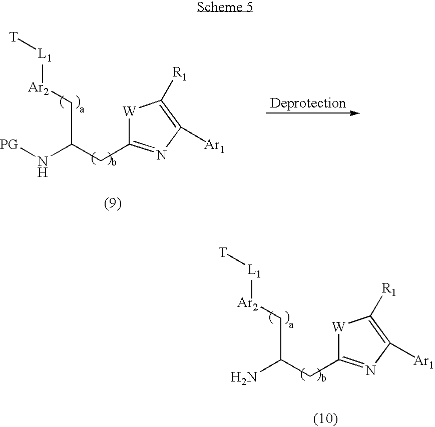

In another embodiment (Scheme 6), the free amine group in compound (10) may be acylated employing a carboxylic acid R₃₀CO₂H or a suitable derivative thereof. This transformation can be accomplished using standard methods. These methods include converting the desired acid into activated acid and reacting with amine (10). Methods to activate the carboxylic acid include reacting the acid with one or more equivalents of dicyclohexylcarbodiimide/diisopropylethyl amine or DIEA/HBTU with or without one or more molar equivalents of hydroxy benzotriazole in a suitable solvent such as dichloromethane or dimethylformamide at temperartures ranging from 0° C. to room temperature, affords compound (11). In this instance R₃₀ is a group such as, but not limited to, -alkyl or -alkylene-aryl.

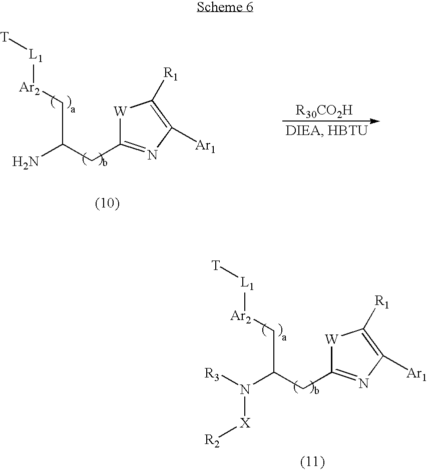

In another embodiment,(Scheme 7) amine group in compound (10) interacts with an aliphatic or aromatic aldehydes such as acetaldehyde, propionaldehyde, or benzaldehyde, and undergoes reduction with hydride source such as sodium borohydride or triacetoxy borohydride in a solvent such as THF or DCE at temperatures ranging from 0° C. to room temperature to afford compound (12).

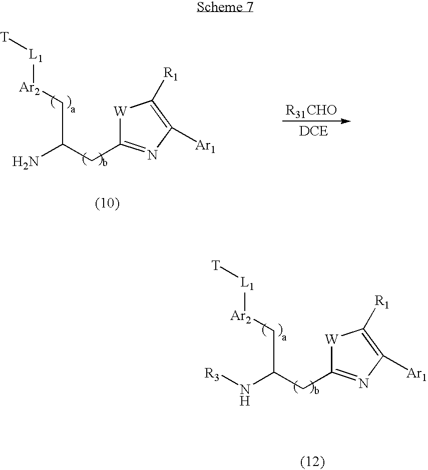

In another embodiment, an ester in compound (13) can be hydrolyzed using a base such as LiOH or NaOH, in a mixture of aqueous and organic solvents such as THF, methanol, at temperature ranging from room temperature to 60° C. to afford, after acidification with, for example, dilute mineral acid, free carboxylic acid (14). In this instance R₃₂ is a group such as, but not limited to, lower alkyl.

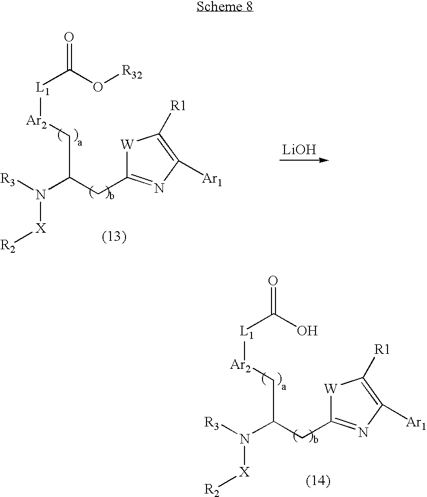

In another embodiment (Scheme 9) the aryl alkyl ether group in compound (15) can be dealkylated using reagents such as BBr₃, in the presence or absence of a cation scavenger such as PhSMe, in a solvent such as dichloromethane or DCMFTFA at temperatures ranging from −20° C. to room temperature to afford compound (16). In this instance, R₃₃ is a group such as, but not limited to, -alkyl or -alkylene-aryl.

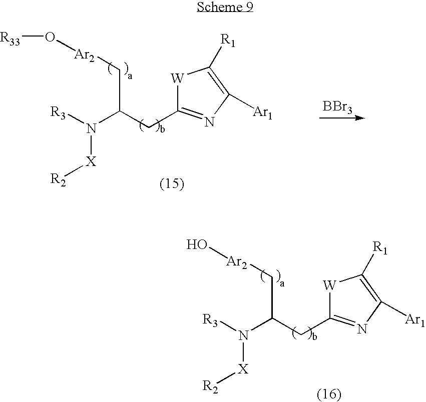

In another embodiment (Scheme 10) the phenol (16) can be alkylated with bromo alkyl carboxylates (17) such as Br—(CH₂)ₙ CO₂—R₃₃, where n may be, for example,1 to 6, in the presence of base such as sodium hydride, potassium tert-butoxide, or potassium carbonate using DMF, THF, acetonitrile as the solvent at temperatures ranging from 50° C. to 100° C. to afford the oxyalkyl esters (18). Subsequent saponification of esters (18) with base such as sodium hydroxide, or lithium hydroxide in aqueous and organic solvents such as THF, methanol, at temperatures ranging from room temperature to 60° C. can produce carboxylic acid (19). In this instance, R₃₃ is a group such as, but not limited to, -alkyl or -alkylene-aryl.

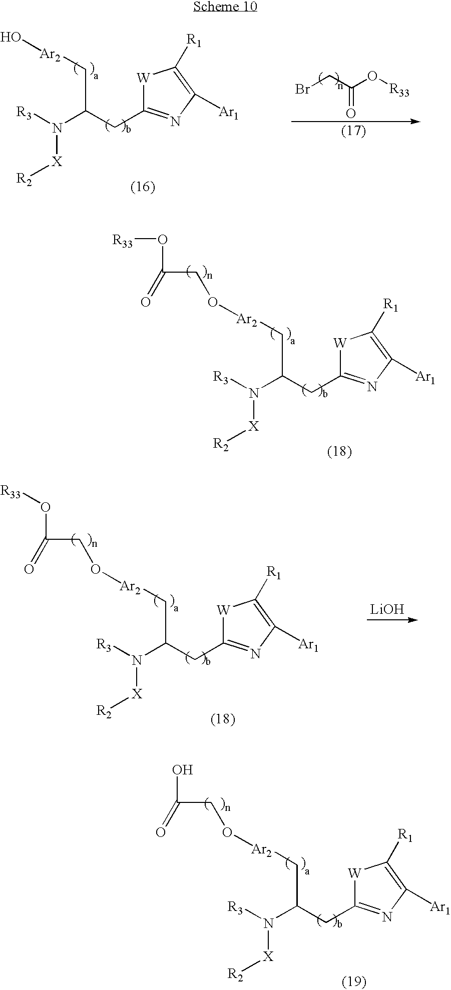

In another embodiment (Scheme 11) imidazole nitrogen in compound (20) can be alkylated with alkyl halides Br—(CH₂)ₙ—R₃₄ where n is, for example, 1 to 6, in the presence of base such as sodium hydride, potassium tert-butoxide, potassium carbonate using DMF, THF, acetonitrile as the solvent at temperatures ranging from 0° C. to 80° C. to afford N-alkylated products (22). In this instance R₃₄ is a group such as but not limited to -alkyl, -aryl, -alkenylene-aryl, or -alkylene-aryl.

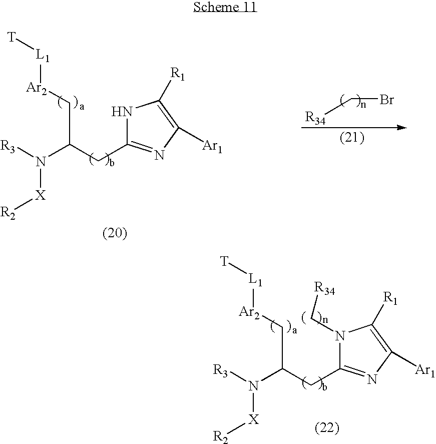

The term “amino protecting group” as used herein refers to substituents of the amino group commonly employed to block or protect the amino functionality while reacting other functional groups on the compound. Examples of such amino-protecting groups include the formyl group, the trityl group, the phthalimido group, the trichloroacetyl group, the chloroacetyl, bromoacetyl and iodoacetyl groups, urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxy-carbonyl, 2-(4-xenyl)iso-propoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxycarbonyl, cyclopentanyloxycarbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfonyl)ethoxycarbonyl, 2(methylsulfonyl)ethoxycarbonyl, 2-(triphenylphosphino)ethoxycarbonyl, 9-fluorenylmethoxycarbonyl (“FMOC”), t-butoxycarbonyl (“BOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonyl and the like; the benzoylmethylsulfonyl group, the 2-(nitro)phenylsulfenyl group, the diphenylphosphine oxide group and like amino-protecting groups. The species of amino-protecting group employed is not critical so long as the derivatized amino group is stable to the condition of subsequent reaction(s) on other positions of the compound of Formula (I) and can be removed at the desired point without disrupting the remainder of the molecule. In an embodiment, amino-protecting groups are the allyloxycarbonyl, the t-butoxycarbonyl, 9-fluorenylmethoxycarbonyl, and the trityl groups. Similar amino-protecting groups used in the cephalosporin, penicillin and peptide art are also embraced by the above terms. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected amino” or “protected amino group” defines an amino group substituted with an amino-protecting group discussed above.

The term “hydroxyl protecting group” as used herein refers to substituents of the alcohol or phenol group commonly employed to block or protect the alcohol or phenol functionality while reacting other functional groups on the compound. Examples of such hydroxyl -protecting groups include the 2-tetrahydropyranyl group, 2-ethoxyethyl group, the trityl group, the trichloroacetyl group, urethane-type blocking groups such as benzyloxycarbonyl, and the trialkylsilyl group, examples of such being trimethylsilyl, tert-butyldimethylsilyl, phenyidimethylsilyl, triiospropylsilyl and thexyldimethylsilyl. The choice of of hydroxyl-protecting group employed is not critical so long as the derivatized hydroxyl group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected hydroxyl” or “protected alcohol” defines a hydroxyl group substituted with a hydroxyl-protecting group as discussed above.

The term “carboxyl protecting group” as used herein refers to substituents of the carboxyl group commonly employed to block or protect the —OH functionality while reacting other functional groups on the compound. Examples of such alcohol-protecting groups include the 2-tetrahydropyranyl group, 2-ethoxyethyl group, the trityl group, the allyl group, the trimethylsilylethoxymethyl group, the 2,2,2-trichloroethyl group, the benzyl group, and the trialkylsilyl group, examples of such being trimethylsilyl, tert-butyldimethylsilyl, phenyldimethylsilyl, triiospropylsilyl and thexyldimethylsilyl. The choice of carboxyl protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected carboxyl” defines a carboxyl group substituted with a carboxyl-protecting group as discussed above.

General Experimental:

LC-MS data was obtained using gradient elution on a Waters 600 controller equipped with a 2487 dual wavelength detector and a Leap Technologies HTS PAL Autosampler using an YMC Combiscreen ODS-A 50×4.6 mm column. A three minute gradient was run from 25% B (97.5% acetonitrile, 2.5% water, 0.05% TFA) and 75% A (97.5% water, 2.5% acetonitrile, 0.05% TFA) to 100% B. The mass spectrometer used was a Micromass ZMD instrument. All data was obtained in the positive mode unless otherwise noted. ¹H NMR data was obtained on a Varian 400 MHz spectrometer.

Abbreviations used in the Examples are as follows:

APCI=atmospheric pressure chemical ionization

BOC=tert-butoxycarbonyl

BOP=(1-benzotriazolyloxy)tris(dimethylamino)phosphonium hexafluorophosphate

d=day

DIAD=diisopropyl azodicarboxylate

DCC=dicyclohexylcarbodiimide

DCM=dichloromethane

DIC=diisopropylcarbodiimide

DIEA=diisopropylethylamine

DMA=N,N-dimethylacetamide

DMAP=dimethylaminopyridine

DME=1,2 dimethoxyethane

DMF=N,N-dimethylformamide

DMPU=1,3-dimethypropylene urea

DMSO=dimethylsulfoxide

EDC=1-ethyl-3-(3-dimethylaminopropyl)-carbodiimide hydrochloride

EDTA=ethylenediamine tetraacetic acid

ELISA=enzyme-linked immunosorbent assay

ESI=electrospray ionization

ether=diethyl ether

EtOAc=ethyl acetate

FBS=fetal bovine serum

g=gram

h=hour

HBTU=O-benzotriazol-1-yl-N,N,N′,N′-tetramethyluronium hexafluorophosphate

HMPA=hexamethylphosphoric triamide

HOBt=1-hydroxybenzotriazole

Hz=hertz

i.v.=intravenous

kD=kiloDalton

L=liter

LC=liquid chromatography

LAH=lithium aluminum hydride

LDA=lithium diisopropylamide

LPS=lipopolysaccharide

M=molar

m/z=mass to charge ratio

mbar=millibar

MeOH=methanol

mg=milligram

min=minute

mL=milliliter

mM=millimolar

mmol=millimole

mol=mole

mp=melting point

MS=mass spectrometry

N=normal

NMM=N-methylmorpholine, 4-methylmorpholine

NMR=nuclear magnetic resonance spectroscopy

p.o.=per oral

PBS=phosphate buffered saline solution

PMA=phorbol myristate acetate

ppm=parts per million

psi=pounds per square inch

Rf=relative TLC mobility

rt=room temperature

s.c.=subcutaneous

SPA=scintillation proximity assay

TEA=triethylamine

TFA=trifluoroacetic acid

THF=tetrahydrofuran

THP=tetrahydropyranyl

TLC=thin layer chromatography

TMSBr=bromotrimethylsilane, trimethylsilylbromide

Tr=retention time

General Procedure A: Formation of Beta Amino Acid

1 equivalent of the desired aromatic aldehyde is dissolved in anhydrous ethanol (0.5-1.0 M). To the solution is added in succession 2 equivalents of ammonium acetate and 2 equivalents of malonic acid and the mixture is heated at reflux for 6 hours. The reaction mixture is cooled, filtered and the solid is washed with a minimal volume of ethanol/methanol to yield the desired beta-amino acid.

General Procedure B: Protection of Amino Group

1 equivalent of an amino acid is suspended in anhydrous THF (0.1-0.5 M), to which was added 1.4 equivalents of DIEA and 1.5 equivalents of di-tert-butyl-dicarbonate. The mixture is stirred for 6 hours and diluted with water and the layers are separated. The aqueous layer is further extracted with EtOAc, the organic layers combined, washed with brine, and the organic layer dried over sodium sulfate. The solvent is removed in vacuo, and the crude product purified by to flash chromatography on silica gel to give the final product.

General Procedure C: Formation of Keto Ester

1 equivalent of a protected amino acid is dissolved in anhydrous DMF (0.2-0.3 M), to which is added DIEA (1 equivalent) and either 1 equivalent of a alpha-bromo- or a alpha-chloro-ketone. The mixture is stirred at room temperature for 30 minutes, diluted with water/ethyl acetate and the layers separated. The aqueous layer is further extracted with EtOAc. The organic layers are combined, washed with saturated citric acid, brine, and the organic layer dried over Na₂SO₄, and the solvent removed in vacuo to give the crude product, which can be used without further purification.

General Procedure D: Formation of Imidazole

To a solution of the keto ester (1 equivalent) obtained according to general procedure C dissolved in a 1:1 mixture of glacial acetic acid: anhydrous DMF (0.2-0.3 M) is added ammonium acetate (3 equivalents) and the mixture was then heated at 100° C. for 4 hours. The mixture is cooled, diluted with water and extracted with ethyl acetate and the layers separated. The aqueous layer is further extracted with EtOAc. The organic layers are combined, washed with saturated sodium bicarbonate, water, brine, and the organic layer dried over Na₂SO₄. The solvent removed in vacuo to yield the crude product. The residue is subjected to silica gel chromatography to yield the desired substituted imidazole.

General Procedure E: Removal of the t-Butyl Carbamate Group

The protected compound is stirred in 4N HCl/dioxane for 1 hour. The solvent removed, and the product triturated several times with ether to afford the desired compound.

General Procedure F: Preparation of Amides

To a solution of carboxylic acid (1.25 eq) in DMF (0.1-0.5 M), HBTU (1.25 eq) is added followed by DIEA (1.25 eq) and the appropriate protected amine (1 eq.). The reaction mixture is then stirred at room temperature for 4 hours. The reaction mixture is diluted with water/EtOAc and the layers separated. The aqueous layer is reextracted with EtOAc and the organic layers combined, washed with saturated Na₂CO₃ and brine. The organic phase is then dried over Na₂SO₄, filtered, and the filtrate is concentrated and purified by silica gel chromatography to afford the amide derivative.

General Procedure G: Protection of Formylsalicylic Acid

To a solution of formylsalicylic acid (1 equivalent) in anhydrous DMF (0.8-1.2 M) 1-bromobutane (2.2 equivalents) is added followed by freshly ground K₂CO₃ (2.2 equivalents). The reaction mixture is heated at 80° C. for 4 hours. It is then cooled and diluted with water/EtOAc and the layers separated. The aqueous layer is further extracted with EtOAc, and the organic layers combined and dried over Na₂SO₄. The solvent is removed in vacuo and the residue purified by silica gel chromatography to yield the final product.

General Procedure H: Alkylation of Imidazole Nitrogen

To a solution of imidazole (1 equivalent) in anhydrous DMF (0.8-1.2 M) is added an alkyl or aryl halide (1.1 equivalents) followed by freshly ground K₂CO₃ (1.5 equivalents). The reaction mixture is stirred at room temperature for 30 minutes. It is then diluted with water/EtOAc and the layers separated. The aqueous layer is further extracted with EtOAc, and the organic layers combined and dried over Na₂SO₄. The solvent is removed in vacuo and the residue purified by silica gel chromatography to yield the final product.

General Procedure I: Hydrolysis of Ester

The ester (1 eq.) is suspended in a mixture of MeOH:THF:H₂0 (1:1:1; 0.1-0.2 M). LiOH (10-15 eq) was added and the mixture stirred at 40° C. for 3 hours. The solution is acidified with 10% citric acid, and extracted with ethyl acetate. The organic extracts are combined, washed with brine, dried over Na₂SO₄, and the solvent removed in vacuo. The residue is purified by silica gel chromatography to yield the final compound.

General Procedure J: Preparation of Aryl Acyl Bromide

1 equivalent of the desired substituted acetophenone is dissolved in THF (0.5-0.75 M). To the mixture is added pyrrolidine hydrotribromide (1.1 equivilents). The mixture is refluxed for 30 minutes, cooled, diluted with water/EtOAc, and the layers separated. The aqueous layer is further extracted with EtOAc, and the organic layers combined, washed with water and brine, and dried over Na₂SO₄. The solvent is removed in vacuo and the residue subjected to silica gel chromatography to yield the final product.

General Procedure K: Dealkylation

To 1 equivalent of the alkyl phenolic ether in DCM at −78° C. is added 10 equivalents of BBr₃. The solution is warmed to room temperature over 30 minutes, and the reaction mixture quenched with MeOH. The reaction mixture is then diluted with water/EtOAc and the layers separated. The aqueous layer is further extracted with EtOAc, and the organic layers combined, washed with water and brine, and dried over Na₂SO₄. The solvent is removed in vacuo and the residue subjected to silica gel chromatography to yield the final product.

General Procedure L: Formation of the Amino-Nitrile

To 1 equivalent of the aldehyde in ammonium hydroxide is added 2.5 equivilents of both potassium cyanide and ammonium chloride. The reaction is stirred overnight at room temperature and the reaction mixture is then diluted with water/EtOAc and the layers separated. The aqueous layer is further extracted with EtOAc, and the organic layers combined, washed with water and brine, and dried over Na₂SO₄. The solvent is removed in vacuo and the residue subjected to silica gel chromatography to yield the final product.

General Procedure M: Hydrolysis of the Amino-Nitrile to the Methyl Ester

1 equivalent of the aminonitrile is dissolved in a 1:1 mixture of 4N HCl/Dioxane:methanol (total of 2.5 equivilents HCl). The reaction mixture is then heated to reflux for three hours, cooled and the solvent removed in vacuo. The resulting solid is washed with diethyl ether to yield the amino acid methyl ester hydrochloride.

General Procedure N: Selective Hydrolysis of a Methyl Ester

1 equivalent of the amino acid methyl ester hydrochloride is dissolved in a 7:3 mixture of THF:H₂O. The mixture is chilled to 0° C. and 2.5 equivilents of LiOH were added. The mixture is kept at 0° C. for 20 minutes, then concentrated in vacuo. The reaction mixture is then acidified with citric acid and extracted with EtOAc and the layers separated. The aqueous layer is further extracted with EtOAc, and the organic layers combined, washed with water and brine, and dried over Na₂SO₄. The solvent is removed in vacuo and the residue subjected to silica gel chromatography to yield the final product.

General Procedure 0: Preparation of Sulfonamides

An amine (0.1 mmol) was dissolved in pyridine (2 mL) and a catalytic amount of DMAP was added. Sulfonyl chloride (0.13 mmol) was added and the mixture was stirred at room temperature overnight. The mixture was diluted with water/EtOAc. Layers were separated, the aqueous layer was further extracted with EtOAc. The combined organic extracts were washed (dil HCl, water, aq NaHCO₃), dried (Na₂SO₄), and evaporated in vacuo. The residue was purified by silica gel chormatography to provide the sulfonamide.

General Procedure P: Formation of Urea

1 equivalent af an amine was dissolved in 1,2-dichloroethane (0.1 M) and a catalytic amount of DMAP was added. 1.3 equivalents of an isocayanate was added and the mixture was stirred at 75° C. for 1 hour. The mixture was then cooled to room temperature and diluted with water/EtOAc. Layers were separated, the aqueous layer was further extracted with EtOAc, and the combined organic extracts were washed with brine, dried over Na₂SO₄, and evaporated in vacuo. The residue was purified by silica gel chormatography to provide the urea product.

General Procedure Q : Diaryl Ether Synthesis

To a solution of desired phenol (1 equivalent) in anhydrous NMP (0.8-1.2 M) is added cesium carbonate (1 equivalent). The slurry was degassed and filled with nitrogen 3 times. The aryl halide (ArX; X═I, Br) (1 equivalent) and 2,2,6,6,-tetramethylheptane-3-5-dione (0.005%, catalytic amount) were added followed by copper (I) chloride (0.5 equivalents). The reaction mixture was degassed and filled with nitrogen 3 times and then warmed to 120° C. under nitrogen. Reactions were monitored by LC/MS and stopped after complete conversion. The reaction mixture was cooled to room temperature and diluted with EtOAc. The slurry was filtered through celite and filter cake was washed with EtOAc several times. Combined filtrates were washed with water and the aqueous layer was further extracted with EtOAc. The organic layers were combined and dried over Na₂SO₄. The solvent was removed in vaco and the residue purified by silica gel chromatography to yield the final product.

General Procedure R: Formation of Tetrazole

1 equivalent of a nitrile was dissolved in anhydrous DMF (0.1-0.3 M) in a heavy wall glass tube. 4 equivalents each of sodium azide and ammonium chloride were added, the tube was flushed with nitrogen, sealed and stirred at 125° C. for 4 to 12 hours. The mixture was then cooled, acidified with 10% acetic acid, and diluted with water/EtOAc. Layers were separated, the aqueous layer was further extracted with EtOAC, and the combined organic extracts were washed with brine, dried over Na₂SO₄, and evaporated in vacuo. The residue was purified by silica gel chormatography to afford the tetrazole product.

General Procedure S: Protection of Immidazole Nitrogen

1 equivalent of an immidazole was suspended in anhydrous THF (0.1-0.5 M), to which was added 1.4 equivalents of TEA and 1.5 equivalents of di-tert-butyl-dicarbonate. The mixture was stirred for 2 hours and diluted with water and the layers were separated. The aqueous layer was further extracted with EtOAc, the organic layers combined, washed with brine, and the organic layer dried over sodium sulfate. The solvent was removed in vacuo, and the crude product purified by to flash chromatography on silica gel to give the final product.

General Drocedure T: Reduction of Aryl Nitro Group

To a suspension of aryl nitro compound (1 eq) in HOAc (0.1-0.5 M), iron powder (325 mesh, 4 eq) is added and the mixture is then heated at 120° C. under nitrogen for 3 to 4 hours. At completion, the reaction mixture is diluted with water/EtOAc and the leftover iron powder is filtered and washed with EtOAc. The combined organic layer is washed with water, saturated NaHCO₃ and brine. The organic phase is then dried over Na₂SO₄, filtered, and the filtrate is concentrated and purified by silica gel chromatography to afford the aniline derivative.

General Procedure U: Acylation of Amine

1 equivalent of an amine hydrochloride was dissolved in dichloromethane. 2 equivalents of diisopropylethylamine were added, then 1.2 equivalents of acid chloride were added with stirring. Stirring continued overnight, then the mixture was diluted with water/EtOAc and layers were separated. The organic layer was washed successively with water, aqueous NaHCO₃, and brine, and dried over Na₂SO₄. After evaporation in vacuo the residue was purified by silica gel chromatography to provide the amide.

### EXAMPLE 1

### 2-[(Tert-butoxycarbonyl)amino]-2-(4′-n-butoxy-3′-n-butoxycarbonylphenyl)-2-ethyl [4-(4′-nitrophenyl)]imidazole

3-[(Tert-butoxycarbonyl)amino]-3-(4′-n-butoxy-3′-n-butoxycarbonylphenyl)propanoic acid (2.2 g, 52%) was synthesized from 5-formylsalicyclic acid ester (2.78 g, 10 mmol) following general procedures A and B.

LCMS: m/z 438 (M+H)⁺, ¹H NMR (CD₃OD, 400 MHz): δ 0.90 (s, 9H), 0.95 (dt, 6H), 1.54 (m, 4H), 1.84 (m, 4H), 3.31 (d, 2H), 4.05 (t, 2H), 4.32 (t, 2H), 6.99 (d, 4 Hz), 2H), 7.59 (dd 1H), 7.92 (d,1H) ppm.

3-[(Tert-butoxycarbonyl)amino]-3-(4′-n-butoxy-3′-n-butoxycarbonylphenyl)propanoic acid (4.37 g, 10 mmol) was treated with 4-nitrophenacyl bromide according to general procedure C to afford the desired keto ester. The imidazole (2.32 g, 40%) was formed from the crude keto ester according to General procedure D.

LCMS: m/z 581 (M+H)⁺; ¹H NMR (400 MHz, Methanol-d₄): δ 0.90 (S, 9H), 0.95 (dt, 6H), 1.52 (m, 4H), 1.81 (m, 4H), 3.31 (d, 2H), 4.01 (t, 2H), 4.21 (t, 2H), 5.48 (m, 1H), 7.02 (br d, 1H), 7.33 (d, 1H), 7.79 (s, 1H), 8.01 (d, 1H), 8.26 (d, 1H), 8.35 (m, 3H),

### EXAMPLE 2

### 5-{1-Amino-2-[4-(4-nitro-phenyl)-1H-imidazol-2-yl]-ethyl}-2-butoxy-benzoic acid butyl ester hydrochloride

2- [(Tert-butoxycarbonyl)amino]-2-(4′-n-butoxy-3′-n-butoxycarbonyl phenyl)-2-ethyl[4-(4′-nitro phenyl)]imidazole (5.80 g, 10 mmol) was treated with HCl in dioxane following general procedure E to afford 5-{1-amino-2-[4-(4-nitro-phenyl)-1H-imidazol-2-yl]-ethyl}-2-butoxy-benzoic acid butyl ester hydrochloride (3.6 g, 76%).

LCMS: m/z 481 (M+)⁺.

### EXAMPLE 3

### 2-[(Tert-butyl-methyl-carbonyl)amino]-2-(4′-n-butoxy-3′-n-butoxycarbonyl phenyl)-2-ethyl [4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with tert-butylacetic acid as described in general procedure F to afford 2-[(tert-butyl-methyl-carbonyl)amino]-2-(4′-n-butoxy-3′-n-butoxycarbonyl phenyl)-2-ethyl [4-(4′-nitrophenyl)]imidazole (380 mg, 65%).

LCMS: m/z 579 (M+H)⁺; ¹H NMR (Acetone-d6, 400 MHz): δ 0.90 (s, 9H), 0.95 (dt, 6H), 1.52 (m, 4H), 1.81 (m, 4H), 2.13 (s, 2H), 3.33 (d, 2H), 4.02 (t, 2H), 4.22 (t, 2H), 5.48 (m, 1H), 7.02 (br d, 1H), 7.33 (d, 1H), 7.79 (s, 1H), 8.01 (d, 1H), 8.26 (d, 1H), 8.35 (m, 3H) ppm

### EXAMPLE 4

### 2-[(Tert-butyl-methyl-carbonyl)amino]-2-(4′-n-butoxy-3′-carboxyl phenyl)-2-ethyl[4-(4′-nitrophenyl)]imidazole

2-[(Tert-butyl-methyl-carbonyl)amino]-2-(4′-n-butoxy-3′-n-butoxycarbonyl phenyl)-2-ethyl [4-(4′-nitrophenyl)]imidazole (289 mg, 0.5 mmol) was hydrolyzed as described in general procedure I to afford 2-[(tert-butyl-methyl-carbonyl)amino]-2-(4′-n-butoxy-3′-carboxyl phenyl)-2-ethyl [4-(4′-nitrophenyl)]imidazole (229 mg, 88%) as a solid.

LCMS: m/z 523 (M+H)⁺; ¹H NMR (CD₃OD, 400 MHz): δ 0.88 (s, 9H), 0.95 (t, 3H), 1.48 (m, 2H), 1.72 (dt, 2H), 2.03 (s, 2H), 3.21 (d, 2H), 3.96 (t, 2H), 5.35 (m, 1H), 6.86 (d, 1H), 7.18 (d, 1H), 7.49 (s, 1H), 7.87 (d, 1H), 8.13 (d, 1H), 8.21 (m, 3H) ppm.

### EXAMPLE 5

### 2-[(4-Cyclohexyl-butyryl)amino]-2-(4′-n-butoxy-3′-n-butoxycarbonyl phenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-cyclohexylbutyric acid as described in general procedure F to afford 2-[(4-cyclohexyl-butyryl)amino]-2-(4′-n-butoxy-3′-n-butoxycarbonyl phenyl)-2-ethyl [4-(4′-nitrophenyl)]imidazole (401 mg, 63%).

LCMS: m/z 633 (M+H)⁺; ¹H NMR (Acetone-d6, 400 MHz): δ 0.97 (dt, 6H), 1.03-1.91 (m, 23H), 2.16 (t, 2H), 3.46 (d, 2H), 4.00 (t, 2H), 4.24 (t, 2H), 5.49 (m, 1H), 7.13 (m, 1H), 7.26 (d, 1H), 7.55 (m, 1H), 7.83 (s, 1H), 8.16 (m, 2H), 8.27 (d, 2H) ppm.

### EXAMPLE 6

### 2-[(4-Cyclohexyl-butyryl)amino]-2-(4′-n-butoxy-3′-carboxyphenyl)-2-ethyl[4-(4′-nitrophenyl)]imidazole

2-[(4-Cyclohexyl-butyryl)amino]-2-(4′-n-butoxy-3′-n-butoxycarbonylphenyl)-2-ethyl[4-(4′-nitrophenyl)]imidazole (316 mg, 0.5 mmol) was hydrolyzed as described in general procedure I to afford 2-[(4-cyclohexyl-butyryl)amino]-2-(4′-n-butoxy-3′-carboxyphenyl)-2-ethyl[4-(4′-nitrophenyl)]imidazole (221 mg, 77%).

LCMS: m/z 577 (M+H)⁺; ¹H NMR (Acetone-d6, 400 MHz): δ 0.96 (t, 3H), 1.03-1.91 (m, 19H), 2.15 (t, 2H), 3.36 (d, 2H), 4.24 (t, 2H), 5.47 (m, 1H), 7.13 (m, 1H), 7.26 (d, 1H), 7.55 (m, 1H), 7.84 (s, 1H), 8.15 (m, 2H), 8.28 (d, 2H) ppm.

### EXAMPLE 7

### 2-[(3-Cyclohexyl-propionyl)amino]-2-(4′-n-butoxy-3′-n-butoxycarbonyl phenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg,1 mmol) was treated with cyclohexanepropionic acid as described in general procedure F to afford 2-[(3-cyclohexyl-propionyl) amino]-2-(4′-n-butoxy-3′-n-butoxycarbonyl phenyl)-2-ethyl[4-(4′-nitrophenyl)]imidazole (481 mg, 78%).

LCMS: m/z 619 (M+H)⁺; ¹H NMR (Acetone-d6, 400 MHz): δ 0.97 (dt, 6H), 1.10-1.86 (m, 21H), 2.17 (t, 2H), 3.36 (d, 2H), 4.00 (t, 2H), 4.24 (t, 2H), 5.49 (m, 1H), 7.01 (d, 1H), 7.38 (dd, 1H), 7.56 (m, 1H), 7.61 (d, 1H), 7.90 (m, 2H), 8.19 (d, 2H) ppm.

### EXAMPLE 8

### 2-[(3-Cyclohexyl-propionyl)amino]-2-(4′-n-butoxy-3′-carboxyphenyl)-2-ethyl[4-(4′-nitrophenyl)]imidazole

2-[(3-Cyclohexyl-propionyl)amino]-2-(4′-n-butoxy-3′-n-butoxycarbonylphenyl)-2-ethyl[4-(4′-nitrophenyl)]imidazole (309 mg, 1 mmol) was treated as described in general procedure I to afford 2-[(3-cyclohexyl-propionyl) amino]-2-(4′-n-butoxy-3′-carboxy phenyl)-2-ethyl[4-(4′-nitrophenyl)]imidazole (232 mg, 83%).

LCMS: m/z 563 (M+H)⁺; ¹H NMR (CD₃OD, 400 MHz): δ 0.96 (t, 3H), 1.11-1.81 (m, 17H), 2.19 (t, 2H), 3.21 (d, 2H), 4.06 (t, 2H), 5.37 (m, 1H), 7.02 (d, 1H), 7.38 (dd, 1H), 7.57 (s, 1H), 7.75 (d, 1H), 7.90 (d, 2H), 8.22 (d, 2H) ppm.

### EXAMPLE 9

### 2-[(2-Cyclohexyl-acetyl)amino]-2-(4′-n-butoxy-3′-n-butoxycarbonyl phenyl)-2-ethyl[4-(4′-nitrophenyl)]imidazole

5-{1-Amino-2-[4-(4-nitro-phenyl)- 1H-imidazol-2-yl]-ethyl}-2-butoxy-benzoic acid butyl ester hydrochloride (480 mg, 1 mmol) was treated with cyclohexylacetic acid as described in general procedure F to afford 2-[(2-cyclohexyl-acetyl)amino]-2-(4′-n-butoxy-3′-n-butoxycarbonyl phenyl)-2-ethyl[4-(4′-nitrophenyl)]imidazole (480 mg, 80%).

LCMS: m/z 605 (M+H)⁺; ¹H NMR (Acetone-d6,400 MHz): δ 0.96 (dt, 6H), 1.10-1.86 (m, 19H), 2.19 (d, 2H), 3.36 (d, 2H), 4.00 (t, 2H), 4.24 (t, 2H), 5.49 (m, 1H), 7.14 (m, 1H), 7.27 (d, 1H), 7.55 (d, 1H), 7.83 (s, 1H), 7.95 (d, 1H), 8.14 (d, 1H), 8.26 (m, 2H) ppm.

### EXAMPLE 10

### 2-[(2-Cyclohexyl-acetyl)amino]-2-(4′-n-butoxy-3′-carboxyphenyl)-2-ethyl[4-(4′- nitrophenyl)]imidazole

2-[(2-Cyclohexyl-acetyl)amino]-2-(4′-n-butoxy-3′-n-butoxycarbonyl phenyl)-2-ethyl [4-(4′-nitrophenyl)]imidazole (302 mg, 1 mmol) was hydrolyzed as described in general procedure I to afford 2-[(2-cyclohexyl-acetyl) amino]-2-(4′-n-butoxy-3′-carboxy phenyl)-2-ethyl[4-(4′-nitrophenyl)]imidazole (246 mg, 89%).

LCMS: m/z 549 (M+H)⁺; ¹H NMR (Acetone-d6, 400 MHz): δ 0.96 (t, 3H), 1.11-1.81 (m, 15H), 2.19 (d, 2H), 3.29 (d, 2H), 4.25 (t, 2H), 5.44 (m, 1H), 7.13 (m, 1H), 7.26 (d, 1H), 7.55 (d, 1H), 7.84 (s, 1H), 7.97 (d, 1H), 8.15 (d, 1H), 8.28 (m, 2H) ppm.

### EXAMPLE 11

### 2-[(4-tert-butyl-phenyl)-carbonylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-tert-butylbenzoic acid as described in general procedure F to afford 2-[(4-tert-butyl-phenyl)-carbonyl amino]-2-(4′-n-butoxy-3′-n-butoxycarbonylphenyl)-2-ethyl[4-(4′-nitrophenyl)]imidazole (521 mg, 82%).

LCMS: m/z 641 (M+H)⁺; ¹H NMR (Acetone-d6, 400 MHz): δ 0.95 (dt, 6H), 1.28 (s, 9H), 1.51 (m, 4H), 1.82 (m, 4H), 3.44 (d, 2H), 4.00 (t, 2H), 4.19 (t, 2H), 5.61 (m, 1H), 7.11 (d, 1H), 7.53 (d, 2H), 7.76 (s, 1H), 7.91 (d, 2H), 7.98 (s, 1H), 8.06 (d, 2H), 8.22 (m, 1H), 8.29 (d, 1H), 8.36 (d, 1H) ppm.

### EXAMPLE 12

### 2-Butoxy-5-{1-(4-tert-butyl-benzoylamino)-2-[4-(4-nitro-phenyl)-1H-imidazol-2-yl]-ethyl}-benzoic acid

2-[(4-tert-butyl-phenyl)-carbonylamino]-2-(4′-n-butoxy-3′-n-butoxycarbonyl-phenyl)-2-ethyl[4-(4′-nitrophenyl)]imidazole (320 mg, 0.5 mmol) was treated as described in general procedure I to afford 2-butoxy-5-{1-(4-tert-butyl-benzoylamino)-2-[4-(4-nitro-phenyl)-1H-imidazol-2-yl]-ethyl}-benzoic acid (251 mg, 85%).

LCMS: m/z 585 (M+H)⁺; ¹H NMR (Acetone-d6, 400 MHz): δ 0.95 (t, 3H), 1.29 (s, 9H), 1.50 (m, 2H), 1.81 (dt, 2H), 3.43 (d, 2H), 4.19 (t, 2H), 5.62 (m, 1H), 7.10 (d, 1H), 7.53 (d, 2H), 7.75 (s, 1H), 7.91 (d, 2H), 7.99 (s, 1H), 8.08 (d, 2H), 8.24 (m, 1H), 8.28 (d, 1H), 8.36 (d,1H) ppm.

### EXAMPLE 13

### 2-[4-(4′-Methoxyphenyl)-butyrylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480, 1 mmol) was treated with 4-(4-methoxyphenyl)butyric acid as described in general procedure F to afford 2-[4-(4′-methoxyphenyl)-butyrylamino]-2-(4′-n-butoxy-3′-n-butoxycarbonylphenyl)-2-ethyl[4-(4′-nitrophenyl)]imidazole (581 mg, 88%).

LCMS: m/z 657 (M+H)⁺, ¹H NMR (Acetone-d6, 400 MHz): δ 0.95 (dt, 6H), 1.50 (m, 4H), 1.75 (m, 6H), 2.41 (t, 2H), 2.81 (t, 2H), 3.43 (d, 2H), 3.79 (s, 3H), 4.02 (t, 2H), 4.22 (t, 2H), 5.43 (m, 1H), 6.71 (d, 2H), 6.91 (d, 2H), 7.05 (d, 1H), 7.45 (m, 7.58 (brs, 1H), 7.68 (s, 1H), 7.85 (m, 2H), 8.16 (d, 2H), ppm.

### EXAMPLE 14

### 2-[4-(4′-Methoxyphenyl)-butyrylamino]-2-(4′-n-butoxy-3′-carboxyphenyl)-2-ethyl[4-(4′-nitrophenyl)]imidazole

2-[4-(4′-Methoxyphenyl)-butyrylamino]-2-(4′-n-butoxy-3′-n-butoxycarbonylphenyl)-2-ethyl[4-(4′-nitrophenyl)]imidazole (328 mg, 0.5 mmol) was hydrolyzed as described in general procedure I to afford 2-[4-(4′-methoxyphenyl)-butyrylamino]-2-(4′-n-butoxy-3′-carboxyphenyl)-2-ethyl[4-(4′-nitrophenyl)]imidazole (259 mg, 86%).

LCMS: m/z 601 (M+H)⁺, ¹H NMR (CD₃OD, 400 MHz): δ 0.95 (t, 3H), 1.50 (m, 2H), 1.73 (m, 4H), 2.16 (m, 2H), 2.34 (m, 2H), 3.20 (d, 2H), 3.70 (s, 3H), 4.02 (t, 2H), 5.40 (m, 1H), 6.75 (d, 2H), 6.89 (d, 2H), 6.98 (d, 1H), 7.33 (m, 1H), 7.52 (s, 1H), 7.64 (m, 1H), 7.81 (d, 2H), 8.14 (d, 2H) ppm.

### EXAMPLE 15

### 2-[3-(4′-Methoxyphenyl)-propionylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 3-(4-methoxyphenyl)propionic acid as described in general procedure F to afford 2-[3-(4′-methoxyphenyl)-propionylamino]-2-(4′-n-butoxy-3′-n-butoxycarbonylphenyl)-2-ethyl[4-(4′-nitrophenyl)]imidazole (541 mg, 84%).

LCMS: m/z 643 (M+H)⁺; ¹H NMR (Acetone-d6, 400 MHz): δ 0.95 (dt, 6H), 1.51 (m, 4H), 1.81 (m, 4H), 2.46 (t, 2H), 2.86 (t, 2H), 3.43 (d, 2H), 3.79 (s, 3H), 4.00 (t, 2H), 4.22 (t, 2H), 5.43 (m, 1H), 6.72 (d, 2H), 6.90 (d, 2H), 7.05 (d,1H), 7.46 (m, 1H), 7.58 (brs, 1H), 7.68 (s, 1H), 7.85 (m, 2H), 8.17 (d, 2H) ppm.

### EXAMPLE 16

### 2-[3-(4′-Methoxyphenyl)-propionylamino]-2-(4′-n-butoxy-3′-carboxyphenyl)-2-ethyl[4-(4′-nitrophenyl)]imidazole

2-[3-(4′-Methoxyphenyl)-propionylamino]-2-(4′-n-butoxy-3′-n-butoxycarbonyl-phenyl)-2-ethyl[4-(4′-nitrophenyl)]imidazole (321 mg, 0.5 mmol) was treated as described in general procedure I to afford 2-[3-(4′-methoxyphenyl)-propionylamino]-2-(4′-n-butoxy-3′-carboxyphenyl)-2-ethyl[4-(4′-nitrophenyl)]imidazole (261 mg, 89%) LCMS: m/z 587 (M+H)⁺; ¹H NMR (CD₃OD, 400 MHz): δ 0.95 (t, 3H), 1.51 (m, 2H), 1.79 (dt, 2H), 2.46 (t, 2H), 2.86 (t, 2H), 3.43 (d, 2H), 3.80 (s, 3H), 4.22 (t, 2H), 5.43 (m, 1H), 6.76 (d, 2H), 6.89 (d, 2H), 6.98 (d, 1H), 7.33 (m, 1H), 7.52 (s, 1H), 7.64 (m, 1H), 7.80 (d, 2H), 8.13 (d, 2H) ppm.

### EXAMPLE 17

### 2-[3-(3′-Methoxyphenyl)-propionylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)- 1H-imidazol-2-yl]-ethyl}-2-butoxy-benzoic acid butyl ester hydrochloride (480 mg, 1 mmol) was treated with 3-(3-methoxyphenyl)-propionic acid as described in general procedure F to afford the desired product (569 mg, 88%).

LCMS: m/z 643 (M+H)⁺; ¹H NMR (Acetone-d6, 400 MHz): δ 0.95 (dt, 6H), 1.51 (m, 4H), 1.81 (m, 4H), 2.45 (t, 2H), 2.85 (t, 2H), 3.43 (d, 2H), 3.79 (s, 3H), 4.00 (t, 2H), 4.22 (t, 2H), 5.43 (m, 1H), 6.77 (d, 1H), 6.80 (d, 2H), 7.05 (s, 1H), 7.17 (m, 1H), 7.81 (s, 1H), (m, 1H), 8.04 (d, 2H), 8.25 (m, 1H), 8.27 (d, 1H), 8.39 (m, 1H) ppm.

### EXAMPLE 18

### 2-[3-(3′-Methoxyphenyl)-propionylamino]-2-(4′-n-butoxy-3′-carboxyphenyl)-2-ethyl[4-(4′-nitrophenyl)]imidazole

2-[3-(3′-Methoxyphenyl)-propionylamino]-2-(4′-n-butoxy-3′-n-butoxycarbonyl-phenyl)-2-ethyl[4-(4′-nitrophenyl)]imidazole (321 mg, 0.5 mmol) was hydrolyzed as described in general procedure I to afford 2-[3-(3′-methoxyphenyl)-propionylamino]-2-(4′-n-butoxy-3′-carboxyphenyl)-2-ethyl[4-(4′-nitrophenyl)]imidazole (241 mg, 83%).

LCMS: m/z 587 (M+H)⁺; ¹H NMR (Acetone-d6, 400 MHz): δ 0.95 (t, 3H), 1.51 (m, 2H), 1.79 (dt, 2H), 2.46 (t, 2H), 2.86 (t, 2H), 3.43 (d, 2H), 3.78 (s, 3H), 4.22 (t, 2H), 5.43 (m, 1H), 6.77 (d, 1H), 6.80 (d, 2H), 7.05 (s, 1H), 7.17 (m, 1H), 7.80 (s, 1H), 7.95 (m, 1H), 8.08 (d, 2H), 8.24 (d,1H), 8.28 (d,1H), 8.36 (m, 1H) ppm.

### EXAMPLE 19

### 2-[3-(4′-Methoxy-phenyl)-acryl amino]-2-(4′-n-butoxy-3′-n-butoxycarbonyl phenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-methoxycinnamic acid as described in general procedure F to afford 2-[3-(4′-methoxy-phenyl)-acryl amino]-2-(4′-n-butoxy-3′-n-butoxycarbonyl phenyl)-2-ethyl[4-(4′-nitrophenyl)]imidazole (510 mg, 79%).

LCMS: m/z 641 (M+H)⁺; ¹H NMR (Acetone-d6, 400 MHz): δ 0.97 (dt, 6H), 1.54 (m, 4H), 1.82 (m, 4H), 3.47 (d, 2H), 3.83 (s, 3H), 4.00 (t, 2H), 4.22 (t, 2H), 5.54 (m, 1H), 6.59 (d, 1H), 6.77 (d, 1H), 6.97 (d, 1H), 7.05 (s, 1H), 7.17 (m, 1H), 7.52 (m, 2H), 7.54 (d, 1H), 7.81 (s, 1H), 7.95 (m, 1H), 8.04 (d, 2H), 8.25 (m, 1H), 8.37 (m, 1H) ppm.

### EXAMPLE 20

### 2-[3-(4′-Methoxy-Phenyl)-acrylamino]-2-(4′-n-butoxy-3′-carboxyphenyl)-2-ethyl[4-(4′-nitrophenyl)]imidazole

2-[3-(4′-Methoxy-phenyl)-acrylamino]-2-(4′-n-butoxy-3′-n-butoxycarbonylphenyl)-2-ethyl[4-(4′-nitrophenyl)]imidazole (320 mg, 0.5 mmol) was hydrolyzed as described in general procedure I to afford 2-[3-(4′-methoxy-phenyl)-acrylamino]-2-(4′-n-butoxy-3′-carboxyphenyl)-2-ethyl[4-(4′-nitrophenyl)]imidazole (241 mg, 82%).

LCMS: m/z 585 (M+H)⁺; ¹H NMR (Acetone-d6, 400 MHz): δ 0.97 (t, 3H), 1.53 (m, 2H), 1.82 (dt, 2H), 3.48 (d, 2H), 3.83 (s, 3H), 4.22 (t, 2H), 5.53 (m, 1H), (m, 1H), 6.59 (d, 1H), 6.77 (d, 1H), 6.97 (d, 1H), 7.05 (s, 1H), 7.17 (m, 1H), 7.52 (m, 2H), 7.54 (d, 1H), 7.80 (s, 1H), 7.95 (t, 1H), 8.08 (d, 2H), 8.24 (m, 1H), 8.36 (m, 1H) ppm.

### EXAMPLE 21

### 2-[3-(4′-Chloro-phenyl)-acrylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)- 1H-imidazol-2-yl]-ethyl}-2-butoxy-benzoic acid butyl ester hydrochloride (480 mg, 1 mmol) was treated with 4-chlorocinnamic acid as described in general procedure F to afford 2-[3-(4′-chloro-phenyl)-acrylamino]-2-(4′-n-butoxy-3′-n-butoxycarbonylphenyl)-2-ethyl[4-(4′-nitrophenyl)]imidazole (541 mg, 82%).

LCMS: m/z 645 (M+H)⁺; ¹H NMR (Acetone-d6, 400 MHz): δ 0.97 (dt, 6H), 1.54 (m, 4H), 1.82 (m, 4H), 3.47 (d, 2H), 4.00 (t, 2H), 4.22 (t, 2H), 5.54 (m, 1H), 6.77 (d, 1H), 6.97 (d, 1H), 7.05 (s, 1H), 7.17 (m, 1H), 7.64 (m, 2H), 7.66 (d, 1H), 7.83 (s, 1H), 7.95 (m, 1H), 8.04 (d, 2H), 8.25 (m, 1H), 8.39 (m, 1H) ppm.

### EXAMPLE 22

### 2-[3-(4′-Chloro-phenyl)-acrylamino]-2-(4′-n-butoxy-3′-carboxyphenyl)-2-ethyl[4-(4′-nitrophenyl)]imidazole

2-[3-(4′-Chloro-phenyl)-acrylamino]-2-(4′-n-butoxy-3′-n-butoxycarbonylphenyl)-2-ethyl[4-(4′-nitrophenyl)]imidazole (322 mg, 0.5 mmol) was hydrolyzed as described in general procedure I to afford 2-[3-(4′-chloro-phenyl)-acrylamino]-2-(4′-n-butoxy-3′-carboxyphenyl)-2-ethyl[4-(4′-nitrophenyl)]imidazole (249 mg, 85%).

LCMS: m/z 589 (M+H)⁺; ¹H NMR (Acetone-d6, 400 MHz): δ 0.97 (t, 3H), 1.53 (m, 2H), 1.82 (dt, 2H), 3.48 (d, 2H), 4.22 (t, 2H), 5.53 (m, 1H), 6.69 (d, 1H), 7.77 (d, 1H), 6.97 (d, 1H), 7.05 (s, 1H), 7.17 (m, 1H), 7.64 (m, 2H), 7.66 (d, 1H), 7.84 (s, 1H), 7.95 (m, 1H), 8.08 (d, 2H), 8.26 (m, 1H), 8.41 (m, 1H) ppm.

### EXAMPLE 23

### 2-[2-(4-Methoxy-phenyl)-acetylamino]-2-(4′-n-butoxy-3′-n-butoxycarbonylphenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-methoxyphenylacetic acid as described in general procedure F to afford 2-[2-(4′-Methoxy-phenyl)-acetylamino]-2-(4′-n-butoxy-3′-n-butoxy(arbonylphenyl)-2-ethyl[4-(4′-nitrophenyl)]imidazole (512 mg, 81%).

LCMS: m/z 629 (M+H)⁺; ¹H NMR (Acetone-d6, 400 MHz): δ 0.97 (dt, 6H), 1.53 (m, 4H), 1.83 (m, 4H), 3.43 (d, 2H), 3.55 (s, 2H), 3.78 (s, 3H), 4.00 (t, 2H), 4.22 (t, 2H), 5.40 (m, 1H), 6.75 (d, 2H), 6.89 (d, 2H), 7.17 (m, 1H), 7.81 (s, 1H), 7.95 (m, 1H), 8.04 (d, 2H), 8.15 (m, 1H), 8.21 (m, 1H), 8.38 (d, 1H) ppm.

### EXAMPLE 24

### 2-[2-(4′-Methoxy-phenyl)-acetylamino]-2-(4′-n-butoxy-3′-carboxyphenyl)-2-ethyl[4-(4′-nitrophenyl)]imidazole

2-[2-(4′-Methoxy-phenyl)-acetylamino]-2-(4′-n-butoxy-3′-n-butoxycarbonyl-phenyl)-2-ethyl[4-(4′-nitrophenyl)]imidazole (314 mg, 0.5 mmol) was hydrolyzed as described in general procedure I to afford 2-[2-(4′-methoxy-phenyl)-acetylamino]-2-(4′-n-butoxy-3′-carboxyphenyl)-2-ethyl[4-(4′-nitrophenyl)]imidazole (239 mg, 84%).

LCMS: m/z 573 (M+H)⁺; ¹H NMR (Acetone-d6, 400 MHz): δ 0.97 (t, 3H), 1.54 (m, 2H), 1.84 (m, 2H), 3.43 (d, 2H), 3.57 (s, 2H), 3.77 (s, 3H), 4.27 (t, 2H), 5.38 (m, 1H), 6.74 (d, 2H), 6.89 (d, 2H), 7.17 (m, 1H), 7.80 (s, 1H), 7.95 (m, 1H), 8.08 (d, 2H), 8.24 (d, 1H), 8.28 (d, 1H), 8.35 (m, 1H) ppm.

### EXAMPLE 25

### 2-[2-(4′-Chloro-phenyl)-acetyl amino]-2-(4′-n-butoxy-3′-n-butoxycarbonyl phenyl)-2-ethyl[4-(4′-nitrophenyl)imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-chlorophenylacetic acid as described in general procedure F to afford 2-[2-(4′-chloro-phenyl)-acetyl amino]-2-(4′-n-butoxy-3′-n-butoxycarbonylphenyl)-2-ethyl[4-(4′-nitrophenyl)]imidazole (521 mg, 83%).

LCMS: m/z 633 (M+H)⁺; ¹H NMR (Acetone-d6, 400 MHz): δ 0.97 (dt, 6H), 1.53 (m, 4H), 1.83 (m, 4H), 3.47 (d, 2H), 3.59 (s, 2H), 4.01 (t, 2H), 4.22 (t, 2H), 5.40 (m, 2H), 7.15 (m, 2H), 7.37 (m, 2H), 7.81 (s, 1H), 7.95 (m, 1H), 8.04 (d, 2H), 8.15 (m, 2H), 8.24 (m, 1H), 8.38 (d,1H) ppm.

### EXAMPLE 26

### 2-[2-(4′-Chloro-phenyl)-acetylamino]-2-(4′-n-butoxy-3′-carboxyphenyl)-2-ethyl[(4′-nitrophenyl)]imidazole

2-[2-(4′-Chloro-phenyl)-acetylamino]-2-(4′-n-butoxy-3′-n-butoxycarbonylphenyl)-2-ethyl[4-(4′-nitrophenyl)]imidazole (316 mg, 0.5 mmol) was hydrolyzed as described in general procedure I to afford 2-[2-(4′-chloro-phenyl)-acetylamino]-2-(4′-n-butoxy-3′-carboxyphenyl)-2-ethyl[(4′-nitrophenyl)]imidazole (251 mg, 88%)

LCMS: m/z 577 (M+H)⁺; ¹H NMR (Acetone-d6, 400 MHz): δ 0.97 (t, 3H), 1.54 (m, 2H), 1.84 (m, 2H), 3.46 (d, 2H), 3.61 (s, 2H), 4.27 (t, 2H), 5.38 (m, 1H), 7.15 (m, 2H), 7.37 (m, 2H), 7.80 (s, 1H), 7.95 (m, 1H), 8.08 (d, 2H), 8.24 (d,1H), 8.28 (d, 1H), 8.33 (m, 1H), 8.37 (d, 1H) ppm.

### EXAMPLE 27

### 2-[2-(4′-Methylsulphonyl-phenyl)-acetylamino]-2-(4′-n-butoxy-3′-n-butoxycarbonyl phenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480 mg, 1 mmol) was treated with 4-methylsulphonylphenyl-acetic acid as described in general procedure F to afford 2-[2-(4′-methylsulphonyl-phenyl)-acetylamino]-2-(4′-n-butoxy-3′-n-butoxycarbonylphenyl)-2-ethyl[4-(4′-nitrophenyl)]-imidazole (517 mg, 77%).

LCMS: m/z 677 (M+H)⁺; ¹H NMR (Acetone-d6, 400 MHz): δ 0.97 (dt, 6H), 1.53 (m, 4H), 1.83 (m, 4H), 3.08 (s, 3H), 3.47 (d, 2H), 3.76 (s, 2H), 4.01 (t, 2H), 4.22 (t, 2H), 5.40 (m, 1H), 7.15 (m, 2H), 7.37 (m, 2H), 7.81 (s, 1H), 7.95 (m, 1H), 8.04 (d, 2H), 8.15 (m, 2H), 8.28 (m, 1H), 8.38 (d, 1H) ppm.

### EXAMPLE 28

### 2-[2-(4′-Methylsulphonyl-phenyl)-acetyl amino]-2-(4′-n-butoxy-3′-carboxy phenyl)-2-ethyl[4-(4′-nitrophenyl)]imidazole

2-[2-(4′-Methylsulphonyl-phenyl)-acetylamino]-2-(4′-n-butoxy-3′-n-butoxycarbonyl phenyl)-2-ethyl[4-(4′-nitrophenyl)]imidazole (338 mg, 0.5 mmol) was hydrolyzed as described in general procedure I to afford 2-[2-(4′-methylsulphonyl-phenyl)-acetylamino]-2-(4′-n-butoxy-3′-carboxy phenyl)-2-ethyl[4-(4′-nitrophenyl)]imidazole (256 mg, 82%).

LCMS: m/z 621 (M+H)⁺; ¹H NMR (Acetone-d6, 400 MHz): δ 0.97 (t, 3H), 1.54 (m, 2H), 1.84 (m, 2H), 3.10 (s, 3H), 3.46 (d, 2H), 3.79 (s, 2H), 4.27 (t, 2H), 5.38 (m, 1H), 7.15 (m, 2H), 7.37 (m, 2H), 7.80 (s, 1H), 7.95 (m, 1H), 8.08 (d, 2H), 8.30 (m, 1H), 8.33 (m, 2H), 8.37 (d, 1H) ppm.

### EXAMPLE 29

### 2-[4-(4′-chloro-2′-methyl-phenoxy)-butyrylamino]-2-(4′-n-butoxy-3′-n-butoxycarbonyl phenyl)-2-ethyl[4-(4′-nitrophenyl)]imidazole

5-{1-Amino-2-[4-(4-nitro-phenyl)-1H-imidazol-2-yl]-ethyl}-2-butoxy-benzoic acid butyl ester hydrochloride (480, 1 mmol) was treated with 4-(4-chloro-2-methyl-phenoxy)butyric acid as described in general procedure F to afford 2-[4-(4′-chloro-2′-methyl-phenoxy)-butyryl amino]-2-(4′-n-butoxy-3′-n-butoxycarbonyl phenyl)-2-ethyl[4-(4′-nitrophenyl)]imidazole (589 mg, 86%).

LCMS: m/z 691 (M+H)⁺; ¹H NMR (Acetone-d6, 400 MHz): δ 0.94 (dt, 6H), 1.51 (m, 4H), 1.82 (m, 4H), 1.96-2.14 (m, 4H), 2.39 (s, 3H), 3.29 (d, 2H), 4.11-4.22 (m, 6H), 5.53 (m, 1H), 6.72 (m, 2H), 7.04 (m, 2H), 7.48 (m, 1H), 7.69 (s, 1H), 8.02 (m, 2H), 8.18 (d, 2H), 8.32 (m, 1H) ppm.

### EXAMPLE 30

### 2-[4-(4′-chloro-2′-methyl-phenoxy)-butyrylamino]-2-(4′-n-butoxy-3′-carboxyphenyl)-2-ethyl[4-(4′-nitrophenyl)]imidazole

2-[4-(4′-chloro-2′-methyl-phenoxy)-butyrylamino]-2-(4′-n-butoxy-3′-n-butoxycarbonylphenyl)-2-ethyl[4-(4′-nitrophenyl)]imidazole (345 mg, 0.5 mmol) was treated as described in general procedure I to afford 2-[4-(4′-chloro-2′-methyl-phenoxy)-butyrylamino]-2-(4′-n-butoxy-3′-carboxyphenyl)-2-ethyl[4-(4′-nitrophenyl)]imidazole (285 mg, 89%).

LCMS: m/z 635 (M+H)⁺; ¹H NMR (Acetone-d₆, 400 MHz): δ 0.95 (t, 3H), 1.51 (m, 2H), 1.82 (m, 2H), 1.96-2.17 (m, 4H), 2.41 (s, 3H), 3.29 (d, 2H), 4.10-4.22 (m, 4H), 5.53 (m, 1H), 6.70 (m, 2H), 7.04 (m, 2H), 7.47 (m, 1H), 7.70 (s, 1H), 8.00 (m, 2H), 8.18 (d, 2H), 8.30 (m, 1H) ppm.

### EXAMPLE 31

### 2-[4-(4′-Methoxyphenyl)-butyrylamino]-2-(4′-n-butoxy-3′-n-butoxycarbonylphenyl)-2-ethyl[4-(4′-chlorophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester (2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 4-chlorophenacyl bromide according to general procedure C to afford the desired keto-ester. The imidazole was formed from the keto ester according to general procedure D. Boc-protected amine was treated as described in general procedure E to give the amine hydrochloride (3.8 g, 82%).

5-{1-Amino-2-[4-(4-chloro-phenyl)-1H-imidazol-2-yl]-ethyl}-2-butoxy-benzoic acid butyl ester hydrochloride (469 mg, 1 mmol) was treated with 4-(4-methoxyphenyl)-butyric acid as described in general procedure F to afford 2-[4-(4′-methoxyphenyl)-butyrylamino]-2-(4′-n-butoxy-3′-n-butoxycarbonylphenyl)-2-ethyl-[4-(4′-chlorophenyl)-imidazole (516 mg, 80%).

LCMS: m/z 646 (M+H)⁺; ¹H NMR (CDCl₃, 400 MHz): δ 0.96 (dt, 6H), 1.48 (m, 4H), 1.68 (m, 2H), 1.77 (m, 2H), 1.87 (m, 2H), 2.22 (m, 2H), 2.48 (t, 2H), 3.18-3.34 (m, 2H), 3.76 (s, 3H), 3.98 (t, 2H), 4.26 (t, 2H), 5.43 (m, 1H), 6.75 (d, 2H), 6.97 (d, 2H), 7.04 (d, 1H), 7.37 (d, 2H), 7.44 (m, 1H), 7.59 (d, 2H), 7.78 (s,1H), 7.89 (d, 1H) ppm.

### EXAMPLE 32

### 2-[4-(4′-Methoxyphenyl)-butyrylamino]-2-(4′-n-butoxy-3′-carboxyphenyl)-2-ethyl[4-(4′-chlorophenyl)]imidazole

2-[4-(4′-Methoxyphenyl)-butyrylamino]-2-(4′-n-butoxy-3′-n-butoxycarbonyl-phenyl)-2-ethyl[4-(4′-chlorophenyl)]imidazole (322 mg, 0.5 mmol) was treated as described in general procedure I to afford 2-[4-(4′-methoxyphenyl)-butyrylamino]-2-(4′-n-butoxy-3′-carboxy phenyl)-2-ethyl[4-(4′-chlorophenyl)]imidazole (251 mg, 85%).

LCMS: m/z 590 (M+H)⁺; ¹H NMR (CD₃OD, 400 MHz): δ 0.98 (t, 3H), 1.51 (m, 2H), 1.79 (m, 4H), 2.18 (m, 2H), 2.38 (m, 2H), 3.19-3.28 (m, 2H), 3.74 (s, 3H), 4.10 (t, 2H), 5.37 (m, 1H), 6.72 (d, 2H), 6.92 (d, 2H), 7.02 (d, 1H), 7.31 (d, 2H), 7.41 (m, 1H), 7.56 (d, 2H), 7.76 (s, 1H), 7.86 (d, 1H) ppm.

### EXAMPLE 33

### 2-[4-(4′-Methoxyphenyl)-butyrylamino]-2-(4′-n-butoxy-3′-n-butoxycarbonylphenyl)-2-ethyl[4-(3′-chlorophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester (2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 3-chlorophenacyl bromide according to general procedure C to afford the desired keto-ester. The imidazole was formed from the keto ester according to general procedure D. Boc-protected amine was treated as described in general procedure E to give the amine hydrochloride (3.8 g, 82%).

5-{1-Amino-2-[4-(3-chloro-phenyl)-1H-imidazol-2-yl]-ethyl}-2-butoxy-benzoic acid butyl ester hydrochloride (469 mg, 1 mmol) was treated with 4-(4-methoxyphenyl)butyric acid as described in general procedure F to afford 2-[4-(4′-methoxyphenyl)-butyryl amino]-2-(4′-n-butoxy-3′-n-butoxycarbonylphenyl)-2-ethyl[4-(3′-chlorophenyl)]imidazole (521 mg, 80%).

LCMS: m/z 646 (M+H)⁺; ¹H NMR (CDCl₃, 400 MHz): δ 0.97 (dt, 6H), 1.50 (m, 4H), 1.67 (m, 2H), 1.77 (m, 2H), 1.86 (m, 2H), 2.21 (m, 2H), 2.49 (t, 2H), 3.18-3.34 (m, 2H), 3.77 (s, 3H), 3.98 (t, 2H), 4.26 (t, 2H), 5.42 (m, 1H), 6.75 (d, 2H), 6.96 (d, 2H), 7.06 (d, 1H), 7.39 (m, 3H), 7.44 (m, 1H), 7.62 (m, 1H), 7.78 (s, 1H), 7.92 (d, 1H), ppm.

### EXAMPLE 34

### 2-[4-(4′-Methoxyphenyl)-butyrylamino]-2-(4′-n-butoxy-3′-carboxyphenyl)-2-ethyl[4-(3′-chlorophenyl)]imidazole

2-[4-(4′-Methoxyphenyl)-butyrylamino]-2-(4′-n-butoxy-3′-n-butoxycarbonyl-phenyl)-2-ethyl[4-(3′-chlorophenyl)]imidazole (322 mg, 0.5 mmol) was hydrolyzed as described in general procedure I to afford 2-[4-(4′-methoxyphenyl)-butyrylamino]-2-(4′-n-butoxy-3′-carboxyphenyl)-2-ethyl[4-(3′-chlorophenyl)]imidazole (218 mg, 74%).

LCMS: m/z 590 (M+H)⁺; ¹H NMR (CD₃OD, 400 MHz): δ 0.97 (t, 3H), 1.52 (m, 2H), 1.79 (m, 4H), 2.19 (m, 2H), 2.39 (m, 2H), 3.17-3.28 (m, 2H), 3.74 (s, 3H), 4.10 (t, 2H), 5.37 (m, 1H), 6.71 (d, 2H), 6.93 (d, 2H), 7.03 (d, 1H), 7.35 (m, 3H), 7.40 (m, 1H), 7.59 (m, 1H), 7.76 (s, 1H), 7.88 (d, 1H) ppm.

### EXAMPLE 35

### 2-[4-(4′-Methoxyphenyl)-butyrylamino]-2-(4′-n-butoxy-3′-n-butoxycarbonylphenyl)-2-ethyl[4-(3′,4′-dichlorophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formyl salicyclic acid ester (2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 3,4-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 76%).

5-{1-Amino-2-[4-(3,4-dichloro-phenyl)-1H-imidazol-2-yl]-ethyl}-2-butoxy-benzoic acid butyl ester hydrochloride (504 mg, 1 mmol) was treated with 4-(4-methoxyphenyl)butyric acid as described in general procedure F to afford 2-[4-(4′-methoxyphenyl)-butyryl amino]-2-(4′-n-butoxy-3′-n-butoxycarbonyl phenyl)-2-ethyl [4-(3′, 4′-dichlorophenyl)]imidazole (519 mg, 76%).

LCMS: m/z 680 (M+H)⁺; ¹H NMR (CDCl₃, 400 MHz): δ 0.96 (dt, 6H), 1.47 (m, 4H), 1.68 (m, 2H), 1.79 (m, 2H), 1.87 (m, 2H), 2.20 (m, 2H), 2.47 (t, 2H), 3.18-3.31 (m, 2H), 3.76 (s, 3H), 3.96 (t, 2H), 4.25 (t, 2H), 5.43 (m, 1H), 6.74 (d, 2H), 6.94 (d, 2H), 7.11 (d, 1H), 7.60 (m, 2H), 7.69 (bs, 1H), 7.72 (s, 1H), 7.81 (bs, 1H), 7.93 (bs, 1H) ppm.

### EXAMPLE 36

### 2-[4-(4′-Methoxyphenyl)-butyryl amino]-2-(4′-n-butoxy-3′-carboxy phenyl)-2-ethyl [4-(3′, 4′-dichlorophenyl) imidazole

2-[4-(4′-Methoxyphenyl)-butyrylamino]-2-(4′-n-butoxy-3′-n-butoxycarbonyl-phenyl)-2-ethyl [4-(3′,4′-dichlorophenyl)]imidazole (340 mg, 0.5 mmol) was treated as described in general procedure I to afford 2-[4-(4′-methoxyphenyl)-butyrylamino]-2-(4′-n-butoxy-3′-carboxyphenyl)-2-ethyl[4-(3′,4′-dichlorophenyl)]imidazole (270 mg, 86%).

LCMS: m/z 624 (M+H)⁺; ¹H NMR (CD₃OD, 400 MHz): δ 0.99 (t, 3H), 1.53 (m, 2H), 1.79 (m, 4H), 2.21 (m, 2H), 2.39 (m, 2H), 3.37-3.57 (m, 2H), 3.76 (s, 3H), 4.12 (t, 2H), 5.45 (m, 1H), 6.73 (d, 2H), 6.92 (d, 2H), 7.08 (d, 1H), 7.57 (m, 2H), 7.67 (bs, 1H), 7.69 (s, 1H), 7.79 (bs, 1H), 7.90 (bs, 1H) ppm.

### EXAMPLE 37

### 2-[4-(4′-Methoxyphenyl)-butyryl amino]-2-(4′-n-butoxy-3′-n-butoxycarbonyl phenyl)-2-ethyl [4-(2′,4′-dichlorophenyl)l imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 2,4-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 76%).

5-{1-Amino-2-[4-(2,4-dichloro-phenyl)-1H-imidazol-2-yl]-ethyl}-2-butoxy-benzoic acid butyl ester hydrochloride (504 mg, 1 mmol) was treated with 4-(4-methoxyphenyl)-butyric acid as described in general procedure F to afford 2-[4-(4′-methoxyphenyl)-butyrylamino]-2-(4′-n-butoxy-3′-n-butoxycarbonylphenyl)-2-ethyl[4-(2′,4′-dichlorophenyl)] imidazole (547 mg, 80%).

LCMS: m/z 680 (M+H)⁺; ¹H NMR (CDCl₃, 400 MHz): δ 0.96 (dt, 6H), 1.46 (m, 4H), 1.67 (m, 2H), 1.78 (m, 2H), 1.87 (m, 2H), 2.21 (m, 2H), 2.48 (t, 2H), 3.19-3.33 (m, 2H), 3.76 (s, 3H), 3.96 (t, 2H), 4.25 (t, 2H), 5.43 (m, 1H), 6.76 (d, 2H), 6.82 (m, 2H), 6.98 (d, 2H), 7.22 (m, 2H), 7.47 (d, 1H), 7.64 (d, 1H), 7.98 (bs, 1H) ppm.

### EXAMPLE 38

### 2-[4-(4′-Methoxyphenyl)-butyryl amino]-2-(4′-n-butoxy-3′-carboxy phenyl)-2-ethyl [4-(2′,4′-dichlorophenyl)]imidazole

2-[4-(4′-Methoxyphenyl)-butyrylamino]-2-(4′-n-butoxy-3′-n-butoxycarbonyl phenyl)-2-ethyl[4-(2′,4′-dichlorophenyl)]imidazole (340 mg, 0.5 mmol) was treated as described in general procedure I to afford 2-[4-(4′-methoxyphenyl)-butyrylamino]-2-(4′-n-butoxy-3′-carboxyphenyl)-2-ethyl[4-(2′,4′-dichlorophenyl)]imidazole (271 mg, 86%).

LCMS: m/z 624 (M+H)⁺; ¹H NMR (CD₃OD, 400 MHz): δ 0.99 (t, 3H), 1.52 (m, 2H), 1.81 (m, 4H), 2.19 (m, 2H), 2.42 (m, 2H), 3.19 (d, 2H), 3.77 (s, 3H), 4.14 (t, 2H), 5.34 (t, 1H), 6.75 (d, 2H), 6.96 (d, 2H), 7.02 (d, 1H), 7.26 (m, 1H), 7.42 (m, 2H), 7.47 (s, 1H), 7.71 (d, 1H), 7.90 (bs, 1H) ppm.

### EXAMPLE 39

### 2-[4-(4′-Methoxyphenyl)-butyrylamino]-2-(4′-n-butoxy-3′-n-butoxycarbonylphenyl)-2-ethyl [4-(2′,5′-dichlorophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 2,5-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 82%).

5-{1-Amino-2-[4-(2,5-dichloro-phenyl)-1H-imidazol-2-yl]-ethyl}-2-butoxy-benzoic acid butyl ester hydrochloride (504 mg, 1 mmol) was treated with 4-(4-methoxyphenyl)-butyric acid as described in general procedure F to afford 2-[4-(4′-methoxyphenyl)-butyrylamino]-2-(4′-n-butoxy-3′-n-butoxycarbonylphenyl)-2-ethyl[4-(2′,4′-dichlorophenyl)] imidazole (531 mg, 78%).

LCMS: m/z 680 (M+H)⁺; ¹H NMR (CDCl₃, 400 MHz): δ 0.97 (dt, 6H), 1.48 (m, 4H), 1.67 (m, 2H), 1.77 (m, 2H), 1.87 (m, 2H), 2.22 (m, 2H), 2.47 (m, 2H), 3.17-3.36 (m, 2H), 3.77 (s, 3H), 3.99 (t, 2H), 4.24 (t, 2H), 5.43 (m, 1H), 6.77 (d, 2H), 6.87 (m, 2H), 6.99 (d, 2H), 7.26 (m, 2H), 7.49 (d, 1H), 7.68 (m, 1H), 7.97 (bs, 1H) ppm.

### EXAMPLE 40

### 2-[4-(4′-Methoxyphenyl)-butyrylamino]-2-(4′-n-butoxy-3′-carboxyphenyl)-2-ethyl[4-(2′,5′-dichlorophenyl)]imidazole

2-[4-(4′-Methoxyphenyl)-butyrylamino]-2-(4′-n-butoxy-3′-n-butoxycarbonyl phenyl)-2-ethyl [4-(2′,5′-dichlorophenyl)]imidazole (340 mg, 0.5 mmol) was treated as described in general procedure I to afford 2-[4-(4′-methoxyphenyl)-butyrylamino]-2-(4′-n-butoxy-3′-carboxyphenyl)-2-ethyl[4-(2′,5′-dichlorophenyl)] imidazole (219 mg, 70%).

LCMS: m/z 624 (M+H)⁺; ¹H NMR (CD₃OD, 400 MHz): δ 0.98 (t, 3H), 1.51 (m, 2H), 1.81 (m, 4H), 2.20 (m, 2H), 2.42 (m, 2H), 3.22 (d, 2H), 3.77 (s, 3H), 4.13 (t, 2H), 5.38 (t, 1H), 6.74 (d, 2H), 6.96 (d, 2H), 7.02 (d, 1H), 7.29 (m, 1H), 7.47 (m, 2H), 7.49 (s, 1H), 7.73 (d, 1H), 7.92 (bs, 1H) ppm.

### EXAMPLE 41

### 2-[4-(4′-Methoxyphenyl)-butyrylamino]-2-(4′-n-butoxy-3′-n-butoxycarbonylphenyl)-2-ethyl [4-(2′,64 -dichlorophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 2,6-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 82%).

5-{1-Amino-2-[4-(2,6-dichloro-phenyl)-1H-imidazol-2-yl]-ethyl}-2-butoxy-benzoic acid butyl ester hydrochloride (504 mg, 1 mmol) was treated with 4-(4-methoxyphenyl)butyric acid as described in general procedure F to afford 2-[4-(4′-methoxyphenyl)-butyryl amino]-2-(4′-n-butoxy-3′-n-butoxycarbonyl phenyl)-2-ethyl [4-(2′, 6′-dichlorophenyl)]imidazole ( 531 mg, 78%).

LCMS: m/z 680 (M+H)⁺; ¹H NMR (CDCl₃, 400 MHz): δ 0.95 (dt, 6H), 1.46 (m, 4H), 1.66 (m, 2H), 1.76 (m, 2H), 1.89 (m, 2H), 2.24 (t, 2H), 2.51 (t, 2H), 3.18-3.38 (m, 2H), 3.77 (s, 3H), 3.97 (t, 2H), 4.24 (t, 2H), 5.41 (m, 1H), 6.78 (d, 2H), 6.84 (d, 1H), 6.96 (m, 2H), 7.04 (d, 2H), 7.20 (m, 3H), 7.62 (d, 1H) ppm.

### EXAMPLE 42

### 2-[4-(4′-Methoxyphenyl)-butyryl amino]-2-(4′-n-butoxy-3′-carboxy phenyl)-2-ethyl [4-(2′, 6′-dichlorophenyl) imidazole

2-[4-(4′-Methoxyphenyl)-butyrylamino]-2-(4′-n-butoxy-3′-n-butoxycarbonyl phenyl)-2-ethyl[4-(2′,6′-dichlorophenyl)]imidazole (340 mg, 0.5 mmol) was hydrolyzed as described in general procedure I to afford 2-[4-(4′-methoxyphenyl)-butyrylamino]-2-(4′-n-butoxy-3′-carboxy phenyl)-2-ethyl [4-(2′,6′-dichlorophenyl)]imidazole (268 mg, 85%).

LCMS: m/z 624 (M+H)⁺; ¹H NMR (CD₃OD, 400 MHz): δ 0.99 (t, 3H), 1.52 (m, 2H), 1.82 (m, 4H), 2.19 (d, 2H), 2.44 (m, 2H), 3.21 (d, 2H), 3.76 (s, 3H), 4.13 (t, 2H), 5.32 (t, 1H), 6.75 (d, 2H), 6.98 (d, 2H), 7.01 (m, 3H), 7.28 (m, 2H), 7.42 (m, 1H), 7.89 (d, 1 H) ppm.

### EXAMPLE 43

### 2-[4-(4′-Methoxyphenyl)-butyrylamino]-2-(4′-n-butoxy-3′-n-butoxycarbonylphenyl)-2-ethyl[4-(4′-cyanophenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formyl salicyclic acid ester (2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 4-cynophenacyl bromide according to general procedure C to afford the desired keto-ester. The imidazole was formed from the keto ester according to general procedure D. Boc-protected amine was treated as described in general procedure E to give the amine hydrochloride (3.8 g, 82%).

5-{1-Amino-2-[4-(4-cyano-phenyl)-1H-imidazol-2-yl]-ethyl}-2-butoxy-benzoic acid butyl ester hydrochloride (460 mg, 1 mmol) was treated with 4-(4-methoxyphenyl)butyric acid as described in general procedure F to afford 2-[4-(4′-methoxyphenyl)-butyrylamino]-2-(4′-n-butoxy-3′-n-butoxycarbonylphenyl)-2-ethyl[4-(4′-cyanophenyl)]-imidazole (513 mg, 80%).

LCMS: m/z 637 (M+H)⁺; ¹H NMR (CD₃OD, 400 MHz): δ 0.95 (dt, 6H), 1.48 (m, 4H), 1.68-1.73 (m, 4H), 1.86 (m, 2H), 2.13 (m, 2H), 2.34 (t, 2H), 3.15-3.19 (m, 2H), 3.71 (s, 3H), 3.93 (t, 2H), 4.19 (t, 2H), 5.41 (m, 1H), 6.69 (d, 2H), 6.80 (m, 2H), 6.91 (d, 2H), 7.07 (m, 2H), 7.38 (d, 1H), 7.59 (d, 1H), 7.75 (d, 1H) ppm.

### EXAMPLE 44

### 2-[4-(4′-Methoxyphenyl)-butyrylamino]-2-(4′-n-butoxy-3′-carboxyphenyl)-2-ethyl[4-(4′-cynophenyl)]imidazole

2-[4-(4′-Methoxyphenyl)-butyrylamino]-2-(4′-n-butoxy-3′-carboxyphenyl)-2-ethyl[4-(4′-cynophenyl)]imidazole (226 mg, 78%) was prepared by the hydrolysis of 2-[4-(4′-methoxyphenyl)-butyrylamino]-2-(4′-n-butoxy-3′-n-butoxycarbonylphenyl)-2-ethyl[4-(4′-cynophenyl)]imidazole (318 mg, 0.5 mmol) following the general procedure I.

LCMS: m/z 581 (M+H)⁺, ¹H NMR (CD₃OD, 400 MHz): δ 0.95 (dt, 3H), 1.48 (m, 2H), 1.68 (m, 4H), 1.86 (m, 2H), 2.13 (m, 2H), 2.34 (m, 2H), 3.15-3.19 (m, 2H), 3.71 (s, 3H), 3.93 (t, 2H), 5.41 (m, 1H), 6.69 (d, 2H), 6.80 (m, 2H), 6.91 (d, 2H), 7.07 (m, 2H), 7.38 (d, 1H), 7.59 (d, 1H), 7.75 (d, 1H) ppm.

### EXAMPLE 45

### 2-[4-(4′-Methoxyphenyl)-butyrylamino]-2-(4′-n-butoxy-3′-n-butoxycarbonylphenyl)-2-ethyl[4-(naphthyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with bromomethyl 2-naphthyl ketone according to general procedure C to afford the desired keto-ester. The imidazole was formed from the keto ester according to general procedure D. Boc-protected amine was treated as described in general procedure E to give the amine hydrochloride (3.8 g, 78%).

5-[1-Amino-2-(4-naphthalen-1-yl-1H-imidazol-2-yl)-ethyl]-2-butoxy-benzoic acid butyl ester hydrochloride (485 mg, 1 mmol) was treated with 4-(4-methoxyphenyl)butyric acid as described in general procedure F to afford 2-[4-(4′-methoxyphenyl)-butyrylamino]-2-(4′-n-butoxy-3′-n-butoxycarbonylphenyl)-2-ethyl[4-(naphth-1-yl)]imidazole (501 mg, 75%).

LCMS: m/z 662 (M+H)⁺; ¹H NMR (DMSO, 400 MHz): δ 0.97 (dt, 6H), 1.46 (m, 4H), 1.52 (m, 2H), 1.65 (m, 2H), 1.77 (m, 2H), 2.23 (m, 2H), 2.40 (t, 2H), 2.99-3.23 (m, 2H), 3.66 (s, 3H), 4.01 (t, 2H), 4.22 (t, 2H), 5.50 (m, 1H), 6.64 (d, 2H), 6.90 (m, 2H), 7.03 (d, 2H), 7.49-7.39 (m, 4H), 7.63 (d, 2H), 7.83 (d, 1H), 8.17 (bs, 1H) ppm.

### EXAMPLE 46

### 2-[4-(4′-Methoxyphenyl)-butyrylamino]-2-(4′-n-butoxy-3′-carboxyphenyl)-2-ethyl[4-(1-naphthyl)]imidazole

2-[4-(4′-Methoxyphenyl)-butyrylamino]-2-(4′-n-butoxy-3′-carboxyphenyl)-2-ethyl[4-(1-naphthyl)]imidazole (234 mg, 78%) was prepared by hydrolysis of 2-[4-(4′-methoxyphenyl)-butyrylamino]-2-(4′-n-butoxy-3′-n-butoxycarbonylphenyl)-2-ethyl[4-(naphthyl)]imidazole (330 mg, 0.5 mmol) following the general procedure I.

LCMS: m/z 606 (M+H)⁺; ¹H NMR (DMSO, 400 MHz): δ 0.90 (dt, 3H), 1.10 (m, 4H), 1.14 (m, 2H), 1.23 (m, 2H), 1.43 (m, 2H), 1.70 (m, 2H), 2.03 (t, 2H), 3.09-2.64 (m, 2H), 3.57 (s, 3H), 3.99 (t, 2H), 5.36 (m, 1H), 6.64 (d, 2H), 6.89 (m, 2H), 7.06 (d, Hz, 2H), 7.46-7.36 (m, 4H), 7.63 (d, 2H), 7.82 (d, 1H), 8.17 (bs, 1H) ppm.

### EXAMPLE 47

### 2-[4-(4′-Methoxyphenyl)-butrylamino]-2-(4′-n-butoxy-3′-n-butoxycarbonylphenyl)-2-ethyl [4-(4′-bromo phenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid ( 4.3 g, 10 mmol) was treated with 4-bromophenacyl bromide according to general procedure C to afford the desired keto-ester. The imidazole was formed from the keto ester according to general procedure D. Boc-protected amine was treated as described in general procedure E to give the amine hydrochloride (3.8 g, 75%).

5-{1-Amino-2-[4-(4-bromo-phenyl)-1H-imidazol-2-yl]-ethyl}-2-butoxy-benzoic acid butyl ester hydrochloride (514 mg, 1 mmol) was treated with 4-(4-methoxyphenyl)-butyric acid as described in general procedure F to afford 2-[4-(4′-methoxyphenyl)-butyryl amino]-2-(4′-n-butoxy-3′-n-butoxycarbonylphenyl)-2-ethyl[4-(4′-bromophenyl)]imidazole (571 mg, 75%).

LCMS: m/z 691 (M+H)⁺; ¹H NMR (CD₃OD, 400 MHz): δ 0.90 (dt, 6H), 1.10 (m, 4H), 1.14 (m, 2H), 1.23 (m, 2H), 1.43 (m, 2H), 1.70 (m, 2H), 2.03 (t, 2H), 3.09-2.64 (m, 2H), 3.57 (s, 3H), 4.12 (t, 2H), 4.36 (t, 2H), 5.36 (m, 1H), 6.64 (d, 2H), 6.89 (m, 2H), 7.06 (d, 2H), 7.46-7.36 (m, 2H), 7.63 (d, 2H), 7.82 (d, 1H), 8.17 (bs, 1H) ppm.

### EXAMPLE 48

### 2-[4-(4′-Methoxyphenyl)-butyrylamino]-2-(4′-n-butoxy-3′-carboxyphenyl)-2-ethyl[4-(4′-bromophenyl)]imidazole

2-[4-(4′-Methoxyphenyl)-butyrylamino]-2-(4′-n-butoxy-3′-carboxyphenyl)-2-ethyl[4-(4′-bromophenyl)]imidazole (281 mg, 88%) was prepared by hydrolysis of 2-[4-(4′-methoxyphenylbutyrylamino]-2-(4′-n-butoxy-3′-n-butoxycarbonylphenyl)-2-ethyl [4-(4′-bromophenyl)]imidazole (345 mg, 0.5 mmol) following the general procedure I.

LCMS: m/z 635 (M+H)⁺, ¹H NMR (CD₃OD, 400 MHz): δ 0.92 (dt, 3H), 1.10 (m, 4H), 1.15 (m, 2H), 1.24 (m, 2H), 1.44 (m, 2H), 1.72 (m, 2H), 2.03 (t, 2H), 3.69 (s, 3H), 5.36 (m, 1H), 6.68 (d, 2H), 6.88 (m, 2H), 7.08 (d, 2H), 7.46-7.36 (m, 2H), 7.59 (d, 2H), 7.72 (d, 1H), 7.98 (bs, 1H) ppm.

### EXAMPLE 49

### 2-[4-(4′-Cyclohexyl)-propanoyl amino]-2-(4′-n-butoxy-3′-n-butoxycarbonylphenyl)-2-ethyl [4-(2′-4′-dichloro phenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formylsalicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid ( 4.3 g, 10 mmol) was treated with 2,4-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 75%).

5-{1-Amino-2-[4-(2,4-dichloro-phenyl)-1H-imidazol-2-yl]-ethyl}-2-butoxy-benzoic acid butyl ester hydrochloride (504 mg, 1 mmol) was treated with 4-(4-cyclohexyl)-propionic acid as described in general procedure F to afford 2-[4-(4′-cyclohexyl)-propanoylamino]-2-(4′-n-butoxy-3′-n-butoxycarbonylphenyl)-2-ethyl[4-(2′,4′-dichlorophenyl)]imidazole (502 mg, 78%).

LCMS: m/z 642 (M+H)⁺; ¹H NMR (CD₃OD, 400 MHz): δ 0.96 (dt, 6H), 1.09-1.75 (m, 23H), 2.14 (t, 2H), 3.33 (d, 2H), 3.99 (t, 2H), 4.24 (t, 2H), 5.38 (m, 1H), 7.05 (m, 1H), 7.47-7.38 (m, 1H), 7.56 (d, 2H), 7.63 (s, 1H), 7.68 (m, 1H) ppm.

### EXAMPLE 50

### 2-[4-(4′-Cyclohexyl)-propanoylamino]-2-(4′-n-butoxy-3′-carboxyphenyl)-2-ethyl[4-(2′,4′-dichlorophenyl)]imidazole

2-[4-(4′-Cyclohexyl)-propanoylamino]-2-(4′-n-butoxy-3′-carboxyphenyl)-2-ethyl[4-(2′,4′-dichlorophenyl)] imidazole (253 mg, 86%) was prepared by hydrolysis of 2-[4-(4′-cyclohexyl)propanoylamino]-2-(4′-n-butoxy-3′-n-butoxycarbonylphenyl)-2-ethyl[4-(2′-4′-dichlorophenyl)]imidazole (321 mg, 0.5 mmol) following the general procedure I.

LCMS: m/z 586 (M+H)⁺; ¹H NMR (CD₃OD, 400 MHz): δ 0.96 (dt, 3H), 1.09-1.75 (m, 19H), 2.14 (t, 2H), 3.33 (d, 2H), 4.24 (t, 2H), 5.38 (m, 1H), 7.05 (m, 1H), 7.47-7.38 (m, 1H), 7.56 (d, 2H), 7.63 (s, 1H), 7.68 (m, 1H) ppm.

### EXAMPLE 51

### 2-[4-(4′-Cyclopentyl)-propylamino]-2-(4′-n-butoxy-3′-n-butoxycarbonylphenyl)-2-ethyl [4-(2′,4′-dichloro phenyl)]imidazole

3-[(Tert-butoxycarbonyl)amino]-3-(4′-n-butoxy-3′-n-butoxycarbonylphenyl)-propanoic acid (2.2 g, 52%) was synthesized from 5-formyl salicyclic acid ester ( 2.78 g, 10 mmol) following general procedures A and B. 3-[(Tert-butoxycarbonyl)amino]-3-(4′-n-butoxy-3′-n-butoxycarbonylphenyl)propanoic acid (4.3 g, 10 mmol) was treated with 2,4-dichlorophenacyl bromide according to general procedure C to afford the desired keto-ester. The imidazole was formed from the keto ester according to general procedure D. Boc-protected amine was treated as described in general procedure E to give the amine hydrochloride (3.8 g, 75%).

5-{1-Amino-2-[4-(2,4-dichloro-phenyl)-1H-imidazol-2-yl]-ethyl}-2-butoxy-benzoic acid butyl ester hydrochloride (504 mg, 1 mmol) was treated with 4-(4-cyclopentyl)-propionic acid as described in general procedure F to afford 2-[4-(4′-cyclopentyl)-propylamino]-2-(4′-n-butoxy-3′-n-butoxycarbonylphenyl)-2-ethyl [4-(2′,4′-dichloro phenyl)] imidazole (511 mg, 81%).

LCMS: m/z 628 (M+H)⁺; ¹H NMR (CD₃OD, 400 MHz): δ 0.96 (dt, 6H), 1.25-1.69 (m, 19H), 2.19 (t, 2H), 3.41 (t, 2H), 4.02 (t, 2H), 4.25 (t, 2H), 5.38 (m, 1H), 7.06 (d, 2H), 7.47 (m, 2H), 7.59 (d, 1H), 7.62 (s, 1H), 7.65 (bs, 1H) ppm.

### EXAMPLE 52

### 2-[4-(4′-Cyclopentyl)-propanoylamino]-2-(4′-n-butoxy-3′-carboxyphenyl)-2-ethyl[4-(2′,4′-dichlorophenyl)]imidazole

2-[4-(4′-Cyclopentyl)-propylamino]-2-(4′-n-butoxy-3′-n-butoxycarbonylphenyl)-2-ethyl [4-(2′,4′-dichlorophenyl)]imidazole (217 mg, 75%) was prepared by hydrolysis of 2-[4-(4′-cyclopentyl)propanoylamino]-2-(4′-n-butoxy-3′-n-butoxycarbonylphenyl)-2-ethyl[4-(2′,4′-dichlorophenyl)]imidazole (314 mg, 0.5 mmol) following the general procedure I.

LCMS: m/z 572 (M+H)⁺, ¹H NMR (CD₃OD, 400 MHz): δ 0.96 (dt, 3H), 1.23-1.65 (m, 15H), 2.16 (t, 2H), 3.26 (t, 2H), 4.05 (t, 2H), 5.38 (m, 1H), 7.06 (d, 2H), 7.47 (m, 2H), 7.59 (d, 1H), 7.62 (s, 1H), 7.65 (bs, 1H) ppm.

### EXAMPLE 53

### N-{(1S)-2-(4-Butoxyphenyl)-1-[4-(4-nitrophenyl)-1H-imidazol-2-yl]ethyl}4-tert-butylcyclohexanecarboxamide

4-O-Butyl-N-boc-L-tyrosine (3.3 g, 1 mmole) was treated with 4-nitrophenacyl bromide following the general procedure C to afford the desired keto ester. The imidazole was formed from the keto ester according to general procedure D. Boc-protected amine was treated as described in general procedure E to give 2-(4-butoxy-phenyl)-1-[4-(4-nitro-phenyl)-1H-imidazol-2-yl]-ethylamine hydrochloride (2.9 g, 76%). 2-(4-Butoxy-phenyl)-1-[4-(4-nitro-phenyl)-1H-imidazol-2-yl]-ethylamine hydrochloride (380 mg, 1 mmol) was treated with 4-tert-butylcyclohexylcarboxylic acid as described in general procedure F to afford N-{(1S)-2-(4-butoxyphenyl)-1-[4-(4-nitrophenyl)-1H-imidazol-2-yl]ethyl}-4-tert-butylcyclohexanecarboxamide (423 mg, 78%).

LCMS: m/z 547 (M+H)⁺.

### EXAMPLE 54

### N-{(1S)-2-(4-butoxyphenyl)-1-[4-(4-nitrophenyl)-1H-1-butylimidazol-2-yl]ethyl}-4-butylcyclohexanecarboxamide

N-{(1S)-2-(4-butoxyphenyl)-1-[4-(4-nitrophenyl)-1H-1-butylimidazol-2-yl]ethyl}-4-tert-butylcyclohexanecarboxamide (491 mg, 81%) was prepared from N-{(1S)-2-(4-butyoxyphenyl)-1-[4-(4-nitrophenyl)-1H-imidazol-2-yl]ethyl)-4-tert-butylcyclohexane-carboxamide (546 mg, 1 mmol) and 1-bromobutane following the general procedure H.

LCMS: m/z 603 (M+H)⁺; ¹H NMR (CDCl₃, 400 MHz): δ 0.80-2.00 (m, 32H), 2.05 (m, 1H), 3.19 (m, 2H), 3.50 (m, 1H), 3.60 (m, 1H), 3.88 (t, 2H), 5.30 (m, 1H), 6.47 (d, 1H), 6.73 (d, 2H), 6.94 (d, 2H), 7.16 (s, 1H), 7.89 (d, 2H), 8.24 (d, 2H) ppm.

### EXAMPLE 55

### N-{(1S)-2-(4-Butoxyphenyl)-1-[4-(2,4-dichlorophenyl)-1H-imidazol-2-ylethyl}-4-tert-butylcyclohexanecarboxamide

4-O-Butyl-N-boc-L-tyrosine (3.3 g, 1 mmole) was treated with 2,4-dichlorophenacyl bromide following the general procedure C to afford the desired keto ester. The imidazole was formed from the keto ester according to general procedure D. Boc-protected amine was treated as described in general procedure E to give 2-(4-butoxy-phenyl)-1-[4-(2,4-dichlorophenyl)-1H-imidazol-2-yl]-ethylamine hydrochloride (3.2 g, 79%).

2-(4-Butoxy-phenyl)-1-[4-(2,4-dichloro-phenyl)-1H-imidazol-2-yl]-ethylamine hydrochloride (404 mg, 1 mmol) was treated from 4-tert-butylcyclohexylcarboxylic acid as described in general procedure F to afford N-{(1S)-2-(4-butoxyphenyl)-1-[4-(2,4-dichlorophenyl)-1H-imidazol-2-yl]ethyl}-4-tert-butylcyclohexanecarboxamide (482 mg, 85%).

LCMS: m/z 570 (M+H)⁺.

### EXAMPLE 56

### N-{(1S)-2-(4-butoxyphenyl)-1-[4-(2,4-dichlorophenyl)-1H-1-butylimidazol-2-yl]ethyl}-4-tert-butylcyclohexanecarboxamide

N-{(1S)-2-(4-Butoxyphenyl)-1-[4-(2,4-dichlorophenyl)-1H-1-butylimidazol-2-yl]-ethyl}-4-tert-butylcyclohexanecarboxamide (519 mg, 82%) was prepared from N-{(1S)-2-(4-butoxyphenyl)-1-[4-(2,4-dichlorophenyl)-1H-imidazol-2-yl]ethyl}-4-tert-butylcyclohexane-carboxamide (570 mg, 1 mmol) and 1-bromobutane following the general procedure H.

LCMS: m/z 626 (M+H)⁺; ¹H NMR (CDCl₃, 400 MHz): δ 0.80-1.90 (m, 32H), 2.05 (m, 1H), 3.18 (m, 2H), 3.52 (m, 1H) 3.58 (m, 1H), 3.88 (t, 2H), 5.25 (m, 1H), 6.45 (d, 1H), 6.72 (d, 2H), 6.93 (d, 2H), 7.30 (d, 1H), 7.39 (s, 1H), 7.43 (s, 1H), 8.10 (d, 1H) ppm.

### EXAMPLE 57

### N-{(1S)-2-(4-Butoxyphenyl)-1-[4-(2,4-dichlorophenyl)-1H-1-propoxyimidazol-2-yl]ethyl}-4-tert-butylcyclohexanecarboxamide

N-{(1S)-2-(4-Butoxyphenyl)-1-[4-(2,4-dichlorophenyl)-1H-1-propylimidazol-2-yl]ethyl}-4-tert-butylcyclohexanecarboxamide (489 mg, 79%) was prepared from N-{(1S)-2-(4-butoxyphenyl)-1-[4-(2,4-dichlorophenyl)-1H-imidazol-2-yl]ethyl}4-tert-butylcyclohexane-carboxamide (570 mg, 1 mmol) and 1-bromopropane following the general procedure H.

LCMS: m/z 612 (M+H)⁺; ¹H NMR (CDCl₃, 400 MHz): δ 0.80-1.90 (m, 30H), 1.99 (m, 1H), 3.18 (t, 2H), 3.48 (m, 1H) 3.54 (m, 1H), 3.89 (t, 2H), 5.25 (m, 1H), 6.41 (d, 1H), 6.73 (d, 2H), 6.94 (d, 2H), 7.31 (d, 1H), 7.41 (s, 1H), 7.43 (s, 1H), 8.10 (d, 1H) ppm.

### EXAMPLE 58

### N-{(1S)-2-(4-Butoxyphenyl)-1-[4-(2,4-dichlorophenyl)-1H-1-pentylimidazo;-2-yl]ethyl}-4-tert-butylcyclohexanecarboxamide

N-{(1S)-2-(4-Butoxyphenyl)-1-[4-(2,4-dichlorophenyl)-1H-1-pentylimidazol-2-yl]ethyl}-4-tert-butylcyclohexanecarboxamide (510 mg, 80%) was prepared from N-{(1S)-2-(4-butoxyphenyl)-1-[4-(2,4-dichlorophenyl)-1H-imidazol-2-yl]ethyl}-4-tert-butylcyclohexane-carboxamide (570 mg, 1 mmol) and 1-bromopentane following the general procedure H.

LCMS: m/z 640 (M+H)⁺; ¹H NMR (CDCl₃, 400 MHz): δ 0.80-1.90 (m, 34), 2.00 (m, 1 H), 3.18 (m, 2 H), 3.51 (m, 1 H), 3.58 (m, 1 H), 3.88 (t, 2H), 5.25 (m, 1 H), 6.45 (d, 1H), 6.72 (d, 2H), 6.93 (d, 2 H), 7.31 (d, 1 H), 7.39 (s, 1 H), 7.42 (s, 1 H), 8.09 (d, 1H) ppm.

### EXAMPLE 59

### N-{(1S)-2-(4-Butoxyphenyl)-1-[4-(2,4-dichlorophenyl)-1H-1-hexylimidazol-2-yl]ethyl}-4-tert-butylcyclohexanecarboxamide

N-{(1S)-2-(4-Butoxyphenyl)-1-[4-(2,4-dichlorophenyl)-1H-1-hexylimidazol-2-yl]ethyl}-4-tert-butylcyclohexanecarboxamide (512 mg, 78%) was prepared from N-{(1S)-2-(4-butoxyphenyl)-1-[4-(2,4-dichlorophenyl)-1H-imidazol-2-yl]ethyl}-4-tert-butylcyclohexane-carboxamide (570 mg, 1 mmol) and 1-bromohexane following the general procedure H.

LCMS: m/z 654 (M+H)⁺; ¹H NMR (CDCl₃, 400 MHz): δ 0.80-1.90 (m, 36 H), 2.03 (m, 1H), 3.19 (m, 2 H), 3.22-3.17 (m, 4H), 3.50 (m, 1 H), 3.57 (m, 1 H), 3.88 (t, 2 H), 5.23 ( m, 1 H), 6.45 (d, 1 H), 6.71 (d, 2 H), 6.93 (d, 2 H), 7.31 (d, 2 H), 7.39 (s, 1 H), 7.43 (s, 1 H), 8.10 (d, 1 H) ppm.

### EXAMPLE 60

### N-{(1S)-2-(4-Butoxyphenyl)-1-[4-(2,4-dichlorophenyl)-1H-1-(2-methoxy-1-ethyl)imidazol-2-yl]ethyl}-4-tert-butylcyclohexanecarboxamide

N-{(1S)-2-(4-Butoxyphenyl)-1-[4-(2,4-dichlorophenyl)-1H-1-(2-methoxy-1-ethyl)imidazol-2-yl]ethyl}-4-tert-butylcyclohexanecarboxamide (499 mg, 79%) was prepared from N-{(1S)-2-(4-butoxyphenyl)-1-[4-(2,4-dichlorophenyl)-1H-imidazol-2-yl]ethyl}-4-tert-butylcyclohexane-carboxamide (570 mg, 1 mmol) and 1-bromo-2-methoxyethane following the general procedure H.

LCMS: m/z 628 (M+H)⁺; ¹H NMR (CDCl₃, 400 MHz): δ 8.07 (d, 1H), 7.50 (s, 1H), 7.42 (s, 1H), 7.29 (d, 1H), 6.96 (d, 2H), 6.74 (d, 2H), 6.46 (d, 1H), 5.27 (m, 1H), 3.89 (t, 2H), 3.72 (m, 1H), 3.32 (m, 1H), 3.18 (m, 2H), 2.05 (m, 1H), 1.90-0.80 (m, 25H) ppm.

### EXAMPLE 61

### N-{(1S)-2-(4-butoxyphenyl)-1-[4-(2,4-dichlorophenyl)-1H-1-(2-oxo-1-butyl)imidazol-2-yl]ethyl}-4-tert-butylcyclohexanecarboxamide

N-{(1S)-2-(4-Butoxyphenyl)-1-[4-(2,4-dichlorophenyl)-1H-1-(2-oxo-1-butyl)imidazol-2-yl]ethyl}-4-tert-butylcyclohexanecarboxamide (520 mg, 81%) was prepared from N-{(1S)-2-(4-butoxyphenyl)-1-[4-(2,4-dichlorophenyl)-1H-imidazol-2-yl]ethyl}-4-tert-butylcyclo-hexanecarboxamide (570 mg, 1 mmol) and 1-bromo-2-butanone following the general procedure H.

LCMS: m/z 640 (M+H)⁺; ¹H NMR (CDCl3, 400 MHz): δ 8.13 (d, 1H), 7.41 (s, 1H), 7.40 (s, 1H), 7.31 (d, 1H), 7.01 (d, 2H), 6.76 (d, 2H), 6.50 (d, 1H), 5.08 (m, 4.68 (d, 1H), 4.60 (d, 1H), 3.89 (t, 2H), 3.26 (m, 2H), 2.45 (q, 2H), 2.05 (m, 1H), 1.90-0.80 (m, 28H) ppm.

### EXAMPLE 62

### N-{(1S)-2-(4-Butoxyphenyl)-1-[4-(pentafluorophenyl)-1H-imidazol-2-yl]ethyl}-4-tert-butylcyclohexanecarboxamide

4-O-Butyl-N-boc-L-tyrosine (3.3 g, 1 mmol) was treated with pentafluorophenacyl bromide following the general procedure C to afford the desired keto ester. The imidazole was formed from the keto ester according to general procedure D. Boc-protected amine was treated as described in general procedure E to give 2-(4-butoxy-phenyl)-1-[4-(2,4-dichlorophenyl)-1H-imidazol-2-yl]-ethylamine hydrochloride (3.2 g, 75%).

2-(4-Butoxy-phenyl)-1-(4-pentafluorophenyl-1H-imidazol-2-yl)-ethylamine hydrochloride (425 mg, 1 mmol) was treated with 4-tert-butylcyclohexylcarboxylic acid as described in general procedure F to afford N-{(1S)-2-(4-butoxyphenyl)-1-[4-(pentafluorophenyl)-1H-imidazol-2-yl]ethyl}-4-tert-butylcyclohexanecarboxamide (473 mg, 80%).

LCMS: m/z 592 (M+H)⁺.

### EXAMPLE 63

### N-{(1S)-2-(4-Butoxyphenyl)-1-[4-(Pentafluorophenyl)-1H-1-propylimidazol-2-yl]ethyl}-4-tert-butylcyclohexanecarboxamide

N-{(1S)-2-(4-Butoxyphenyl)-1-[4-(pentafluorophenyl)-1H-1-propylimidazol-2-yl]ethyl}-4-tert-butylcyclohexanecarboxamide (513 mg, 81%) was prepared from N-{(1S)-2-(4-butoxyphenyl)-1-[4-(pentafluorophenyl)-1H-imidazol-2-yl]ethyl}-4-tert-butylcyclohexane-carboxamide (591 mg, 1 mmol) and 1-bromopropane following the general procedure H.

LCMS: m/z 634 (M+H)⁺; ¹H NMR (CDCl₃, 400 MHz): δ 0.70-1.98 (m, 30H), 2.00 (m, 1H), 3.20 ((t, 2H), 3.53 (m, 1H) 3.60 (m, 1H), 3.89 (t, 2H), 5.25 (m, 1H), 6.38 (d, 1H), 6.73 (d, 2H), 6.97 (d, 2H), 7.06 (s, 1H) ppm.

### EXAMPLE 64

### N-{(1S)-2-(4-Butoxyphenyl)-1-[4-(Pentafluorophenyl)-1H-1-butylimidazol-2-yl]ethyl}-4-tert-butylcyclohexanecarboxamide

N-{(1S)-2-(4-Butoxyphenyl)-1-[4-(pentafluorophenyl)-1H-1-butylimidazol-2-yl]ethyl}-4-tert-butylcyclohexanecarboxamide (517 mg, 80%) was prepared from N-{(1S)-2-(4-butoxyphenyl)-1-[4-(pentafluorophenyl)-1H-imidazol-2-yl]ethyl}-4-tert-butylcyclohexane-carboxamide (591 mg, 1 mmol) and 1-bromobutane following the general procedure H.

LCMS: m/z 648 (M+H)⁺; ¹H NMR (CDCl₃, 400 MHz): δ 0.70-1.98 (m, 32H), 2.10 (m, 1H), 3.19 ((t, 2H), 3.58 (m, 1H), 3.74 (m, 1H), 3.88 (t, 2H), 5.34 (m, 1H), 6.60 (d, 1H), 6.73 (d, 2H), 6.99 (d, 2H), 7.06 (s, 1H) ppm.

### EXAMPLE 65

### N-{(1S)-2-(4-Butoxyphenyl)-1-[4-(pentafluorophenyl)-1H-1-pentylimidazol-2-yl]ethyl}-4-tert-butylcyclohexanecarboxamide

N-{(1S)-2-(4-Butoxyphenyl)-1-[4-(pentafluorophenyl)-1H-1-pentylimidazol-2-yl]ethyl}-4-tert-butylcyclohexanecarboxamide (527 mg, 79%) was prepared from N-{(1S)-2-(4-butoxyphenyl)-1-[4-(pentafluorophenyl)-1H-imidazol-2-yl]ethyl}-4-tert-butylcyclohexane-carboxamide (591 mg, 1 mmol) and 1-bromopentane following the general procedure H.

LCMS: m/z 662 (M+H)⁺; ¹H NMR (CDCl₃, 400 MHz): δ 0.70-1.98 (m, 34H), 2.05 (m, 1H), 3.19 (t, 2H), 3.53 (m, 1H), 3.68 (m, 1H), 3.88 (t, 2H), 5.34 (m, 1H), 6.48 (d, 1H), 6.70 (d, 2H), 6.96 (d, 2H), 7.05 (s, 1H) ppm.

### EXAMPLE 66

### N-{(1S)-2-(4-Butoxyphenyl)-1-[4-(pentafluorophenyl)-1H-1-hexylimidazol-2-yl]ethyl}-4-tert-butylcyclohexanecarboxamide

N-{(1S)-2-(4-Butoxyphenyl)-1-[4-(pentafluorophenyl)-1H-1-hexylimidazol-2-yl]ethyl}-4-tert-butylcyclohexanecarboxamide (527 mg, 78%) was prepared from N-{(1S)-2-(4-butoxyphenyl)-1-[4-(pentafluorophenyl)-1H-imidazol-2-yl]ethyl}-4-tert-butylcyclohexane-carboxamide (591 mg, 1 mmol) and 1-bromohexane following the general procedure H.

LCMS: m/z 676 (M+H)⁺; ¹H NMR (CDCl₃, 400 MHz): δ 0.70-1.98 (m, 36), 2.05 (m, 1H), 3.19 (t, 2H), 3.55 (m, 1H), 3.65 (m, 1H), 3.88 (t, 2H), 5.24 (m, 1H), 6.48 (d, 1H), 6.71 (d, 1 H), 6.96 (d, 1 H), 7.06 (s, 1 H) ppm.

### EXAMPLE 67

### N-{(1S)-2-(4-Butoxyphenyl)-1-[4-(pentafluorophenyl)-1H-1-(2-methoxy-1-ethyl)imidazol-2-yl]ethyl}-4-tert-butylcyclohexanecarboxamide

N-{(1S)-2-(4-Butoxyphenyl)-1-[4-(pentafluorophenyl)-1H-1-(2-methoxy-1-ethyl)imidazol-2-yl]ethyl}-4-tert-butylcyclohexanecarboxamide (531 mg, 81%) was prepared from N-{(1S)-2-(4-butoxyphenyl)-1-[4-(pentafluorophenyl)-1H-imidazol-2-yl]ethyl}-4-tert-butylcyclohexane-carboxamide (591 mg, 1 mmol) and 1-bromo-2-methoxyethane following the general procedure H.

LCMS: m/z 650 (M+1)⁺; ¹H NMR (CDCl₃, 400 MHz): δ 0.80-1.98 (m, 25H), 2.06 (m, 1H), 3.01 (m, 2H), 3.07 (m, 2H), 3.16 (s, 3H), 3.35 (m, 1H), 3.74 (m, 1H), 3.89 (t, 2H), 5.27 (m, 1H), 6.40 (d, 2H), 6.75 (d, 2H), 6.97 (d, 2H), 7.18 (s, 1H) ppm.

### EXAMPLE 68

### N-{(1S)-2-(4-Butoxyphenyl)-1-[4-(2,4-dichlorophenyl)-1H-1-(3-phenoxy-1-propyl)-imidazol-2-yl]ethyl}-4-tert-butylcyclohexanecarboxamide

N-{(1S)-2-(4-Butoxyphenyl)-1-[4-(2,4-dichlorophenyl)-1H-1-(3-phenoxy-1-propyl)-imidazol-2-yl]ethyl}-4-tert-butylcyclohexanecarboxamide (529 mg, 75%) was prepared from N-{(1S)-2-(4-butoxyphenyl)-1-[4-(pentafluorophenyl)-1H-imidazol-2-yl]ethyl}-4-tert-butylcyclohexane-carboxamide (570 mg, 1 mmol) and 1-bromo-3-phenoxypropane following the general procedure H.

LCMS: m/z 704 (M+H)⁺; ¹H NMR (CDCl₃, 400 MHz): δ 0.80-1.98 (m, 25H), 2.06 (m,1H), 3.01 (m, 2H), 3.07 (m, 2H), 3.16 (s, 3H), 3.35 (m, 1H), 3.74 (m, 1H), 3.89 (t, 2H), 5.27 (m, 1H), 6.40 (d, 2H), 6.75 (d, 2H), 6.97 (d, 2H), 7.18 (s, 1H) ppm.

### EXAMPLE 69

### 4-tert-Butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide

N-{(1S)-2-(4-Butoxyphenyl)-(1S)-1-[4-(2,4-dichlorophenyl)-1H-1-butylimidazol-2-yl]ethyl}-4-tert-butylcyclohexanecarboxamide (626 mg, 1 mmol) was selectively O-dealkylated following the general procedure K to afford 4-tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (410 mg, 71%).

LCMS: m/z 570 (M+H)⁺.

### EXAMPLE 70

### N-{(1S)-2-(4-Cyclopropoxyphenyl)-1-[4-(2,4-dichlorophenyl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was reacted with cyclopropyl bromide following the general procedure G to afford N-{(1S)-2-(4-cyclopropoxyphenyl)-1-[4-(2,4-dichlorophenyl)-1H-1-(1-butyl)imidazol-2-yl]ethyl}-4-tert-butylcyclohexane-carboxamide (489 mg, 80%).

LCMS: m/z 610 (M+H)⁺; ¹H NMR (CDCl₃, 400 MHz): δ 0.75-2.00 (m, 29H), 2.10 (m, 1H), 3.20 (m, 2H), 3.52 (m, 1H), 3.62 (m, 1H), 4.47 (m, 1H), 5.25 (m, 1H), 6.37 (d, 1H), 6.73 (m, 2H), 6.99 (m, 2H), 7.33 (m, 1H), 7.40 (s, 1H), 7.43 (d, 1H), 8.11 (d, 1H) ppm.

### EXAMPLE 71

### N-{(1S)-2-(4-Isobutoxyphenyl)-1-[4-(2,4-dichlorophenvl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was reacted with 1-bromo-2-methylpropane following the general procedure G to afford N-{(1S)-2-(4-isobutoxy-phenyl)-1-[4-(2,4-dichlorophenyl)-1H-1-(1-butyl)imidazol-2-yl]ethyl}-4-tert-butylcyclo-hexanecarboxamide (519 mg, 82%).

LCMS: m/z 626 (M+H)⁺; ¹H NMR (CDCl₃, 400 MHz): δ 0.750-2.00 (m, 32H), 2.10 (m, 1H), 3.20 (t, 2H), 3.55 (m, 1H), 3.64 (d, 2H), 3.67 (m, 1H), 5.31 (m, 1H ), 6.51 (d, 1H), 6.73 (d, 2H), 6.98 (d, 2H), 7.31 (dd, 1H), 7.40 (s, 1H), 7.43 (d, 1H), 8.10 (d, 1H) ppm.

### EXAMPLE 72

### N-{(1S)-2-(4-(4-Methyl-3-pentene-1-yloxy)phenyl)-1-[4-(2,4-dichlorophenyl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was reacted with 5-bromo 2-methyl 2-pentene following the general procedure G to afford N-{(1S)-2-(4-(4-methyl-3-pentene-1-yloxy)phenyl)-1-[4-(2,4-dichlorophenyl)-1H-1-(1-butyl)imidazol-2-yl]ethyl}-4-tert-butylcyclohexanecarboxamide (526 mg, 80%).

LCMS: m/z 652 (M+H)⁺; ¹H NMR (CDCl₃, 400 MHz): δ 0.85-2.00 (m, 31H), 2.05 (m, 1H), 2.35 (m, 2H), 3.20 (m, 2H), 3.53 (m, 1H), 3.65 (m, 1H), 3.85 (t, 2H), 5.15 (m, 1H), 5.30 (m, 1H), 6.53 (d, 1H), 6.73 (m, 2H), 6.94 (m, 2H), 7.30 (dd, 1H), 7.40 (s, 1H), 7.42 (d, 1H), 8.08 (d, 1H) ppm.

### EXAMPLE 73

### N-{(1S)-2-(4-(3-Carboxy-1-propoxy)phenyl)-1-[4-(2,4-dichlorophenyl)-1H-1-(1-butyl)-imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with 4-bromobutyric acid methyl ester following the general procedure G then the resulted ester was hydrolyzed following general procedure N to afford N-{(1S)-2-(4-(3-carboxy-1-propoxy)phenyl)-1-[4-(2,4-dichlorophenyl)-1H-1-(1-butyl)-imidazol-2-yl]ethyl}-4-tert-butylcyclohexanecarboxamide (459 mg, 70%).

LCMS: m/z 656 (M+H)⁺; ¹H NMR (CDCl₃, 400 MHz): δ 0.8-2.00 (m, 25H), 2.05 (m, 1H), 2.10 (m, 2H), 2.54 (m, 2H), 3.10 (m, 2H), 3.70 (m, 1H), 3.95 (m, 1H), 3.98 (t, 2H), 5.40 (m, 1H), 6.71 (d, 2H), 6.90 (d, 1H), 6.94 (d, 2H), 7.25 (s,1 H), 7.42 (d, 1H), 7.56 (d, 1H), 7.78 (d, 1H) ppm.

### EXAMPLE 74

### N-{(1S)-2-(4-(1,1-Dicarboxymethoxy)phenyl)-1-[4-(2,4-dichlorophenyl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with dimethyl bromomalonate following the general procedure G then the resulted ester was hydrolyzed following general procedure N to afford N-{(1S)-2-(4-(1,1-dicarboxy-methoxy)phenyl)-1-[4-(2,4-dichlorophenyl)-1H-1-(1-butyl)imidazol-2-yl]ethyl}-4-tert-butylcyclohexanecarboxamide (412 mg, 61%).

LCMS: m/z 672 (M+H)⁺; ¹H NMR (CDCl₃, 400 MHz): δ 0.85-2.00 (m, 25H), 2.05 (m, 1H), 3.20 (m, 2H), 3.53 (m, 1H), 3.65 (m, 1H), 5.15 (s, 1H), 5.30 (m, 1H), 6.53 (d, 1H), 6.73 (m, 2H), 6.94 (m, 2H), 7.30 (dd, 1H), 7.40 (s, 1H), 7.42 (d, 1H), 8.08 (d, 1H) ppm.

### EXAMPLE 75

### N-{(1S)-2-(4-(1-carboxy-1-phenylmethoxy)phenyl)-1-[4-(2,4-dichlorophenyl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with methyl alpha-bromo-alpha-phenylacetate following the general procedure G then the resulted ester was hydrolyzed following general procedure N to afford N-{(1S)-2-(4-(1-carboxy-1-phenyl-methoxy)phenyl)-1-[4-(2,4-dichlorophenyl)-1H-1-(1-butyl)imidazol-2-yl]ethyl}-4-tert-butylcyclohexanecarboxamide (479 mg, 68%).

LCMS: m/z 704 (M+H)⁺; ¹H NMR (CDCl₃/CD₄OD; 400 MHz): δ 0.85-2.00 (m, 25H), 2.05 (m, 1H), 3.11 (m, 2H), 3.53 (m, 1H), 3.65 (m, 1H), 5.15 (m, 1H), 5.39 (s, 1H), 6.79 (d, 2H), 6.92 (m, 3H), 7.20-7.40 (m, 4H), 7.40-7.60 (m, 3H), 7.88 (d, 1H) ppm.

### EXAMPLE 76

### N-{(1S)-2-(4-((2-Phenyl-1-carboxy)1-ethoxy)phenyl)-1-[4-(2,4-dichlorophenyl)-1H-1-(1-butyl)imidazol-2-yl]ethyl}-4-tert-butylcyclohexanecarboxamide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with 2-bromo-3-phenylpropionic acid methyl ester following the general procedure G then the resulted ester was hydrolyzed following general procedure N to afford N-{(1S)-2-(4-((2-phenyl-1-carboxy)1-ethoxy)phenyl)-1-[4-(2,4-dichlorophenyl)-1H-1-(1-butyl)imidazol-2-yl]ethyl}-4-tert-butylcyclohexanecarboxamide (499 mg, 70%).

LCMS: m/z 718 (M+H)⁺; ¹H NMR (CDCl₃/CD₄OD; 400 MHz): δ 0.85-2.00 (m, 25H), 2.05 (m, 1H), 3.13 (m, 2H), 3.20 (m, 2H), 3.63 (m, 1H), 3.73 (m, 1H), 4.71 (m, 1H), 6.71 (d, 2H), 6.94 (d, 2H), 7.20-7.35(m, 7H), 7.47 (d, 1H), 7.63 (s, 1H), 7.85 (d, 1H) ppm.

### EXAMPLE 77

### 4-(4-{(2S)-2-[(4-tert-Butyl-cyclohexanecarbonyl)-amino]-2-{1-butyl-4-(2,4-dichloro-phenyl)-1H-imidazol-2-yl]-ethyl}-phenoxymethyl)-benzoic acid acid

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with methyl 4-(bromomethyl)benzoate following the general procedure G then the resulted ester was hydrolyzed following general procedure N to afford 4-(4-{(2S)-2-[(4-tert-butyl-cyclohexane-carbonyl)-amino]-2-[1-butyl-4-(2,4-dichloro-phenyl)-1H-imidazol-2-yl]-ethyl}-phenoxy-methyl)-benzoic acid (512 mg, 72%).

LCMS: m/z 704 (M+H)⁺; ¹H NMR (CDCl₃, 400 MHz): δ 0.8-2.00 (m, 25H), 2.10 (m,1H), 3.21 (t, 2H), 3.65 (m, 1H), 3.76 (m, 1H), 5.07 (s, 2H), 5.40 (m, 1H), 6.78 (d, 2H), 6.98 (d, 2H), 7.32 (m, 2H), 7.45 (m, 3H), 7.75 (d,1 H), 7.90 (d,1 H), 8.07 (d, 2H) ppm.

### EXAMPLE 78

### {2-(4-Butoxy-phenyl)-(1S)-1-[1-butyl-4-(2,4-dichloro-phenyl)-1H-imidazol-2-yl]-ethyl}-carbamic acid tert-butyl ester

N-Boc-L-tyrosine (2.8 g, 10 mmol) was treated with 2,4-dichlorophenacyl bromide following the general procedure C to afford the desired keto ester. The imidazole was formed from the keto ester according to general procedure D and was alkylated following general procedure H to afford {2-(4-butoxy-phenyl)-(1S)-1-[1-butyl-4-(2,4-dichloro-phenyl)-1H-imidazol-2-yl]-ethyl}-carbamic acid tert-butyl ester (3.17 g, 80%).

LCMS: m/z 560 (M+H)⁺; ¹H NMR (CDCl₃, 400 MHz): δ 0.90 (s, 9H), 0.95 (dt, 6H), 1.54 (m, 4H), 1.85 (m, 4H), 3.18 (m, 2H), 3.52 (m, 2H), 3.88 (m, 2H), 5.25 (m, 1), 6.45 (d, 1H), 6.72 (d, 2H), 6.93 (d, 2H), 7.30 (d, 1H), 7.39 (s, 1H), 7.43 (s, 1H), 8.10 (d, 1H) ppm.

### EXAMPLE 79

### [(1S)-1-[1-Butyl-4-(2,4-dichloro-phenyl)-1H-imidazol-2-yl]-2-(4-hydroxy-phenyl)-ethyl]-carbamic acid tert-butyl ester

{2-(4-Butoxy-phenyl)-(1S)-1-[1-butyl-4-(2,4-dichloro-phenyl)-1H-imidazol-2-yl]-ethyl}-carbamic acid tert-butyl ester (5.6 g, 10 mmol) was treated with BBr₃ as described in general procedure K to obtain phenol-amine hydrochloride. The resulted amine hydrochloride was treated with boc-anhydride following general procedure B to afford [(1S)-1-[1-butyl-4-(2,4-dichloro-phenyl)-1H-imidazol-2-yl]-2-(4-hydroxy-phenyl)-ethyl]-carbamic acid tert-butyl ester (2.9 g, 80%).

LCMS: m/z 504 (M+H)⁺.

### EXAMPLE 80

### 4-(4-{(2S)-2-tert-Butoxycarbonylamino-2-[1-butyl-4-(2,4-dichlorophenyl)-1H-imidazol-2-yl]-ethyl}-phenoxymethyl)-benzoic acid methyl ester

[1-[1-Butyl-4-(2,4-dichloro-phenyl)-1H-imidazol-2-yl]-(2S)-2-(4-hydroxy-phenyl)-ethyl]-carbamic acid tert-butyl ester (504 mg, 1 mmol) was reacted with methyl 4-(bromomethyl)benzoate following the general procedure G to afford 4-(4-{(2S)-2-tert-butoxycarbonylamino-2-[1-butyl-4-(2,4-dichlorophenyl)-1H-imidazol-2-yl]-ethyl}-phenoxymethyl)-benzoic acid methyl ester (489 mg, 75%).

LCMS: m/z 652 (M+H)⁺; ¹H NMR (CDCl₃, 400 MHz): δ 0.8 (s, 9H), 0.94 (m, 2H), 1.28 (m, 2H), 1.45 (m, 2H), 3.19 (m, 2H), 3.52 (q, 2H), 3.72 (s, 3H), 4.96 (s, 1H), 5.25 (m, 1H), 6.52 (d, 2H), 6.78 (d, 2H), 6.93 (d, 2H), 7.31 (d, 2H), 7.40 (s, 1H), 7.62 (d, 1H), 7.42 (s, 1H), 7.49 (d, 2H), 8.09 (d, 1H) ppm.

### EXAMPLE 81

### 4-(4-{(2S)-2-tert-Butoxycarbonylamino-2-[1-butyl-4-(2,4-dichloro-phenyl)-1H-imidazol-2-yl]-ethyl}-phenoxymethyl)-benzoic acid

4-(4-{(2S)-2-Tert-butoxycarbonylamino-2-[1-butyl-4-(2,4-dichlorophenyl)-1H-imidazol-2-yl]-ethyl}-phenoxymethyl)-benzoic acid methyl ester (326 mg, 0.5 mmol) was hydrolyzed following general procedure N to afford 4-(4-{(2S)-2-tert-butoxycarbonylamino-2-[1-butyl-4-(2,4-dichloro-phenyl)-1H-imidazol-2-yl]-ethyl}-phenoxymethyl)-benzoic acid (276 mg, 86%).

LCMS: m/z 638 (M+H)⁺; ¹H NMR (CDCl₃, 400 MHz): δ 0.92 (s, 9H), 1.10 (m, 1H), 1.26 (m, 2H), 1.41 (m, 2H), 3.24 (m, 2H), 3.53 (q, 2H), 4.96 (m, 1 H), 5.25 (s, 1H), 5.90 (d, 2H), 6.81 (d, 2H), 7.01 (d, 2H), 7.29 (s, 1H), 7.30 (d, 2H), 7.36 (s, 1H), 7.43 (s, 1H), 7.47 (d, 2H), 8.05 (d, 2H), 8.10 (s, 1H) ppm.

### EXAMPLE 82

### 4-(4-{(2S)-2-Amino-2-[1-butyl-4-(2,4-dichloro-phenyl)-1H-imidazol-2-yl]-ethyl}-phenoxymethyl)-benzoic acid methyl ester hydrochloride

4-(4-{(2S)-2-Tert-butoxycarbonylamino-2-[1-butyl-4-(2,4-dichlorophenyl)-1H-imidazol-2-yl]-ethyl}-phenoxymethyl)-benzoic acid methyl ester (319 mg, 0.5 mmol) was treated with HCl in dioxane to afford 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (191 mg mg, 70%).

LCMS: m/z 552 (M+H)⁺; ¹H NMR (DMSO, 400 MHz): δ 0.73 (t, 3H), 0.93-0.97 (m, 1H), 1.17-1.18 (m 1H), 3.24-3.27 (m, 1H), 3.33-3.42 (m, 1H), 3.46-3.49 (m, 1H), 3.56-3.59 (m, 1H), 3.67-3.69 (m, 1H), 3.71-3.74 (m, 1H), 3.84 (s, 3H), 4.66 (m, 1H), 5.13 (s, 1H), 6.88 (d, 2H), 6.94 (d, 2H), 7.51-7.56 (m, 4H), 7.67 (s, 1H), 7.75 (d, 2H), 8.21 (d, 1H), 8.8 (s, 1H) ppm.

### EXAMPLE 83

### 4-(4-{2-[1-Butyl-4-(2,4-dichloro-phenyl)-1H-imidazol-2-yl]-(2S)-2-[2-(4-methoxy-phenyl)-acetylamino]-ethyl}-phenoxymethyl)-benzoic acid

4-(4-{2-[1-Butyl-4-(2,4-dichloro-phenyl)-1H-imidazol-2-yl]-(2S)-2-[2-(4-methoxy-phenyl)-acetylamino]-ethyl}-phenoxymethyl)-benzoic acid (251 mg, 73%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 4-methoxyphenylacetic acid via amide formation followed by ester hydrolysis following general procedures F and N respectively.

LCMS: m/z 686 (M+H)⁺; ¹H NMR (CDCl₃, 400 MHz): δ 0.88 (t, 3H), 1.22 (m, 2H), 1.47 (m, 2H), 3.22 (d, 2H), 3.49 (s, 2H), 3.64 (m,1 H), 3.79 (s, 3H), 3.82 (m, 1H), 5.29 (m, 1H), 5.37 (s, 1H), 6.82 (d, 2H), 6.87 (d, 2H), 6.92 (d, 2H), 7.01 (d, 2H), 7.16 (d, 2H), 7.22 (d, 1H), 7.31 (d, 1H), 7.37 (s, 1H), 7.44 (s, 1H), 7.86 (d, 1H), 8.01 (d, 2H), 8.11 (s, 1H) ppm.

### EXAMPLE 84

### 4-(4-{2-[1-Butyl-4-(2,4-dichloro-phenyl)-1H-imidazol-2-yl]-(2S)-2-[2-(4-tert-butyl-phenyl)-acetylaminol-ethyl]-phenoxymethyl)-benzoic acid

4-(4-{2-[1-Butyl-4-(2,4-dichloro-phenyl)-1H-imidazol-2-yl]-(2S)-2-[2-(4-butyl-phenyl)-acetylamino]-ethyl}-phenoxymethyl)-benzoic acid (269 mg, 75%) was prepared from 4-(4-{(2S -2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 4-tert-butylphenylacetic acid via amide formation followed by ester hydrolysis following general procedures F and N respectively.

LCMS: m/z 712 (M+H)⁺; ¹H NMR (CDCl₃, 400 MHz): δ 0.81 (s, 9H), 0.89 (t, 3H), 1.21 (m, 2H), 1.45 (m, 2H), 3.21 (d, 2H), 3.47 (s, 2H), 3.68 (m, 1H), 3.84 (m, 1H), 5.27 (m, 1H), 5.39 (s, 1H), 6.81 (d, 2H), 6.88 (d, 2H), 6.91 (d, 2H), 7.04 (d, 2H), 7.18 (d, 2H), 7.24 (d, 1H), 7.32 (d, 1H), 7.36 (s, 1H), 7.45 (s, 1H), 7.87 (d, 1H), 8.03 (d, 2H), 8.10 (s, 1H) ppm.

### EXAMPLE 85

### 4-{4-[2-1-Butyl-4-(2,4-dichloro-phenyl)-1H-imidazol-2-yl]-(2S)-2-(2-naphthalen-1-yl-acetylamino)-ethyl]-phenoxymethyl}benzoic acid

4-{4-[2-[1-Butyl-4-(2,4-dichloro-phenyl)-1H-imidazol-2-yl]-(2S)-2-(2-naphthalen-1-yl-acetylamino)-ethyl]-phenoxymethyl}benzoic acid (249 mg, 70%) was prepared from 4-(4-{(2S)-2-amino-2-[1-butyl-4-(2,4-dichloro-phenyl)-1H-imidazol-2-yl]-phenoxymethyl)-benzoic acid methyl ester hydrochloride (276 mg, 0.5 mmol) and 1-naphthylacetic acid via amide formation followed by ester hydrolysis following general procedures F and N respectively.

LCMS: m/z 706 (M+H)⁺; ¹H NMR (CDCl₃, 400 MHz): δ 0.88 (t, 3H), 1.22 (m, 2H), 1.47 (m, 2H), 3.22 (d, 2H), 3.49 (s, 2H), 3.64 (m,1 H), 3.82 (m,1 H), 5.29 (m, 1H), 5.37 (s, 1H), 6.82 (d, 2H), 6.87 (d, 2H), 6.92 (d, 2H), 7.01 (d, 2H), 7.16 (d, 2H), 7.22 (d, 1H), 7.31 (d, 1H), 7.32-7.41 (m, 4H), 7.44 (s, 1H), 7.86 (d, 1H), 8.01 (d, 2H), 8.11 (s, 1H) ppm.

### EXAMPLE 86

### 4-[4-(2-[1-Butyl-4-(2,4-dichloro-phenyl)-1H-imidazol-2-yl]-(2S)-2-{[1-(4-methoxy-phenyl)-cyclopentanecarbonyl]-amino}-ethyl)-phenoxymethyl]-benzoic acid

4-[4-(2-[1-Butyl-4-(2,4-dichloro-phenyl)-1H-imidazol-2-yl]-(2S)-2-{[1-(4-methoxy-phenyl)-cyclopentanecarbonyl]-amino}-ethyl)-phenoxymethyl]-benzoic acid (259 mg, 70%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 1-(4-methoxyphenyl)cyclopentanecarboxylic acid via amide formation followed by ester hydrolysis following general procedures F and N respectively.

LCMS: m/z 740 (M+H)⁺.

### EXAMPLE 87

### 4-(4-{2-[1-Butyl-4-(2,4-dichloro-phenyl)-1H-imidazol-2-yl]-(2S)-2-[2-(4-chloro-phenyl)-2-methyl-propionylamino]-ethyl}-phenoxymethyl)-benzoic acid

4-(4-{2-[1-Butyl-4-(2,4-dichloro-phenyl)-1H-imidazol-2-yl]-(2S)-2-[2-(4-chloro-phenyl)-2-methyl-propionylamino]-ethyl}-phenoxymethyl)-benzoic acid (256 mg, 71%) was prepared from 4-(4-{(2S)-2-amino-2-[1-butyl-4-(2,4-dichloro-phenyl)-1H-imidazo-2-yl]-ethyl}-phenoxymethyl)-benzoic acid methyl ester hydrochloride (276 mg, 0.5 mmol) and 2-(4-chloro-phenyl)-2-methyl-propanoic acid via amide formation followed by ester hydrolysis following general procedures F and N respectively.

LCMS: m/z 718 (M+H)⁺.

### EXAMPLE 88

### 4-(4-{2-[1-Butyl-4-(2,4-dichloro-phenyl)-1H-imidazol-2-yl]-(2S)-2-phenylacetylamino-ethyl}-phenoxymethyl)-benzoic acid

4-(4-{2-[1-Butyl-4-(2,4-dichloro-phenyl)-1H-imidazol-2-yl]-(2S)-2-phenylacetylamino-ethyl}-phenoxymethyl)-benzoic acid (241 mg, 73%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and phenylacetic acid via amide formation followed by ester hydrolysis following general procedures F and N respectively.

LCMS: m/z 656 (M+H)⁺.

### EXAMPLE 89

### 4-(4-{2-[1-Butyl-4-(2,4-dichloro-phenyl)-1H-imidazol-2-yl]-(2S)-2-[2-(1-methyl-1H-indol-2-yl)-acetylamino]-ethyl}-phenoxymethyl)-benzoic acid

4-(4-{2-[1-Butyl-4-(2,4-dichloro-phenyl)-1H-imidazol-2-yl]-(2S)-2-[2-(1-methyl-1H-indol-2-yl)-acetylamino]-ethyl}-phenoxymethyl)-benzoic acid (226 mg, 64%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and (1-methyl-1H-indol-3-yl)acetic acid via amide formation followed by ester hydrolysis following general procedures F and N respectively.

LCMS: m/z 709 (M+H)⁺.

### EXAMPLE 90

### 4-(4-{2-[1-Butyl-4-(2,4-dichloro-phenyl)-1H-imidazol-2-yl]-(2S)-2-[4-(4-methoxy-phenyl)-butyrylamino]-ethyl}-phenoxymethyl)-benzoic acid

4-(4-{2-[1-Butyl-4-(2,4-dichloro-phenyl)-1H-imidazol-2-yl]-(2S)-2-[4-(4-methoxy-phenyl)-butyrylamino]-ethyl}-phenoxymethyl)-benzoic acid (248 mg, 70%) was prepared from 4-(4-{(2S)-2-amino-2-[1-butyl-4-(2,4-dichloro-phenyl)-1H-imidazol-2-yl]-ethyl}-phenoxymethyl)benzoic acid methyl ester hydrochloride (276 mg, 0.5 mmol) and 4-(4-methoxy-phenyl)-butyric acid via amide formation followed by ester hydrolysis following general procedures F and N respectively.

LCMS: m/z 714 (M+H)⁺; ¹H NMR (CDCl₃, 400 MHz): δ 0.89 (t, 3H), 1.21 (m, 2H), 1.48 (m, 2H), 1.65 (m, 2H), 2.05 (m, 4H), 3.22 (d, 2H), 3.63 (m, 1 H), 3.78 (s, 3H), 3.81 (m, 1H), 5.25 (m, 1H), 5.39 (s, 1H), 6.81 (d, 2H), 6.85 (d, 2H), 6.91 (d, 2H), 7.01 (d, 2H), 7.15 (d, 2H), 7.21 (d, 1H), 7.34 (d, 1H), 7.37 (s, 1H), 7.41 (s, 1H), 7.85 (d, 1H), 8.04 (d, 2H), 8.11 (s, 1H) ppm.

### EXAMPLE 91

### 4-(4-{2-[1-Butyl-4-(2,4-dichloro-phenyl)-1H-imidazol-2-yl]-(2S)-2-[3-(4-methoxy-phenyl)-propionyl-amino]-ethyl}-phenoxymethyl)-benzoic acid

4-(4-{2-[1-Butyl-4-(2,4-dichloro-phenyl)-1H-imidazol-2-yl]-(2S)-2-[3-(4-methoxy-phenyl)-propionylamino]-ethyl}-phenoxymethyl)-benzoic acid (261 mg, 74%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 3-(4-methoxy-phenyl)-propanoic acid via amide formation followed by ester hydrolysis following general procedures F and N respectively.

LCMS: m/z 700 (M+H)⁺; ¹H NMR (CDCl₃, 400 MHz): δ 0.88 (t, 3H), 1.22 (m, 2H), 1.47 (m, 2H), 1.82 (m, 2H), 3.22 (d, 2H), 3.49 (s, 2H), 3.64 (m, 1H), 3.79 (s, 3H), 3.82 (m, 1H), 5.29 (m, 1H), 5.37 (s, 1H), 6.82 (d, 2H), 6.87 (d, 2H), 6.92 (d, 2H), 7.01 (d, 2H), 7.16 (d, 2H), 7.22 (d, 1H), 7.31 (d, 1H), 7.37 (s, 1H), 7.44 (s, 1H), 7.86 (d, 1H), 8.01 (d, 2H), 8.11 (s, 1H) ppm.

### EXAMPLE 92

### 4-(4-{2-[1-Butyl-4-(2,4-dichloro-phenyl)-1H-imidazol-2-yl]-(2S)-2-[3-(2,4-difluoro-phenyl)-acryloylamino]-ethyl}-phenoxymethyl)-benzoic acid

4-(4-{2-[1-Butyl-4-(2,4-dichloro-phenyl)-1H-imidazol-2-yl]-(2S)-2-[3-(2,4-difluoro-phenyl)-acryloylamino]-ethyl}-phenoxymethyl)-benzoic acid (251 mg, 69%) was prepared from 4-(4-{(2S)-2-amino-2-[1-butyl-4-(2,4-dichloro-phenyl)-1H-imidazol-2-yl]-ethyl)phenoxymethyl)-benzoic acid methyl ester hydrochloride (276 mg, 0.5 mmol) and 3-(2,4-difluoro-phenyl)-acrylic acid via amide formation followed by ester hydrolysis following general procedures F and N respectively.

LCMS: m/z 704 (M+H)⁺.

### EXAMPLE 93

### 4-(4-{2-[1-Butyl-4-(2,4-dichloro-phenyl)-1H-imidazol-2-yl]-(2S)-2-[3-(4-hydroxy-phenyl)-acryloylamino]-ethyl}-phenoxymethyl)-benzoic acid

4-(4-{2-[1-Butyl-4-(2,4-dichloro-phenyl)-1H-imidazol-2-yl]-(2S)-2-[3-(4-hydroxy-phenyl)-acryloylamino]-ethyl}-phenoxymethyl)-benzoic acid (212 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 3-(4-hydroxy-phenyl)-acrylic acid via amide formation followed by ester hydrolysis following general procedures F and N respectively.

LCMS: m/z 684 (M+H)⁺.

### EXAMPLE 94

### 4-(4-{2-[1-Butyl-4-(2,4-dichloro-phenyl)-1H-imidazol-2-yl]-(2S)-2-[3-(4-ethoxy-phenyl)-acryloylamino]-ethyl}-phenoxymethyl)-benzoic acid

4-(4-{2-[1-Butyl-4-(2,4-dichloro-phenyl)-1H-imidazol-2-yl]-(2S)-2-[3-(4-ethoxy-phenyl)-phenyl)-acryloylamino]-ethyl}-phenoxymethyl)-benzoic acid (261 mg, 73%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 3-(4-ethoxy-phenyl)-acrylic acid via amide formation followed by ester hydrolysis following general procedures F and N respectively.

LCMS: m/z 712 (M+H)⁺.

### EXAMPLE 95

### 4-(4-{2-[3-(4-Butoxy-phenyl)-acryloylamino]-(2S)-2-[1-butyl-4-(2,4-dichloro-phenyl)-1H-imidazol-2-yl]-ethyl}-phenoxymethyl)-benzoic acid

4-(4-{2-[3-(4-Butoxy-phenyl)-acryloylamino]-(2S)-2-[1-butyl-4-(2,4-dichloro-phenyl)-1H-imidazol-2-yl]-ethyl}-phenoxymethyl)-benzoic acid (112 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 3-(4-butoxy-phenyl)-acrylic acid via amide formation followed by ester hydrolysis following general procedures F and N respectively.

LCMS: m/z 740 (M+H)⁺.

### EXAMPLE 96

### 4-(4-{2-[3-(4-tert-Butoxycarbonylamino-phenyl)-acryloylamino]-(2S)-2-[1-butyl-4-(2,4-dichloro-phenyl)-1H-imidazol-2-yl]-ethyl}-phenoxymethyl)-benzoic acid

4-(4-{2-[3-(4-tert-Butoxycarbonylamino-phenyl)-acryloylamino]-(2S)-2-[1-butyl-4-(2,4-dichloro-phenyl)-1H-imidazol-2-yl]-ethyl}-phenoxymethyl)-benzoic acid (269 mg, 68%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (276 mg, 0.5 mmol) and 3-(4-tert-butoxycarbonylamino-phenyl)-acrylic acid via amide formation followed by ester hydrolysis following general procedures F and N respectively.

LCMS: m/z 783 (M+H)⁺; ¹H NMR (CDCl₃, 400 MHz): δ 0.83 (s, 9H), 0.89 (t, 3H), 1.14 (m, 1H), 1.28 (m, 1H), 1.34 (m, 1H), 1.42 (m, 1H), 1.46 (m, 1H), 3.67 (m, 1H), 3.82 (m, 1H), 5.07 (s 1H), 5.34 (m, 1H), 6.54 (d, 2H), 6.84 (d, 2H), 7.06 (d, 2H), 7.33 (d, 2H), 7.34 (d, 2H), 7.38 (d, 1H), 7.43-7.47 (m, 4H), 7.48-7.49 (m, 4H), 7.95 (s, 1H), 7.97-8.0 (m, 2H) ppm.

### EXAMPLE 97

### 4-(4-{2-[3-(4-Amino-phenyl)-acryloylamino]-(2S)-2-[1-butyl-4-(2,4-dichloro-phenyl)-1H-imidazol-2-yl]-ethyl}-phenoxymethyl)-benzoic acid hydrochloride

4-(4-{2-[3-(4-Amino-phenyl)-acryloylamino]-(2S)-2-[1-butyl-4-(2,4-dichloro-phenyl)-1H-imidazol-2-yl]-ethyl}-phenoxymethyl)-benzoic acid hydrochloride (266 mg, 78%) was prepared from 4-(4-{(2S)-2-[3-(4-tert-butoxycarbonylamino-phenyl)-acryloylamino]-2-[1-butyl-4-(2,4-dichloro-phenyl)-1H-imidazol-2-yl]-ethyl}-phenoxymethyl)-benzoic acid (391 mg, 0.5 mmol) via removal of boc-group following general procedure E.

LCMS: m/z 683 (M+H)⁺; ¹H NMR (CDCl₃, 400 MHz): δ 0.89 (t, 3H), 1.14 (m, 1H), 1.28 (m, 1H), 1.34 (m, 1H), 1.42 (m, 1H), 1.46 (m, 1H), 3.67 (m, 1H), 3.82 (m, 1H), 5.07 (s 1H), 5.34 (m, 1H), 6.54 (d, 2H), 6.84 (d, 2H), 7.06 (d, 2H), 7.33 (d, 2H), 7.34 (d, 2H), 7.38 (d, 1H), 7.43-7.47 (m, 4H), 7.48-7.49 (m, 4H), 7.95 (s, 1H), 7.97-8.0 (m, 2H) ppm.

### EXAMPLE 98

### 4-{4-[2-[1-Butyl-4-(2,4-dichloro-phenyl)-1H-imidazol-2-yl]-(2S)-2-(4-(1H-indol-2-yl)-butyrylamino)-ethyl]-phenoxymethyl}-benzoic acid

4-{4-[2-[1-Butyl-4-(2,4-dichloro-phenyl)-1H-imidazol-2-yl]-(2S)-2-(4-1H-indol-2-yl-butyrylamino)-ethyl]-phenoxymethyl}-benzoic acid (121 mg, 67%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-(indol-3-yl)butyric acid via amide formation followed by ester hydrolysis following general procedures F and N respectively.

LCMS: m/z 723 (M+H)⁺.

### EXAMPLE 99

### 4-{4-[2-[1-Butyl-4-(2,4-dichloro-phenyl)-1H-imidazol-2-yl]-(2S)-2-(3-fluoro-benzoylamino)-ethyl]-phenoxymethyl}-benzoic acid

4-{4-[2-[1-Butyl-4-(2,4-dichloro-phenyl)-1H-imidazol-2-yl]-(2S)-2-(3-fluoro-benzoylamino)-ethyl]-phenoxymethyl}-benzoic acid (48 mg, 72%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and 3-fluorobenzoic acid via amide formation followed by ester hydrolysis following general procedures F and N respectively.

LCMS: m/z 660 (M+H)⁺.

### EXAMPLE 100

### 4-[4-[2-[1-Butyl-4-(2,4-dichloro-phenyl)-1H-imidazol-2-yl]-(2S)-2-(3-cyano-benzoylamino)-ethyl]-phenoxymethyl}-benzoic acid

4-{4-[2-[1-Butyl-4-(2,4-dichloro-phenyl)-1H-imidazol-2-yl]-(2S)-2-(3-cyano-benzoylamino)-ethyl]-phenoxymethyl}-benzoic acid (44 mg, 65%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and 3-cyanobenzoic acid via amide formation followed by ester hydrolysis following general procedures F and N respectively.

LCMS: m/z 667 (M+H)⁺.

### EXAMPLE 101

### 4-(4-{2-(4-tert-Butyl-benzoylamino)-(2S)-2-[1-butyl-4-(2,4-dichloro-phenyl)-1H-imidazol-2-yl]-ethyl}-phenoxymethyl)-benzoic acid

4-(4-{2-(4-tert-Butyl-benzoylamino)-(2S)-2-[1-butyl-4-(2,4-dichloro-phenyl)-1H-imidazol-2-yl]-ethyl}-phenoxymethyl)-benzoic acid (54 mg, 77%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and 4-tert-butylbenzoic acid via amide formation followed by ester hydrolysis following general procedures F and N respectively.

LCMS: m/z 698 (M+H)⁺.

### EXAMPLE 102

### 4-{4-[2-[1-Butyl-4-(2,4-dichloro-phenyl)-1H-imidazol-2-yl]-(2S)-2-(3,4-difluoro-benzoylamino)-ethyl]-phenoxymethyl}-benzoic acid

4-{4-[2-[1-Butyl-4-(2,4-dichloro-phenyl)-1H-imidazol-2-yl]-(2S)-2-(3,4-difluoro-benzoylamino)-ethyl]-phenoxymethyl}-benzoic acid (48 mg, 71%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and 3,4-difluorobenzoic acid via amide formation followed by ester hydrolysis following general procedures F and N respectively.

LCMS: m/z 678 (M+H)⁺.

### EXAMPLE 103

### 4-{4-[2-[1-Butyl-4-(2,4-dichloro-phenyl)-1H-imidazol-2-yl]-(2S)-2-(2-chloro-4-fluoro-benzoylamino)-ethyl]-phenoxymethyl}-benzoic acid

4-{4-[2-[1-Butyl-4-(2,4-dichloro-phenyl)-1H-imidazol-2-yl]-(2S)-2-(2-chloro-4-fluoro-benzoyl-amino)-ethyl]-phenoxymethyl)benzoic acid (121 mg, 70%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 2-chloro-4-fluorobenzoic acid via amide formation followed by ester hydrolysis following general procedures F and N respectively.

LCMS: m/z 694 (M+H)⁺.

### EXAMPLE 104

### 4-{4-[2-[1-Butyl-4-(2,4-dichloro-phenyl)-1H-imidazol-2-[1-(2S)-2-(4-phenoxy-benzoylamino)-ethyl]-phenoxymethyl}-benzoic acid

4-{4-[2-[1-Butyl-4-(2,4-dichloro-phenyl)-1H-imidazol-2-yl]-(2S)-2-(4-phenoxy-benzoylamino)-ethyl]-phenoxymethyl}-benzoic acid (117 mg, 64%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-phenoxybenzoic acid via amide formation followed by ester hydrolysis following general procedures F and N respectively.

LCMS: m/z 734 (M+H)⁺.

### EXAMPLE 105

### 4-(4-{2-(4-Butoxy-benzoylamino)-(2S)-2-[1-butyl-4-(2,4-dichloro-phenyl)-1H-imidazol-2-yl]-ethyl}-phenoxymethyl)-benzoic acid

4-(4-{2-(4-Butoxy-benzoylamino)-(2S)-2-[1-butyl-4-(2,4-dichloro-phenyl)-1H -imidazol-2-yl]-ethyl}-phenoxymethyl)-benzoic acid (178 mg, 72%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-butoxybenzoic acid via amide formation followed by ester hydrolysis following general procedures F and N respectively.

LCMS: m/z 714 (M+H)⁺.

### EXAMPLE 106

### 4-(4-{2-[1-Butyl-4-(2,4-dichloro-phenyl)-1H-imidazol-2-yl]-(2S)-2-[(pyridin-3-carbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(pyridine-3-carbonyl)-amino]-ethyl}-phenoxymethyl)-benzoic acid (99 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and pyridine-3-carboxylic acid via amide formation followed by ester hydrolysis following general procedures F and N respectively.

LCMS: m/z 643 (M+H)⁺.

### EXAMPLE 107

### 4-(4-{2-[1-Butyl-4-(2,4-dichloro-phenyl)-1H-imidazol-2-yl]-(2S)-2-[(isoquinoline-3-carbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(isoquinoline-3-carbonyl)-amino]-ethyl}-phenoxymethyl)-benzoic acid (112 mg, 65%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and isoquinoline-3-carboxylic acid via amide formation followed by ester hydrolysis following general procedures F and N respectively.

LCMS: m/z 693 (M+H)⁺.

### EXAMPLE 108

### 4-{4-[2-[1-Butyl-4-(2,4-dichloro-phenyl)-1H-imidazol-2-yl]-(2S)-2-(2-cyclopentyl-acetylamino)-ethyl]-phenoxymethyl}-benzoic acid

4-{4-[2-[1-Butyl-4-(2,4-dichloro-phenyl)-1H-imidazol-2-yl]-(2S)-2-(2-cyclopentyl-acetylamino)-ethyl]-phenoxymethyl}-benzoic acid (45 mg, 69%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and cyclopentylacetic acid via amide formation followed by ester hydrolysis following general procedures F and N respectively.

LCMS: m/z 648 (M+H)⁺.

### EXAMPLE 109

### 4-{4-[2-[1-Butyl-4-(2,4-dichloro-phenyl)-1H-imidazol-2-yl]-(2S)-2-(cyclohexanecarbonyl-amino)-ethyl]-phenoxymethyl}-benzoic acid

4-{4-[2-[1-Butyl-4-(2,4-dichloro-phenyl)-1H-imidazol-2-yl]-(2S)-2-(cyclohexanecarbonyl-amino)-ethyl]-phenoxymethyl}-benzoic acid (40 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and cyclohexanecarboxylic acid via amide formation followed by ester hydrolysis following general procedures F and N respectively.

LCMS: m/z 648 (M+H)⁺.

### EXAMPLE 110

### 4-{4-[2-[1-Butyl-4-(2,4-dichloro-phenyl)-1H-imidazol-2-yl]-(2S)-2-(cyclopropanecarbonyl amino)-ethyl]-phenoxymethyl}-benzoic acid

4-{4-[2-[1-Butyl-4-(2,4-dichloro-phenyl)-1H-imidazol-2-yl]-(2S)-2-(cyclopropanecarbonyl-amino)-ethyl]-phenoxymethyl)benzoic acid (98 mg, 65%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and cyclopropanecarboxylic acid via amide formation followed by ester hydrolysis following general procedures F and N respectively.

LCMS: m/z 606 (M+H)⁺.

### EXAMPLE 111

### 4-(4-{2-[1-Butyl-4-(2,4-dichloro-phenyl)-1H-imidazol-2-yl]-(2S)-2-[(trans-4-methyl-cyclohexanecarbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(trans-4-methyl-cyclo-hexanecarbonyl)-amino]-ethyl}-phenoxymethyl)-benzoic acid (43 mg, 65%) was prepared from 4-(4-{(2S)-2-amino-2-[1-butyl-4-(2,4-dichloro-phenyl)-1H-imidazo-2-yl]-ethyl}-phenoxymethyl)-benzoic acid methyl ester hydrochloride (59 mg, 0.1 mmol) and trans-4-methylcyclohexanecarboxylic acid via amide formation followed by ester hydrolysis following general procedures F and N respectively.

LCMS: m/z 662 (M+H)⁺.

### EXAMPLE 112

### 4-(4-{2-[1-Butyl-4-(2,4-dichloro-phenyl)-1H-imidazol-2-yl]-(2S)-2-[(trans-4-methyl-cyclohexanecarbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(trans-4-ethyl-cyclo-hexanecarbonyl)-amino]-ethyl}-phenoxymethyl)-benzoic acid (47 mg, 69%) was prepared from 4-(4-{(2S)-2-amino-2-[1-butyl-4-(2,4-dichloro-phenyl)-1H-imidazol-2yl]-ethyl}-phenoxymethyl)-benzoic acid methyl ester hydrochloride (59 mg, 0.1 mmol) and trans-4-ethylcyclohexanecarboxylic acid via amide formation followed by ester hydrolysis following general procedures F and N respectively.

LCMS: m/z 676 (M+H)⁺.

### EXAMPLE 113

### 4-{4-[2-[1-Butyl-4-(2,4-dichloro-phenyl)-1H-imidazol-2-yl]-(2S)-2-(3-cyclohexyl-propionyl-amino)-ethyl]-phenoxymethyl}-benzoic acid

4-{4-[2-[1-Butyl-4-(2,4-dichloro-phenyl)-1H-imidazol-2-yl]-(2S)-2-(3cyclohexyl-propionyl-amino)-ethyl]-phenoxymethyl}-benzoic acid (49 mg, 72%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and 3-cyclohexylpropanoic acid via amide formation followed by ester hydrolysis following general procedures F and N respectively.

LCMS: m/z 676 (M+H)⁺.

### EXAMPLE 114

### 4-(4-{2-[1-Butyl-4-(2,4-dichloro-phenyl)-1H-imidazol-2-yl]-(2S)-2-[(trans-4-pentyl-cyclohexanecarbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(trans-4-pentyl-cyclohexanecarbonyl)-amino]-ethyl}-phenoxymethyl)-benzoic acid (42 mg, 58%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (59 mg, 0.1 mmol) and trans-4-pentylcyclohexanecarboxylic acid via amide formation followed by ester hydrolysis following general procedures F and N respectively.

LCMS: m/z 718 (M+H)⁺.

### EXAMPLE 115

### 4-(4-{2-[1-Butyl-4-(2,4-dichloro-phenyl)-1H-imidazol-2-yl]-(2S)-2-[(trans-2-phenyl-cyclopropanecarbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(trans-2-phenyl-cyclopropanecarbonyl)-amino]-ethyl}-phenoxymethyl)-benzoic acid (36 mg, 53%) was prepared from 4-(4-{(2S)-2-amino-2-[1-butyl-4-(2,4-dichloro-phenyl)-1H-imidazo-2-yl]-ethyl}-phenoxymethyl)-benzoic acid methyl ester hydrochloride (59 mg, 0.1 mmol) and trans-2-phenylcyclopropanecarboxylic acid via amide formation followed by ester hydrolysis following general procedures F and N respectively.

LCMS: m/z 682 (M+H)⁺.

### EXAMPLE 116

### 4-{4-[2-[1-Butyl-4-(2,4-dichloro-phenyl)-1H-imidazol-2-yl]-(2S)-2-(2-cyclohexyl-acetylamino)-ethyl]-phenoxymethyl}-benzoic acid

4-{4-[2-[1-Butyl-4-(2,4-dichloro-phenyl)-1H-imidazol-2-yl]-(2S)-2-(2-cyclohexyl-acetylamino)-ethyl]-phenoxymethyl}-benzoic acid (107 mg, 64%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and cyclohexylacetic acid via amide formation followed by ester hydrolysis following general procedures F and N respectively.

LCMS: m/z 662 (M+H)⁺; ¹H NMR (CDCl₃, 400 MHz): δ 0.8-1.91 (m, 14H), 1.92 (m, 2H), 2.10 (m, 2H), 3.21 (t, 2H), 3.65 (m, 1H), 3.76 (m, 1H), 5.07 (s, 2H), 5.40 (m, 1H), 6.78 (d, 2H), 6.98 (d, 2H), 7.32 (m, 2H), 7.39-7.51 (m, 4H), 7.75 (d, 2H), 7.90 (d, 1H), 8.07 (d, 2H) ppm.

### EXAMPLE 117

### 4-(4-{2-[1-Butyl-4-(2,4-dichloro-phenyl)-1H-imidazol-2-yl]-(2S)-2-[(4-methoxy-cyclohexanecarbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(4-methoxy-cyclohexanecarbonyl)-amino]-ethyl}-phenoxymethyl)-benzoic acid (113 mg, 67%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-methoxycyclohexanecarboxylic acid via amide formation followed by ester hydrolysis following general procedures F and N respectively.

LCMS: m/z 678 (M+H)⁺.

### EXAMPLE 118

### 4-(4-{2-[1-Butyl-4-(2,4-dichloro-phenyl)-1H-imidazol-2-yl]-(2S)-2-isobutyrylamino-ethyl}-phenoxymethyl)-benzoic acid

4-(4-{2-[1-Butyl-4-(2,4-dichloro-phenyl)-1H-imidazol-2-yl]-(2S)-2-isobutyrylamino-ethyl}-phenoxymethyl)-benzoic acid (95 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and isobutyric acid via amide formation followed by ester hydrolysis following general procedures F and N respectively.

LCMS: m/z 608 (M+H)⁺.

### EXAMPLE 119

### 4-(4-{(2S)-2-(4-tert-Butyl-benzenesulfonylamino)-2-[1-butyl-4-(2,4-dichloro-phenyl)-1H-imidazol-2-yl]-ethyl}-phenoxymethyl)-benzoic acid

4-(4-{(2S)-2-(4-tert-Butyl-benzenesulfonylamino)-2-[1-butyl-4-(2,4-dichloro-phenyl)-1H-imidazol-2-yl]-ethyl}-phenoxymethyl)-benzoic acid (113 mg, 62%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-tert-butylbenzene-sulfonyl chloride via sulfonamide formation followed by ester hydrolysis following general procedures O and N respectively.

LCMS: m/z 734 (M+H)⁺.

### EXAMPLE 120

### 4-{4-[2-[1-Butyl-4-(2,4-dichloro-phenyl)-1H-imidazol-2-yl]-(2S)-2-(naphthalene-1-sulfonylamino)-ethyl]-phenoxymethyl}-benzoic acid

4-{4-[2-[1-Butyl-4-(2,4-dichloro-phenyl)-1H-imidazol-2-yl]-(2S)-2-(naphthalene-1-sulfonylamino)-ethyl]-phenoxymethyl}-benzoic acid ( 107 mg, 59%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 1-naphthalenesulfonyl chloride via sulfonamide formation followed by ester hydrolysis following general procedures O and N respectively.

LCMS: m/z 728 (M+H)⁺; ¹H NMR (CDCl₃, 400 MHz): δ 1.0-1.28 (m, 4H), 3.21 (t, 2H), 3.11 (d, 1H), 3.25 (m, 1H), 4.64 (m, 1H), 5.04 (s, 2H), 5.30 (m, 1H), 6.64 (d, 2H), 6.72 (d, 2H), 6.85 (s, 1H), 7.05 (d, 1H), 7.12 (d, 2H), 7.15 (d, 1H), 7.27-7.42 (m, 4H), 7.43-7.52 (m, 4H), 7.71 (d, 1H), 7.82 (d, 2H), 8.02 (d, 1H), 8.64 (d, 1H) ppm.

### EXAMPLE 121

### 4-{4-[2-[1-Butyl-4-(2,4-dichloro-phenyl)-1H-imidazol-2-yl]-(2S)-2-(4-methoxy-benzene-sulfonylamino)-ethyl]-phenoxymethyl}-benzoic acid

4-{4-[2-[1-Butyl-4-(2,4-dichloro-phenyl)-1H-imidazol-2-yl]-(2S)-2-(4-methoxy-benzene-sulfonylamino)-ethyl]-phenoxymethyl}-benzoic acid (117 mg, 66%) was prepared from 4-(4-{(2S)-2-amino-2-[1-butyl-4-(2,4-dichloro-phenyl)-1H-imidazol-2yl]-ethyl}-phenoxymethyl)-benzoic acid methyl ester hydrochloride (138 mg, 0.25 mmol) and 4-methoxybenzene-sulfonyl chloride via sulfonamide formation followed by ester hydrolysis following general procedures O and N respectively.

LCMS: m/z 708 (M+H)⁺; ¹H NMR (CDCl₃, 400 MHz): δ 0.78-0.8 (m, 2H), 0.90-0.98 (m, 2H), 1.03-1.19 (m, 2H), 3.16 (m, 1H), 3.30 (m, 1H), 3.35 (m, 1H), 3.67 (s, 3H), 3.87 (m, 1H), 4.64 (m, 1H), 5.04 (s, 2H), 6.70 (d, 2H), 6.85 (s, 1H), 7.05 (d, 1H), 7.12 (d, 1H), 7.15 (d, 1H), 7.27-7.42 (m, 4H), 7.43-7.52 (m, 2H), 7.71 (d, 1H), 7.82 (d, 2H), 8.02 (d, 2H), 8.64 (d, 1H) ppm.

### EXAMPLE 122

### 4-(4-{(2S)-2-(4-Butyl-benzenesulfonylamino)-2-[1-butyl-4-(2,4-dichloro-phenyl)-1H-imidazol-2-yl]-ethyl}-phenoxymethyl)-benzoic acid

4-(4-{(2S)-2-(4-Butyl-benzenesulfonylamino)-2-[1-butyl-4-(2,4-dichloro-phenyl)-1H-imidazol-2-yl]-ethyl}-phenoxymethyl)-benzoic acid (119 mg, 65%) was prepared from 4-(4-{(2S)-2-amino-2-[1-butyl-4-(2,4-dichloro-phenyl)-1H-imidazol-2-yl]ethyl}-phenoxymethyl)-benzoic acid methyl ester hydrochloride (138 mg, 0.25 mmol) and 4-butylbenzenesulfonyl chloride via sulfonamide formation followed by ester hydrolysis following general procedures O and N respectively.

LCMS: m/z 734 (M+H)⁺.

### EXAMPLE 123

### 4-{(1S)-1-[1-Butyl-4-(2,4-dichloro-phenyl)-1H-imidazol-2-yl]-2-[4-(4-carboxy-benzyloxy)-phenyl]-ethylcarbamoyl}-piperidine-1-carboxylic acid tert-butyl ester

4-{(1S)-1-[1-Butyl-4-(2,4-dichloro-phenyl)-1H-imidazol-2-yl]-2-[4-(4-carboxy-benzyloxy)-phenyl]-ethylcarbamoyl)piperidine-1-carboxylic acid tert-butyl ester (95 mg, 50%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and N-boc-pipiridine-4-carboxylic acid via amide formation followed by ester hydrolysis following general procedures F and N respectively.

LCMS: m/z 749 (M+H)⁺; ¹H NMR (CDCl₃, 400 MHz): δ 0.81-0.88 (m, 9H), 1.11-1.18 (m, 2H), 1.25-1.28 (m, 2H), 1.30-1.40 (m, 4H), 1.44-1.62 (m, 4H), 2.35 (m, 2H), 3.12 (m, 1H), 3.23 (m, 1H), 3.27 (m, 1H), 3.65 (m, 1H), 3.76 (m, 1H), 4.02 (m, 1H), 5.04 (s, 2H), 5.21 (m, 1H), 6.81 (d, 2H), 7.01 (d, 2H), 7.34 (d, 2H), 7.36-7.47 (m, 4H), 7.94 (d, 2H) ppm.

### EXAMPLE 124

### 4-(4-{2-[1-Butyl-4-(2,4-dichloro-phenyl)-1H-imidazol-2-yl]-(2S)-2-[(4-trifluoromethyl-cyclohexanecarbonyl)-amino]-ethyl}-phenoxymethyl)-benzoic acid

4-(4-{2-[1-Butyl-4-(2,4-dichloro-phenyl)-1H-imidazol-2-yl]- (2S)-2-[(4-trifluoromethyl-cyclohexanecarbonyl)-amino]-ethyl}-phenoxymethyl)-benzoic acid (129 mg, 70%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-trifluoromethylcyclohexanecarboxylic acid via amide formation followed by ester hydrolysis following general procedures F and N respectively.

LCMS: m/z 716 (M+H)⁺; ¹H NMR (DMSO, 400 MHz): δ 0.84 (t, 3H), 1.15-1.24 (m, 4H), 1.33-1.36 (m, 2H), 1.40-1.51 (m, 3H), 1.53-1.75 (m, 2H), 2.33-2.49 (m, 3H), 3.09-3.15 (m, 2H), 3.25-3.27 (m, 2H), 3.88 (m 1H), 3.95 (m, 1H), 5.11 (s, 1H), 5.25 (m, 1H), 6.86 (d, 2H), 7.17 (d, 2H), 7.48-7.52 (m, 2H), 7.61 (s, 1H), 7.78 (s, 1H), 7.94 (d, 2H), 8.20 (d, 1H), 8.34 (d, 1H) ppm.

### EXAMPLE 125

### 4-(4-{2-[1-Butyl-4-(2,4-dichloro-phenyl)-1H-imidazol-2-yl]-(2S)-2-[(4-hydroxy-cyclohexane-carbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(4-hydroxy-cyclohexane-carbonyl)-amino]-ethyl}-phenoxymethyl)-benzoic acid (98 mg, 60%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 4-hydroxycyclohexanecarboxylic acid via amide formation followed by ester hydrolysis following general procedures F and N respectively.

LCMS: m/z 664 (M+H)⁺; ¹H NMR (DMSO, 400 MHz): δ 0.78 (t, 3H), 1.12-1.59 (m, 12H), 2.72-2.88 (m, 2H), 3.09-3.15 (m, 2H), 3.25-35 (m, 2H), 3.84 (m 1H), 3.91 (m, 1H), 5.12 (s, 1H), 5.16 (m, 1H), 6.85 (d, 2H), 7.14 (d, 2H), 7.49-7.52 (m, 2H), 7.61 (s, 1H), 7.76 (s, 1H), 7.94 (d, 2H), 8.20 (d,1 H), 8.34 (d, 1H) ppm.

### EXAMPLE 126

### 4-(4-{2-[1-Butyl-4-(2,4-dichloro-phenyl)-1H-imidazol-2-yl]-(2S)-2-[(2,6,6-trimethyl-bicyclo[3.1.1]heptane-3-carbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(2,6,6-trimethyl-bicyclo[3.1.1]heptane-3-carbonyl)-amino]-ethyl}-phenoxymethyl)-benzoic acid (104 mg, 60%) was prepared from 4-(4-{(2S)-2-amino-2-[1-butyl-4-(2,4-dichloro-phenyl)-1H-imidazol-2-yl]-ethyl}-phenoxymethyl)-benzoic acid methyl ester hydrochloride (138 mg, 0.25 mmol) and 2,6,6-trimethyl-bicyclo[3.1.1]heptane-3-carboxylic acid via amide formation followed by ester hydrolysis following general procedures F and N respectively.

LCMS: m/z 702 (M+H)⁺; ¹H NMR (DMSO, 400 MHz): δ 0.76-0.79 (m, 9H), 0.83 (t, 3H), 0.91-0.93 (m, 2H), 1.17 (s, 3H), 1.48 (m, 1H), 1.62-1.63 (m, 2H), 1.77-1.78 (m, 2H), 1.91-1.96 (m, 2H), 2.18-2.20 (m, 2H), 3.11 (m, 1H), 3.96 (m, 1H), 5.10 (s, 1H), 5.23 (m, 1H), 6.84 (d, 2H), 7.15 (d, 2H), 7.45-7.51 (m, 4H), 7.61 (s, 1H), 7.77 (s, 1H), 7.91 (d, 2H), 8.23 (d, 1H), 8.43 (d, 1H) ppm.

### EXAMPLE 127

### 4-(4-{(2S)-2-[(Bicyclo[2.2.1 ]heptane-5-ene-2-carbonyl)-amino]-2-[1-butyl-4-(2,4-dichloro-phenyl)-1H-imidazol-2-yl]-ethyl}-phenoxymethyl)-benzoic acid

4-(4-{(2S)-2-[(Bicyclo[2.2.1]heptane-5-ene-2-carbonyl)-amino]-2-[1-butyl-4-(2,4-dichloro-phenyl)-1H-imidazol-2-yl]-ethyl}-phenoxymethyl)-benzoic acid (101 mg, 62%) was prepared from 4-(4-{(2S)-2-amino-2-[1-butyl-4-(2,4-dichloro-phenyl)-1H-imidazo-2-yl]-ethyl}-phenoxy-methyl)-benzoic acid methyl ester hydrochloride (138 mg, 0.25 mmol) and bicyclo[2.2.1]heptane-5-ene-2-carboxylic acid via amide formation followed by ester hydrolysis following general procedures F and N respectively.

LCMS: m/z 658 (M+H)⁺.

### EXAMPLE 128

### 4-(4-{(2S)-2-(2-Bicyclo[2.2.1]hept-2-yl-acetylamino)-2-[1-butyl-4-(2,4-dichloro-phenyl)-1H-imidazol-2-yl]-ethyl}-phenoxymethyl)-benzoic acid

4-(4-{(2S)-2-(2-Bicyclo[2.2.1]hept-2-yl-acetylamino)-2-[1-butyl-4-(2,4-dichloro-phenyl)-1H-imidazol-2-yl]-ethyl}-phenoxymethyl)-benzoic acid (113 mg, 67%) was prepared from 4-(4-{(2S)-2-amino-2-[1-butyl-4-(2,4-dichloro-phenyl)-1H-imidazol-2-yl]-ethyl)phenoxy-methyl)-benzoic acid methyl ester hydrochloride (138 mg, 0.25 mmol) and bicyclo[2.2.1]hept-2-ylacetic acid via amide formation followed by ester hydrolysis following general procedures F and N respectively.

LCMS: m/z 674 (M+H)⁺; ¹H NMR (DMSO, 400 MHz): δ 0.79-0.99(m, 9H), 1.01-1.18 (m, 2H), 1.21-1.45 (m, 3H), 1.46-1.81 (m, 4H), 1.83-1.20 (m, 2H), 3,22 (m, 1H), 3.34 (m, 1H), 3.91 (m, 1H), 3.94 (m, 1H), 5.14 (s, 1H), 5.18 (m, 1H), 6.85 (d, 2H), 7.13 (d, 2H), 7.48-7.52 (m, 4H), 7.61 (s, 1H), 7.75 (s, 1H), 7.94 (d, 2H), 8.22 (d, 1H), 8.42 (d, 1H) ppm.

### EXAMPLE 129

### 4-(4-{2-[1-Butyl-4-(2,4-dichloro-phenyl)-1H-imidazol-2-yl]-(2S)-2-[(pyrrolidine-1-carbonyl)-amino]-ethyl}-phenoxymethyl)-benzoic acid

4-(4-{2-[1-Butyl-4-(2,4-dichloro-phenyl)-1H-imidazol-2-yl]-(2S)-2-[(pyrrolidine-1-carbonyl)-amino]-ethyl}-phenoxymethyl)-benzoic acid (40 mg, 62%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (59 mg, 0.1 mmol) and pyrrolidine-1-carboxylic acid via amide formation followed by ester hydrolysis following general procedures F and N respectively.

LCMS: m/z 635 (M+H)⁺.

### EXAMPLE 130

### 4-(4-{(2S)-2-[(Trans-2-tert-Butylcarbamoyl-cyclohexanecarbonyl)-amino]-2-[1-butyl-4-(2,4-dichloro-phenyl)-1H-imidazol-2-yl]-ethyl}-phenoxymethyl)-benzoic acid

4-(4-{(2S)-2-[(Trans-2-tert-Butylcarbamoyl-cyclohexanecarbonyl)-amino]-2-[1-butyl-4-(2,4-dichloro-phenyl)-1H-imidazol-2-yl]-ethyl}-phenoxymethyl)-benzoic acid (112 mg, 60%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (138 mg, 0.25 mmol) and trans-2-tert-butylcarbamoyl-cyclohexanecarboxylic acid via amide formation followed by ester hydrolysis following general procedures F and N respectively.

LCMS: m/z 747 (M+H)⁺; ¹H NMR (DMSO, 400 MHz): δ 0.74-0.82(m, 9H), 0.84 (t, 3H), 1.04-1.26 (m, 4H), 1.34-1.38 (m, 2H), 1.39-1.49 (m, 2H), 1.51-1.63 (m, 2H), 2.66-2.69 (m, 2H), 2.71-2.98 (m, 2H), 3.71 (m, 1H), 3.82 (m, 1H), 5.07 (s, 1H), 5.13 (m, 1H), 6.83 (s, 1H), 6.87 (d, 2H), 7.07 (d, 2H), 7.13 (d, 1H), 7.48-7.51 (m, 4H), 7.61 (s, 1H), 7.71 (s, 1H), 7.77 (s, 1H), 7.94 (d, 2H), 8.24 (d, 1H), 8.31 (d, 1H) ppm.

### EXAMPLE 131

### 4-(4-{(2S)-2-{trans-4-(tert-Butoxycarbonylamino-methyl)-cyclohexanecarbonyl-amino}-2-[1-butyl-4-(2,4-dichloro-phenyl)-1H-imidazol-2-yl]-ethyl}-phenoxymethyl)-benzoic acid

4-(4-{(2S)-2-{[trans-4-(tert-Butoxycarbonylamino-methyl)-cyclohexanecarbonyl]-amino}-2-[1-butyl-4-(2,4-dichloro-phenyl)-1H-imidazol-2-yl]-ethyl}-phenoxymethyl)-benzoic acid (124 mg, 64%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (138 mg, 0.25 mmol) and trans-4-(tert-butoxycarbonylamino-methyl)-cyclohexanecarboxylic acid via amide formation followed by ester hydrolysis following general procedures F and N respectively.

LCMS: m/z 777 (M+H)⁺; ¹H NMR (DMSO, 400 MHz): δ 0.75-0.83(m, 9H), 0.85 (t, 3H), 1.12-1.23 (m, 4H), 1.35-1.44 (m, 4H), 1.55-1.64 (m, 4H), 2.66-2.69 (m, 2H), 2.71-2.98 (m, 2H), 3.84 (m, 1H), 3.90 (m, 1H), 5.13 (s, 1H), 5.15 (m, 1H), 6.78 (m, 1H), 6.87 (d, 2H), 7.12 (d, 2H), 7.48-7.51 (m, 4H), 7.52 (d, 2H), 7.62 (s, 1H), 7.77 (s, 1H), 7.94 (d, 2H), 8.26 (d, 1H), 8.41 (d, 1H) ppm.

### EXAMPLE 132

### 4-(4-{(2S)-2-[trans-(4-Aminomethyl-cyclohexanecarbonyl)-amino]-2-[1-butyl-4-(2,4-dichloro-phenyl)-1H-imidazol-2-yl]-ethyl}-phenoxymethyl)-benzoic acid hydrochloride

4-(4-{(2S)-2-[trans-(4-Aminomethyl-cyclohexanecarbonyl)-amino]-2-[1-butyl-4-(2,4-dichloro-phenyl)-1H-imidazol-2-yl]-ethyl}-phenoxymethyl)-benzoic acid hydrochloride (134 mg, 80%) was prepared from 4-(4-{(2S)-2-{[4-(tert-Butoxycarbonylamino-methyl)-cyclohexanecarbonyl]-amino}-2-[1-butyl-4-(2,4-dichloro-phenyl)-1H-imidazol-2-yl]-ethyl}-phenoxymethyl)-benzoic acid (195 mg, 0.25 mmol) via removal of boc-group following general procedure E.

LCMS: m/z 677 (M+H)⁺.

### EXAMPLE 133

### 4-(4-{2-[1-Butyl-4-(2,4-dichloro-phenyl)-1H-imidazol-2-yl]-(2S)-2-hex-2-ynoylamino-ethyl}-phenoxymethyl)-benzoic acid

4-(4-{2-[1-Butyl-4-(2,4-dichloro-phenyl)-1H-imidazol-2-yl]-(2S)-2-hex-2-ynoylamino-ethyl}-phenoxymethyl)-benzoic acid (102 mg, 65%) was prepared from 4-(4-{(2S)-2-amino-2-[1-butyl-4-(2,4-dichloro-phenyl)-1 H-imidazol-2-yl]-ethyl}-phenoxy-methyl)-benzoic acid methyl ester hydrochloride (138 mg, 0.25 mmol) and hex-2-ynoic acid via amide formation followed by ester hydrolysis following general procedures F and N respectively.

LCMS: m/z 632 (M+H)⁺; ¹H NMR (DMSO, 400 MHz): δ 0.76-0.90 (m, 6H), 0.92-0.95 (m, 2H), 1.0-1.16 (m, 2H), 1.39-1.49 (m, 2H), 2.25-2.29 (m, 2H), 2.72-2.88 (m, 2H), 3.64 (m, 1H), 3.79 (m, 1H), 5.12 (s, 1H), 5.19 (m, 1H), 6.56 (s, 1H), 6.86 (d, 2H), 7.13 (d, 2H), 7.49-7.52 (m, 4H), 7.62 (s, 1H), 7.30 (d, 1H), 7.92 (d, 1H), 8.26 (d, 1H) ppm.

### EXAMPLE 134

### 4-(4-{2-[1-Butyl-4-(2,4-dichloro-phenyl)-1H-imidazol-2-yl]-(2S)-2 -hex-5-ynoylamino-ethyl}-phenoxymethyl)-benzoic acid

4-(4-{2-[1-Butyl-4-(2,4-dichloro-phenyl)-1H-imidazol-2-yl]-(2S)-2-hex-5-ynoylamino-ethyl}-phenoxymethyl)-benzoic acid (101 mg, 64%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (138 mg, 0.25 mmol) and hex-5-ynoic acid via amide formation followed by ester hydrolysis following general procedures F and N respectively.

LCMS: m/z 632 (M+H)⁺.

### EXAMPLE 135

### 4-{4-[2-[1-Butyl-4-(2,4-dichloro-phenyl)-1H-imidazol-2-yl]-(2S)-2-(4,4-dimethyl-pentanoyl-amino)-ethyl]-phenoxymethyl}-benzoic acid

4-{4-[2-[1-Butyl-4-(2,4-dichloro-phenyl)-1H-imidazol-2-yl]-(2S)-2-(4,4-dimethyl-pentanoyl-amino)-ethyl]-phenoxymethyl}-benzoic acid (112 mg, 69%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (138 mg, 0.25 mmol) and 4,4-dimethylpentanoic acid via amide formation followed by ester hydrolysis following general procedures F and N respectively.

LCMS: m/z 650 (M+H)⁺; ¹H NMR (DMSO, 400 MHz): δ 0.78-0.94 (m, 12H), 1.07-1.10 (m, 2H), 1.13-1.14 (m, 2H), 1.25-1.45 (m, 4H), 1.90-2.0 (m, 2H), 3.03-3.29 (m, 1H), 3.36-3.47 (m, 1H), 3.80 (m, 1H), 3.95 (m, 1H), 5.12 (s, 1H), 5.16 (m, 1H), 6.57 (s, 1H), 6.85 (d, 2H), 7.10 (d, 2H), 7.52 (d, 2H), 7.62 (s, 1H), 7.74 (s, 1H), 7.92-7.94 (m, 2H), 8.20 (d, 1H), 8.49 (d, 1H) ppm.

### EXAMPLE 136

### 4-(4-{2-[1-Butyl-4-(2,4-dichloro-phenyl)-1H-imidazol-2-yl]-(2S)-2-heptanoylamino-ethyl}-phenoxymethyl)-benzoic acid

4-(4-{2-[1-Butyl-4-(2,4-dichloro-phenyl)-1H-imidazol-2-yl]-(2S)-2-heptanoylamino-ethyl}-phenoxymethyl)-benzoic acid (46 mg, 72%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (59 mg, 0.1 mmol) and heptanoic acid via amide formation followed by ester hydrolysis following general procedures F and N respectively.

LCMS: m/z 650 (M+H)⁺; ¹H NMR (CDCl₃-CD₃OD, 400 MHz): δ 0.85 (m, 6H), 1.12-1.43 (m, 10H), 1.46 (m, 2H), 2.16 (t, 2H), 3.14 (m, 1H), 3.27 (m, 1H), 3.69 (m, 1H), 3.81 (m, 1H), 5.10 (s, 2H), 5.23 (m, 1H), 6.86 (d, 2H), 7.05 (d, 2H), 7.38 (dd, 1H), 7.48 (d, 1H), 7.50 (m, 3H), 7.97 (d, 1H), 8.01 (d, 2H) ppm.

### EXAMPLE 137

### 4-{4-[2-[1-Butyl-4-(2,4-dichloro-phenyl)-1H-imidazol-2-yl]-(2S)-2-(6-methyl-heptanoylamino)-ethyl]-phenoxymethyl}-benzoic acid

4-{4-[2-[1-Butyl-4-(2,4-dichloro-phenyl)-1H-imidazol-2-yl]-(2S)-2-(6-methyl-heptanoylamino)-ethyl]-phenoxymethyl}-benzoic acid (44 mg, 67%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (59 mg, 0.1 mmol) and 6-methylheptanoic acid via amide formation followed by ester hydrolysis following general procedures F and N respectively.

LCMS: m/z 664 (M+H)⁺; ¹H NMR (CDCl₃-CD₃OD, 400 MHz): δ 0.81-0.87 (m, 9H), 1.13-1.54 (m, 11H), 2.16 (t, 2H), 3.14 (m, 1H), 3.27 (m, 1H), 3.69 (m, 1H), 3.81 (m, 1H), 5.10 (s, 2H), 5.23 (m, 1H), 6.86 (d, 2H), 7.05 (d, 2H), 7.38 (dd, 1H), 7.48 (d, 1H), 7.50 (m, 3H), 7.97 (d, 1H), 8.00 (d, 2H) ppm.

### EXAMPLE 138

### 4-{4-[2-[1-Butyl-4-(2,4-dichloro-phenyl)-1H-imidazol-2-yl]-(2S)-2-(6,6-dimethylheptanoyl-amino)-ethyl]-phenoxymethyl}-benzoic acid

4-{4-[2-[1-Butyl-4-(2,4-dichloro-phenyl)-1H-imidazol-2-yl]-(2S)-2-(6,6-dimethylheptanoyl-amino)-ethyl]-phenoxymethyl}-benzoic acid (44 mg, 65%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (59 mg, 0.1 mmol) and 6,6-dimethylheptanoic acid via amide formation followed by ester hydrolysis following general procedures F and N respectively.

LCMS: m/z 678 (M+H)⁺, ¹H NMR (CDCl₃-CD₃OD, 400 MHz): δ 0.82 (s, 9H), 0.84 (t, 3H), 1.13-1.58 (m, 10H), 2.16 (t, 2H), 3.14 (m, 1H), 3.27 (m, 1H), 3.69 (m, 1H), 3.81 (m, 1H), 5.10 (s, 2H), 5.23 (m, 1H), 6.86 (d, 2H), 7.05 (d, 2H), 7.38 (dd, 1H), 7.48 (d, 1H), 7.50 (m, 3H), 7.97 (d, 1H), 8.03 (d, 2H) ppm.

### EXAMPLE 139

### 4-(4-{(2S)-2-[(4-tert-Butyl-cyclohexanecarbonyl)-amino]-2-[4-(2,4-dichloro-phenyl)-1H-imidazol-2-yl]-ethyl}-phenoxymethyl)-benzoic acid

L-Tyrosine methyl ester (1.95 g 1 mmol) was reacted with 4-tert-butyl-cyclohexane carboxylic acid via amide formation and selective hydrolysis using general procedure F & N respectively. The obtained (2S)-2-[(4-tert-butyl-cyclohexane carbonyl)-amino]-3-(-4-hydroxy-phenyl)-propionic acid was treated with 2,4-dichlorophenacyl bromide according to general procedure C to afford the desired keto-ester. 4-tert-butyl-cyclohexane carboxylic acid [(1S)-1-[4-(2,4-dichloro-phenyl)-1H-imidazol-2-yl]-2-(4-hydroxyl-phenyl)-ethyl]-amide imidazole was formed from the keto ester according to general procedure D. The resulted 4-tert-butyl-cyclohexane carboxylic acid [(1S)-1-[4-(2,4-dichloro-phenyl)-1H-imidazol-2-yl]-2-(4-hydroxyl-phenyl)-ethyl]-amide was protected and alkylated with 4-bromomethyl-benzoic acid methyl ester using general procedures S and G respectively. The obtained 4-(4-{(2S)-2-[(4-tert-butyl-cyclohexanexarbonyl)-amino]-2-[4-(2,4-dichloro-phenyl)-1-(2,2-dimethyl-propionyloxy)-1H-imidazol-2-yl]-ethyl}-phenyl methyl)-benzoic acid methyl ester was selectively deprotected and hydrolysis according to general procedure E and N respectively to give 4-(4-{(2S)-2-[(4-tert-Butyl-cyclohexanecarbonyl)-amino]-2-[4-(2,4-dichloro-phenyl)-1H-imidazol-2-yl]-ethyl}-phenoxymethyl)-benzoic acid (82 mg, 50%).

LCMS: m/z 648 (M+H)⁺.

### EXAMPLE 140

### 4-(4-{2-[1-Benzyl-4-(2,4-dichloro-phenyl)-1H-imidazol-2-yl]-(2S)-2-[(4-tert-butyl-cyclohexane-carbonyl)-amino]-ethyl}-phenoxymethyl)-benzoic acid

4-(4-{2-[1-Benzyl-4-(2,4-dichloro-phenyl)-1H-imidazol-2-yl]-(2S)-2-[(4-tert-butyl-cyclohexane-carbonyl)-amino]-ethyl}-phenoxymethyl)-benzoic acid (119 mg, 65%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and benzyl bromide via N-alkylation followed by ester hydrolysis following general procedures H and N respectively.

LCMS: m/z 738 (M+H)⁺.

### EXAMPLE 141

### 4-(4-{(2S)-2-[(4-tert-Butyl-cyclohexanecarbonyl)-amino]-2-[4-(2,4-dichloro-phenyl)-1-(2-oxo-butyl)-1H-imidazol-2-yl]-ethyl}-phenoxymethyl)-benzoic acid

4-(4-{(2S)-2-[(4-tert-Butyl-cyclohexanecarbonyl)-amino]-2-[4-(2,4-dichloro-phenyl)-1-(2-oxo-butyl)-1H-imidazol-2-yl]-ethyl}-phenoxymethyl)-benzoic acid (112 mg, 63%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and 1-chlorobutan-2-one via N-alkylation followed by ester hydrolysis following general procedures H and N respectively.

LCMS: m/z 718 (M+H)⁺.

### EXAMPLE 142

### 4-(4-{(2S)-2-[(4-tert-Butyl-cyclohexanecarbonyl)-amino]-2-[4-(2,4-dichloro-phenyl)-1-(3-methyl-butyl)-1H-imidazol-2-yl]-ethyl}-phenoxymethyl)-benzoic acid

4-(4-{(2S)-2-[(4-tert-Butyl-cyclohexanecarbonyl)-amino]-2-[4-(2,4-dichloro-phenyl)-1-(3-methyl-butyl)-1H-imidazol-2-yl]-ethyl}-phenoxymethyl)-benzoic acid (119 mg, 66%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and 1-bromo-3-methylbutane via N-alkylation followed by ester hydrolysis following general procedures H and N respectively.

LCMS: m/z 718 (M+H)⁺.

### EXAMPLE 143

### 4-(4-{(2S)-2-[(4-tert-Butyl-cyclohexanecarbonyl)-amino]-2-[4-(2,4-dichloro-phenyl)-1-(3-hydroxy-propyl)-1H-imidazol-2-yl]-ethyl}-phenoxymethyl)-benzoic acid

4-(4-{(2S)-2-[(4-tert-Butyl-cyclohexanecarbonyl)-amino]-2-[4-(2,4-dichloro-phenyl)-1-(3-hydroxy-propyl)-1H-imidazol-2-yl]-ethyl}-phenoxymethyl)-benzoic acid (99 mg, 57%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and 3-bromopropanol via N-alkylation followed by ester hydrolysis following general procedures H and N respectively.

LCMS: m/z 706 (M+H)⁺.

### EXAMPLE 144

### 4-(4-{(2S)-2-[(4-tert-Butyl-cyclohexanecarbonyl)-amino]-2-[4-(2,4-dichloro-phenyl)-1-ethyl-1H-imidazol-2-yl]-ethyl}-phenoxymethyl)-benzoic acid

4-(4-{(2S)-2-[(4-tert-Butyl-cyclohexanecarbonyl)-amino]-2-[4-(2,4-dichloro-phenyl)-1-ethyl-1H-imidazol-2-yl]-ethyl}-phenoxymethyl)-benzoic acid (117 mg, 70%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and bromoethane via N-alkylation followed by ester hydrolysis following general procedures H and N respectively.

LCMS: m/z 676 (M+H)⁺.

### EXAMPLE 145

### 4-(4-{(2S)-2-[(4-tert-Butyl-cyclohexanecarbonyl)-amino]-2-[4-(2,4-dichloro-phenyl)-(E)-1-pent-2-enyl-1H-imidazol-2-yl]-ethyl}-phenoxymethyl)-benzoic acid

4-(4-{(2S)-2-[(4-tert-Butyl-cyclohexanecarbonyl)-amino]-2-[4-(2,4-dichloro-phenyl)-(E)-1-pent-2-enyl-1H-imidazol-2-yl]-ethyl}-phenoxymethyl)-benzoic acid (109 mg, 60%) was prepared from 4-(4-{(2S)-2-[(4-tert-butyl-cyclohexanecarbonyl)-amino]-2-[4-(2,4-dichloro-phenyl)-1H-imidazol-2-yl]-ethyl}-phenoxymethyl)-benzoic acid methyl ester (165 mg, 0.25 mmol) and trans-1-bromo-2-petene via N-alkylation followed by ester hydrolysis following general procedures H and N respectively.

LCMS: m/z 716 (M+H)⁺; ¹H NMR (DMSO, 400 MHz): δ 0.78-0.94 (m, 12H), 1.12-1.23 (m, 2H), 1.51-1.68 (m, 4H), 1.90-1.95 (m, 2H), 2.68-2.72 (m, 2H), 2.8 (d, 1H), 3.07 (m, 1H), 3.19 (m, 1H), 4.49 (m, 1H), 4.56 (m, 1H), 5.14 (s, 1H), 5.18 (m, 1H), 5.39 (m, 1H), 5.49 (m, 1H), 6.56 (s, 1H), 6.87 (d, 2H), 7.14 (d, 2H), 7.48-7.52 (m, 4H), 7.61 (d, 1H), 7.71 (s, 1H), 7.94 (d, 2H), 8.22 (d, 1H), 8.30 (d, 1H) ppm.

### EXAMPLE 146

### 4-(4-{2-[4-(2,4-Dichloro-phenyl)-(E)-1-pent-2-enyl-1H-imidazol-2-yl]-(2S)-2-[(trans-4-ethyl-cyclohexanecarbonyl)-amino]-ethyl}-phenoxymethyl)-benzoic acid

Step A: Boc-L-tyrosine was treated with 2,4-dichlorophenacyl bromide following the general procedure C to afford the desired keto ester. The imidazole was formed from the keto ester according to general procedure D. Deprotection of amine following general procedure E afforded 4-{(2S)-2-amino-2-[4-(2,4-dichloro-phenyl)-1H-imidazol-2-yl]-ethyl}-phenol hydrochloride. This hydrochloride salt was treated with trans-4-ethylcyclohexane carboxylic acid following general procedure F to obtain trans-4-ethyl-cyclohexanecarboxylic acid [(1S)-1-[4-(2,4-dichloro-phenyl)-1H-imidazol-2-yl]-2-(4-hydroxy-phenyl)-ethyl]-amide.

Trans-4-ethyl-cyclohexanecarboxylic acid [(1S)-1-[4-(2,4-dichloro-phenyl)-1-pent-2-enyl-1H-imidazol-2-yl]-2-(4-hydroxy-phenyl)-ethyl]-amide (384 mg, 69%) was prepared via N, O-alkylation of trans-4-ethyl-cyclohexanecarboxylic acid [(1S)-1-[4-(2,4-dichloro-phenyl)-1H-imidazol-2-yl]-2-(4-hydroxy-phenyl)-ethyl]-amide (486 g, 1 mmol) with (E)-1-bromo-2-pentene following general procedure H followed by ether cleavage using general procedure K.

LCMS: m/z 554 (M+H)⁺; ¹H NMR (DMSO, 400 MHz): δ 0.76-86 (m, 6H), 0.88 (t, 3H), 0.90-1.01 (m, 2H), 1.12-1.17 (m, 2H), 1.23-1.27 (m, 2H), 1.54-1.57 (m, 2H), 1.66-1.69 (m, 2H), 1.92-2.02 (m, 2H), 3.02 (m, 1H), 3.16 (m, 1H), 5.13 (m, 1H), 5.39 (m, 1H), 5.50 (m, 1H), 6.60 (d, 2H), 6.97 (d, 2H), 7.48-7.50 (m, 2H), 7.61 (s, 1H), 7.70 (s, 1H), 8.19 (d, 1H), 8.30 (d, 1H), 9.14 (s, 1H) ppm.

Step B: Trans-4-ethyl-cyclohexanecarboxylic acid [(1S)-1-[4-(2,4-dichloro-phenyl)-(E)-1-pent-2-enyl-1H-imidazol-2-yl]-2-(4-hydroxy-phenyl)-ethyl]-amide (277 mg, 0.5 mmol) was first reacted with methyl 4-(bromomethyl)benzoate following the general procedure G then the resulted ester was hydrolyzed following general procedure N to afford 4-(4-{2-[4-(2,4-dichloro-phenyl)-(E)-1-pent-2-enyl-1H-imidazol-2-yl]-(2S)-2-[(trans-4-ethyl-cyclohexanecarbonyl)-amino]-ethyl}-phenoxymethyl)-benzoic acid (255 mg, 76%).

LCMS: m/z 688 (M+H)⁺; ¹H NMR (DMSO, 400 MHz): δ 0.71-0.93 (m, 9H), 0.99-1.15 (m, 2H), 1.22-1.27 (m, 2H), 1.46-1.65 (m, 2H), 1.91-2.02 (m, 4H), 3.04 (m, 1H), 3.36 (m, 1H), 4.46 (m, 1H), 4.56 (d, 1H), 5.13 (s, 1H), 5.18 (m, 1H), 5.35 (m, 1H), 5.53 (m, 1H), 6.87 (d, 2H), 7.11 (d, 2H), 7.42 (d, 1H), 7.45-7.50 (m, 2H), 7.61 (s, 1H), 7.71 (s, 1H), 7.73-7.94 (m, 4H), 8.22 (d, 1H), 8.33 (d, 1H) ppm.

### EXAMPLE 147

### Trans-4-Ethyl-cyclohexanecarboxylic acid [(1S)-1-[(E)-1-but-2-enyl-4-(2,4-dichloro-phenyl)-1H-imidazol-2-yl[-2-(4-hydroxy-phenyl)-ethyl]-amide

Trans-4-ethyl-cyclohexanecarboxylic acid [(1S)-1-[(E)-1-but-2-enyl-4-(2,4-dichloro-phenyl)-1H-imidazol-2-yl]-2-(4-hydroxy-phenyl)-ethyl]-amide (319 mg, 59%) was prepared via N, O-alkylation of trans-4-ethyl-cyclohexanecarboxylic acid [1-[4-(2,4-dichloro-phenyl)-1H-imidazol-2-yl]-(2S)-2-(4-hydroxy-phenyl)-ethyl]-amide (486 g, 1 mmol) with (E)-1-bromo-2-butene following general procedure H followed by ether cleavage using general procedure K.

LCMS: m/z 540 (M+H)⁺; ¹H NMR (DMSO, 400 MHz): δ 0.76-83 (m, 6H), 1.11-1.26 (m, 4H), 1.54-1.58 (m, 2H), 1.67-1.70 (m, 2H), 1.89-2.02 (m, 2H), 2.99-3.03 (m, 1H), 3.15-3.20 (m, 1H), 4.46 (m, 1H), 4.52 (m, 1H), 5.11 (m, 1H), 5.42 (m, 1H), 6.57 (d, 2H), 6.99 (d, 2H), 7.50 (d, 2H), 7.61 (s, 1H), 7.69 (s, 1H), 8.20 (d, 2H), 8.30 (d, 1H), 9.14 (s, 1H) ppm.

### EXAMPLE 148

### 4-(4-{2-[(E)-1-But-2-enyl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid

Trans-4-ethyl-cyclohexanecarboxylic acid [(1S)-1-[(E)-1-but-2-enyl-4-(2,4-dichloro-phenyl)-1H-imidazol-2-yl]-2-(4-hydroxy-phenyl)-ethyl]-amide (270 mg, 0.5 mmol) was first reacted with methyl 4-(bromomethyl)benzoate following the general procedure G then the resulted ester was hydrolyzed following general procedure N to afford 4-(4-{2-[(E)-1-But-2-enyl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid (249 mg, 72%).

LCMS: m/z 674 (M+H)⁺; ¹H NMR (DMSO, 400 MHz): δ 0.74-0.83 (m, 6H), 0.99-1.10 (m, 2H), 1.12-1.1.23 (m, 2H), 1.46-1.57 (m, 2H), 1.64-1.67 (m, 2H), 1.91-1.98 (m, 2H), 3.07 (m, 1H), 3.21 (m, 1H), 3.37 (m, 1H), 4.48 (m, 1H), 4.56 (m, 1H), 5.14 (s, 1H), 5.36 (m, 1H), 5.43 (m, 1H), 6.88 (d, 2H), 7.13 (d, 2H), 7.43 (d, 2H), 7.48-7.51 (m, 3H), 7.61 (s, 1H), 7.70 (s, 1H), 7.88-7.94 (m, 3H), 8.22 (d, 1H), 8.31 (d, 1H) ppm.

### EXAMPLE 149

### 4-(4-{2-[(Z)-But-2-en-1-yl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid

Trans-4-ethyl-cyclohexanecarboxylic acid [(1S)-1-[4-(2,4-dichloro-phenyl)-1H-imidazol-2-yl]-2-(4-hydroxy-phenyl)-ethyl]-amide (486 mg, 1 mmol) was reacted with cis-1-bromo-2-butene following general procedure H followed by ether cleavage using general procedure K to give trans-4-ethyl-cyclohexanecarboxylic acid [(1S)-1-[cis-but-2-en-1-yl-4-(2,4-dichloro-phenyl)-1H-imidazol-2-yl]-2-(4-hydroxy-phenyl)-ethyl]-amide.

Trans-4-ethyl-cyclohexanecarboxylic acid [(1S)-1-[cis-but-2-en-1-yl-4-(2,4-dichloro-phenyl)-1H-imidazol-2-yl]-2-(4-hydroxy-phenyl)-ethyl]-amide (270 mg, 0.5 mmol) was first reacted with methyl 4-(bromomethyl)benzoate following the general procedure G then the resulted ester was hydrolyzed following general procedure N to afford 4-(4-{2-[(Z)-but-2-en-1-yl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid (189 mg, 79%).

LCMS: m/z 674 (M+H)⁺.

### EXAMPLE 150

### Trans-4-Ethyl-cyclohexanecarboxylic acid [(1S)-1-[1-but-2-ynyl-4-(2,4-dichloro-phenyl)-1H-imidazol-2-yl]-2-(4-hydroxy-phenyl)-ethyl]-amide

Trans-4-Ethyl-cyclohexanecarboxylic acid [(1S)-1-[1-but-2-ynyl-4-(2,4-dichloro-phenyl)-1H-imidazol-2-yl]-2-(4-hydroxy-phenyl)-ethyl]-amide (319 mg, 59%) was prepared via N, O-alkylation of 4-ethyl-cyclohexanecarboxylic acid [1-[4-(2,4-dichloro-phenyl)-1H-imidazol-2-yl]-2-(4-hydroxy-phenyl)-ethyl]-amide (486 mg, 1 mmol) with 1-bromo-2-butyne following general procedure H followed by ether cleavage using general procedure K.

LCMS: m/z 538 (M+H)⁺.

### EXAMPLE 151

### 4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid

trans-4-Ethyl-cyclohexanecarboxylic acid [(1S)-1-[1-but-2-ynyl-4-(2,4-dichloro-phenyl)-1H-imidazol-2-yl]-2-(4-hydroxy-phenyl)-ethyl]-amide (269 mg, 0.5 mmol) was first reacted with methyl 4-(bromomethyl)benzoate following the general procedure G then the resulted ester was hydrolyzed following general procedure N to afford 4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid (229 mg, 68%).

LCMS: m/z 672 (M+H)⁺; ¹H NMR (DMSO, 400 MHz): δ 0.72-0.89 (m, 6H), 0.99-1.11 (m, 2H), 1.12-1.1.28 (m, 2H), 1.44-1.57 (m, 2H), 1.61-1.77 (m, 2H), 1.89-1.98 (m, 1H), 2.27-2.41 (m, 1H), 3.09 (m, 1H), 3.22 (m, 1H), 3.39 (m, 1H), 5.16 (s, 1H), 5.39 (m, 1H), 6.89 (d, 2H), 7.16 (d, 2H), 7.44 (d, 2H), 7.47-7.52 (m, 3H), 7.62 (s, 1H), 7.71 (s, 1H), 7.87-7.93 (m, 2H), 8.21 (d, 1H), 8.39 (d, 1H) ppm.

### EXAMPLE 152

### 4-(4-{2-[1-Butyl-4-(2,4-dichloro-phenyl)-1H-imidazol-2-yl]-(2S)-2-[(trans-4-ethyl-cyclohexane-carbonyl)-amino]-ethyl}-2-nitro-phenoxymethyl)-benzoic acid

3-Nitro-L-tyrosine-N-boc (3.2 g, 10 mmole) was treated with 2,4-dichlorophenacyl bromide following the General procedure C to afford the desired keto ester. The imidazole was formed from the keto ester according to general procedure D. Boc-protected amine was treated as described in general procedure E to give (2S)-2-amino-4-{-2-nitro-2-[4-(2,4-dichloro-phenyl)-1H-imidazol-2-yl]-ethyl}-phenol hydrochloride (2.99 g, 76%).

(2S)-2-amino-4-{-2-nitro-2-[4-(2,4-dichloro-phenyl)-1H-imidazol-2-yl]-ethyl}-phenol hydrochloride (393 mg, 1 mmol) was treated with trans-4-ethyl-cyclohexylcarboxylic acid as described in general procedure F to afford trans-4-ethyl-cyclohexanecarboxylic acid {2-(3-nitro-4-hydroxy-phenyl)-(1S)-1-[4-(2,4-dichloro-phenyl)-1H-imidazol-2-yl]-ethyl}-amide (391 mg, 74%).

Trans-4-ethyl-cyclohexanecarboxylic acid {2-(3-nitro-4-hydroxy-phenyl)-(1S)-1-[4-(2,4-dichloro-phenyl)-1H-imidazol-2-yl]-ethyl}-amide (265 mg, 0.5 mmol) was reacted with 1-bromobutane following general procedure H followed by ether cleavage using general procedure K to give trans-4-ethyl-cyclohexanecarboxylic acid {2-(3-nitro-4-hydroxy-phenyl)-(1S)-1-[1-butyl-4-(2,4-dichloro-phenyl)-1H-imidazol-2-yl]-ethyl}-amide (201 mg, 69%).

4-(4-{2-[1-Butyl-4-(2,4-dichloro-phenyl)-1H-imidazol-2-yl]-(2S)-2-[(4-ethyl-cyclohexane-carbonyl)-amino]-ethyl}-2-nitro-phenoxymethyl)-benzoic acid (119 mg, 66%) was synthesized from trans-4-ethyl-cyclohexanecarboxylic acid {2-(3-nitro-4-hydroxy-phenyl)-(1S)-1-[1-butyl-4-(2,4-dichloro-phenyl)-1H-imidazol-2-yl]-ethyl}-amide (146 mg, 0.25 mmol) and methyl 4-(bromomethyl)benzoate following the general procedure G then the resulted ester was hydrolyzed following general procedure N.

LCMS: m/z 721 (M+H)⁺.

### EXAMPLE 153

### 4-(2-Amino-4-{2-[4-(2,4-dichloro-phenyl)-oxazol-2-yl]-(2S)-2-[(trans-4-ethyl-cyclohexanecarbonyl)-amino]-ethyl}-phenoxymethyl)-benzoic acid

3-Nitro-L-tyrosine-N-Boc (3.2 g, 10 mmole) was treated with 2,4-dichlorophenacyl bromide following the general procedure C to afford the desired keto ester. (2S)-2-amino-4-{-2-nitro-2-[4-(2,4-dichloro-phenyl)-1H-oxazol-2-yl]-ethyl}-phenol (3.0 g, 76%) was formed from the keto ester according to general procedure D.

2-amino-4-{-2-nitro-2-[4-(2,4-dichloro-phenyl)-1H-oxazol-2-yl]-ethyl}-phenol (394 mg, 1 mmol) was treated with trans-4-ethylcyclohexylcarboxylic acid as described in general procedure F to afford trans-4-ethyl-cyclohexanecarboxylic acid {2-(3-nitro-4-hydroxy-phenyl)-(1S)-1-[4-(2,4-dichloro-phenyl)-1H-oxazol-2-yl]-ethyl}-amide (392 mg, 74%).

Trans-4-ethyl-cyclohexanecarboxylic acid {2-(3-nitro-4-hydroxy-phenyl)-(1S)-1-[4-(2,4-dichloro-phenyl)-1H-oxazol-2-yl]-ethyl}-amide (266 mg, 0.5 mmol) was reacted with methyl 4-(bromomethyl)benzoate following the general procedure G then the resulted nitro-ester was reduced following general procedure T and was hydrolyzed following general procedure N to afford 4-(2-amino-4-{2-[4-(2,4-dichloro-phenyl)-oxazol-2-yl]- (2S)-2-[(trans-4-ethyl-cyclohexanecarbonyl)-amino]-ethyl}-phenoxymethyl)-benzoic acid (226 mg, 71%).

LCMS: m/z 636 (M+H)⁺.

### EXAMPLE 154

### 4-(4-{2-[4-(2,4-Dichloro-phenyl)-oxazol-2-yl]-(2S)-2-[2-(4-methoxy-phenyl)-acetylamino]-ethyl}-2-nitro-phenoxymethyl)-benzoic acid

3-Nitro-L-tyrosine-N-Boc (3.2 g, 10 mmole) was treated with 2,4-dichlorophenacyl bromide following the General procedure C to afford the desired keto ester. (2S)-2-Amino-4-{-2-nitro-2-[4-(2,4-dichloro-phenyl)-1H-oxazol-2-yl]-ethyl}-phenol (3.0 g, 76%) was formed from the keto ester according to general procedure D.

(2S)-2-Amino-4-{-2-nitro-2-[4-(2,4-dichloro-phenyl)-1H-oxazol-2-yl]-ethyl}-phenol (394 mg, 1 mmol) was treated with 4-methoxy phenylacetic acid as described in general procedure F to afford N-{2-(3-nitro-4-hydroxy-phenyl)-1-[4-(2,4-dichloro-phenyl)-oxazol-2-yl]-ethyl}-(2S)-2-(4-methoxy-phenyl)-acetamide (392 mg, 72%).

N-{2-(3-nitro-4-hydroxy-phenyl)-1-[4-(2,4-dichloro-phenyl)-oxazol-2-yl]-ethyl}-(2S)-2-(4-methoxy-phenyl)-acetamide (271 mg, 0.5 mmol) was reacted with methyl 4-(bromomethyl)benzoate following the general procedure G then the resulted nitro-ester was hydrolyzed following general procedure N to afford 4-(4-{2-[4-(2,4-Dichloro-phenyl)-oxazol-2-yl]-(2S)-2-[2-(4-methoxy-phenyl)-acetylamino]-ethyl}-2-nitro-phenoxymethyl)-benzoic acid (226 mg, 67%).

LCMS: m/z 676 (M+H)⁺, ¹H NMR (DMSO, 400 MHz): δ 3.48 (s, 2H), 3.79 (s, 3H), 4.96 (m, 1H), 5.29 (m, 1H), 7.37 (d, 2H), 7.52-7.69 (m, 6H), 7.72 (d, 2H), 7.76-7.97 (m, 6H), 7.99 (d, 2H), 8.01 (s, 1H) ppm.

### EXAMPLE 155

### 4-{4-[2-[1-Butyl-4-(2,4-dichloro-phenyl)-1H-imidazol-2-yl]-(2S)-2-(4-dimethyl-carbamoyl-butyrylamino)-ethyl]-phenoxymethyl}-benzoic acid

4-{4-[2-[1-Butyl-4-(2,4-dichloro-phenyl)-1H-imidazol-2-yl]-(2S)-2-(4-dimethyl-carbamoyl-butyrylamino)-ethyl]-phenoxymethyl)benzoic acid (35 mg, 52%) was prepared from 4-(4-{(2S)-2-amino-2-[1-butyl-4-(2,4-dichloro-phenyl)-1H-imidazol-2-yl]-ethyl}-phenoxy-methyl)-benzoic acid methyl ester hydrochloride (59 mg, 0.1 mmol) and 4-dimethylcarbamoyl-butyric acid via amide formation followed by ester hydrolysis following general procedures F and N respectively.

LCMS: m/z 679 (M+H)⁺; ¹H NMR (DMSO-d6, 400 MHz): δ 0.82 (t, 3H), 1.17 (m, 2H), 1.48 (m, 2H), 1.65 (m, 2H), 2.05 (m, 4H), 2.74 (s, 3H), 2.80 (s, 3H), 3.18 (m, 2H), 3.86 (m, 1H), 3.98 (m, 1H), 5.11 (s, 2H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H) ppm.

### EXAMPLE 156

### 4-(4-{(2S)-2-tert-Butoxycarbonylamino-2-[1-butyl-4-(2,4-dichloro-phenyl)-1H-imidazol-2-yl]-ethyl}-phenoxy)-benzoic acid methyl ester

4-(4-{(2S)-2-tert-Butoxycarbonylamino-2-[1-butyl-4-(2,4-dichloro-phenyl)-1H-imidazol-2-yl]-ethyl}-phenoxy)-benzoic acid methyl ester (201 mg, 63%) was prepared from [(1S)-1-[1-butyl-4-(2,4-dichloro-phenyl)-1H-imidazol-2-yl]-2-(4-hydroxy-phenyl)-ethyl]-carbamic acid tert-butyl ester (504 mg, 1 mmol) and methyl 4-iodobenzoate (262 mg, 1 mmol) following the general procedure Q.

LCMS: m/z 638 (M+H)⁺; ¹H NMR (CDCl₃, 400 MHz): δ 0.85-0.97 (m, 12H), 1.19-1.26 (m, 2H), 1.32-1.42 (m, 2H), 1.49-1.58 (m, 2H), 3.28 (m, 1H), 3.65 (m, 1H), 3.89 (s, 3H), 4.98 (m, 1H), 5.30 (m, 1H), 6.90 (d, 2H), 7.13 (d, 2H), 7.29 (d, 1H), 7.42 (d, 2H), 7.46 (d, 2H), 7.95 (d, 2H), 8.14 (d, 2H) ppm.

### EXAMPLE 157

### 4-(4-{(2S)-2-tert-Butoxycarbonylamino-2-[1-butyl-4-(2,4-dichloro-phenyl)-1H-imidazol-2-yl]-ethyl}-phenoxy)-benzoic acid

4-(4-{(2S)-2-tert-Butoxycarbonylamino-2-[1-butyl-4-(2,4-dichloro-phenyl)-1H-imidazol-2-yl]-ethyl}-phenoxy)-benzoic acid (249 mg, 80%) was prepared from 4-(4-{(2S)-2-tert-butoxycarbonylamino-2-[1-butyl-4-(2,4-dichloro-phenyl)-1H-imidazol-2-yl]-ethyl)phenoxy)-benzoic acid methyl ester (319 mg, 0.5 mmol) following the general procedure N.

LCMS: m/z 624 (M+H)⁺; ¹H NMR (DMSO, 400 MHz): δ 0.74-0.88 (m, 12H), 1.23-1.25 (m, 1H), 1.30-1.45 (m, 1H), 1.53-1.57 (m, 1H), 3.20 (m, 1H), 3.30 (m, 1H), 3.89 (m, 1H), 4.00 (m, 1H), 4.91 (m, 1H), 6.57 (s, 1H), 6.92 (s, 2H), 7.02 (d, 2H), 7.32 (d, 2H), 7.48 (d, 2H), 7.52 (s, 1H), 7.81 (s, 1H), 7.90 (d, 2H), 8.21 (d, 1H) ppm.

### EXAMPLE 158

### 4-(4-{(2S)-2-[(4-tert-Butyl-cyclohexanecarbonyl)-amino]-2-[1-butyl-4-(2,4-dichloro-phenyl)-1H-imidazol-2-yl]-ethyl}-phenoxy)-benzoic acid

Step 1: 4-(4-{(2S)-2-Amino-2-[1-butyl-4-(2,4-dichloro-phenyl)-1H-imidazol-2-yl]-ethyl}-phenoxy)-benzoic acid methyl ester hydrochloride (231 mg, 85%) was prepared from 4-(4-{(2S)-2-tert-butoxycarbonylamino-2-[1-butyl-4-(2,4-dichloro-phenyl)-1H-imidazol-2-yl]-ethyl}-phenoxy)-benzoic acid methyl ester (319 mg, 0.5 mmol) following the general procedure E.

LCMS: m/z 538 (M+H)⁺; ¹H NMR (DMSO, 400 MHz): δ 0.79 (t, 3H), 1.03-1.18 (m, 3H), 1.33-1.40 (m, 1H), 3.28-3.40 (m, 2H), 3.65-3.75 (m, 2H), 3.82 (s, 3H), 4.78 (m, 1H), 6.96 (d, 2H), 7.02 (d, 2H), 7.15 (d, 2H), 7.54 (d, 2H), 7.66 (d, 1H), 7.81 (s, 1H), 7.93 (d, 2H), 8.20 (d, 1H), 8.70 (s, 1H) ppm.

Step 2: 4-(4-{(2S)-2-[(4-tert-Butyl-cyclohexanecarbonyl)-amino]-2-[1-butyl-4-(2,4-dichloro-phenyl)-1H-imidazol-2-yl]-ethyl}-phenoxy)-benzoic acid (137 mg, 20%) was prepared from 4-tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) and methyl 4-iodobenzoate via diaryl ether coupling followed by ester hydrolysis following general procedures Q and I respectively.

LCMS: m/z 690 (M+H)⁺, ¹H NMR (DMSO, 400 MHz): δ 0.78 (s, 9H), 0.80 (t, 3H), 1.10-1.80 (m, 12H), 1.90 (m, 1H), 3.10-3.25 (m, 3H), 3.90 (m, 2H), 5.25 (m, 1H), 6.87 (d, 2H), 6.95 (d, 2H), 7.26 (d, 2H), 7.44 (dd, 1H), 7.56 (d, 1H), 7.76 (s, 1H), 7.83 (d, 2H), 8.15 (d, 1H), 8.27 (d, 1H) ppm.

### EXAMPLE 159

### 4-(4-{2-[1-Butyl-4-(2,4-dichloro-phenyl)-1H-imidazol-2-yl]-(2S)-2-[(trans-4-ethyl-cyclohexane-carbonyl)-amino]-ethyl}-phenoxy)-benzoic acid

4-(4-{2-[1-Butyl-4-(2,4-dichloro-phenyl)-1H-imidazol-2-yl]-(2S)-2-[(trans-4-ethyl-cyclohexane-carbonyl)-amino]-ethyl}-phenoxy)-benzoic acid (46 mg, 6.9%) was prepared from trans-4-ethylcyclohexanecarboxylic acid [(1S)-1-[1-butyl-4-(2,4-dichloro-phenyl)-1H-imidazol-2-yl]-2-(4-hydroxy-phenyl)-ethyl]-amide (542 mg, 1 mmol) and methyl 4-iodobenzoate via diaryl ether coupling followed by ester hydrolysis following general procedures Q and I respectively.

LCMS: m/z 662 (M+H)⁺, ¹H NMR (DMSO, 400 MHz): 0.83 (m, 6H), 1.05-1.30 (m, 8H), 1.50 (m, 4H), 1.65 (m, 2H), 2.00 (m, 1H), 3.13-3.25 (m, 3H), 3.90 (m, 2H), 5.25 (m, 1H), 6.87 (d, 2H), 6.95 (d, 2H), 7.28 (d, 2H), 7.48 (dd, 1H), 7.60 (d, 1H), 7.79 (s, 1H), 7.86 (d, 2H), 8.19 (d, 1H), 8.33 (d, 1H) ppm.

### EXAMPLE 160

### 4-(4-{2-[4-(2,4-Dichloro-phenyl)-1-pent-2-enyl-1H-imidazol-2-yl]-(2S)-2-[(trans-4-ethyl-cyclohexane-carbonyl)-amino]-ethyl}-phenoxy)-benzoic acid

Trans-4-ethyl-cyclohexanecarboxylic acid [(1S)-1-[4-(2,4-dichloro-phenyl)-1-pent-2-enyl-1H-imidazol-2-yl]-2-(4-hydroxy-phenyl)-ethyl]-amide (554 mg, 1 mmol) was first reacted with methyl 4-iodobenzoate (262 mg, 1 mmol) following the general procedure Q and then was hydrolyzed following general procedure N to afford 4-(4-{2-[4-(2,4-Dichloro-phenyl)-1-pent-2-enyl-1H-imidazol-2-yl]-(2S)-2-[(trans-4-ethyl-cyclohexane-carbonyl)-amino]-ethyl}-phenoxy)-benzoic acid (221 mg, 65%).

LCMS: m/z 674 (M+H)⁺; ¹H NMR (DMSO, 400 MHz): δ 0.72-0.95 (m, 9H), 0.97-1.15 (m, 1H), 1.22-1.27 (m, 1H), 1.34-1.59 (m, 4H), 1.67-2.32 (m, 4H), 3.19 (m, 1H), 3.36 (m, 1H), 4.57 (d, 1H), 5.28 (m, 1H), 5.44 (m, 1H), 5.63 (m, 1H), 6.92 (d, 2H), 6.96 (d, 2H), 7.02 (d, 1H), 7.30 (d, 2H), 7.33 (s, 1H), 7.50 (s, 1H), 7.64 (d, 2H), 7.78 (d, 2H), 8.17 (d, 1H), 8.38 (1, 1H) ppm.

### EXAMPLE 161

### 4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxy)-benzoic acid

Trans-4-ethyl-cyclohexanecarboxylic acid [(1S)-1-[1-but-2-enyl-4-(2,4-dichloro-phenyl)-1H-imidazol-2-yl]-2-(4-hydroxy-phenyl)-ethyl]-amide (269 mg, 0.5 mmol) was first reacted with reacted with methyl 4-iodobenzoate (131 mg, 1 mmol) following the general procedure Q and then hydrolyzed following general procedure N to afford 4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxy)-benzoic acid (226 mg, 69%).

LCMS: m/z 658 (M+H)⁺; ¹H NMR (DMSO, 400 MHz): δ 0.74-0.83 (m, 6H), 0.99-1.10 (m, 2H), 1.12-1.1.23 (m, 2H), 1.46-1.57 (m, 2H), 1.64-1.67 (m, 2H), 1.91-198 (m, 2H), 3.07 (m, 1H), 3.21 (m, 1H), 3.37 (m, 1H), 4.56 (m, 1H), 5.36 (m, 1H), 5.43 (m, 1H), 6.88 (d, 2H), 7.13 (d, 2H), 7.43 (d, 2H), 7.48-7.51 (m, 2H), 7.61 (s, 1H), 7.70 (s, 1H), 7.88-7.94 (m, 2H), 8.22 (d, 1H), 8.31 (d, 1H) ppm.

### EXAMPLE 162

### 4-(4-{2-[4-(2,4-Dichloro-phenyl)-1-prop-2-ynyl-1H-imidazol-2-yl]-(2S)-2-[(trans-4-ethyl-cyclohexane-carbonyl)-amino]-ethyl}-phenoxy)-benzoic acid

Trans-4-ethyl-cyclohexanecarboxylic acid [(1S)-1-[4-(2,4-dichloro-phenyl)-1-prop-2-ynyl-1H-imidazol-2-yl]-2-(4-hydroxy-phenyl)-ethyl]-amide (319 mg, 60%) was prepared via N, O-alkylation of trans-4-ethyl-cyclohexanecarboxylic acid [(1S)-1-[4-(2,4-dichloro-phenyl)-1H-imidazol-2-yl]-2-(4-hydroxy-phenyl)-ethyl]-amide (486 mg, 1 mmol) with propargyl bromide following general procedure H followed by ether cleavage using general procedure K.

Trans-4-ethyl-cyclohexanecarboxylic acid [(1S)-1-[4-(2,4-dichloro-phenyl)-1-prop-2-ynyl-1H-imidazol-2-yl]-2-(4-hydroxy-phenyl)-ethyl]-amide (262 mg, 0.5 mmol) was first reacted with methyl 4-iodobenzoate (66 mg, 0.5 mmol) following the general procedure Q and then hydrolyzed following general procedure N to afford 4-(4-{2-[4-(2,4-dichloro-phenyl)-1-prop-2-ynyl-1H-imidazol-2-yl]-(2S)-2-[(trans-4-ethyl-cyclohexane-carbonyl)-amino]-ethyl}-phenoxy)-benzoic acid (128 mg, 79%).

LCMS: m/z 644 (M+H)⁺; ¹H NMR (DMSO, 400 MHz): δ 0.72-0.89 (m, 4H), 0.99-1.11 (m, 2H), 1.12-1.1.28 (m, 2H), 1.44-1.57 (m, 2H), 1.61-1.77 (m, 4H), 1.89-1.98 (m, 1H), 2.27-2.41 (m, 1H), 3.09 (m, 1H), 3.22 (m, 1H), 3.39 (m, 1H), 5.39 (m, 1H), 6.89 (d, 2H), 7.16 (d, 2H), 7.44 (d, 2H), 7.47-7.52 (m, 2H), 7.62 (s, 1H), 7.71 (s, 1H), 7.87-7.93 (m, 2H), 8.21 (d, 1H), 8.39 (d, 1H) ppm.

### EXAMPLE 163

### 4-(4-{(2S)-2-tert-Butoxycarbonylamino-2-[1-[3-(1S)-1-carboxy-3-methylsulfanyl-propylcarbamoyl)-propyl]-4-(2,4-dichloro-phenyl)-1H-imidazol-2-yl]-ethyl}-phenoxy)-benzoic acid

4-(4-{(2S)-2-tert-Butoxycarbonylamino-2-[1-[3-((1S)-1-carboxy-3-methylsulfanyl-propylcarbamoyl)-propyl]-4-(2,4-dichloro-phenyl)-1H-imidazol-2-yl]-ethyl}-phenoxy)-benzoic acid (112 mg, 57%) was prepared from [(1S)-1-[4-(2,4-dichloro-phenyl)-1H-imidazol-2-yl]-2-(4-hydroxy-phenyl)-ethyl]-carbamic acid tert-butyl ester (449 mg, 1 mmol) by N,O alkylation with methyl 4-bromobutyrate following general procedure H, followed by ether cleavage using general procedure K. The ester group was hydrolyzed using general procedure N, and L-methionine methyl ester was coupled to the acid following general procedure F. The obtained 2-{4-[2-[1-tert-butoxycarbonylamino-2-(4-hydroxy-phenyl)-ethyl]-4-(2,4-dichloro-phenyl)-imidazol-1 yl]-butyrylamino}-4-methylsulfanyl-butyric acid methyl ester (170 mg, 0.25 mmol) was first reacted with methyl 4-iodobenzoate (33 mg, 0.25 mmol) following the general procedure Q and then hydrolyzed following general procedure N.

LCMS: m/z 785 (M+H)⁺.

### EXAMPLE 164

### 4-(4-{(2S)-2-tert-Butoxycarbonylamino-2-[1-[5-((1S)-1-carboxy-3-methylsulfanyl-propylcarbamoyl)-pentyl]-4-(2,4-dichloro-phenyl)-1H-imidazol-2-yl]-ethyl}-phenoxy)-benzoic acid

4-(4-{(2S)-2-tert-Butoxycarbonylamino-2-[1-[5-((1S)-1-carboxy-3-methylsulfanyl-propylcarbamoyl)-pentyl]-4-(2,4-dichloro-phenyl)-1H-imidazol-2-yl]-ethyl}-phenoxy)-benzoic acid (110 mg, 54%)was preparedfrom [(1S)-1-[4-(2,4-Dichloro-phenyl)-1H-imidazol-2-yl]-2-(4-hydroxy-phenyl)-ethyl]-carbamic acid tert-butyl ester (449 mg, 1 mmol) by N,O alkylation with 6-bromo-methyl hexanoate following general procedure H, followed by ether cleavage using general procedure K. The ester group was hydrolyzed using general procedure N, and L-methionine methyl ester was coupled to the acid following general procedure F. The obtained 2-{6-[(2S)-2-[(1S)-1-tert-butoxycarbonylamino-2-(4-hydroxy-phenyl)-ethyl]-4-(2,4-dichloro-phenyl)-imidazol-1yl]-hexanoylamino}-4-methylsulfanyl-butyric acid methyl ester (176 mg, 0.25 mmol) was first reacted with methyl 4-iodobenzoate (33 mg, 0.25 mmol) following the general procedure Q and then hydrolyzed following general procedure N.

LCMS: m/z 813 (M+H)⁺.

### EXAMPLE 165

### 4-(4-{2-[1-[5-((1S)-1-Carboxy-3-methylsulfanyl-propylcarbamoyl)-pentyl]-4-(2,4-dichloro-phenyl)-1H-imidazol-2-yl]-(2S)-2-[(trans-4-ethyl-cyclohexanecarbonyl)-amino]-ethyl}-phenoxy)-benzoic acid

4-(4-{2-[1-[5-((1S)-1-Carboxy-3-methylsulfanyl-propylcarbamoyl)-pentyl]-4-(2,4-dichloro-phenyl)-1H-imidazol-2-yl]-(2S)-2-[(trans-4-ethyl-cyclohexanecarbonyl)-amino]-ethyl}-phenoxy)-benzoic acid (109 mg, 51%) was prepared from trans-4-ethyl-cyclohexanecarboxylic acid [(1S)-1-[4-(2,4-dichloro-phenyl)-1H-imidazol-2-yl]-2-(4-hydroxy-phenyl)-ethyl]-amide (486 mg, 1 mmol) by N, O alkylation with 6-bromo-methyl hexanoate following general procedure H, followed by ether cleavage using general procedure K. The ester group was hydrolyzed using general procedure N, and L-methionine methyl ester was coupled to the acid following general procedure F. The obtained (2S)-2-{6-[4-[2,4-dichloro-phenyl)-2-[(1S)-1-[(4-ethyl-cyclohexane carbonyl)-amino]-2-(4-hydroxy-phenyl)-ethyl]-imidazol-1-yl}-hexanoylamino)-4-methyl sulfanyl-butyric acid methyl ester (186 mg, 0.25 mmol) was first reacted with methyl 4-iodobenzoate (33 mg, 0.25 mmol) following the general procedure Q and then hydrolyzed following general procedure N.

LCMS: m/z 851 (M+H)⁺.

### EXAMPLE 166

### 4-(4-{(2S)-2-tert-Butoxycarbonylamino-2-[1-[4-((1S)-1-carboxy-3-methylsulfanyl-propylcarbamoyl)-butyl]-4-(2,4-dichloro-phenyl)-1H-imidazol-2-yl]-ethyl}-phenoxy)-benzoic acid

4-(4-{(2S)-2-tert-Butoxycarbonylamino-2-[1-[4-((1S)-1-carboxy-3-methylsulfanyl-propylcarbamoyl)-butyl]-4-(2,4-dichloro-phenyl)-1H-imidazol-2-yl]-ethyl}-phenoxy)-benzoic acid (4.1 mg, 0.5%) was prepared from [(1S)-1-[4-(2,4-Dichloro-phenyl)-1H-imidazol-2-yl]-2-(4-hydroxy-phenyl)-ethyl]-carbamic acid tert-butyl ester (449 mg, 1 mmol) by N,O alkylation with 7-bromo-heptanoic acid ethyl ester following general procedure H, followed by ether cleavage using general procedure K. The ester group was hydrolyzed using general procedure I, and L-methionine methyl ester was coupled to the acid following general procedure F. Finally, methyl 4-iodobenzoate was coupled to the phenol using general procedure Q and ester hydrolysis was performed using general procedure I.

LCMS: m/z 799 (M+H)⁺, ¹H NMR (DMSO, 400 MHz): 1.28 (s, 9H), 1.43 (m, 2H), 1.55 (m, 2H), 1.78 (m, 1H), 1.89 (m, 1H), 1.95 (s, 3H), 2.12 (m, 2H), 2.40 (m, 2H), 3.18 (m, 1H), 3.27 (m, 1H), 3.94 (m, 2H), 4.18 (m, 1H), 4.90 (m, 1H), 6.89 (d, 2H), 6.96 (d, 2H), 7.29 (d, 2H), 7.45 (dd, 1H), 7.58 (d, 1H), 7.77 (s, 1H), 7.88 (d, 2H), 8.18 (d, 1H) ppm.

### EXAMPLE 167

### 4-(4-{2-[1-Butyl-4-(2,4-dichloro-phenyl)-1H-imidazol-2-yl]-(2S)-2-[5-(1-carboxy-3-methylsulfanyl-propylcarbamoyl)-pentanoclamino]-ethyl}-phenoxy)-benzoic acid

4-(4-{2-[1-Butyl-4-(2,4-dichloro-phenyl)-1H-imidazol-2-yl]-(2S)-2-[5-(1-carboxy-3-methylsulfanyl-propylcarbamoyl)-pentanoylamino]-ethyl}-phenoxy)-benzoic acid (25 mg, 32%) was prepared from 4-(4-{(2S)-2-amino-2-[1-butyl-4-(2,4-dichlorophenyl)-1H-imidazol-2-yl]-ethyl}-phenoxy)-benzoic acid methyl ester hydrochloride (58 mg, 0.1 mmol) via 2-step sequential amide formation with adipic acid and L-methionine methyl ester both following general procedure F, followed by ester hydrolysis following general procedure N.

LCMS: m/z 783 (M+H)⁺; ¹H NMR (DMSO-d6, 400 MHz): δ 0.83 (t, 3H), 1.18 (m, 2H), 1.49 (m, 2H), 1.66 (m, 4H), 2.06 (m, 4H), 2.10 (s, 3H), 2.22 (m, 2H), 2.62 (m, 2H), 3.18 (m, 2H), 3.86 (m, 1H), 3.98 (m, 1H), 4.47 (m, 1H), 5.23 (m, 1H), 6.93 (d, 2H), 6.98 (d, 2H), 7.28 (d, 2H), 7.30 (d, 2H), 7.50 (m, 2H), 7.62 (d, 1H), 7.79 (s, 1H), 8.21 (d, 1H), 8.51 (d, 1H) ppm.

### EXAMPLE 168

### 4-{4-[2-[1-Butyl-4-(2,4-dichloro-phenyl)-1H-imidazol-2-yl]-(2S)-2-(4-dimethylcarbamoyl-butyrylamino)-ethyl]-phenoxy}-benzoic acid

Step 1:

To a solution of 4-(4-{(2S)-2-amino-2-[1-butyl-4-(2,4-dichloro-phenyl)-1H-imidazol-2-yl]-ethyl}-phenoxy)-benzoic acid methyl ester hydrochloride (574 mg, 1 mmol) in DMF (0.1-0.5 M), glutaric anhydride (228 mg, 2 mmol) was added, followed by DIEA (0.5 mL, 3 mmol). The reaction mixture is then stirred at 120° C. for 3 hours. At completion the reaction mixture is acidified with 10% citric acid, and extracted with ethyl acetate. The organic extracts are combined, washed with brine, dried over Na₂SO₄, and the solvent removed in vacuo. The residue was purified by silica gel chromatography to afford 4-{4-[2-[1-Butyl-4-(2,4-dichloro-phenyl)-1H-imidazol-2-yl]-(2S)-2-(4-carboxy-butyrylamino)-ethyl]-phenoxy}-benzoic acid methyl ester (440 mg, 68%).

LCMS: m/z 652 (M+H)⁺; ¹H NMR (DMSO-d6, 400 MHz): δ 0.82 (t, 3H), 1.17 (m, 2H), 1.48 (m, 2H), 1.65 (m, 2H), 2.05 (m, 4H), 3.18 (m, 2H), 3.80 (s, 3H), 3.86 (m, 1H), 3.98 (m, 1H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H) ppm.

Step 2:

4-{4-[2-[1-Butyl-4-(2,4-dichloro-phenyl)-1H-imidazol-2-yl]-(2S)-2-(4-dimethylcarbamoyl-butyrylamino)-ethyl]-phenoxy)benzoic acid (44 mg, 67%) was prepared from 4-{4-[2-[1-Butyl-4-(2,4-dichloro-phenyl)-1H-imidazol-2-yl]-(2S)-2 (4-carboxy-butyrylamino)-ethyl]-phenoxy}-benzoic acid methyl ester (65 mg, 0.1 mmol) and dimethylamine via amide formation followed by ester hydrolysis following general procedures F and N respectively.

LCMS: m/z 665 (M+H)⁺; ¹H NMR (DMSO-d6, 400 MHz): δ 0.82 (t, 3H), 1.17 (m, 2H), 1.48 (m, 2H), 1.65 (m, 2H), 2.05 (m, 4H), 2.74 (s, 3H), 2.80 (s, 3H), 3.18 (m, 2H), 3.86 (m, 1H), 3.98 (m, 1H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H), ppm.

### EXAMPLE 169

### 4-(4-{(2S)-2-(4-tert-Butylcarbamoyl-butyrylamino)-2-[1-butyl-4-(2,4-dichloro-phenyl)-1H-imidazol-2-yl]-ethyl}-phenoxy)-benzoic acid

4-(4-{(2S)-2-(4-tert-Butylcarbamoyl-butyrylamino)-2-[1-butyl-4-(2,4-dichloro-phenyl)-1H-imidazol-2-yl]-ethyl}-phenoxy)-benzoic acid (43 mg, 62%) was prepared from 4-{4-[2-[1-butyl-4-(2,4-dichloro-phenyl)-1H-imidazol-2-yl]-(2S)-2-(4-carboxy-butyrylamino)-ethyl]-phenoxy}-benzoic acid methyl ester (65 mg, 0.1 mmol) and tert-butylamine via amide formation followed by ester hydrolysis following general procedures F and N respectively.

LCMS: m/z 693 (M+H)⁺; ¹H NMR (DMSO-d6, 400 MHz): δ 0.81 (t, 3H), 1.06 (s, 9H), 1.16 (m, 2H), 1.47 (m, 2H), 1.65 (m, 2H), 2.03 (m, 4H), 3.17 (m, 2H), 3.84 (m, 1H), 3.96 (m, 1H), 5.20 (m, 1H), 6.95 (d, 2H), 6.99 (d, 2H), 7.28 (d, 2H), 7.31 (d, 2H), 7.49 (dd, 1H), 7.63 (m, 2H), 7.80 (s, 1H), 8.23 (d, 1H), 8.55 (d, 1H) ppm.

### EXAMPLE 170

### 4-(4-{(2S)-2-(4-Benzylcarbamoyl-butyrylamino)-2-[1-butyl-4-(2,4-dichloro-phenyl)-1H-imidazol-2-yl]-ethyl}-phenoxy)-benzoic acid

4-(4-{2S)-2-(4-Benzylcarbamoyl-butyrylamino)-2-[1-butyl-4-(2,4-dichloro-phenyl)-1H-imidazol-2-yl]-ethyl}-phenoxy)-benzoic acid (51 mg, 70%) was prepared from 4-{4-[2-[1-butyl-4-(2,4-dichloro-phenyl)-1H-imidazol-2-yl]-(2S)-2-(4-carboxy-butyrylamino)-ethyl]-phenoxy}-benzoic acid methyl ester (65 mg, 0.1 mmol) and benzylamine via amide formation followed by ester hydrolysis following general procedures F and N respectively.

LCMS: m/z 727 (M+H)⁺; ¹H NMR (DMSO-d6, 400 MHz): δ 0.83 (t, 3H), 1.17 (m, 2H), 1.50 (m, 2H), 1.66 (m, 2H), 2.06 (m, 4H), 3.17 (m, 2H), 3.86 (m, 1H), 3.98 (m, 1H), 4.23 (d, 2H), 5.24 (m, 1H), 6.94 (d, 2H), 6.99 (d, 2H), 7.21 (m, 3H), 7.27 (d, 2H), 7.30 (d, 2H), 7.49 (dd, 1H), 7.62 (d, 1H), 7.79 (s, 1H), 7.91 (d, 2H), 8.22 (d, 1H), 8.27 (m, 1H), 8.53 (d, 1H) ppm.

### EXAMPLE 171

### 4-(4-{2-[1-Butyl-4-(2,4-dichloro-phenyl)-1H-imidazol-2-yl]-(2S)-2-[4-(4-methoxy-benzylcarbamoyl)-butyrylamino]-ethyl}-phenoxy)-benzoic acid

4-(4-{2-[1-Butyl-4-(2,4-dichloro-phenyl)-1H-imidazol-2-yl]-(2S)-2-[4-(4-methoxy-benzylcarbamoyl)-butyrylamino]-ethyl}-phenoxy)-benzoic acid (45 mg, 60%) was prepared from 4-{4-[2-[1-butyl-4-(2,4-dichloro-phenyl)-1H-imidazol-2-yl]-(2S)-2-(4-carboxy-butyrylamino)-ethyl]-phenoxy}-benzoic acid methyl ester (65 mg, 0.1 mmol) and 4-methoxybenzylamine via amide formation followed by ester hydrolysis following general procedures F and N respectively.

LCMS: m/z 757 (M+H)⁺; ¹H NMR (CDCl₃—CD₃OD, 400 MHz): δ 0.83 (t, 3H), 1.15 (m, 2H), 1.34 (m, 2H), 1.86 (m, 2H), 2.50 (m, 4H), 3.17 (m, 2H), 3.56 (m, 1H), 3.68 (m, 1H), 3.77 (s, 3H), 3.80 (d, 2H), 5.19 (m, 1H), 6.84 (m, 5H), 6.97 (d, 2H), 7.19 (m, 2H), 7.34 (m, 1H), 7.47 (m, 1H), 7.49 (m, 2H), 7.93 (d, 1H), 8.04 (d, 2H) ppm.

### EXAMPLE 172

### 4-(4-{2-[1-Butyl-4-(2,4-dichloro-phenyl)-1H-imidazol-2-yl]-(2S)-2-[4-(3-fluorobenzylcarbamoyl)-butyrylamino]-ethyl}-phenoxy)-benzoic acid

4-(4-{2-[1-Butyl-4-(2,4-dichloro-phenyl)-1H-imidazol-2-yl]-(2S)-2-[4-(3-fluoro-benzylcarbamoyl)-butyrylamino]-ethyl}-phenoxy)-benzoic acid (58 mg, 78%) was prepared from 4-{4-[2-[1-butyl-4-(2,4-dichloro-phenyl)-1H-imidazol-2-yl]-(2S)-2-(4-carboxy-butyrylamino)-ethyl]-phenoxy}-benzoic acid methyl ester (65 mg, 0.1 mmol) and 3-fluorobenzylamine via amide formation followed by ester hydrolysis following general procedures F and N respectively.

LCMS: 745 (M+H)⁺; ¹H NMR (CDCl₃—CD₃OD, 400 MHz): δ 0.84 (t, 3H), 1.16 (m, 2H), 1.37 (m, 2H), 1.88 (m, 2H), 2.53 (m, 4H), 3.19 (m, 2H), 3.62 (m, 1H), 3.78 (m, 1H), 3.84 (m, 2H), 5.20 (m, 1H), 6.86 (m, 5H), 6.98 (d, 2H), 7.20 (m, 2H), 7.34 (m, 1H), 7.51 (m, 1H), 7.59 (m, 2H), 7.93 (d, 1H), 8.10 (d, 2H) ppm.

### EXAMPLE 173

### 4-{4-[2-[4-(2,4-Dichloro-phenyl)-1-ethyl-1H-imidazol-2-yl]-(2S)-2-(4-phenylcarbamoyl-butyrylamino)-ethyl]-phenoxy}-benzoic acid

Step A:

To a solution of 4-(4-{(2S)-2-Amino-2-[4-(2,4-dichloro-phenyl)-1-ethyl-1H-imidazol-2-yl]-ethyl}-phenoxy)-benzoic acid methyl ester hydrochloride (546 mg, 1 mmol) in DMF (0.1-0.5 M), glutaric anhydride (228 mg, 2 mmol) was added, followed by DIEA (0.5 mL, 3 mmol). The reaction mixture is then stirred at 120° C. for 3 hours. At completion the reaction mixture is acidified with 10% citric acid, and extracted with ethyl acetate. The organic extracts are combined, washed with brine, dried over Na₂SO₄, and the solvent removed in vacuo. The residue was purified by silica gel chromatography to afford 4-(4-{(2S)-2-(4-carboxy-butyrylamino)-2-[4-(2,4-dichloro-phenyl)-1-ethyl-1H-imidazol-2-yl]-ethyl}-phenoxy)-benzoic acid methyl ester (456 mg, 73%).

LCMS: m/z 624 (M+H)⁺; ¹H NMR (DMSO-d6, 400 MHz): δ 1.18 (t, 3H), 1.65 (m, 2H), 2.05 (m, 4H), 3.18 (m, 2H), 3.80 (s, 3H), 3.98 (m, 2H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H) ppm.

Step B:

4-{4-[2-[4-(2,4-Dichloro-phenyl)-1-ethyl-1H-imidazol-2-yl]-(2S)-2-(4-phenylcarbamoyl-butyrylamino)-ethyl]-phenoxy}-benzoic acid (49 mg, 71%) was prepared from 4-(4-{(2S)-2-(4-carboxy-butyrylamino)-2-[4-(2,4-dichloro-phenyl)-1-ethyl-1H-imidazol-2-yl]-ethyl}-phenoxy)-benzoic acid methyl ester (62 mg, 0.1 mmol) and aniline via amide formation followed by ester hydrolysis following general procedures F and N respectively.

LCMS: m/z685 (M+H)⁺; ¹H NMR (DMSO-d6, 400 MHz): δ 1.17 (t, 3H), 1.73 (m, 2H), 2.11 (m, 2H), 2.24 (m, 2H), 3.16 (m, 2H), 3.95 (m, 2H), 5.22 (m, 1H), 6.79 (d, 2H), 6.90 (d, 2H), 7.00 (m, 1H), 7.26 (m, 4H), 7.47 (dd, 1H), 7.58 (d, 2H), 7.61 (d, 1H), 7.82 (m, 3H), 8.20 (d, 1H), 8.55 (d, 1H), 9.90 (s, 1H) ppm.

### EXAMPLE 174

### 4-(4-{(2S)-2-(4-Benzylcarbamoyl-butyrylamino)-2-[4-(2,4-dichloro-phenyl)-1-ethyl-1H-imidazol-2-yl]-ethyl}-phenoxy)-benzoic acid

4-(4-{(2S)-2-(4-Benzylcarbamoyl-butyrylamino)-2-[4-(2,4-dichloro-phenyl)-1-ethyl-1H-imidazol-2-yl]-ethyl}-phenoxy)-benzoic acid (53 mg, 76%) was prepared from 4-(4-{(2S)-2-(4-carboxy-butyrylamino)-2-[4-(2,4-dichloro-phenyl)-1-ethyl-1H-imidazol-2-yl]-ethyl}-phenoxy)-benzoic acid methyl ester (62 mg, 0.1 mmol) and benzylamine via amide formation followed by ester hydrolysis following general procedures F and N respectively.

LCMS: m/z 699 (M+H)⁺; ¹H NMR (DMSO-d6, 400 MHz): δ 1.18 (t, 3H), 1.66 (m, 2H), 2.06 (m, 4H), 3.17 (m, 2H), 3.95 (m, 2H), 4.23 (d, 2H), 5.23 (m, 1H), 6.90 (d, 2H), 6.97 (d, 2H), 7.21 (m, 3H), 7.28 (m, 4H), 7.49 (dd, 1H), 7.62 (d, 1H), 7.81 (s, 1H), 7.89 (d, 2H), 8.21 (d, 1H), 8.28 (m, 1H), 8.53 (d, 1H) ppm.

### EXAMPLE 175

### 4-(4-{2-[4-(2,4-Dichloro-phenyl)-1-ethyl-1H-imidazol-2-yl]-(2S)-2-[4-(3-fluoro-benzyl-carbamoyl)-butyrylamino]-ethyl}-phenoxy)-benzoic acid

4-(4-{2-[4-(2,4-Dichloro-phenyl)-1-ethyl-1H-imidazol-2-yl]-(2S)-2-[4-(3-fluoro-benzyl-carbamoyl)-butyrylamino]-ethyl}-phenoxy)-benzoic acid (52 mg, 73%) was prepared from 4-(4-{(2S)-2-(4-carboxy-butyrylamino)-2-[4-(2,4-dichloro-phenyl)-1-ethyl-1H-imidazol-2-yl]-ethyl}-phenoxy)-benzoic acid methyl ester (62 mg, 0.1 mmol) and 3-fluorobenzylamine via amide formation followed by ester hydrolysis following general procedures F and N respectively.

LCMS: m/z 717 (M+H)⁺; ¹H NMR (DMSO-d6, 400 MHz): δ 1.18 (t, 3H), 1.65 (m, 2H), 2.05 (m, 4H), 3.17 (m, 2H), 3.95 (m, 2H), 4.21 (d, 2H), 5.22 (m, 1H), 6.85 (d, 2H), 6.93 (d, 2H), 7.10 (m, 2H), 7.26 (m, 4H), 7.48 (dd, 1H), 7.62 (d, 1H), 7.81 (s, 1H), 7.86 (d, 2H), 8.20 (d, 1H), 8.30 (m, 1H), 8.53 (d, 1H) ppm.

### EXAMPLE 176

### 4-{4-[2-[1-Butyl-4-(2,4-dichloro-phenyl)-1H-imidazol-2-yl]-(2S)-2-(3-dimethylcarbamoyl-propionylamino)-ethyl]-phenoxy}-benzoic acid

Step A:

To a solution of 4-(4-{(2S)-2-amino-2-[1-butyl-4-(2,4-dichloro-phenyl)-1H-imidazol-2-yl]-ethyl}-phenoxy)-benzoic acid methyl ester hydrochloride (574 mg, 1 mmol) in DMF (0.1-0.5 M), succinic anhydride (200 mg, 2 mmol) was added, followed by DIEA (0.5 mL, 3 mmol). The reaction mixture is then stirred at 120° C. for 3 hours. At completion the reaction mixture is acidified with 10% citric acid, and extracted with ethyl acetate. The organic extracts are combined, washed with brine, dried over Na₂SO₄, and the solvent removed in vacuo. The residue was purified by silica gel chromatography to afford 4-{4-[2-[1-butyl-4-(2,4-dichloro-phenyl)-1H-imidazol-2-yl]-(2S)-2-(3-carboxy-propionylamino)-ethyl]-phenoxy}-benzoic acid methyl ester (517 mg, 81%).

LCMS: m/z 638 (M+H)⁺; ¹H NMR (DMSO-d6, 400 MHz): δ 0.82 (t, 3H), 1.17 (m, 2H), 1.48 (m, 2H), 2.05 (m, 4H), 3.18 (m, 2H), 3.80 (s, 3H), 3.86 (m, 1H), 3.98 (m, 1H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H) ppm.

Step B:

4-{4-[2-[1-Butyl-4-(2,4-dichloro-phenyl)-1H-imidazol-2-yl]-(2S)-2-(3-dimethylcarbamoyl-propionylamino)-ethyl]-phenoxy}-benzoic acid (47 mg, 72%) was prepared from 4-{4-[2-[1-butyl-4-(2,4-dichloro-phenyl)-1H-imidazol-2-yl]-(2S)-2-(3-carboxy-propionylamino)-ethyl]-phenoxy}-benzoic acid methyl ester (64 mg, 0.1 mmol) and dimethylamine via amide formation followed by ester hydrolysis following general procedures F and N respectively.

LCMS: m/z 651 (M+H)⁺; ¹H NMR (DMSO-d6, 400 MHz): δ 0.82 (t, 3H), 1.17 (m, 2H), 1.48 (m, 2H), 2.05 (m, 4H), 2.74 (s, 3H), 2.80 (s, 3H), 3.18 (m, 2H), 3.86 (m, 1H), 3.98 (m, 1H), 5.22 (m, 1H), 6.94 (d, 2H), 6.99 (d, 2H), 7.27 (d, 2H), 7.30 (d, 2H), 7.49 (dd, 1H), 7.62 (d, 1H), 7.79 (s, 1H), 8.22 (d, 1H), 8.53 (d, 1H) ppm.

### EXAMPLE 177

### 4-(4-{2-[1-Butyl-4-(2,4-dichloro-phenyl)-1H-imidazol-2-yl]-(2S)-2-methanesulfonylamino-ethyl}-phenoxy)-benzoic acid

4-(4-{2-[1-Butyl-4-(2,4-dichloro-phenyl)-1H-imidazol-2-yl]-(2S)-2-methanesulfonylamino-ethyl}-phenoxy)-benzoic acid (39 mg, 65%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and methanesulfonyl chloride via sulfonamide formation followed by ester hydrolysis following general procedures O and N respectively.

LCMS: m/z 602 (M+H)⁺; ¹H NMR (CDCl₃, 400 MHz): δ 0.89 (t, 3H), 1.23 (m, 2H), 1.46 (m, 2H), 3.26 (s, 3H), 3.29 (m, 2H), 3.67 (m, 1H), 3.82 (m, 1H), 5.41 (m, 1H), 6.91 (d, 2H), 6.93 (d, 2H), 7.09 (d, 2H), 7.31 (dd, 1H), 7.33 (s, 1H), 7.45 (d, 1H), 7.65 (d, 1H), 7.91 (d, 1H), 8.02 (d, 2H) ppm.

### EXAMPLE 178

### 4-(4-{2-[1-Butyl-4-(2,4-dichloro-phenyl)-1H-imidazol-2-yl]-(2S)-2-phenylmethanesulfonyl-amino-ethyl}-phenoxy)-benzoic acid

4-(4-{2-[1-Butyl-4-(2,4-dichloro-phenyl)-1H-imidazol-2-yl]-(2S)-2-phenylmethane-sulfonyl-amino-ethyl}-phenoxy)-benzoic acid (239 mg, 70%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (269 mg, 0.5 mmol) and alpha-toluenesulfonyl chloride via sulfonamide formation followed by ester hydrolysis following general procedures O and N respectively.

LCMS: m/z 678 (M+H)⁺.

### EXAMPLE 179

### 4-(4-{(2S)-2-(Butane-1-sulfonylamino)-2-[1-butyl-4-(2,4-dichloro-phenyl)-1H-imidazol-2-yl]-ethyl}-phenoxy)-benzoic acid

4-(4-{(2S)-2-(Butane-1-sulfonylamino)-2-[1-butyl-4-(2,4-dichloro-phenyl)-1H-imidazol-2-yl]-ethyl}-phenoxy)-benzoic acid (218 mg, 68%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (269 mg, 0.5 mmol) and n-butanesulfonyl chloride via sulfonamide formation followed by ester hydrolysis following general procedures O and N respectively.

LCMS: m/z 644 (M+H)⁺.

### EXAMPLE 180

### 4-{4-[2-[1-Butyl-4-(2,4-dichloro-phenyl)-1H-imidazol-2-yl]-(2S)-2-(4-ethyl-benzenesulfonyl-amino)-ethyl]-phenoxy}-benzoic acid

4-{4-[2-[1-Butyl-4-(2,4-dichloro-phenyl)-1H-imidazol-2-yl]-(2S)-2-(4-ethyl-benzene-sulfonyl-amino)-ethyl]-phenoxy}-benzoic acid (48 mg, 70%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-ethylbenzenesulfonyl chloride via sulfonamide formation followed by ester hydrolysis following general procedures O and N respectively.

LCMS: m/z 692 (M+H)⁺; ¹H NMR (CDCl₃—CD₃OD, 400 MHz): δ 0.89 (t, 3H), 1.14 (t, 3H), 1.27 (m, 2H), 1.50 (m, 2H), 2.54 (q, 2H), 3.45 (m, 2H), 3.77 (m, 1H), 3.98 (m, 1H), 5.67 (m, 1H), 6.91 (d, 2H), 6.93 (d, 2H), 7.19 (d, 2H), 7.26 (d, 2H), 7.31 (dd, 1H), 7.37 (s, 1H), 7.45 (d, 1H), 7.83 (d, 2H), 7.95 (d, 1H), 8.02 (d, 2H) ppm.

### EXAMPLE 181

### 4-(4-{(2S)-2-(4-Acetylamino-benzenesulfonylamino)-2-[1-butyl-4-(2,4-dichloro-phenyl)-1H-imidazol-2-yl]-ethyl}-phenoxy)-benzoic acid

4-(4-{(2S)-2-(4-Acetylamino-benzenesulfonylamino)-2-[1-butyl-4-(2,4-dichloro-phenyl)-1H-imidazol-2-yl]-ethyl}-phenoxy)-benzoic acid (241 mg, 67%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (269 mg, 0.5 mmol) and N-acetyl sulfanyl chloride via amide formation followed by ester hydrolysis following general procedures O and N respectively.

LCMS: m/z 721 (M+H)⁺.

### EXAMPLE 182

### 4-(4-{(2S)-2-Acetylamino-2-[1-butyl-4-(2,4-dichloro-phenyl)-1H-imidazol-2-yl]-ethyl}-phenoxy)-benzoic acid

4-(4-{(2S)-2-Acetylamino-2-[1-butyl-4-(2,4-dichloro-phenyl)-1H-imidazol-2-yl]-ethyl}-phenoxy)-benzoic acid (23 mg, 80%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (29 mg, 0.05 mmol) and acetic acid via amide formation followed by ester hydrolysis following general procedures F and N respectively.

LCMS: m/z 566 (M+H)⁺; ¹H NMR (CDCl₃, 400 MHz): δ 0.89 (t, 3H), 1.23 (m, 2H), 1.46 (m, 2H), 2.03 (s, 3H), 3.28 (m, 2H), 3.67 (m, 1H), 3.82 (m, 1H), 5.41 (m, 1H), 6.91 (d, 2H), 6.93 (d, 2H), 7.09 (d, 2H), 7.31 (dd, 1H), 7.33 (s, 1H), 7.45 (d, 1H), 7.65 (d, 1H), 7.91 (d, 1H), 8.02 (d, 2H) ppm.

### EXAMPLE 183

### 4-(4-{2-[1-Butyl-4-(2,4-dichloro-phenyl)-1H-imidazol-2-yl]-(2S)-2-propionylamino-ethyl}-phenoxy)-benzoic acid

4-(4-{2-[1-Butyl-4-(2,4-dichloro-phenyl)-1H-imidazol-2-yl]-(2S)-2-propionylamino-ethyl}-phenoxy)-benzoic acid (24 mg, 84%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (29 mg, 0.05 mmol) and propanoic acid via amide formation followed by ester hydrolysis following general procedures Q and N respectively.

LCMS: m/z 580 (M+H)⁺; ¹H NMR (CDCl₃, 400 MHz): δ 0.88 (t, 3H), 1.20 (t, 3H), 1.25 (m, 2H), 1.48 (m, 2H), 2.25 (q, 2H), 3.30 (m, 2H), 3.69 (m, 1H), 3.83 (m, 1H), 5.42 (m, 1H), 6.92 (d, 2H), 6.95 (d, 2H), 7.11 (d, 2H), 7.31 (dd, 1H), 7.34 (s, 1H), 7.45 (d, 1H), 7.66 (d, 1H), 7.93 (d, 1H), 8.03 (d, 2H) ppm.

### EXAMPLE 184

### 4-{4-[2-[1-Butyl-4-(2,4-dichloro-phenyl)-1H-imidazol-2-yl]-(2S)-2-(2-tetrahydro-furan-2-yl-acetylamino)-ethyl]-phenoxy}-benzoic acid

4-{4-[2-[1-Butyl-4-(2,4-dichloro-phenyl)-1H-imidazol-2-yl]-(2S)-2-(2-tetrahydro-furan-2-yl-acetylamino)-ethyl]-phenoxy}-benzoic acid (22 mg, 68%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (29 mg, 0.05 mmol) and tetrahydrofuran-2-yl-acetic acid via amide formation followed by ester hydrolysis following general procedures F and N respectively.

LCMS: m/z 636 (M+H)⁺; ¹H NMR (CDCl₃, 400 MHz): δ 0.84 (m, 3H), 1.18 (m, 2H), 1.38 (m, 2H), 1.82 (m, 2H), 2.21 (m, 2H), 3.27 (m, 2H), 3.59 (m, 1H), 3.70 (m, 1H), 3.88 (m, 2H), 4.33 (m, 2H), 5.09 (m, 1H), 5.33 (m, 1H), 6.81 (m, 2H), 7.00 (d, 1H), 7.06 (d, 1H), 7.31 (m, 1H), 7.36 (d, 1H), 7.43 (s, 1H), 7.48 (d, 2H), 7.85 (m, 1H), 8.03 (m, 1H), 8.09 (d, 2H) ppm.

### EXAMPLE 185

### 4-{4-[2-[1-Butyl-4-(2,4-dichloro-phenyl)-1H-imidazol-2-yl]-(2S)-2-(2-methyl-pentanoylamino)-ethyl]-phenoxy}-benzoic acid

4-{4-[2-[1-Butyl-4-(2,4-dichloro-phenyl)-1H-imidazol-2-yl]-2-(2-methyl-pentanoylamino)-ethyl]-phenoxy}-benzoic acid (24 mg, 78%) was prepared from 4-(4-{2-amino-2-[1-butyl-4-(2,4-dichloro-phenyl)-1H-imidazol-2-yl]-ethyl phenoxy)-benzoic acid methyl ester hydrochloride (29 mg, 0.05 mmol) and 2-methylpentanoic acid via amide formation followed by ester hydrolysis following general procedures F and N respectively.

LCMS: m/z 622 (M+H)⁺; ¹H NMR (CDCl₃, 400 MHz): δ 0.75 (t, 3H), 0.83 (m, 3H), 0.92 (m, 3H), 1.12 (m, 4H), 1.34 (m, 2H), 1.61 (m, 2H), 2.21 (m, 1H), 3.21 (m, 2H), 3.63 (m, 1H), 3.80 (m, 1H), 5.36 (m, 1H), 6.78 (d, 2H), 6.98 (m, 2H), 7.27 (d, 1H), 7.32 (d, 1H), 7.45 (s, 1H), 7.47 (d, 2H), 7.88 (m, 2H), 8.06 (d, 2H) ppm.

### EXAMPLE 186

### 4-(4-{2-[1-Butyl-4-(2,4-dichloro-phenyl)-1H-imidazol-2-yl]-(2S)-2-(2-(4-methoxy-phenyl)-acetylamino]-ethyl}-phenoxy)-benzoic acid

4-(4-{2-[1-Butyl-4-(2,4-dichloro-phenyl)-1H-imidazol-2-yl]-2-[(2S)-2-(4-methoxy-phenyl)-acetylamino]-ethyl}-phenoxy)-benzoic acid (54 mg, 80%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-methoxyphenylacetic acid via amide formation followed by ester hydrolysis following general procedures F and N respectively.

LCMS: m/z 672 (M+H)⁺; ¹H NMR (CDCl₃, 400 MHz): δ 0.88 (t, 3H), 1.23 (m, 2H), 1.49 (m, 2H), 3.23 (d, 2H), 3.51 (s, 2H), 3.66 (m, 1H), 3.77 (s, 3H), 3.81 (m, 1H), 5.37 (m, 1H), 6.84 (d, 2H), 6.88 (d, 2H), 6.93 (d, 2H), 7.03 (d, 2H), 7.14 (d, 2H), 7.21 (d, 1H), 7.30 (d, 1H), 7.38 (s, 1H), 7.43 (bs, 1H), 7.87 (d, 1H), 8.02 (d, 2H) ppm.

### EXAMPLE 187

### 4-(4-{2-[1-Butyl-4-(2,4-dichloro-phenyl)-1H-imidazol-2-yl]-(2S)-2-[3-(4-methoxy-phenyl)-propionylamino]-ethyl}-phenoxy)-benzoic acid

4-(4-{2-[1-Butyl-4-(2,4-dichloro-phenyl)-1H-imidazol-2-yl]-(2S)-2-[3-(4-methoxy-phenyl)-propionylamino]-ethyl}-phenoxy)-benzoic acid (56 mg, 81%) was prepared from 4-(4-{(2S)-2-amino-2-[1-butyl-4-(2,4-dichlorophenyl)-1H-imidazol-2-yl]-ethyl}-phenoxy)-benzoic acid methyl ester hydrochloride (58 mg, 0.1 mmol) and 2-(4-methoxyphenyl)propanoic acid via amide formation followed by ester hydrolysis following general procedures F and N respectively.

LCMS: m/z 686 (M+H)⁺; ¹H NMR (DMSO-d6, 400 MHz): δ 0.83 (t, 3H), 1.17 (m, 2H), 1.47 (m, 2H), 2.29 (t, 2H), 2.64 (m, 2H), 3.14 (m, 2H), 3.65 (s, 3H), 3.81 (m, 1H), 3.96 (m, 1H), 5.22 (m, 1H), 6.76 (d, 2H), 6.85 (d, 2H), 6.94 (d, 2H), 7.02 (d, 2H), 7.26 (d, 2H), 7.49 (dd, 1H), 7.62 (d, 1H), 7.78 (s, 1H), 7.86 (d, 2H), 8.21 (d, 1H), 8.54 (d, 1H) ppm.

### EXAMPLE 188

### 4-(4-{2-[1-Butyl-4-(2,4-dichloro-phenyl)-1H-imidazol-2-yl]-(2S)-2-[4-(4-methoxy-phenyl)-butyrylamino]-ethyl}-phenoxy)-benzoic acid

4-(4-{2-[1-Butyl-4-(2,4-dichloro-phenyl)-1H-imidazol-2-yl]-(2S)-2-[4-(4-methoxy-phenyl)-butyrylamino]-ethyl}-phenoxy)-benzoic acid (49 mg, 70%) was prepared from 4-(4-{(2S)-2-amino-2-[1-butyl-4-(2,4-dichlorophenyl)-1H-imidazol-2-yl]-ethyl}-phenxoy)-benzoic acid methyl ester hydrochloride (58 mg, 0.1 mmol) and 3-(4-methoxyphenyl)butyric acid via amide formation followed by ester hydrolysis following general procedures F and N respectively.

LCMS: m/z 700 (M+H)⁺; ¹H NMR (DMSO-d6, 400 MHz): δ 0.83 (t, 3H), 1.18 (m, 2H), 1.51 (m, 2H), 1.64 (m, 2H), 2.02 (t, 2H), 2.34 (t, 2H), 3.18 (m, 2H), 3.68 (s, 3H), 3.88 (m, 1H), 4.00 (m, 1H), 5.27 (m, 1H), 6.78 (d, 2H), 6.87 (d, 2H), 6.96 (d, 2H), 7.00 (d, 2H), 7.30 (d, 2H), 7.49 (dd, 1H), 7.62 (d, 1H), 7.80 (s, 1H), 7.86 (d, 2H), 8.21 (d, 1H), 8.51 (d, 1H) ppm.

### EXAMPLE 189

### 4-(4-{2-[1-Butyl-4-(2,4-dichloro-phenyl)-1H-imidazol-2-yl]-(2S)-2-[2-(3-methoxy-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(3-methoxy-phenyl)-acetylamino]-ethyl}-phenoxy)-benzoic acid (245 mg, 73%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (287 mg, 0.5 mmol) and 3-methoxyphenylacetic acid via amide formation followed by ester hydrolysis following general procedures F and N respectively.

LCMS: m/z 672 (M+H)⁺, ¹H NMR (CDCl₃, 400 MHz): δ 0.88 (t, 3H), 1.21 (m, 2H), 1.45 (m, 2H), 3.22 (m, 2H), 3.51 (s, 2H), 3.68 (m, 1H), 3.77 (s, 3H), 3.83 (m, 1H), 5.31 (m, 1H), 6.82 (m, 2H), 6.89 (d, 2H), 6.92 (d, 2H), 7.04 (d, 2H), 7.23 (m, 1H), 7.33 (dd, 1H), 7.38 (d, 2H), 7.46 (d, 1H), 7.67 (d, 1H), 7.85 (d, 1H), 7.99 (d, 2H) ppm.

### EXAMPLE 190

### 4-(4-{2-[4-(2,4-Dichloro-phenyl)-1-ethyl-1H-imidazol-2-yl]-(2S)-2-[2-(3-methoxy-phenyl)-acetylamino]-ethyl}-phenoxy)-benzoic acid

4-(4-{2-[4-(2,4-Dichloro-phenyl)-1-ethyl-1H-imidazol-2-yl]-(2S)-2-[2-(3-methoxy-phenyl)-acetylamino]-ethyl}-phenoxy)-benzoic acid (224 mg, 69%) was prepared from 4-(4-{(2S)-2-amino-2-[4-(2,4-dichloro-phenyl)-1-ethyl-1H-imidazol-2-yl]-ethyl}-phenoxy)-benzoic acid methyl ester hydrochloride (255 mg, 0.5 mmol) and 3-methoxy phenylacetic acid via amide formation followed by ester hydrolysis following general procedures F and N respectively.

LCMS: m/z 644 (M+H)⁺; ¹H NMR (DMSO, 400 MHz): δ 1.12-1.16 (m, 3H), 3.17-3.22 (m, 1H), 3.33-3.36 (m, 2H), 3.66 (s, 3H), 3.88-3.91 (m, 1H), 3.95-3.98 (m, 1H), 5.22 (m, 1H), 6.70 (d, 1H), 6.75 (d, 2H), 6.91-6.95 (m, 2H), 7.11-7.13 (m, 3H), 7.15 (d, 2H), 7.28 (d, 2H), 7.50 (s, 1H), 7.63 (s, 1H), 7.90 (d, 2H), 8.19 (d, 1H), 8.82 (d, 1H) ppm.

### EXAMPLE 191

### 4-(4-{2-[1-Butyl-4-(2,4-dichloro-phenyl)-1H-imidazol-2-yl]-(2S)-2-[2-(2-methoxy-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-methoxy-phenyl)-acetylamino]-ethyl}-phenoxy)-benzoic acid (42 mg, 63%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and (2-methoxyphenyl)acetic acid via amide formation followed by ester hydrolysis following general procedures F and N respectively.

LCMS: m/z 672 (M+H)⁺, ¹H NMR (CDCl₃, 400 MHz): δ 0.86 (t, 3H), 1.19 (m, 2H), 1.43 (m, 2H), 3.20 (m, 2H), 3.57 (m, 2H), 3.64 (m, 1H), 3.75 (s, 3H), 3.80 (m, 1H), 5.30 (m, 1H), 6.89 (m, 6H), 7.02 (d, 2H), 7.18 (d, 1H), 7.31 (m, 2H), 7.39 (d, 2H), 7.45 (d, 1H), 7.89 (d, 1H), 7.97 (d, 2H) ppm.

### EXAMPLE 192

### 4-(4-{2-[1-Butyl-4-(2,4-dichloro-phenyl)-1H-imidazol-2-yl]-(2S)-2-[2-(4-ethoxy-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(4-ethoxy-phenyl)-acetylamino]-ethyl}-phenoxy)-benzoic acid (55 mg, 80%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-ethoxyphenylacetic acid via amide formation followed by ester hydrolysis following general procedures F and N respectively.

LCMS: m/z 686 (M+H)⁺; ¹H NMR (CDCl₃—CD₃OD, 400 MHz): δ 0.89 (t, 3H), 1.24 (m, 2H), 1.37 (s, 3H), 1.48 (m, 2H), 3.24 (m, 2H), 3.44 (s, 2H), 3.76 (m, 1H), 3.89 (m, 1H), 3.96 (q, 2H), 5.31 (m, 1H), 6.82 (d, 2H), 6.89 (d, 2H), 6.92 (d, 2H), 7.11 (d, 2H), 7.13 (d, 2H), 7.35 (dd, 1H), 7.46 (s, 1H), 7.47 (d, 1H), 7.88 (d, 1H), 7.99 (d, 2H) ppm.

### EXAMPLE 193

### 4-(4-{2-[1-Butyl-4-(2,4-dichloro-phenyl)-1H-imidazol-2-yl]-(2S)-2-[2-(3,4,5-trimethoxy-phenyl)-acetylamino-ethyl}-phenoxy)-benzoic acid

4-(4-{2-[1-Butyl-4-(2,4-dichloro-phenyl)-1H-imidazol-2-yl]-(2S)-2-[2-(3,4,5-trimethoxy-phenyl)-acetylamino]-ethyl}-phenoxy)-benzoic acid (45 mg, 62%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 3,4,5-trimethoxyphenylacetic acid via amide formation followed by ester hydrolysis following general procedures F and N respectively.

LCMS: m/z 732 (M+H)⁺; ¹H NMR (CDCl₃, 400 MHz): δ 0.88 (t, 3H), 1.23 (m, 2H), 1.48 (m, 2H), 3.23 (m, 2H), 3.51 (s, 2H), 3.66 (m; 1H), 3.77 (s, 6H), 3.81 (s, 3H), 3.84 (m, 1H), 5.38 (m, 1H), 6.47 (s, 2H), 6.88 (d, 2H), 6.92 (d, 2H), 7.02 (d, 2H), 7.30 (dd, 1H), 7.37 (s, 1H), 7.43 (d, 1H), 7.47 (bs, 1H), 7.86 (d, 1H), 8.00 (d, 2H) ppm.

### EXAMPLE 194

### 4-[4-(2-[1-Butyl-4-(2,4-dichloro-phenyl)-1H-imidazol-2-yl]-(2S)-2-{1-(4-methoxy-phenyl)-cyclopropanecarbonyl]-amino}-ethyl)-phenoxy]-benzoic acid

4-[4-(2-[1-Butyl-4-(2,4-dichloro-phenyl)-1H-imidazol-2-yl]-(2S)-2-{[1-(4-methoxy-phenyl)-cyclopropanecarbonyl]-amino}-ethyl)-phenoxy]-benzoic acid (48 mg, 69%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 1-(4-methoxy-phenyl)-cyclopropanecarboxylic acid via amide formation followed by ester hydrolysis following general procedures F and N respectively.

LCMS: m/z 698 (M+H)⁺; ¹H NMR (DMSO-d6, 400 MHz): δ 0.85 (m, 2H), 0.89 (t, 3H), 1.19 (m, 2H), 1.21 (m, 2H), 1.55 (m, 2H), 3.15 (m, 2H), 3.72 (s, 3H), 3.85 (m, 2H), 5.25 (m, 1H), 6.87 (d, 2H), 6.89 (d, 2H), 6.95 (d, 1H), 6.97 (d, 2H), 7.13 (d, 2H), 7.18 (d, 2H), 7.40 (dd, 1H), 7.57 (d, 1H), 7.78 (s, 1H), 7.87 (d, 2H), 7.96 (d, 1H) ppm.

### EXAMPLE 195

### 4-(4-{2-[1-Butyl-4-(2,4-dichloro-phenyl)-1H-imidazol-2-yl]-(2S)-2-[2-(3-fluoro-4-methoxy-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(3-fluoro-4-methoxy-phenyl)-acetylamino]-ethyl}-phenoxy)-benzoic acid (28 mg, 41%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 3-fluoro-4-methoxyphenylacetic acid via amide formation followed by ester hydrolysis following general procedures F and N respectively.

LCMS: m/z 690 (M+H)⁺; ¹H NMR (CDCl₃, 400 MHz): δ 0.89 (t, 3H), 1.25 (m, 2H), 1.49 (m, 2H), 3.24 (d, 2H), 3.49 (m, 2H), 3.68 (m, 1H), 3.83 (s, 3H), 3.84 (m, 1H), 5.39 (m, 1H), 6.80 (m, 3H), 6.93 (m, 3H), 7.01 (m, 3H), 7.31 (dd, 1H), 7.36 (s, 1H), 7.44 (d, 1H), 7.55 (d, 1H), 7.86 (d, 1H), 8.01 (d, 2H) ppm.

### EXAMPLE 196

### 4-(4-{2-[1-Butyl-4-(2,4-dichloro-phenyl)-1H-imidazol-2-yl]-(2S)-2-[2-(2,4-difluorophenyl)-acetylamino]-ethyl}-phenoxy)-benzoic acid

4-(4-{2-[1-Butyl-4-(2,4-dichloro-phenyl)-1H-imidazol-2-yl]-(2S)-2-[2(2,4-difluorophenyl)-acetylamino]-ethyl}-phenoxy)-benzoic acid (48 mg, 71%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 2,4-difluorophenylacetic acid via amide formation followed by ester hydrolysis following general procedures F and N respectively.

LCMS: m/z 678 (M+H)⁺; ¹H NMR (CDCl₃, 400 MHz): δ 0.88 (t, 3H), 1.24 (m, 2H), 1.49 (m, 2H), 3.27 (d, 2H), 3.59 (d, 2H), 3.69 (m, 1H), 3.85 (m, 1H), 5.40 (m, 1H), 6.80 (d, 2H), 6.88 (d, 2H), 6.91 (d, 2H), 7.06 (d, 2H), 7.20 (m, 1H), 7.31 (dd, 1H), 7.35 (s, 1H), 7.45 (d, 1H), 7.74 (d, 1H), 7.87 (d, 1H), 8.01 (d, 2H) ppm.

### EXAMPLE 197

### 4-(4-{2-[4-(2,4-Dichloro-phenyl)-1-ethyl-1H-imidazol-2-yl]-(2S)-2-[2-(2,4-difluoro-phenyl)-acetylamino]-ethyl}-phenoxy)-benzoic acid

4-(4-{2-[4-(2,4-Dichloro-phenyl)-1-ethyl-1H-imidazol-2-yl]-(2S)-2-[2-(2,4-difluoro-phenyl)-acetylamino]-ethyl}-phenoxy)-benzoic acid (224 mg, 69%) was prepared from 4-(4-{(2S)-2-amino-2-[4-(2,4-dichloro-phenyl)-1-ethyl-1H-imidazol-2-yl]-ethyl}-phenoxy)-benzoic acid methyl ester hydrochloride (255 mg, 0.5 mmol) and 2,4-difluorophenylacetic acid via amide formation followed by ester hydrolysis following general procedures F and N respectively.

LCMS: m/z 650 (M+H)⁺; ¹H NMR (DMSO, 400 MHz): δ 1.15-1.19 (m, 4H), 3.24-3.26 (m, 1H), 3.34-3.36 (m, 1H), 3.38-3.45 (m, 1H), 3.92 (m, 1H), 3.98 (m, 1H), 5.23 (m, 1H), 6.91-7.00 (m, 3H), 7.13 (m, 1H), 7.22 (m, 1H), 7.30 (d, 2H), 7.51 (d, 2H), 7.53 (s, 1H), 7.64 (s, 1H), 7.84-7.92 (m, 3H), 8.21 (s, 1H), 8.88 (d, 1H) ppm.

### EXAMPLE 198

### 4-(4-{2-[1-Butyl-4-(2,4-dichloro-phenyl)-1H-imidazol-2-yl]-(2S)-2-[2-(3-fluoro-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(3-fluoro-phenyl)-acetylamino]-ethyl}-phenoxy)-benzoic acid (48 mg, 72%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 3-fluorophenylacetic acid via amide formation followed by ester hydrolysis following general procedures F and N respectively.

LCMS: m/z 660 (M+H)⁺; ¹H NMR (DMSO-d6, 400 MHz): δ 0.79 (t, 3H), 1.14 (m, 2H), 1.47 (m, 2H), 3.18 (m, 2H), 3.41 (m, 2H), 3.81 (m, 1H), 3.93 (m, 1H), 5.21 (m, 1H), 6.89-7.03 (m, 8H), 7.28 (d, 2H), 7.51 (dd, 1H), 7.63 (d, 1H), 7.80 (s, 1H), 7.90 (m, 2H), 8.22 (d, 1H), 8.86 (d, 1H) ppm.

### EXAMPLE 199

### 4-(4-{2-[1-Butyl-4-(2,4-dichloro-phenyl)-1H-imidazol-2-yl]-(2S)-2-[2-(3,5-difluoro-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(3,5-difluorophenyl)-acetylamino]-ethyl}-phenoxy)-benzoic acid (47 mg, 70%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 3,5-difluorophenylacetic acid via amide formation followed by ester hydrolysis following general procedures F and N respectively.

LCMS: m/z 678 (M+H)⁺; ¹H NMR (CDCl₃, 400 MHz): δ 0.88 (t, 3H), 1.25 (m, 2H), 1.50 (m, 2H), 3.26 (d, 2H), 3.53 (d, 2H), 3.69 (m, 1H), 3.84 (m, 1H), 5.41 (m, 1H), 6.83 (d, 2H), 6.89 (d, 2H), 6.92 (d, 2H), 7.07 (d, 2H), 7.26 (m, 1H), 7.33 (dd, 1H), 7.37 (s, 1H), 7.46 (d, 1H), 7.74 (d, 1H), 7.88 (d, 1H), 8.02 (d, 2H) ppm.

### EXAMPLE 200

### 4-(4-{2-[1-Butyl-4-(2,4-dichloro-phenyl)-1H-imidazol-2-yl]-(2S)-2-[2-(4-methanesulfonyl-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(4-methanesulfonyl-phenyl)-acetylamino]-ethyl}-phenoxy)-benzoic acid (40 mg, 56%) was prepared from 4-(4-{(2S)-2-amino-2-[1-butyl-4-(2,4-dichloro-phenyl)-1H-imidazol-2-yl]-ethyl}-benzoic acid methyl ester hydrochloride (58 mg, 0.1 mmol) and 4-methanesulfonylphenylacetic acid via amide formation followed by ester hydrolysis following general procedures F and N respectively.

LCMS: m/z 720 (M+H)⁺; ¹H NMR (DMSO-d6, 400 MHz): δ 0.78 (t, 3H), 1.13 (m, 2H), 1.46 (m, 2H), 3.15 (s, 3H), 3.20 (m, 2H), 3.52 (m, 2H), 3.82 (m, 1H), 3.94 (m, 1H), 5.22 (m, 1H), 6.93 (d, 2H), 6.95 (d, 2H), 7.28 (d, 2H), 7.42 (d, 2H), 7.51 (dd, 1H), 7.63 (d, 1H), 7.79 (d, 2H), 7.80 (s, 1H), 7.91 (d, 2H), 8.22 (d, 1H), 8.94 (d, 1H) ppm.

### EXAMPLE 201

### 4-(4-{2-[1-Butyl-4-(2,4-dichloro-phenyl)-1H-imidazol-2-yl]-(2S)-2-[2-(4-fluoro-phenyl)-acetylamino]-ethyl}-phenoxy)-benzoic acid

4-(4-{2-[1-Butyl-4-(2,4-dichloro-phenyl)-1H-imidazol-2-yl]-(2S) -2-[2(4-fluoro-phenyl)-acetylamino]-ethyl}-phenoxy)-benzoic acid (22 mg, 67%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (29 mg, 0.05 mmol) and 4-fluorophenylacetic acid via amide formation followed by ester hydrolysis following general procedures F and N respectively.

LCMS: m/z 660 (M+H)⁺; ¹H NMR (DMSO-d6, 400 MHz): δ 0.79 (t, 3H), 1.13 (m, 2H), 1.46 (m, 2H), 3.20 (m, 2H), 3.38 (s, 2H), 3.81 (m, 1H), 3.94 (m, 1H), 5.20 (m, 1H), 6.92 (d, 2H), 6.95 (d, 2H), 7.04 (d, 2H), 7.17 (d, 2H), 7.28 (d, 2H), 7.51 (dd, 1H), 7.62 (d, 1H), 7.79 (s, 1H), 7.90 (m, 2H), 8.22 (d, 1H), 8.82 (d, 1H) ppm.

### EXAMPLE 202

### 4-(4-{2-[1-Butyl-4-(2,4-dichloro-phenyl)-1H-imidazol-2-yl]-(2S)-2-[2-(2,5-dimethoxy-phenyl)-acetylamino]-ethyl}-phenoxy)-benzoic acid

4-(4-{2-[1-Butyl-4-(2,4-dichloro-phenyl)-1H-imidazol-2-yl]-(2S)-2-[2-(2,5-dimethoxy-phenyl)-acetylamino]-ethyl}-phenoxy)-benzoic acid (46 mg, 66%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 2,5-dimethoxyphenylacetic acid via amide formation followed by ester hydrolysis following general procedures F and N respectively.

LCMS: m/z 702 (M+H)⁺; ¹H NMR (CDCl₃—CD₃OD, 400 MHz): δ 0.86 (t, 3H), 1.20 (m, 2H), 1.43 (m, 2H), 3.21 (m, 2H), 3.52 (m, 2H), 3.63 (m, 1H), 3.71 (s, 3H), 3.74 (s, 3H), 3.79 (m, 1H), 5.30 (m, 1H), 6.80 (m, 2H), 6.86 (d, 2H), 6.91 (d, 2H), 7.02 (d, 2H), 7.25 (d, 1H), 7.32 (d, 1H), 7.39 (s, 1H), 7.44 (d, 1H), 7.93 (d, 1H), 7.99 (d, 2H), ppm.

### EXAMPLE 203

### 4-(4-{2-[1-Butyl-4-(2,4-dichloro-phenyl)-1H-imidazol-2-yl]-(2S)-2-[2-(2,5-dioxo-imidazolidin-4-yl)-acetylamino]-ethyl}-phenoxy)-benzoic acid

4-(4-{2-[1-Butyl-4-(2,4-dichloro-phenyl)-1H-imidazol-2-yl]-(2S)-2-[2-(2,5-dioxo-imidazolidin-4-yl)-acetylamino]-ethyl}-phenoxy)-benzoic acid (46 mg, 69%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and (2,5-dioxo-imidazolidin-4-yl)-acetic acid via amide formation followed by ester hydrolysis following general procedures F and N respectively.

LCMS: m/z 664 (M+H)⁺; ¹H NMR (CDCl₃—CD₃OD, 400 MHz): δ 0.88 (m, 3H), 1.23 (m, 2H), 1.44 (m, 2H), 2.56 (m, 1H), 2.80 (m, 1H), 3.26 (m, 2H), 3.65 (m, 1H), 3.80 (m, 1H), 4.36-4.52 (m, 1H), 5.27 (m, 1H), 6.93 (m, 4H), 7.10 (m, 2H), 7.35 (m, 2H), 7.47 (m, 1H), 7.91 (m, 1H), 8.00 (m, 2H) ppm.

### EXAMPLE 204

### 4-{4-[2-[1-Butyl-4-(2,4-dichloro-phenyl)-1H-imidazol-2-yl]-(2S)-2-(4-ethyl-benzoylamino)-ethyl]-phenoxy}-benzoic acid

4-{4-[2-[1-Butyl-4-(2,4-dichloro-phenyl)-1H-imidazol-2-yl]-(2S)-2-(4-ethyl-benzoylamino)-ethyl]-phenoxy}-benzoic acid (53 mg, 80%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-ethylbenzoic acid via amide formation followed by ester hydrolysis following general procedures F and N respectively.

LCMS: m/z 656 (M+H)⁺; ¹H NMR (CDCl₃—CD₃OD, 400 MHz): δ 0.89 (t, 3H), 1.24 (t, 3H), 1.27 (m, 2H), 1.50 (m, 2H), 2.68 (q, 2H), 3.45 (m, 2H), 3.77 (m, 1H), 3.98 (m, 1H), 5.67 (m, 1H), 6.91 (d, 2H), 6.93 (d, 2H), 7.19 (d, 2H), 7.26 (d, 2H), 7.31 (dd, 1H), 7.37 (s, 1H), 7.45 (d, 1H), 7.83 (d, 2H), 7.95 (d, 1H), 8.02 (d, 2H) ppm.

### EXAMPLE 205

### 4-(4-{(2S)-2-(4-tert-Butyl-benzoylamino)-2-[1-butyl-4-(2,4-dichloro-phenyl)-1H-imidazol-2-yl]-ethyl}-phenoxy)-benzoic acid

4-(4-{(2S)-2-(4-tert-Butyl-benzoylamino)-2-[1-butyl-4-(2,4-dichloro-phenyl)-1H-imidazol-2-yl]-ethyl}-phenoxy)-benzoic acid (50 mg, 73%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-tert-butylbenzoic acid via amide formation followed by ester hydrolysis following general procedures F and N respectively.

LCMS: m/z 684 (M+H)⁺; ¹H NMR (CDCl₃—CD₃OD, 400 MHz): δ 0.88 (t, 3H), 1.23 (m, 2H), 1.33 (s, 9H), 1.44 (m, 2H), 3.35 (m, 2H), 3.69 (m, 1H), 3.86 (m, 1H), 5.57 (m, 1H), 6.90 (d, 4H), 7.11 (d, 2H), 7.35 (dd, 2H), 7.38 (d, 1H), 7.46 (s, 1H), 7.47 (d, 1H), 7.81 (d, 2H), 7.92 (d, 1H), 7.94 (d, 2H) ppm.

### EXAMPLE 206

### 4-{4-[2-[1-Butyl-4-(2,4-dichloro-phenyl)-1H-imidazol-2-yl]-(2S)-2-(4-methoxy-benzoylamino)-ethyl]-phenoxy}-benzoic acid

4-{4-[2-[1-Butyl-4-(2,4-dichloro-phenyl)-1H-imidazol-2-yl]-(2S)-2-(4-methoxy-benzoylamino)-ethyl]-phenoxy}-benzoic acid (49 mg, 75%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-methoxybenzoic acid via amide formation followed by ester hydrolysis following general procedures F and N respectively.

LCMS: m/z 658 (M+H)⁺; ¹H NMR (DMSO-d6, 400 MHz): δ 0.77 (t, 3H), 1.17 (m, 2H), 1.50 (m, 2H), 3.46 (m, 2H), 3.79 (s, 3H), 3.92 (m, 1H), 4.06 (m, 1H), 5.47 (m, 1H), 6.78 (d, 2H), 6.91 (d, 2H), 6.95 (d, 2H), 7.33 (d, 2H), 7.50 (dd, 1H), 7.61 (d, 1H), 7.83 (m, 5H), 8.23 (d, 1H), 8.91 (d, 1H) ppm.

### EXAMPLE 207

### 4-(4-{(2S)-2-Benzoylamino-2-[1-butyl-4-(2,4-dichloro-phenyl)-1H-imidazol-2-yl]-ethyl}-phenoxy)-benzoic acid

4-(4-{(2S)-2-Benzoylamino-2-[1-butyl-4-(2,4-dichloro-phenyl)-1H-imidazol-2-yl]-ethyl}-phenoxy)-benzoic acid (26 mg, 84%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (29 mg, 0.05 mmol) and benzoic acid via amide formation followed by ester hydrolysis following general procedures F and N respectively.

LCMS: m/z 628 (M+H)⁺; ¹H NMR (DMSO-d6, 400 MHz): δ 0.79 (t, 3H), 1.20 (m, 2H), 1.53 (m, 2H), 3.37 (m, 2H), 3.89 (m, 1H), 3.98 (m, 1H), 5.42 (m, 1H), 6.92 (d, H), 6.94 (d, 2H), 7.28 (d, 2H), 7.41 (d, 2H), 7.49 (dd, 1H), 7.63 (d, 1H), 7.80 (m, 5H), 7.80 (s, 1H), 8.20 (d, 1H), 8.88 (d, 1H) ppm.

### EXAMPLE 208

### 4-{4-[2-[1-Butyl-4-(2,4-dichloro-phenyl)-1H-imidazol-2-yl]-(2S)-2-(3,5-difluoro-benzoylamino)-ethyl}-phenoxy-benzoic acid

4-{4-[2-[1-Butyl-4-(2,4-dichloro-phenyl)-1H-imidazol-2-yl]-(2S)-2-(3,5-difluoro-benzoylamino)-ethyl]-phenoxy}-benzoic acid (52 mg, 79%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 3,5-difluorobenzoic acid via amide formation followed by ester hydrolysis following general procedures F and N respectively.

LCMS: m/z 664 (M+H)⁺; ¹H NMR (CDCl₃, 400 MHz): δ 0.89 (t, 3H), 1.24 (m, 2H), 1.45 (m, 2H), 3.45 (m, 2H), 3.69 (m, 1H), 3.85 (m, 1H), 5.53 (m, 1H), 6.89 (d, 2H), 6.94 (m, 4H), 7.09 (d, 2H), 7.34 (m, 2H), 7.37 (s, 1H), 7.47 (m, 2H), 7.90 (m, 1H), 7.97 (d, 2H) ppm.

### EXAMPLE 209

### 4-{4-[2-[1-Butyl-4-(2,4-dichloro-phenyl)-1H-imidazol-2-yl]-(2S)-2-(2,4-difluoro-benzoylamino)-ethyl]-phenoxy}-benzoic acid

4-{4-[2-[1-Butyl-4-(2,4-dichloro-phenyl)-1H-imidazol-2-yl]-(2S)-2-(2,4-difluoro-benzoylamino)-ethyl]-phenoxy}-benzoic acid (41 mg, 62%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 2,4-difluorobenzoic acid via amide formation followed by ester hydrolysis following general procedures F and N respectively.

LCMS: m/z 664 (M+H)⁺; ¹H NMR (CDCl₃, 400 MHz): δ 0.90 (t, 3H), 1.26 (m, 2H), 1.46 (m, 2H), 3.44 (m, 2H), 3.70 (m, 1H), 3.85 (m, 1H), 5.52 (m, 1H), 6.89 (d, 2H), 6.97 (m, 4H), 7.09 (d, 2H), 7.33 (d, 2H), 7.38 (s, 1H), 7.45 (m, 2H), 7.91 (m, 1H), 7.98 (d, 2H) ppm.

### EXAMPLE 210

### 4-{4-[2-[1-Butyl-4-(2,4-dichloro-phenyl)-1H-imidazol-2-yl]-(2S)-2-(4-methanesulfonyl-benzoylamino)-ethyl]-phenoxy}-benzoic acid

4-{4-[2-[1-Butyl-4-(2,4-dichloro-phenyl)-1H-imidazol-2-yl]-(2S)-2-(4-methanesulfonyl-benzoylamino)-ethyl]-phenoxy}-benzoic acid (52 mg, 74%) was prepared from 4-(4-{(2S)-2-amino-2-[1-butyl-4-(2,4-dichloro-phenyl)-1H-imidazol-2-yl]-ethyl]-phenoxy)-benzoic acid methyl ester hydrochloride (58 mg, 0.1 mmol) and 4-methanesulfonylbenzoic acid via amide formation followed by ester hydrolysis following general procedures F and N respectively.

LCMS: m/z 706 (M+H)⁺; ¹H NMR (DMSO-d6, 400 MHz): δ 0.78 (t, 3H), 1.19 (m, 2H), 1.51 (m, 2H), 3.25 (s, 3H), 3.47 (m, 2H), 3.93 (m, 1H), 4.03 (m, 1H), 5.50 (m, 1H), 6.93 (d, 2H), 6.95 (d, 2H), 7.28 (d, 2H), 7.42 (d, 2H), 7.51 (dd, 1H), 7.63 (d, 1H), 7.79 (d, 2H), 7.80 (s, 1H), 7.91 (d, 2H), 8.22 (d, 1H), 8.94 (d, 1H) ppm.

### EXAMPLE 211

### 4-{4-[2-[1-Butyl-4-(2,4-dichloro-phenyl)-1H-imidazol-2-yl]-(2S)-2-(4-trifluoromethyl-benzoylamino)-ethyl]-phenoxy}-benzoic acid

4-{4-[2-[1-Butyl-4-(2,4-dichloro-phenyl)-1H-imidazol-2-yl]-(2S)-2-(4-trifluoromethyl-benzoylamino)-ethyl]-phenoxy}-benzoic acid (49 mg, 70%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-(trifluoromethyl)benzoic acid via amide formation followed by ester hydrolysis following general procedures F and N respectively.

LCMS: m/z 696 (M+H)⁺; ¹H NMR (CDCl₃—CD₃OD, 400 MHz): δ 0.90 (t, 3H), 1.25 (m, 2H), 1.46 (m, 2H), 3.38 (m, 2H), 3.71 (m, 1H), 3.86 (m, 1H), 5.55 (m, 1H), 6.89 (d, 2H), 6.91 (d, 2H), 7.10 (d, 2H), 7.34 (dd, 1H), 7.39 (s, 1H), 7.47 (d, 1H), 7.72 (d, 2H), 7.89 (d, 1H), 7.95 (d, 2H), 8.01 (d, 2H) ppm.

### EXAMPLE 212

### 4-{4-[2-[1-Butyl-4-(2,4-dichloro-phenyl)-1H-imidazol-2-yl]-(2S)-2-(2,4-dimethoxy-benzoylamino)-ethyl]-phenoxy}-benzoic acid

4-{4-[2-[4-(2,4-Dichloro-phenyl)-1-ethyl-1H-imidazol-2-yl]-(2S)-2-(4-ethylbenzoyl-amino)-ethyl]-phenoxy}-benzoic acid (249 mg, 80%) was prepared from 4-(4-{(2S)-2-amino-2-[4-(2,4-dichloro-phenyl)-1-ethyl-1H-imidazol-2-yl]-ethyl}-phenoxy)-benzoic acid methyl ester hydrochloride (255 mg, 0.5 mmol) and 4-ethylbenzoic acid via amide formation followed by ester hydrolysis following general procedures F and N respectively.

LCMS: m/z 628 (M+H)⁺; ¹H NMR (DMSO, 400 MHz): δ 1.14-1.21 (m, 6H), 2.61-2.65 (m, 1H), 3.34-3.39 (m, 1H), 3.40-3.46 (m, 1H), 4.01 (m, 1H), 4.08 (m, 1H), 5.52 (m, 1H), 6.84 (d, 2H), 6.98 (d, 2H), 7.26 (d, 2H), 7.39 (d, 2H), 7.48 (d, 1H), 7.51 (s, 1H), 7.61 (s, 2H), 7.75 (d, 2H), 7.84 (d, 2H), 8.24 (d, 1H), 8.96 (d, 1H) ppm.

### EXAMPLE 213

### 4-(4-{2-[1-Butyl-4-(2,4-dichloro-phenyl)-1H-imidazol-2-yl]-(2S)-2-[(pyrazine-2-carbonyl)-amino]-ethyl}-phenoxy)-benzoic acid

4-(4-{2-[1-Butyl-4-(2,4-dichloro-phenyl)-1H-imidazol-2-yl]-(2S)-2-[(pyrazine-2-carbonyl)-amino]-ethyl}-phenoxy)-benzoic acid (32 mg, 51%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and pyrazine-2-carboxylic acid via amide formation followed by ester hydrolysis following general procedures F and N respectively.

LCMS: m/z 630 (M+H)⁺; ¹H NMR (CDCl₃, 400 MHz): δ 0.86 (t, 3H), 1.21 (m, 2H), 1.46 (m, 2H), 3.46 (m, 2H), 3.66 (m, 1H), 3.81 (m, 1H), 5.52 (m, 1H), 6.90 (d, 2H), 6.92 (d, 2H), 7.17 (d, 2H), 7.34 (dd, 1H), 7.43 (d, 1H), 7.48 (s, 1H), 7.97 (d, 2H), 8.18 (d, 1H), 8.56 (m, 2H), 8.76 (d, 1H), 9.39 (d, 1H) ppm.

### EXAMPLE 214

### 4-(4-{2-[1-Butyl-4-(2,4-dichloro-phenyl)-1H-imidazol-2-yl]-(2S)-2-[3-(4-fluoro-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(4-fluoro-phenyl)-ureido]-ethyl}-phenoxy)-benzoic acid (48 mg, 72%) was prepared from 4-(4-{(2S)-2-amino-2-[1-butyl-4-(2,4-dichloro-phenyl)-1H-imidazol-2-yl]-ethyl)phenoxy)-benzoic acid methyl ester hydrochloride (58 mg, 0.1 mmol) and 4-fluorophenyl isocyanate via urea formation followed by ester hydrolysis following general procedures P and N respectively.

LCMS: m/z 661 (M+H)⁺; ¹H NMR (CDCl₃—CD₃OD, 400 MHz): δ 0.90 (t, 3H), 1.25 (m, 2H), 1.46 (m, 2H), 3.38 (m, 2H), 3.71 (m, 1H), 3.86 (m, 1H), 5.55 (m, 1H), 6.89 (d, 2H), 6.91 (d, 2H), 7.10 (d, 2H), 7.34 (dd, 1H), 7.39 (s, 1H), 7.47 (d, 1H), 7.72 (d, 2H), 7.89 (d, 1H), 7.95 (d, 2H), 8.01 (d, 2H) ppm.

### EXAMPLE 215

### 4-(4-{2-[1-Butyl-4-(2,4-dichloro-phenyl)-1H-imidazol-2-yl]-(2S)-2-[3-(4-chloro-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(4-chloro-phenyl)-ureido]-ethyl}-phenoxy)-benzoic acid (47 mg, 69%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 4-chlorophenyl isocyanate via urea formation followed by ester hydrolysis following general procedures P and N respectively.

LCMS: m/z 677 (M+H)⁺; ¹H NMR (CDCl₃-CD₃OD, 400 MHz): δ 0.94 (t, 3H), 1.25 (m, 2H), 1.46 (m, 2H), 3.38 (m, 2H), 3.71 (m, 1H), 3.86 (m, 1H), 5.55 (m, 1H), 6.89 (d, 2H), 6.91 (d, 2H), 7.10 (d, 2H), 7.34 (dd, 1H), 7.39 (s, 1H), 7.47 (d, 1H), 7.72 (d, 2H), 7.89 (d, 1H), 7.95 (d, 2H), 8.03 (d, 2H) ppm.

### EXAMPLE 216

### 4-(4-{2-[1-Butyl-4-(2,4-dichloro-phenyl)-1H-imidazol-2-yl]-(2S)-2-[3-(2,4-difluoro-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(2,4-difluoro-phenyl)-ureido]-ethyl}-phenoxy)-benzoic acid (52 mg, 77%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (58 mg, 0.1 mmol) and 2,4-difluorophenyl isocyanate via urea formation followed by ester hydrolysis following general procedures P and N respectively.

LCMS: m/z 679 (M+H)⁺; ¹H NMR (CDCl₃—CD₃OD, 400 MHz): δ 0.90 (t, 3H), 1.26 (m, 2H), 1.46 (m, 2H), 3.44 (m, 2H), 3.70 (m, 1H), 3.85 (m, 1H), 5.52 (m, 1H), 6.89 (d, 2H), 6.97 (m, 4H), 7.09 (d, 2H), 7.33 (d, 2H), 7.38 (s, 1H), 7.45 (m, 2H), 7.91 (m, 1H), 7.98 (d, 2H) ppm.

### EXAMPLE 217

### 4-(4-{2-[1-Butyl-4-(2,4-dichloro-phenyl)-1H-imidazol-2-yl]-(2S)-2-[3-(2,4-dichloro-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(2,4-dichloro-phenyl)-ureido]-ethyl}-phenoxy)-benzoic acid (51 mg, 71%) was prepared from 4-(4-{(2S)-2-amino-2-[1-butyl-4-(2,4-dichloro-phenyl)-1H-imidazol-2-yl]-ethyl)-phenoxy)-benzoic acid methyl ester hydrochloride (58 mg, 0.1 mmol) and 2,4-dichlorophenyl isocyanate via urea formation followed by ester hydrolysis following general procedures P and N respectively.

LCMS: m/z 712 (M+H)⁺; ¹H NMR (CDCl₃—CD₃OD, 400 MHz): δ 0.92 (t, 3H), 1.26 (m, 2H), 1.46 (m, 2H), 3.44 (m, 2H), 3.70 (m, 1H), 3.85 (m, 1H), 5.52 (m, 1H), 6.89 (d, 2H), 6.97 (m, 4H), 7.09 (d, 2H), 7.33 (d, 2H), 7.38 (s, 1H), 7.45 (m, 2H), 7.93 (m, 1H), 8.01 (d, 2H) ppm.

### EXAMPLE 218

### 4-{2-[1-Butyl-4-(2,4-dichloro-phenyl)-1H-imidazol-2-yl]-(2S)-2-[(trans-4-ethyl-cyclohexanecarbonyl)-amino]-ethoxymethyl}-benzoic acid

4-{2-[1-Butyl-4-(2,4-dichloro-phenyl)-1H-imidazol-2-yl]-(2S)-2-[(4-ethyl-cyclohexanecarbonyl)-amino]-ethoxymethyl}-benzoic acid (2.0 mg, 4.7%) was prepared from 4-ethyl-cyclohexanecarboxylic acid {(1S)-1-[1-butyl-4-(2,4-dichloro-phenyl)-1H-imidazol-2-yl]-2-hydroxy-ethyl}-amide (34 mg, 0.07 mmol) and methyl 4-(bromomethyl)benzoate following general procedure G, then the resulting ester was hydrolyzed following general procedure I.

LCMS: m/z 600 (M+H)⁺, ¹H NMR (CDCl₃, 400 MHz): 0.87 (t, 3H), 0.98 (t, 3H), 1.13-1.30 (m, 6H), 1.43 (m, 4H), 1.83 (m, 4H), 1.94 (m, 1H), 2.17 (m, 1H), 3.73 (m, 2H), 4.09 (m, 1H), 4.21 (m, 1H), 4.58 (s, 2H), 5.63 (m, 1H), 7.24 (m, 3H), 7.44 (m, 1H), 7.82 (m, 3H), 8.04 (s, 1H) ppm.

### EXAMPLE 219

### Trans-4-Ethyl-cyclohexane-carboxylic acid ((1S)-1-[1-butyl-4-(2,4-dichloro-phenyl)-1H-imidazol-2-yl]-2-{4-[4-(1H-tetrazol-5-yl)-phenoxy]-phenyl}-ethyl)-amide

4-Ethyl-cyclohexanecarboxylic acid ((1S)-1-[1-butyl-4-(2,4-dichloro-phenyl)-1H-imidazol-2-yl]-2-{4-[4-(1H-tetrazol-5-yl)-phenoxy]-phenyl}-ethyl)-amide (35 mg, 18%) was prepared from 4-ethyl-cyclohexanecarboxylic acid {(1S)-1-[1-butyl-4-(2,4-dichloro-phenyl)-1H-imidazol-2-yl]-2-[4-(4-cyano-phenoxy)-phenyl]-ethyl}-amide (178 mg, 0.28 mmol) and excess sodium azide and ammonium chloride following general procedure R.

LCMS: m/z 687 (M+H)⁺, ¹H NMR (CD₃OD, 400 MHz): 0.90 (m, 6H), 1.05-1.33 (m, 6H), 1,38 (m, 2H), 1.55 (m, 2H), 1.72 (m, 2H), 1.80 (m, 2H), 2.13 (m, 1H), 3.23-3.42 (m, 3H), 3.88 (m, 1H), 3.95 (m, 1H), 5.38 (t, 1H), 6.98 (d, 2H), 7.08 (d, 2H), 7.26 (d, 2H), 7.39 (dd, 1H), 7.51 (d, 1H), 7.58 (s, 1H), 7.96 (d, 3H), ppm.

### EXAMPLE 220

### 4-{4-[2-[1-Butyl-4-(2,4-dichloro-phenyl)-1H-imidazol-2-yl]-(2S)-2-(3-methoxy-benzoylamino)-ethyl]-phenoxyγ-benzoic acid

4-{4-[2-[1-Butyl-4-(2,4-dichloro-phenyl)-1H-imidazol-2-yl]-(2S)-2-(3-methoxy-benzoylamino)-ethyl]-phenoxy}-benzoic acid (94 mg, 82%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (98 mg, 0.17 mmol) and 3-methoxybenzoic acid via amide formation followed by ester hydrolysis using general procedures F and I respectively.

LCMS: m/z 658 (M+H)⁺, ¹H NMR (DMSO, 400 MHz): 0.77 (t, 3H), 1.17 (m, 2H), 1.51 (m, 2H), 3.38 (m, 1H), 3.50 (m, 1H), 3.75 (s, 3H), 3.93 (m, 1H), 4.05 (m, 1H), 5.50 (q, 1H), 6.86 (d, 2H), 6.97 (d, 2H), 7.04 (dd, 1H), 7.29-7.42 (m, 5H), 7.48 (dd, 1H), 7.60 (d, 1H), 7.82 (s, 1H), 7.85 (d, 2H), 8.22 (d, 1H), 9.04 (d, 1H) ppm.

### EXAMPLE 221

### 4-{4-[2-[1-Butyl-4-(2,4-dichloro-phenyl)-1H-imidazol-2-yl]-(2S)-2-(3,5-dimethoxy-benzoylamino)-ethyl]-phenoxy}-benzoic acid

4-{4-[2-[1-Butyl-4-(2,4-dichloro-phenyl)-1H-imidazol-2-yl]-(2S)-2-(3,5-dimethoxy-benzoylamino)-ethyl]-phenoxy}-benzoic acid (72 mg, 59%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (98 mg, 0.17 mmol) and 3,5-dimethoxybenzoic acid via amide formation followed by ester hydrolysis using general procedures F and I respectively.

LCMS: m/z 688 (M+H)⁺, ¹H NMR (DMSO, 400 MHz): 0.80 (t, 3H), 1.20 (m, 2H), 1.54 (m, 2H), 3.40 (m, 1H), 3.52 (m, 1H), 3.78 (s, 6H), 3.96 (m, 1H), 4.08 (m, 1H), 5.51 (q, 1H), 6.62 (t, 1H), 6.87 (d, 2H), 7.00 (m, 4H), 7.40 (d, 2H), 7.51 (dd, 1H), 7.62 (d, 1H), 7.86 (m, 3H), 8.25 (d, 1H), 9.04 (d, 1H) ppm.

### EXAMPLE 222

### 4-{4-[2-[1-Butyl-4-(2,4-dichloro-phenyl)-1H-imidazol-2-yl]-(2S)-2-(3-cyano-benzoylamino)-ethyl]-phenoxy}-benzoic acid

4-{4-[2-[1-Butyl-4-(2,4-dichloro-phenyl)-1H-imidazol-2-yl]-(2S)-2-(3-cyano-benzoylamino)-ethyl]-phenoxy}-benzoic acid (22 mg, 38%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (52 mg, 0.09 mmol) and 3-cyanobenzoyl chloride via acylation followed by ester hydrolysis using general procedures S and I respectively.

LCMS: m/z 653 (M+H)⁺, ¹H NMR (DMSO, 400 MHz): 0.79 (t, 3H), 1.19 (m, 2H), 1.53 (m, 2H), 3.40 (m, 1H), 3.55 (m, 1H), 3.94 (m, 1H), 4.07 (m, 1H), 5.51 (q, 1H), 6.90 (d, 2H), 6.99 (d, 2H), 7.39 (d, 2H), 7.51 (dd, 1H), 7.62 (d, 1H), 7.68 (t, 1H), 7.84 (s, 1H), 7.89 (d, 2H), 8.01 (m, 1H), 8.15 (m, 1H), 8.25 (d, 1H), 8.29 (m, 1H), 9.34 (d, 1H) ppm.

### EXAMPLE 223

### 4-(4-{2-[1-Butyl-4-(2,4-dichloro-phenyl)-1H-imidazol-2-yl]-(2S)-2-[3-(4-methoxy-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(4-methoxy-phenyl)-ureido]-ethyl}-phenoxy)-benzoic acid (16.2 mg, 22%) was prepared from 4-(4-{(2S)-2-amino-2-[1-butyl-4-(2,4-dichloro-phenyl)-1H-imidazol-2-yl]-ethyl}-phenoxy)-benzoic acid methyl ester (59 mg, 0.11 mmol) and 4-methoxyphenyl isocyanate via urea formation followed by ester hydrolysis using general procedures P and I respectively.

LCMS: m/z 673 (M+H)⁺, ¹H NMR (DMSO, 400 MHz): δ 0.80 (t, 3H), 1.15 (m, 2H), 1.45 (m, 2H), 3.40 (m, 2H), 3.65 (s, 3H), 3.82 (m, 1H), 3.95 (m, 1H), 5.11 (m, 1H), 6.68 (d, 1H), 6.78 (d, 2H), 6.89 (d, 2H), 6.97 (d, 2H), 7.23 (m, 4H), 7.49 (dd, 1H), 7.61 (d, 1H), 7.75 (s, 1H), 7.85 (d, 2H), 8.20 (d, 1H), 8.27 (s, 1H) ppm.

### EXAMPLE 224

### 4-{4-[2-[1-Butyl-4-(2,4-dichloro-phenyl)-1H-imidazol-2-yl]-(2S)-2-(4-dimethylamino-benzoylamino)-ethyl]-phenoxy}-benzoic acid

4-{4-[2-[1-Butyl-4-(2,4-dichloro-phenyl)-1H-imidazol-2-yl]-(2S)-2-(4-dimethylamino-benzoylamino)-ethyl]-phenoxy}-benzoic acid (33 mg, 53%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (53 mg, 0.092 mmol) and 4-dimethylaminobenzoic acid via amide formation followed by ester hydrolysis using general procedures F and I respectively.

LCMS: m/z 671 (M+H)⁺, ¹H NMR (DMSO, 400 MHz): δ 0.79 (t, 3H), 1.18 (m, 2H), 1.52 (m, 2H), 2.95 (s, 6H), 3.37 (m, 1H), 3.50 (m, 1H), 3.95 (m, 1H), 4.09 (m, 1H), 5.50 (q, 1H), 6.66 (d, 2H), 6.85 (d, 2H), 6.96 (d, 2H), 7.37 (d, 2H), 7.51 (dd, 1H), 7.62 (d, 1H), 7.74 (d, 2H), 7.82 (s, 1H), 7.85 (d, 2H), 8.25 (d, 1H), 8.65 (d, 1H) ppm.

### EXAMPLE 225

### 4-(4-{2-[1-Butyl-4-(2,4-dichloro-phenyl)-1H-imidazol-2-yl]-(2S)-2-[3-(3-methoxy-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(3-methoxy-phenyl)-ureido]-ethyl}-phenoxy)-benzoic acid (8.0 mg, 12%) was prepared from 4-(4-{(2S)-2-amino-2-[1-butyl-4-(2,4-dichloro-phenyl)-1H-imidazol-2-yl]-ethyl}-phenoxy)-benzoic acid methyl ester (54 mg, 0.1 mmol) and 3-methoxyphenyl isocyanate via urea formation followed by ester hydrolysis using general procedures P and I respectively.

LCMS: m/z 673 (M+H)⁺, ¹H NMR (DMSO, 400 MHz): δ 0.80 (t, 3H), 1.19 (m, 2H), 1.49 (m, 2H), 3.20 (m, 1H), 3.32 (m, 1H), 3.70 (s, 3H), 3.86 (m, 1H), 3.96 (m, 1H), 5.15 (q, 1H), 6.49 (dd, 1H), 6.79 (t, 2H), 6.92 (d, 2H), 7.00 (d, 2H), 7.11 (m, 2H), 7.26 (d, 2H), 7.51 (dd, 1H), 7.62 (d, 1H), 7.78 (s, 1H), 7.88 (d, 2H), 8.23 (d, 1H), 8.49 (d, 1H), ppm.

### EXAMPLE 226

### 4-(4-{2-[1-Butyl-4-(2,4-dichloro-phenyl)-1H-imidazol-2-yl]-(2S)-2-[3-(2-methoxy-phenyl)-ureido]-ethyl}-phenoxy)-benzoic acid

4-(4-{2-[1-Butyl-4-(2,4-dichloro-phenyl)-1H-imidazol-2-yl]-(2S)-2-[3-(2-methoxy-phenyl)-ureido]-ethyl}-phenoxy)-benzoic acid (12.4 mg, 18%) was prepared from 4-(4-{(2S)-2-amino-2-[1-butyl-4-(2,4-dichloro-phenyl)-1H-imidazol-2-yl]-ethyl}-phenoxy)-benzoic acid methyl ester (54 mg, 0.1 mmol) and 2-methoxyphenyl isocyanate via urea formation followed by ester hydrolysis using general procedures P and I respectively.

LCMS: m/z 673 (M+H)⁺, ¹H NMR (DMSO, 400 MHz): δ 0.80 (t, 3H), 1.15 (m, 2H), 1.47 (m, 2H), 3.20 (m, 1H), 3.34 (m, 1H), 3.80 (s, 3H), 3.88 (m, 1H), 4.01 (m, 1H), 5.13 (q, 1H), 6.80-6.89 (m, 2H), 6.91-6.96 (m, 3H), 7.00 (d, 2H), 7.28 (d, 2H), 7.51 (dd, 1H), 7.59 (d, 1H), 7.63 (d, 1H), 7.78 (s, 1H), 7.88 (d, 2H), 8.01-8.06 (m, 2H), 8.25 (d, 1H), ppm.

### EXAMPLE 227

### 4-(4-{2-[1-Butyl-4-(2,4-dichloro-phenyl)-1H-imidazol-2-yl]-(2S)-2-[4-(3-fluoro-4-methoxybenzoyl-amino)-butyrylamino]-ethyl}-phenoxy)-benzoic acid

4-(4-{2-[1-Butyl-4-(2,4-dichloro-phenyl)-1H-imidazol-2-yl]-(2S)-2-[4-(3-fluoro-4-methoxybenzoyl-amino)-butyrylamino]-ethyl}-phenoxy)-benzoic acid (48 mg, 35%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (103 mg, 0.18 mmol) and N-Boc-4-aminobutyric acid following general procedure F, followed by deprotection using general procedure E. Reaction with 3-fluoro-4-methoxybenzoic acid then followed general procedure F and ester hydrolysis used general procedure I.

LCMS: m/z 761 (M+H)⁺, ¹H NMR (DMSO, 400 MHz): δ 0.82 (t, 3H), 1.19 (m, 2H), 1.51 (m, 2H), 1.66 (m, 2H), 2.10 (t, 2H), 3.18 (m, 3H), 3.37 (m, 1H), 3.88 (m, 4H), 4.00 (m, 1H), 5.25 (q, 1H), 6.93 (d, 2H), 6.99 (d, 2H), 7.21 (t, 1H), 7.30 (d, 2H), 7.49 (dd, 1H), 7.61-7.68 (m, 3H), 7.79 (s, 1H), 7.90 (s, 2H), 8.22 (d, 1H), 8.38 (t, 1H), 8.57 (d, 1H), ppm.

### EXAMPLE 228

### 4-(4-{2-[1-Butyl-4-(2,4-dichloro-phenyl)-1H-imidazol-2-yl]-(2S)-2-[3-(4-chloro-phenyl)-(2S)-2-isobutyryl-amino-propionyl-amino]-ethyl}-phenoxy)-benzoic acid

4-(4-{2-[1-Butyl-4-(2,4-dichloro-phenyl)-1H-imidazol-2-yl]-(2S)-2-[3-(4-chloro-phenyl)-(2S)-2-isobutyryl-amino-propionyl-amino]-ethyl}-phenoxy)-benzoic acid (12.3 mg, 10%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (92 mg, 0.16 mmol) and N-Boc-4-chloro-L-phenylalanine via amide formation using general procedure F. Removal of the protecting group used general procedure E, and acylation with isobutyryl chloride followed by ester hydrolysis used general procedures U and I respectively.

LCMS: m/z 775 (M+H)⁺, ¹H NMR (DMSO, 400 MHz): δ 0.79-0.87 (m, 9H), 1.15 (m, 2H), 1.46 (m, 2H), 2.34 (m, 1H), 2.68 (m, 1H), 2.85 (m, 1H), 3.20 (m, 1H), 3.39 (m, 1H), 3.80 (m, 1H), 3.94 (m, 1H), 4.54 (m, 1H), 5.17 (q, 1H), 6.92-6.99 (m, 4H), 7.09 (d, 2H), 7.20 (d, 2H), 7.29 (d, 2H), 7.50 (dd, 1H), 7.63 (d, 1H), 7.79-7.83 (m, 2H), 7.90 (d, 2H), 8.22 (d, 1H), 8.72 (d, 1H) ppm.

### EXAMPLE 229

### 4-(4-{2-[1-Butyl-4-(2,4-dichloro-phenyl)-1H-imidazol-2-yl]-(2S)-2-[3-(4-trifluoromethyl-phenyl)-2-(2S)-isobutyrylamino-propionylamino]-ethyl}-phenoxy)-benzoic acid

4-(4-{2-[1-Butyl-4-(2,4-dichloro-phenyl)-1H-imidazol-2-yl]-(2S)-2-[3-(4-trifluoromethyl-phenyl)-2-(2S)-isobutyrylamino-propionylamino]-ethyl}-phenoxy)-benzoic acid (41 mg, 51%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (57 mg, 0.1 mmoll) and N-Boc-4-trifluoromethyl-L-phenylalanine via amide formation using general procedure F. Removal of the protecting group used general procedure E, and acylation with isobutyryl chloride followed by ester hydrolysis used general procedures U and I respectively.

LCMS: m/z 809 (M+H)⁺, ¹H NMR (DMSO, 400 MHz): δ 0.75-0.88 (m, 9H), 1.18 (m, 2H), 1.46 (m, 2H), 2.34 (m, 1H), 2.78 (m, 1H), 2.95 (m, 1H), 3.22 (m, 1H), 3.39 (m, 1H), 3.80 (m, 1H), 3.95 (m, 1H), 4.60 (m, 1H), 5.18 (q, 1H), 6.92-6.99 (m, 4H), 7.09 7.29 (d, 4H), 7.48-7.53 (m, 3H), 7.63 (d, 1H), 7.80 (s, 1H), 7.85 (d, 1H), 7.90 (d, 2H), 8.23 (d, 1H), 8.78 (d, 1H) ppm.

### EXAMPLE 230

### 4-(4-{2-[1-Butyl-4-(2,4-dichloro-phenyl)-1H-imidazol-2-yl]-(2S)-2-[3-(4-tert-butyl-phenyl)-(2S)-2-isobutyrylamino-propionylamino]-ethyl}-phenoxy)-benzoic acid

4-(4-{2-[1-Butyl-4-(2,4-dichloro-phenyl)-1H-imidazol-2-yl]-(2S)-2-[3-(4-tert-butyl-phenyl)-(2S)-2-isobutyrylamino-propionylamino]-ethyl}-phenoxy)-benzoic acid (7 mg, 8.8%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (57 mg, 0.1 mmol) and N-Boc-4-tert-butyl-L-phenylalanine via amide formation using general procedure F. Removal of the protecting group used general procedure E, and acylation with isobutyryl chloride followed by ester hydrolysis used general procedures U and I respectively.

LCMS: m/z 797 (M+H)⁺, ¹H NMR (DMSO, 400 MHz): δ 0.75-0.85 (m, 9H), 1.10-1.23 (m, 11H), 1.45 (m, 2H), 2.33 (m, 1H), 2.64 (m, 1H), 2.82 (m, 1H), 3.20 (m, 1H), 3.38 (m, 1H), 3.77 (m, 1H), 3.95 (m, 1H), 4.49 (m, 1H), 5.18 (q, 1H), 6.89-6.97 (m, 6H), 7.11 (d, 2H), 7.27 (d, 2H), 7.47 (dd, 1H), 7.59 (d, 1H), 7.72 (d, 1H), 7.78 (s, 1H), 7.87 (d, 2H), 8.22 (d, 1H), 8.68 (d, 1H) ppm.

### EXAMPLE 231

### 4-(4-{2-[1-Butyl-4-(2,4-dichloro-phenyl)-1H-imidazol-2-yl]-(2S)-2-[4-(4-chloro-phenyl)-(3S)-3-isobutyrylamino-butyrylamino]-ethyl}-phenoxy)-benzoic acid

4-(4-{2-[1-Butyl-4-(2,4-dichloro-phenyl)-1H-imidazol-2-yl]-(2S)-2-[4-(4-chloro-phenyl)-(3S)-3-isobutyrylamino-butyrylamino]-ethyl}-phenoxy)-benzoic acid (28 mg, 35%) was prepared from 4-(4-{(2S)-2-amino-2-[1-butyl-4-(2,4-dichloro-phenyl)-1H-imidazol-2-yl]-ethyl}-phenoxy)-benzoic acid methyl ester hydrochloride (57 mg, 0.1 mmol) and (3S)-3-tert-butoxycarbonylamino-4-(4-chloro-phenyl)-butyric acid via amide formation using general procedure F. Removal of the protecting group used general procedure E, and acylation with isobutyryl chloride followed by ester hydrolysis used general procedures U and I respectively.

LCMS: m/z 789 (M+H)⁺, ¹H NMR (DMSO, 400 MHz): δ 0.75-0.89 (m, 9H), 1.16 (m, 2H), 1.50 (m, 2H), 2.13 (m, 1H), 2.19 (d, 2H), 2.44 (m, 2H), 3.20 (m, 1H), 3.35 (m, 1H), 3.86 (m, 1H), 3.99 (m, 1H), 4.13 (m, 1H), 5.29 (q, 1H), 6.83 (d, 2H), 6.94 (d, 2H), 7.04 (d, 2H), 7.24 (d, 2H), 7.30 (d, 2H), 7.46-7.51 (m, 2H), 7.60 (d, 1H), 7.78 (s, 1H), 7.84 (d, 2H), 8.21 (d, 1H), 8.58 (d, 1H) ppm.

### EXAMPLE 232

### N-[4-(4-{2-[1-Butyl-4-(2,4-dichloro-phenyl)-1H-imidazol-2-yl](2S)-2-[3-(4-methoxy-phenyl)-ureido]-ethyl}-phenoxy)-3-methanesulfonylamino-phenyl]-methanesulfonamide

N-[4-(4-{2-[1-Butyl-4-(2,4-dichloro-phenyl)-1H-imidazol-2-yl](2S)-2-[3-(4-methoxy-phenyl)-ureido]-ethyl}-phenoxy)-3-methanesulfonylamino-phenyl]-methanesulfonamide (4.0 mg, 1.1%) was prepared from 4-{(2S)-2-Amino-2-[1-butyl-4-(2,4-dichloro-phenyl)-1H-imidazol-2-yl]-ethyl}-phenol (182 mg, 0.45 mmol) and 4-methoxyphenyl isocyanate via urea formation using general procedure P, followed by reaction with 4-fluoro-3-nitroaniline using general procedure G. Reduction of the nitro group followed general procedure T, and bis sulfonamide was prepared using general procedure O.

LCMS: m/z 815 (M+H)⁺, ¹H NMR (DMSO, 400 MHz): δ 0.82 (m, 3H), 1.18 (m, 2H), 1.50 (m, 2H), 3.18 (m, 1H), 3.27 (m, 1H), 3.36 (s, 3H), 3.47 (s, 3H), 3.68 (s, 3H), 3.86 (m, 1H), 3.97 (m, 1H), 5.13 (q, 1H), 6.66 (m, 2H), 6.80 (d, 2H), 6.87 (m, 2H), 7.07 (d, 1H), 7.20 (d, 1H), 7.25 (d, 2H), 7.32 (d, 1H), 7.43-7.54 (m, 2H), 7.63 (d, 1H), 7.76 (s, 1H), 8.22 (d, 1H), 8.28 (d, 1H) ppm.

### EXAMPLE 233

### 4-tert-Butyl-cyclohexanecarboxylic acid ((1S)-1-[1-butyl-4-(2,4-dichloro-phenyl)-1H-imidazol-2-yl]-2-{4-[4-(1H-tetrazol-5-yl)-benzyloxy]-phenyl}-ethyl)-amide

4-Tert-butyl-cyclohexanecarboxylic acid [(1S)-1-[1-butyl-4-(2,4-dichloro-phenyl)-1H-imidazol-2-yl]-2-(4-hydroxy-phenyl)-ethyl]-amide (570 mg, 1 mmol) was first reacted with 4-cyanobenzyl bromide following the general procedure G then the resulted nitrile was transformed into 4-tert-butyl-cyclohexanecarboxylic acid ((1S)-1-[1-butyl-4-(2,4-dichloro-phenyl)-1H-imidazol-2-yl]-2-{4-[4-(1H-tetrazol-5-yl)-benzyloxy]-phenyl}-ethyl)-amide (510 mg, 71%) following general procedure R.

LCMS: m/z 728 (M+H)⁺.

Biological Assay

The following assay methods are utilized to identify compounds of formula 1 that are effective in inhibiting the activity of certain phosphatases, an example of which, as used herein, is PTP1B.

PTP1B Assay

The assay for PTP1B inhibition is based on the detection of the complex between Malachite Green dye and free phosphate, liberated from the phosphopeptide substrate by PTPase action. To each well of a flat—bottom assay plate is added 45 μL assay buffer [−50 mM Imidazole, pH 7.2, 100 mM NaCl, 5 mM DTT, and 1 mM EDTA] and 10 μL of peptide substrate [Tyrosine Phosphopeptide −1, END(ₚY)INASL, 80 μM FAC, Promega Cat # V256A] to a total volume of 55 μL. Test compound (10 μL in up to 50% DMSO) is then added. The mixture is incubated for 5 min, at 25° C., and 10 μL of PTP-1B [Protein Tyrosine Phosphatase 1B (PTP-1B); FAC 0.8 nM; Upstate Biotechnology, Cat # 14-109 lot # 19045 ] is then added. The mixture is incubated for 30 min at 25° C. Subsequently, 25 μL of Malachite Green reagent [10% (w/v) Ammonium Molybdate in water, Sigma Cat # A-7302, 0.2% (w/v) Malachite Green in 4 N HCl, Aldrich Cat # 21,302-0] is then added. After incubation for 15 min at 27° C., the reaction endpoint is measured at 640 nM.

The Malachite Green reagent is prepared by mixing one volume of 10% Ammonium Molybdate with 3 volumes of 0.2% Malachite Green solution, stirring at room temperature for 30 min and then filtering and collecting the filtrate. The Malachite Green reagent is treated with 10 μL of 5% Tween 20 per 990 μL of dye solution before use.

Test compounds are typically examined at six concentrations in the above assay. For this assay, the IC50 (microM) of the enzyme inhibition assay represents the concentration of compound at which 50% signal has been inhibited.

As illustrated by the Examples, embodiments of the present invention demonstrate utility in inhibiting protein tyrosine phosphatase PTP 1B. The compounds of the present invention set forth in the present examples are found to inhibit protein tyrosine phosphatase PTP1B with inhibitory potencies (IC50's) of about 0.01 microM to about 20 microM. In general, embodiments of the present invention useful for pharmaceutical applications will have inhibitory potencies (IC50's) for a protein of interest of below about 100, or about 50 microM. For particular applications, lower inhibitory potencies are useful, thus compounds that inhibit protein tyrosine phosphatase PTP1B with inhibitory potencies (IC50's) in a range of about 0.01 microM to about 10 microM may be preferred. More preferable may be compounds that inhibit protein tyrosine phosphatase PTP1B with inhibitory potencies (IC50's) of about 0.01 microM to about 3 microM.

Embodiments of the compounds of the present invention demonstrate utility as inhibitors of protein tyrosine phosphatases (PTPases). Embodiments of the invention described herein are additionally directed to pharmaceutical compositions and methods of inhibiting PTPase activity in a mammal, which methods comprise administering, to a mammal in need of inhibition of PTPase activity, a therapeutically defined amount of a compound of formula (I), defined above, as a single or polymorphic crystalline form or forms, an amorphous form, a single enantiomer, a racemic mixture, a single stereoisomer, a mixture of stereoisomers, a single diastereoisomer, a mixture of diastereoisomers, a solvate, a pharmaceutically acceptable salt, a solvate, a prodrug, a biohydrolyzable ester, or a biohydrolyzable amide thereof.

Thus, the present invention provides a method of inhibiting a PTPase, comprising the step of administering to a mammal in need thereof a pharmacologically effective amount of a compound of the present invention. The invention further provides a pharmaceutical composition comprising a pharmaceutically acceptable carrier and a pharmacologically effective amount of a compound of the present invention sufficient to inhibit a PTPase. A PTPase—inhibiting amount can be an amount that reduces or inhibits a PTPase activity in the subject.

Additionally provided is a pharmaceutical composition comprising a pharmaceutically acceptable carrier and a pharmacologically effective amount of a compound of the present invention sufficient to treat type I diabetes.

Further, the present invention provides a pharmaceutical composition comprising a pharmaceutically acceptable carrier and a pharmacologically effective amount of a compound of the present invention sufficient to treat type II diabetes.

Further, the present invention provides a pharmaceutical composition comprising a pharmaceutically acceptable carrier and a pharmacologically effective amount of a compound of the present invention sufficient to treat immune dysfunction.

Further, the present invention provides a pharmaceutical composition comprising a pharmaceutically acceptable carrier and a pharmacologically effective amount of a compound of the present invention sufficient to treat AIDS.

Further, the present invention provides a pharmaceutical composition comprising a pharmaceutically acceptable carrier and a pharmacologically effective amount of a compound of the present invention sufficient to treat autoimmune diseases

Further, the present invention provides a pharmaceutical composition comprising a pharmaceutically acceptable carrier and a pharmacologically effective amount of a compound of the present invention sufficient to treat glucose intolerance.

Further, the present invention provides a pharmaceutical composition comprising a pharmaceutically acceptable carrier and a pharmacologically effective amount of a compound of the present invention sufficient to treat obesity.

Further, the present invention provides a pharmaceutical composition comprising a pharmaceutically acceptable carrier and a pharmacologically effective amount of a compound of the present invention sufficient to treat cancer.

Further, the present invention provides a pharmaceutical composition comprising a pharmaceutically acceptable carrier and a pharmacologically effective amount of a compound of the present invention sufficient to treat psoriasis.

Further, the present invention provides a pharmaceutical composition comprising a pharmaceutically acceptable carrier and a pharmacologically effective amount of a compound of the present invention sufficient to treat allergic diseases Further, the present invention provides a pharmaceutical composition comprising a pharmaceutically acceptable carrier and a pharmacologically effective amount of a compound of the present invention sufficient to treat infectious diseases.

Further, the present invention provides a pharmaceutical composition comprising a pharmaceutically acceptable carrier and a pharmacologically effective amount of a compound of the present invention sufficient to treat inflammatory diseases.

Further, the present invention provides a pharmaceutical composition comprising a pharmaceutically acceptable carrier and a pharmacologically effective amount of a compound of the present invention sufficient to treat diseases involving the modulated synthesis of growth hormone.

Further, the present invention provides a pharmaceutical composition comprising a pharmaceutically acceptable carrier and a pharmacologically effective amount of a compound of the present invention sufficient to treat diseases involving the modulated synthesis of growth factors or cytokines which affect the production of growth hormone.

Further, the present invention provides a pharmaceutical composition comprising a pharmaceutically acceptable carrier and a pharmacologically effective amount of a compound of the present invention sufficient to treat Alzheimer's disease.

The compounds of the present invention can be administered to subjects in need of inhibition of PTPase activity. Such subjects can include, for example, horses, cows, sheep, pigs, mice, dogs, cats, primates such as chimpanzees, gorillas, and rhesus monkeys. In another embodiment, such subjects include humans.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alchol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Also provided by the present invention are prodrugs of the invention. Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19.

Other salts which are not pharmaceutically acceptable may be useful in the preparation of compounds of the invention and these form a further aspect of the invention.

In addition, some of the compounds of the present invention may form solvates with water or common organic solvents. Such solvates are also encompassed within the scope of the invention.

Thus, in a further embodiment, there is provided a pharmaceutical composition comprising a compound of the present invention, or a pharmaceutically acceptable salt, solvate, or prodrug therof, and one or more pharmaceutically acceptable carriers, excipients, or diluents.

The compounds of the present invention selectively act as inhibitors of one PTPase in preference to one or more other PTPases, and therefore may posess advantage in the treatment of one or more PTPase—mediated disease in preference to others.

Thus, in a further aspect, the present invention provides a method for the inhibition of PTPases. In another embodiment of this aspect, the present invention provides a method for treating a disease states including diabetes, cancer, inflammation, Alzheimer's disease, psoriasis, or graft versus host disease, which comprises administering to a subject in need thereof a compound of the present invention, a pharmacologically effective amount, or a therapeutically effective amount. In another embodiment, at least one compound of Formula (I) is utilized, either alone or in combination with one or more known therapeutic agents. In a further embodiment, the present invention provides method of prevention and/or treatment of PTPase—mediated human diseases, treatment comprising alleviation of one or more symptoms resulting from that disorder, to an outright cure for that particular disorder or prevention of the onset of the disorder, the method comprising administration to a human in need thereof a therapeutically effective amount of a compound of the present invention of Formula (I).

In this method, factors which will influence what constitutes an effective amount will depend upon the size and weight of the subject, the biodegradability of the therapeutic agent, the activity of the therapeutic agent, as well as its bioavailability. As used herein, the phrase “a subject in need thereof” includes mammalian subjects, such as humans, who either suffer from one or more of the aforesaid diseases or disease states or are at risk for such. Accordingly, in the context of the therapeutic method of the invention, this method also is comprised of a method for treating a mammalian subject prophylactically, or prior to the onset of diagnosis such disease(s) or disease state(s).

The following is a non-exhaustive listing of adjuvants and additional therapeutic agents which may be utilized in combination with the PTPase inhibitors of the present invention:

Pharmacologic classifications of anticancer agents:

1. Alkylating agents: Cyclophosphamide, nitrosoureas, carboplatin, cisplatin, procarbazine

2. Antibiotics: Bleomycin, Daunorubicin, Doxorubicin

3. Antimetabolites: Methotrexate, Cytarabine, Fluorouracil

4. Plant alkaloids: Vinblastine, Vincristine, Etoposide, Paclitaxel,

5. Hormones: Tamoxifen, Octreotide acetate, Finasteride, Flutamide

6. Biologic response modifiers: Interferons, Interleukins

Pharmacologic classifications of treatment for Rheumatoid Arthritis (Inflammation)

1. Analgesics: Aspirin

2. NSAIDs (Nonsteroidal anti-inflammatory drugs): Ibuprofen, Naproxen, Diclofenac

3. DMARDs (Disease-Modifying Antirheumatic drugs): Methotrexate, gold preparations, hydroxychloroquine, sulfasalazine

4. Biologic Response Modifiers, DMARDs: Etanercept, Infliximab

5. Glucocorticoids

Pharmacologic classifications of treatment for Diabetes Mellitus

1. Sulfonylureas: Tolbutamide, Tolazamide, Glyburide, Glipizide

2. Biguanides: Metformin

3. Miscellaneous oral agents: Acarbose, PPAR agonists such as Troglitazone, DPP-IV inhibitors, Glucokinase activators

4. Insulin, insulin mimetics, insulin secretagogues, insulin sensitizer

5. GLP-1, GLP-1 mimetics

Pharmacologic classifications of treatment for Alzheimer's Disease

1. Cholinesterase Inhibitor: Tacrine, Donepezil

2. Antipsychotics: Haloperidol, Thioridazine

3. Antidepressants: Desipramine, Fluoxetine, Trazodone, Paroxetine

4. Anticonvulsants: Carbamazepine, Valproic acid

Pharmacologic classifications of treatment for Hyperlipidemia

1. HMG CoA reductase inhibitors: Mevinolin

2. cholestyramine

3. fibrates

In a further embodiment, the present invention provides a method of treating PTPase mediated diseases, the method comprising administering to a subject in need thereof, a therapeutically effective amount of a compound of Formula (I) in combination with therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates. In a further embodiment, the present invention provides the pharmaceutical composition of the invention as described above, further comprising one or more therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates.

The compound of the present invention of Formula (I), can be administered at a dosage level of from about 0.003 to 500 mg/kg of the body weight of the subject being treated, with another dosage range between 0.003 and 200 mg/kg, or 0.1 to 100 mg/kg of body weight per day. The amount of active ingredient that may be combined with the carrier materials to produce a single dosage will vary depending upon the host treated and the particular mode of administration. For example, a formulation intended for oral administration to humans may contain 1 mg to 2 grams of a compound of Formula (I) with an appropriate and convenient amount of carrier material which may vary from about 5 to 95 percent of the total composition. Dosage unit forms will generally contain between from about 5 mg to about 500mg of active ingredient. Also a dosage form intended for topical administration to the skin may be prepared at 0.1 % to 99% compound to topical excipient ratio and a dosage form intended for inhaled administration of 0.01 to 200 mg of compound in a suitable carrier to deliver an inhaled dosage of compound. Dosage unit forms of systemically delivered compound will generally contain between from about 5 mg to about 500 mg of active ingredient. This dosage has to be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness of the mammal being treated for PTPase—mediated disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

What is claimed is:

1. A compound of Formula (I): 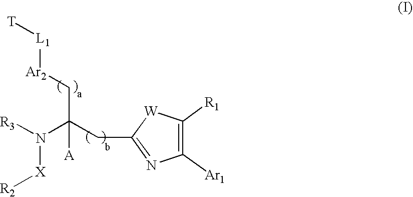
wherein
a and b are equal to 0 and 1; wherein the values of 0 and 1 comprise a direct bond and —CH₂—, respectively, and wherein the —CH₂— group is optionally substituted 1 to 2 times with a substituent group, wherein said substituent group(s) or the term substituted refers to groups comprising: -alkyl, -aryl, -alkylene-aryl, -arylene-alkyl, -alkylene-arylene-alkyl, —O-alkyl, —O-aryl, or -hydroxyl;
W is —O—, —S—, or —N(R₄)—;
wherein
R₄ is
a) -hydrogen;
b) -alkyl;
c) -L₂-D-G;
d) -L₂-D-alkyl;
e) -L₂-D-aryl;
f) -L₂-D-heteroaryl;
g) -L₂-D-cycloalkyl;
h) -L₂-D-heterocyclyl;
i) -L₂-D-arylene-alkyl;
j) -L₂-D-alkylene-cycloalkyl;
k) -L₂-D-alkylene-heterocyclyl;
l) -L₂-D-alkylene-aryl;
m) -L₂-D-alkylene-heteroaryl;
n) -L₂-D-alkylene-arylene-alkyl;
o) -L₂-D-alkylene-heteroarylene-alkyl;
p) -L₂-D-alkyl-G;
q) -L₂-D-aryl-G;
r) -L₂-D-heteroaryl-G;
s) -L₂-D-cycloalkyl-G;
t) -L₂-D-heterocyclyl-G;
u) -L₂-D-arylene-alkyl-G;
v) -L₂-D-alkylene-cycloalkyl-G;
w) -L₂-D-alkylene-heterocyclyl-G;
x) -L₂-D-alkylene-aryl-G;
y) -L₂-D-alkylene-heteroaryl-G;
z) -L₂-D-alkylene-arylene-alkyl-G; or
aa) -L₂-D-alkylene-heteroarylene-alkyl-G;
wherein
L₂ is a direct bond, -alkylene, -alkenylene, or -alkynylene;
D is a direct bond, —CH₂—, —O—, —N(R₅)—, —(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, —N═N—, or —N(R₅)—N(R₆)—,
wherein R₅ and R₆ are independently selected from the group consisting of: -hydrogen, -alkyl, -aryl, -arylene-alkyl, -alkylene-aryl, and -alkylene-arylene-alkyl;
G is —H, -alkyl, —CN, —SO₃H, —P(O)(OH)₂, —P(O)(O-alkyl)(OH), —CO₂H, —CO₂-alkyl, an acid isostere, —NR₇R₈, 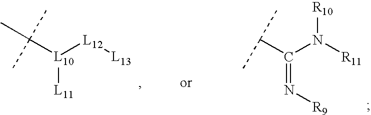
wherein
L₁₀ is alkyline, cycloalkyline, heteroaryline, aryline, or heterocyclyline;
L₁₂ is —O—, —C(O)—N(R₄₀)—, —C(O)—O—, —C(O)—, or —N(R₄₀)—CO—N(R₄₁)—;
L₁₃ is hydrogen, alkyl, alkenyl, alkynyl, heterocyclyl, heteroaryl, or -alkylene-aryl;
L₁₁ is hydrogen, alkyl, alkenyl, alkynyl, -alkylene-aryl, -alkylene -heteroaryl, alkylene-O-alkylene-aryl, -alkylene-S-alkylene-aryl, -alkylene-O-alkyl, -alkylene-S-alkyl, -alkylene—NH₂, -alkylene-OH, -alkylene-SH, -alkylene-C(O)—OR₄₂, -alkylene-C(O)—NR₄₂R₄₃, -alkylene—NR₄₂R₄₃, -alkylene—N(R₄₂)—C(O)—R₄₃, -alkylene—N(R₄₂)—S(O₂)—R₄₃, or the side chain of a natural or non-natural amino acid;
R₄₂ and R₄₃ are independently selected from the group consisting of hydrogen, aryl, alkyl, and alkylene-aryl;
wherein
R₄₂ and R₄₃ may be taken together to form a ring having the formula —(CH₂)_{q}—Y—(CH₂)ᵣ— bonded to the nitrogen atom to which R₁₁ and R₁₂ are attached, wherein q and r are, independently, 1, 2, 3, or 4; Y is —CH₂—, —C(O)—, —O—, —N(H)—, —S—, —S(O)—, —SO₂—, —CON(H)—, —NHC(O)—, —NHCON(H)—, —NHSO₂—, —SO₂N(H)—, —(O)CO—, —NHSO₂NH—, —OC(O)—, —N(R₄₄)—, —N(C(O)R₄₄)—, —N(C(O)NHR₄₄)—, —N(SO₂NHR₄₄)—, —N(SO₂R₄₄)—, or —N(C(O)OR₄₄)—; or
R₄₂ and R₄₃ may be taken together, with the nitrogen atom to which they are attached, to form a heterocyclyl or heteroaryl ring.
R₄₀, R₄₁, and R₄₄ are independently selected from the group consisting of: hydrogen, aryl, alkyl, or alkylene-aryl.
and wherein
R₇ and R₈ are independently selected from the group consisting of hydrogen, -alkyl, -L₃-E-alkyl, -L₃-E-aryl, —C(O)-alkyl, —C(O)-aryl, —SO₂-alkyl, —SO₂-aryl, and 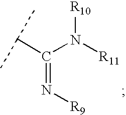
wherein
R₉, R₁₀, and R₁₁ are independently selected from the group consisting of: -hydrogen, -alkyl, -aryl, -arylene-alkyl, -alkylene-aryl, and -alkylene-arylene-alkyl;
L₃ is a direct bond, -alkylene, -alkenylene, or -alkynylene;
E is a direct bond, —CH₂—, —O—, —N(R₁₂)—, —C(O)—, —CON(R₁₂)—, —N(R₁₂)C(O)—, —N(R₁₂)CON(R₁₃)—, —N(R₁₂)C(O)O—, —OC(O)N(R₁₂)—, —N(R₁₂)SO₂—, —SO₂N(R₁₂)—, —C(O)—O—, —O—C(O)—, —S—, —S(O)—, —S(O₂)—, —N(R₁₂)SO₂N(R₁₃)—, —N═N—, or —N(R₁₂)—N(R₁₃)—,
wherein
R₁₂ and R₁₃ are independently selected from the group consisting of: -hydrogen, -alkyl, -aryl, -arylene-alkyl, -alkylene-aryl, and -alkylene-arylene-alkyl;
A is hydrogen, -alkyl, -alkenyl, or -alkynyl;
X is
a) —C(O)—;
b) —CH₂—;
wherein the —CH₂— group is optionally substituted 1 to 2 times with a substituent group, wherein said substituent group(s) are selected from the group consisting of: -alkyl, -aryl, -alkylene-aryl, -arylene-alkyl, -alkylene-arylene-alkyl, —O-alkyl, —O-aryl, and -hydroxyl.
c) a direct bond; or
d) —SO₂—;
R₁ is
a) -hydrogen;
b) -fluoro
c) -chloro
d) -bromo
e) -iodo
f) -cyano
g) -alkyl;
h) -aryl;
i) -alkylene-aryl;
j) -heteroaryl;
k) -alkylkene-heteroaryl;
l) -cycloalkyl;
m) -alkylene-cycloalkyl
n) -heterocyclyl; or
o) -alkylene-heterocyclyl;
R₂ is
a) -perfluoroalkyl;
b) -J-R₁₄;
c) -alkyl;
d) -aryl;
e) -heteroaryl;
f) -heterocyclyl;
g) -cycloalkyl;
h) -L₄-aryl;
i) -L₄-arylene-aryl;
j) -L₄-arylene-alkyl;
k) -arylene-alkyl;
l) -arylene-arylene-alkyl;
m) -J-alkyl;
n) -J-aryl;
o) -J-alkylene-aryl;
p) -J-arylene-alkyl;
q) -J-alkylene-arylene-aryl;
r) -J-arylene-arylene-aryl;
s) -J-alkylene-arylene-alkyl;
t) -L₄-J-alkylene-aryl;
u) -arylene-J-alkyl;
v) -L₄-J-aryl;
w) -L₄-J-heteroaryl;
x) -L₄-J-cycloalkyl;
y) -L₄-J-cycloalkylene-alkyl;
z) -L₄-J-heterocyclyl;
aa) -L₄-J-arylene-alkyl;
bb) -L₄-J-alkylene-arylene-alkyl;
cc) -L₄-J-alkyl;
dd) -L₄-J-R₁₄;
ee) -L₄-J-alkylene-R₁₄;
ff) -J-L₄-R₁₄;
gg) -arylene-J-R₁₄;
hh) -L₄-arylene-J-alkyl;
ii) -L₄-alkylene-J-alkyl;
jj) -L₄-arylene-J-aryl; or
kk) -hydrogen;
wherein
L₄ is a direct bond, -alkylene, -alkenylene, -alkynylene, heterocyclylene, cycloalkylene, arylene, or heteroarylene;
J is a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—,
wherein
R₁₅ and R₁₆ are independently selected from the group consisting of: -hydrogen, -alkyl, -aryl, -arylene-alkyl, -alkylene-aryl, and -alkylene-arylene-alkyl.
R₁₄ is: -hydrogen, -alkyl, -aryl, -arylene-alkyl, -alkylene-aryl, -alkylene-arylene-alkyl, or 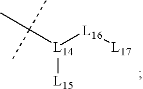
wherein
L₁₄is alkyline, cycloalkyline, heteroaryline, aryline, or heterocyclyline;
L₁₆ is —O—, —C(O)—N(R₄₅)—, —C(O)—O—, —C(O)—, or —N(R₄₅)—CO—N(R₄₆)—;
L₁₇ is hydrogen, alkyl, alkenyl, alkynyl, heterocyclyl, heteroaryl, or -alkylene-aryl;
L₁₅ is hydrogen, alkyl, alkenyl, alkynyl, -alkylene-aryl, -alkylene -heteroaryl, alkylene-O-alkylene-aryl, -alkylene-S-alkylene-aryl, -alkylene-O-alkyl, -alkylene-S-alkyl, -alkylene—NH₂, -alkylene-OH, -alkylene-SH, -alkylene-C(O)—OR₄₇, -alkylene-C(O)—NR₄₇R₄₈, -alkylene—NR₄₇R₄₈, -alkylene—N(R₄₇)—C(O)—R₄₈; -alkylene—N(R₄₇)—S(O₂)—R₄₈, or the side chain of a natural or non-natural amino acid;
R₄₇ and R₄₈ are independently selected from the group consisting of hydrogen, aryl, alkyl, and alkylene-aryl;
R₄₇ and R₄₈ may be taken together, with the nitrogen atom to which they are attached, to form a heterocyclyl or heteroaryl ring.
R₄₅ and R₄₆ are independently selected from the group consisting of hydrogen, aryl, alkyl, and alkylene-aryl;
R₃ is
a) -hydrogen
b) -alkyl
c) -aryl;
d) -alkylene-cycloalkyl;
e) -arylene-alkyl;
f -alkylene-aryl; or
g) -alkylene-heteroaryl;
Ar₁ is an aryl, heteroaryl, fused cycloalkylaryl, fused cycloalkylheteroaryl, fused heterocyclylaryl, or fused heterocyclylheteroaryl group optionally substituted 1 to 7 times;
Ar₂ is an arylene, heteroarylene, fused arylcycloalkylene, fused cycloalkylarylene, fused cycloalkylheteroarylene, fused heterocyclylarylene, or fused heterocyclylheteroarylene group optionally substituted 1 to 7 times;
L₁ is a direct bond, —CH₂—, —O—, alkylene, alkenylene, —O-alkylene-, -alkylene—O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;
wherein
R₂₃ and R₂₄ are independently selected from the group consisting of: -hydrogen, -alkyl, -aryl, -arylene-alkyl, alkylene-aryl, -alkylene-arylene-alkyl, and a direct bond;
T is hydrogen, alkyl, cycloalkyl, heterocyclyl, aryl, heteroaryl, fused cycloalkylaryl, fused cycloalkylheteroaryl, fused heterocyclylaryl, or fused heterocyclylheteroaryl group optionally substituted 1 to 7 times, wherein the substituents are independently selected from the group consisting of:
a) -fluoro;
b) -chloro;
c) -bromo;
d) -iodo;
e) -cyano;
f) -nitro;
g) -perfluoroalkyl;
h) -U-R₂₅;
i) -alkyl;
j) -aryl;
k) -heteroaryl;
l) -heterocyclyl;
m) -cycloalkyl;
n) -L₇-aryl;
o) -L₇-arylene-aryl;
p) -L₇-arylene-alkyl;
q) -arylene-alkyl;
r) -arylene-arylene-alkyl;
s) -U-alkyl;
t) -U-aryl;
u) -U-alkylene-aryl;
v) -U-arylene-alkyl;
w) -U-alkylene-arylene-aryl;
x) -U-arylene-arylene-aryl;
y) -U-alkylene-arylene-alkyl;
z) -L₇-U-alkylene-aryl;
aa) -arylene-U-alkyl;
bb) -L₇-U-aryl;
cc) -L₇-U-heteroaryl;
dd) -L₇-U-cycloalkyl;
ee) -L₇-U-heterocyclyl;
ff) -L₇-U-arylene-alkyl;
gg) -L₇-U-alkylene-arylene-alkyl;
hh) -L₇-U-alkyl;
ii) -L₇-U-alkylene-aryl-R₂₅;
jj) -L₇-U-alkylene-heteroaryl-R₂₅;
kk) -arylene-U-alkylene-R₂₅;
ll) -heteroarylene-U-alkylene-R₂₅;
mm) -L₇-U-aryl-R₂₅;
nn) -L₇-U-heteroarylene-R₂₅;
oo) -L₇-U-heteroaryl-R₂₅;
pp) -L₇-U-cycloalkyl-R₂₅;
qq) -L₇-U-heterocyclyl-R₂₅;
rr) -L₇-U-arylene-alkyl-R₂₅;
ss) -L₇-U-heteroarylene-alkyl-R₂₅;
tt) -L₇-U-alkylene-arylene-alkyl-R₂₅;
uu) -L₇-U-alkylene-heteroarylene-alkyl-R₂₅;
vv) -L₇-U-alkylene-cycloalkylene-alkyl-R₂₅;
ww) -L₇-U-alkylene-heterocyclylene-alkyl-R₂₅;
xx) -L₇-U-alkyl-R₂₅;
yy) -L₇-U-R₂₅;
zz) -arylene-U-R₂₅;
aaa) -heteroarylene-U-R₂₅;
bbb) -heterocyclylene-U-R₂₅;
ccc) -U-alkylene-R₂₅;
ddd) -U-arylene-R₂₅;
eee) -U-heteroarylene-R₂₅;
fff) -U-alkylene-arylene-R₂₅;
ggg) -U-alkylene-heteroarylene-R₂₅;
hhh) -U-heteroarylene-alkylene-R₂₅;
iii) -U-arylene-alkylene-R₂₅;
jjj) -U-cycloalkylene-alkylene-R₂₅;
kkk) -U-heterocyclylene-alkylene-R₂₅;
lll) -U-alkylene-arylene-alkyl-R₂₅;
mmm) -U-alkylene-heteroarylene-alkyl-R₂₅; 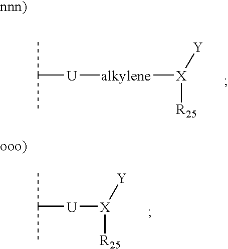
and
ppp) -hydrogen;
wherein
L₇ is a direct bond, -alkylene, -alkenylene, or -alkynylene;
U is a direct bond, —CH₂—, —O—, —N(R₂₆)—, —C(O)—, —CON(R₂₆)—, —N(R₂₆)C(O)—, —N(R₂₆)CON(R₂₇)—, —N(R₂₆)C(O)O—, —OC(O)N(R₂₆)—, —N(R₂₆)SO₂—, —SO₂N(R₂₆)—, —C(O)—O—, —O—C(O)—, —S—, —S(O)—, —S(O₂)—, —N(R₂₆)SO₂N(R₂₇)—, N═N—, or —N(R₂₆)—N(R₂₇)—;
wherein
R₂₆ and R₂₇ are independently selected from the group consisting of: -hydrogen, -alkyl, -aryl, -arylene-alkyl, -alkylene-aryl, and -alkylene-arylene-alkyl; 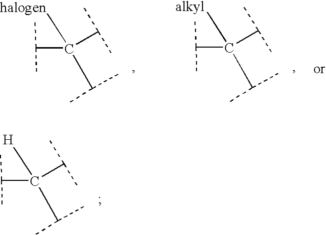
X is or
Y is hydrogen, -CO₂H, -alkylene-aryl, -alkyl, -aryl, -heteroaryl, -heterocyclyl, -cycloalkyl, -alkylene-heteroaryl, or -alkylene-cycloalkyl;
R₂₅ is —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, or -alkylene-arylene-alkyl.

2. The compound according to claim 1, wherein W is —N(R₄)—, wherein R₄ is -alkyl, -L₂-D-alkyl, or -L₂-D-aryl, wherein L₂ is alkylene, and D is a direct bond, —C(O)— or —O—.

3. The compound according to claim 1, wherein W is —N(R₄)—, wherein R₄ is hydrogen.

4. The compound according to claim 1, wherein W is —N(R₄)—, wherein R₄ is -L₂-D-G, wherein L₂ is alkenyl or alkynyl, D is a direct bond, and G is hydrogen or alkyl.

5. The compound according to claim 1, wherein X is —C(O)— or CH₂.

6. The compound according to claim 1, wherein R₁ is hydrogen or aryl.

7. The compound according to claim 1, wherein R₂ is: -alkyl, -aryl, -L₄-J-cycloalkyl, arylene-alkyl, -L₄-arylene-J-alkyl, or -J-alkyl, wherein L₄ is alkylene or alkenylene, and J is a direct bond or —O—.

8. The compound according to claim 1, wherein R₃ is —H; X is —C(O)—; R₂ is -L₄-arylene-J-alkyl, -L₄-J-cycloalkylene-alkyl or -L₄-J-alkylene-aryl, wherein L₄ is alkylene, alkenylene, or a direct bond; and J is a direct bond, —O—, or —NH—.

9. The compound according to claim 1, wherein R₃ is hydrogen.

10. The compound according to claim 1, wherein Ar₁ is a phenyl or naphthyl group optionally having 1 to 5 substituents, wherein the substituents are independently selected from the group consisting of:
a) -fluoro;
b) -chloro;
c) -bromo;
d) -iodo;
e) -cyano;
f) -nitro;
g) -perfluoroalkyl;
h) -K—R₁₇;
i) -alkyl;
j) -aryl;
k) -heteroaryl;
l) -heterocyclyl;
m) -cycloalkyl;
n) -L₅-aryl;
o) -L₅-arylene-aryl;
p) -L₅-arylene-alkyl;
q) -arylene-alkyl;
r) -arylene-arylene-alkyl;
s) -K-alkyl;
t) -K-aryl;
u) -K-alkylene-aryl;
v) -K-arylene-alkyl;
w) -K-alkylene-arylene-aryl;
x) -K-arylene-arylene-aryl;
y) -K-alkylene-arylene-alkyl;
z) -L₅-K-alkylene-aryl;
aa) -arylene-K-alkyl;
bb) -L₅-K-aryl;
cc) -L₅-K-heteroaryl;
dd) -L₅-K-cycloalkyl;
ee) -L₅-K-heterocyclyl;
ff) -L₅-K-arylene-alkyl;
gg) -L₅-K-alkylene-arylene-alkyl;
hh) -L₅-K-alkyl;
ii) -L₅-K-R₁₇;
jj) -arylene-K-R₁₇; or
kk) -hydrogen;
wherein
L₅ is a direct bond, -alkylene, -alkenylene, or -alkynylene;
K iss a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₈)SO₂N(R₁₉)—, —N═N—, or —N(R₁₈)—N(R₁₉)—,
wherein
R₁₇, R₁₈, and R₁₉ are independently selected from the group consisting of: -hydrogen, -alkyl, -aryl, -arylene-alkyl, -alkylene-aryl, or -alkylene-arylene-alkyl.

11. The compound according to claim 1, wherein Ar₁ is a phenyl group substituted 1 to 5 times with substituents independently selected from the group consisting of:
a) -fluoro;
b) -chloro;
c) -bromo;
d) -iodo; or
e) -nitro.

12. The compound according to claim 1, wherein Ar₂ comprises sa phenylene or naphthylene group optionally having 1 to 5 substituents, wherein the substituents are independently selected from the group consisting of:
a) -fluoro;
b) -chloro;
c) -bromo;
d) -iodo;
e) -cyano;
f) -nitro;
g) -perfluoroalkyl;
h) -Q-R₂₀;
i) -alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s) -Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y) -Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L6-Q-arylene-alkyl;
gg) -L₆-Q-alkylene-arylene-alkyl;
hh) -L₆-Q-alkyl;
ii) -L₆-Q-alkylene-aryl-R₂₀;
jj) -L₆-Q-alkylene-heteroaryl-R₂₀;
kk) -arylene-Q-alkylene-R₂₀;
ll) -heteroarylene-Q-alkylene-R₂₀;
mm) -L₆-Q-aryl-R₂₀;
nn) -L₆-Q-heteroarylene-R₂₀;
oo) -L₆-Q-heteroaryl-R₂₀;
pp) -L₆-Q-cycloalkyl-R₂₀;
qq) -L₆-Q-heterocyclyl-R₂₀;
rr) -L₆-Q-arylene-alkyl-R₂₀;
ss) -L₆-Q-heteroarylene-alkyl-R₂₀;
tt) -L₆-Q-alkylene-arylene-alkyl-R₂₀;
uu) -L₆-Q-alkylene-heteroarylene-alkyl-R₂₀;
vv) -L₆-Q-alkylene-cycloalkylene-alkyl-R₂₀;
ww) -L₆-Q-alkylene-heterocyclylene-alkyl-R₂₀;
xx) -L₆-Q-alkyl-R₂₀;
yy) -L₆-Q-R₂₀;
zz) -arylene-Q-R₂₀;
aaa) -heteroarylene-Q-R₂₀;
bbb) -heterocyclylene-Q-R₁₈;
ccc) -Q-alkylene-R₂₀;
ddd) -Q-arylene-R₂₀;
eee) -Q-heteroarylene-R₂₀;
fff) -Q-alkylene-arylene-R₂₀;
ggg) -Q-alkylene-heteroarylene-R₂₀;
hhh) -Q-heteroarylene-alkylene-R₂₀;
iii) -Q-arylene-alkylene-R₂₀;
jjj) -Q-cycloalkylene-alkylene-R₂₀;
kkk) -Q-heterocyclylene-alkylene-R₂₀;
lll) -Q-alkylene-arylene-alkyl-R₂₀; or
mmm) -Q-alkylene-heteroarylene-alkyl-R₂₀; 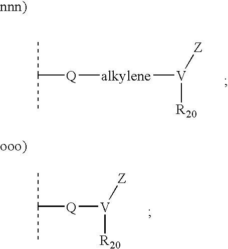
and
ppp) -hydrogen,
wherein
L₆ is a direct bond, -alkylene, -alkenylene , or -alkynylene;
Q is a direct bond, —CH₂—, —O—, —N(R₂₁)—, —C(O)—, —CON(R₂₁)—, —N(R₂₁)C(O)—, —N(R₂₁)CON(R₂₂)—, —N(R₂₁)C(O)O—, —OC(O)N(R₂₁)—, —N(R₂₁)SO₂—, —SO₂N(R₂₁)—, —C(O)—O—, —O—C(O)—, —S—, —S(O)—, —S(O₂)—, —N(R₂₁)SO₂N(R₂₂)—, N═N—, or —N(R₂₁)—N(R₂₂)—;
wherein
R₂, and R₂₂ are independently selected from the group consisting of: -hydrogen, -alkyl, -aryl, -arylene-alkyl, -alkylene-aryl, or -alkylene-arylene-alkyl;
V is 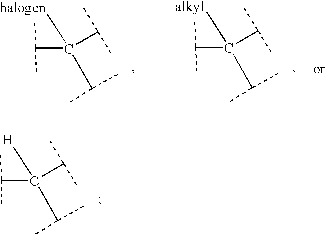
Z is hydrogen, —CO₂H, -alkylene-aryl, -alkyl, -aryl, -heteroaryl, -heterocyclyl, -cycloalkyl, -alkylene-heteroaryl, or -alkylene-cycloalkyl;
R₂₀ is —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, or -alkylene-arylene-alkyl.

13. The compound according to claim 1, wherein Ar₂ comprises a phenyl or naphthyl group optionally substituted 1 to 5 times, wherein the substituents are independently selected from the group consisting of:
a) -fluoro;
b) -chloro;
c) -bromo;
d) -iodo;
e) -Q-R₂₀;
f) -alkyl;
g) -aryl;
h) -arylene-alkyl;
i) -Q-alkyl; and
j) -arylene-Q-alkyl;
wherein
Q is: —CH₂—, —O—, —C(O)—, or —C(O)—O—; and
R₂₀ is: -hydrogen, -alkyl, -aryl, cycloalkyl, -alkenyl, —CO₂H, or an acid isostere.

14. The compound according to claim 1, wherein Ar₂ is a phenyl group substituted 1 to 5 times, wherein the substituents are independently selected from the group consisting of
a) -fluoro;
b) -chloro;
c) -bromo;
d) -iodo;
e) -Q-R₂₀;
f) -alkyl;
g) -phenyl;
h) -phenylene-alkyl;
i) -Q-alkyl; or
j) -phenylene-Q-alkyl;
wherein
Q is: —CH₂—, —O—, —C(O)—, or —C(O)—O—; and
R₂₀ is: -hydrogen, -alkyl, -phenyl, -cycloalkyl, alkenyl, or —CO₂H.

15. The compound according to claim 1, wherein Ar₂ is a phenyl group substituted 1 to 5 times, wherein the substituents are independently selected from the group consisting of:
a) -Q-alkyl;
b) -Q-arylene-R₂₀;
c) -Q-alkylene-arylene-R₂₀; and 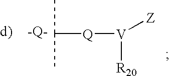
wherein
Q is: —CH₂—, —O—, —C(O)—, or —C(O)—O—;
Z is —CO₂H and an acid isostere;
V is 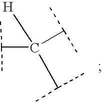
and
R₂₀ is: —CO₂H or an acid isostere.

16. The compound according to claim 1, wherein L₁ is —O-alkylene- or a direct bond.

17. The compound according to claim 1, wherein T is an aryl group substituted by —U-alkylene-R₂₅, wherein U is —O— or a direct bond and R₂₅ is —CO₂H or an acid isostere.

18. The compound according to claim 1, wherein X and R₂ together form a group selected from the group consisting of:
tert-butoxycarbonyl, tert-butyl-methyl-carbonyl, 4-cyclohexyl-butyryl, 3-cyclohexyl-propionyl, 2-cyclohexyl-acetyl,4-tert-butyl-phenyl)-carbonyl, 4-(4′-methoxyphenyl)-butyryl, 4-(4′-methoxyphenyl)-butyryl, 3-(4′-methoxyphenyl)-propionyl, 3-(3′-methoxyphenyl)-propionyl, 3-(4′-methoxy-phenyl)-acryl, 3-(4′-chloro-phenyl)-acryl, 2-(4′-methoxy-phenyl)-acetyl, 2-(4′-chloro-phenyl)-acetyl, 2-(4′-methylsulfonyl-phenyl)-acetyl, 2-(4′-methylsulfonyl-phenyl)-acetyl, 4-(4′-chloro-2′-methyl-phenoxy)-butyryl, 4-(4′-methoxyphenyl)-butyryl, and 4-(4′-cyclohexyl)-propyl.

19. The compound according to claim 1, wherein a equals 0, and the groups T, L₁ and Ar₂ together form a group selected from the group consisting of: 4′-n-butoxy-3′-n-butoxy carbonyl phenyl, or 4′-n-butoxy-3′-carboxyl phenyl.

20. The compound according to claim 1, wherein Ar, is selected from the group consisting of phenyl, naphthyl, 4-nitrophenyl, 4-chlorophenyl, 3-chlorophenyl, 3,4-dichlorophenyl, 2,4-dichlorophenyl, 2,5-dichlorophenyl, 2,6-dichlorophenyl, 4-cynophenyl, 4-bromophenyl, or pentafluorophenyl.

21. The compound according to claim 1, where the compound of Formula (I) comprises:
2-[3-(4′-Methoxyphenyl)-propionylamino]-2-(4′-n-butoxy-3′-carboxyphenyl)-2-ethyl[4-(4′-nitrophenyl)]imidazole;
2-[3-(4′-Methoxy-phenyl)-acrylamino]-2-(4′-n-butoxy-3′-carboxyphenyl)-2-ethyl[4-(4′-nitrophenyl)]imidazole;
2-[4-(4′-Methoxyphenyl)-butyryl amino]-2-(4′-n-butoxy-3′-carboxy phenyl)-2-ethyl [4-(2′,4′-dichlorophenyl)]imidazole;
2-[4-(4′-Cyclohexyl)-propanoylamino]-2-(4′-n-butoxy-3′-carboxyphenyl)-2-ethyl[4-(2′,4′-dichlorophenyl)]imidazole;
N-{(1S)-2-(4-(1,1-Dicarboxymethoxy)phenyl)-1-[4-(2,4-dichlorophenyl)-1H-1-(1-butyl)imidazol-2-yl]ethyl)4-tert-butylcyclohexanecarboxamide;
4-(4-{(2S)-2-[(4-tert-Butyl-cyclohexanecarbonyl)-amino]-2-[1-butyl-4-(2,4-dichloro-phenyl)-1H-imidazol-2-yl]-ethyl)phenoxymethyl)-benzoic acid;
4-(4-{2-[1-Butyl-4-(2,4-dichloro-phenyl)-1H-imidazol-2-yl]-(2S)-2-[2-(4-methoxy-phenyl)-acetylamino]-ethyl}-phenoxymethyl)-benzoic acid;
4-{4-[2-[1-Butyl-4-(2,4-dichloro-phenyl)-1H-imidazol-2-yl]-(2S)-2-(2-cyclopentyl-acetylamino)-ethyl]-phenoxymethyl)benzoic acid;
4-(4-{2-[1-Butyl-4-(2,4-dichloro-phenyl)-1H-imidazol-2-yl]-(2S)-2-[(trans-4-methyl-cyclohexanecarbonyl)-amino]-ethyl)phenoxymethyl)-benzoic acid;
4-(4-{2-[1-Butyl-4-(2,4-dichloro-phenyl)-1H-imidazol-2-yl]-(2S)-2-[(trans4-ethyl-cyclohexanecarbonyl)-amino]-ethyl}-phenoxymethyl)-benzoic acid;
4-(4-{(2S)-2-[(4-tert-Butyl-cyclohexanecarbonyl)-amino]-2-[4-(2,4-dichloro-phenyl)-(E)-1-pent-2-enyl-1H-imidazol-2-yl]-ethyl}-phenoxymethyl)-benzoic acid;
4-(4-{2-[4-(2,4-Dichloro-phenyl)-(E)-1-pent-2-enyl-1H-imidazol-2-yl]-(2S)-2-[(trans-4-ethyl-cyclohexanecarbonyl)-amino]-ethyl}-phenoxymethyl)-benzoic acid;
4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxymethyl)-benzoic acid;
4-(4-{(2S)-2-[(4-tert-Butyl-cyclohexanecarbonyl)-amino]-2-[1-butyl-4-(2,4-dichloro-phenyl)-1H-imidazol-2-yl]-ethyl)phenoxy)-benzoic acid;
4-(4-{2-[1-Butyl-4-(2,4-dichloro-phenyl)-1H-imidazol-2-yl]-(2S)-2-[(trans-4-ethyl-cyclohexane-carbonyl)-amino]-ethyl)phenoxy)-benzoic acid;
4-(4-{2-[4-(2,4-Dichloro-phenyl)-1-pent-2-enyl-1H-imidazol-2-yl]-(2S)-2-[(trans-4-ethyl-cyclohexane-carbonyl)-amino]-ethyl)phenoxy)-benzoic acid;
4-(4-{2-[1-But-2-ynyl-4-(2,4-dichloro-phenyl)-1H-imidazol-2-yl]-(2S)-2-[(trans-4-ethyl-cyclohexanecarbonyl)-amino]-ethyl}-phenoxy)-benzoic acid;
4-(4-{2-[1-Butyl-4-(2,4-dichloro-phenyl)-1H-imidazol-2-yl]-(2S)-2-[4-(3-fluorobenzylcarbamoyl)-butyrylamino]-ethyl)phenoxy)-benzoic acid;
4-(4-{2-[1-Butyl-4-(2,4-dichloro-phenyl)-1H-imidazol-2-yl]-(2S)-2-[2-(4-methoxy-phenyl)-acetylamino]-ethyl)phenoxy)-benzoic acid;
4-(4-{2-[1-Butyl-4-(2,4-dichloro-phenyl)-1H-imidazol-2-yl]-(2S)-2-[2-(2,4-difluorophenyl)-acetylamino]-ethyl)phenoxy)-benzoic acid;
4-{4-[2-[1-Butyl-4-(2,4-dichloro-phenyl)-1H-imidazol-2-yl]-(2S)-2-(4-methoxy-benzoylamino)-ethyl]-phenoxy}-benzoic acid;
4-{4-[2-[1-Butyl-4-(2,4-dichloro-phenyl)-1H-imidazol-2-yl]-(2S)-2-(3,5-difluoro-benzoylamino)-ethyl]-phenoxy}-benzoic acid;
4-(4-{2-[1-Butyl-4-(2,4-dichloro-phenyl)-1H-imidazol-2-yl]-(2S)-2-[3-(2,4-difluoro-phenyl)-ureido]-ethyl}-phenoxy)-benzoic acid;
Trans-4-Ethyl-cyclohexane-carboxylic acid((1S)-1-[1-butyl-4-(2,4-dichloro-phenyl)-1H-imidazol-2-yl]-2-{4-[4-(1H-tetrazol-5-yl)-phenoxy]-phenyl}-ethyl)-amide;
4-(4-{2-[1-Butyl-4-(2,4-dichloro-phenyl)-1H-imidazol-2-yl]-(2S)-2-[3-(4-methoxy-phenyl)-ureido]-ethyl}-phenoxy)-benzoic acid;
4-(4-{2-[1-Butyl-4-(2,4-dichloro-phenyl)-1H-imidazol-2-yl]-(2S)-2-[3-(3-methoxy-phenyl)-ureido]-ethyl}-phenoxy)-benzoic acid;
4-(4-{2-[1-Butyl-4-(2,4-dichloro-phenyl)-1H-imidazol-2-yl]-(2S)-2-[3-(4-trifluoromethyl-phenyl)-2-(2S)-isobutyrylamino-propionylamino]-ethyl}-phenoxy)-benzoic acid;
4-(4-{2-[1-Butyl-4-(2,4-dichloro-phenyl)-1H-imidazol-2-yl]-(2S)-2-[3-(4-tert-butyl-phenyl)-(2S)-2-isobutyrylamino-propionylamino]-ethyl}-phenoxy)-benzoic acid;
4-(4-{2-[1-Butyl-4-(2,4-dichloro-phenyl)-1H-imidazol-2-yl]-(2S)-2-[4-(4-chloro-phenyl)-(3S)-3-isobutyrylamino-butyrylamino]-ethyl)phenoxy)-benzoic acid; and
4-tert-Butyl-cyclohexanecarboxylic acid ((1S)-1-[1-butyl-4-(2,4-dichloro-phenyl)-1H-imidazol-2-yl]-2-{4-[4-(1H-tetrazol-5-yl)-benzyloxy]-phenyl}ethyl)-amide.

22. A pharmaceutically acceptable salt, solvate, or prodrug of a compound of Formula (I) according to claim 1.

23. The pharmaceutical composition of claim 22, wherein said compound is applied to the skin.

24. The pharmaceutical composition of claim 23, wherein said compoun is administered in a formulation ration of 0.1% to 99% of compound to topical excipient.

25. A pharmaceutical composition comprising a pharmaceutically acceptable carrier and a pharmacologically effective amount of the compound as claimed in claim 1 sufficient to inhibit protein tyrosine phosphatase.

26. The pharmaceutical composition of claim 25, in the form of an oral dosage or parenteral dosage unit.

27. The pharmaceutical composition of claim 25, wherein said compound is administered as a dose in a range from about 0.003 to 500 mg/kg of body weight per day.

28. The pharmaceutical composition of claim 25, wherein said compound is administered as a dose in a range from about 0.1 to 200 mg/kg of body weight per day.

29. The pharmaceutical composition of claim 25, wherein said compound is administered as a dose in a range from about 0.1 to 100 mg/kg of body weight per day.

30. The pharmaceutical composition of claim 25, further comprising one or more therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates.

31. A pharmaceutical composition comprising a pharmaceutically acceptable carrier and a pharmacologically effective amount of the compound as claimed in claim 1, sufficient to treat type I diabetes.

32. A pharmaceutical composition comprising a pharmaceutically acceptable carrier and a pharmacologically effective amount of the compound as claimed in claim 1, sufficient to treat type II diabetes.

33. A pharmaceutical composition comprising a pharmaceutically acceptable carrier and a pharmacologically effective amount of the compound as claimed in claim 1, sufficient to treat immune dysfunction.

34. A pharmaceutical composition comprising a pharmaceutically acceptable carrier and a pharmacologically effective amount of the compound as claimed in claim 1, sufficient to treat AIDS.

35. A pharmaceutical composition comprising a pharmaceutically acceptable carrier and a pharmacologically effective amount of the compound as claimed in claim 1, sufficient to treat autoimmune diseases.

36. A pharmaceutical composition comprising a pharmaceutically acceptable carrier and a pharmacologically effective amount of the compound as claimed in claim 1, sufficient to treat glucose intolerance.

37. A pharmaceutical composition comprising a pharmaceutically acceptable carrier and a pharmacologically effective amount of the compound as claimed in claim 1, sufficient to treat obesity.

38. A pharmaceutical composition comprising a pharmaceutically acceptable carrier and a pharmacologically effective amount of the compound as claimed in claim 1, sufficient to treat cancer.

39. A pharmaceutical composition comprising a pharmaceutically acceptable carrier and a pharmacologically effective amount of the compound as claimed in claim 1, sufficient to treat psoriasis.

40. A pharmaceutical composition comprising a pharmaceutically acceptable carrier and a pharmacologically effective amount of the compound as claimed in claim 1, sufficient to treat allergic diseases.

41. A pharmaceutical composition comprising a pharmaceutically acceptable carrier and a pharmacologically effective amount of the compound as claimed in claim 1, sufficient to treat infectious diseases.

42. A pharmaceutical composition comprising a pharmaceutically acceptable carrier and a pharmacologically effective amount of the compound as claimed in claim 1, sufficient to treat inflammatory diseases.

43. A pharmaceutical composition comprising a pharmaceutically acceptable carrier and a pharmacologically effective amount of the compound as claimed in claim 1, sufficient to treat diseases involving the modulated synthesis of growth hormone.

44. A pharmaceutical composition comprising a pharmaceutically acceptable carrier and a pharmacologically effective amount of the compound as claimed in claim 1, sufficient to treat diseases involving the modulated synthesis of growth factors or cytokines which affect the production of growth hormone.

45. A pharmaceutical composition comprising a pharmaceutically acceptable carrier and a pharmacologically effective amount of the compound as claimed in claim 1, sufficient to treat Alzheimer's disease.

46. A method of inhibition protein tyrosine phosphatases which comprises administering to a subject in need thereof a pharmacologically effective amount of a compound as claimed in claim 1.

47. A method of prevention and/or treatment of PTPase mediated human diseases, treatment comprising alleviation of one or more symptoms resulting from that disorder, to an outright cure for that particular disorder or prevention of the onset of the disorder, the method comprising administration to a human in need thereof a therapeutically effective amount of a compound of Formula (I) as claimed in claim 1.

48. The method of claim 46, further comprising administering to a subject in need thereof at least one adjuvant and/or additional therapeutic agent(s).

49. A method of treating PTPase mediated diseases, the method comprising administering to a subject in need thereof, a therapeutically effective amount of a compound of Formula (I) as claimed in claim 1, in combination with one or more therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates.

50. A method for treating acute and/or chronic inflammation, which comprises administering to a subject in need thereof a therapeutically effective amount of a compound of Formula (I) as defined in claim 1.

51. A method for treating type I or type II diabetes, which comprises administering to a subject in need thereof a therapeutically effective amount of a compound of Formula (I) as defined in claim 1.

52. A method for treating immune dysfunction, which comprises administering to a subject in need thereof a therapeutically effective amount of a compound of Formula (I) as defined in claim 1.

53. A method for treating AIDS, which comprises administering to a subject in need thereof a therapeutically effective amount of a compound of Formula (I) as defined in claim 1.

54. A method for treating autoimmune disease, which comprises administering to a subject in need thereof a therapeutically effective amount of a compound of Formula (I) as defined in claim 1.

55. A method for treating glucose intolerance, which comprises administering to a subject in need thereof a therapeutically effective amount of a compound of Formula (I) as defined in claim 1.

56. A method for treating cancer, which comprises administering to a subject in need thereof a therapeutically effective amount of a compound of Formula (I) as defined in claim 1.

57. A method for treating psoriasis, which comprises administering to a subject in need thereof a therapeutically effective amount of a compound of Formula (I) as defined in claim 1.

58. A method for treating allergic diseases, which comprises administering to a subject in need thereof a therapeutically effective amount of a compound of Formula (I) as defined in claim 1.

59. A method for treating infectious disease, which comprises administering to a subject in need thereof a therapeutically effective amount of a compound of Formula (I) as defined in claim 1.

60. A method for treating diseases involving the modulated synthesis of growth hormone, which comprises administering to a subject in need thereof a therapeutically effective amount of a compound of Formula (I) as defined in claim 1.

61. A method for treating modulated synthesis of growth factors or cytokines which affect the production of growth hormone, which comprises administering to a subject in need thereof a therapeutically effective amount of a compound of Formula (I) as defined in claim 1.

62. A method for treating Alzheimer's disease, which comprises administering to a subject in need thereof a therapeutically effective amount of a compound of Formula (I) as defined in claim 1.
